SEQUENCE LISTING (325 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 126 aa, Homo sapiens [sequence omitted]

SEQ ID NO: 3 - protein, 188 aa, Homo sapiens
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 4 - protein, 56 aa, Homo sapiens
MACLGFQRHKAQLNLATRTWPCTLLFFLLFIPVFCKAPPTEPECEKQFQPYFIPIN

SEQ ID NO: 5 - protein, 58 aa, Homo sapiens
MACLGFQRHKAQLNLATRTWPCTLLFFLLFIPVFCKAKEKKPSYNRGLCENAPNRARM

SEQ ID NO: 6 - protein, 79 aa, Homo sapiens
MACLGFQRHKAQLNLATRTWPCTLLFFLLFIPVFCKAMHVAQPAVVLASSRGIASFVSKEKKPSYNRGLCENAPNRARM

SEQ ID NO: 7 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 8 - DNA, 503 bp, Homo sapiens
ggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatccgaggggagctaccctttactactactactaccggtkggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacac

SEQ ID NO: 9 - protein, 166 aa, Homo sapiens
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYRDVWGQGTTVTVSSASTKGPSVFPLAPCSRSTSESTAALGCLVKDYFPEPVTVSWNSGALTSGVH

SEQ ID NO: 10 - DNA, 417 bp, Homo sapiens
ccatcctccctgtctgcatctgtaggagacagagtcaccatcacttgccgggcaagtcagagcattaacagctatttagattggtatcagcagaaaccagggaaagcccctaaactcctgatctatgctgcatccagtttgcaaagtggggtcccatcaaggttcagtggcagtggatctgggacagatttcactctcaccatcagcagtctgcaacctgaagattttgcaacttactactgtcaacagtattacagtactccattcactttcggccctgggaccaaagtggaaatcaaacgaactgtggctgcaccatctgtcttcatcttcccgccatctgatgagcagttgaaatctggaactgcctctgttgtgtgcctgctgaataacttctatcccagagaggccaaagta

SEQ ID NO: 11 - protein, 139 aa, Homo sapiens
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 12 - protein, 167 aa, Homo sapiens
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 13 - protein, 139 aa, Homo sapiens; identical to SEQ ID NO: 11

SEQ ID NO: 14 - protein, 10 aa, Homo sapiens
GFTFSSYGMH

SEQ ID NO: 15 - protein, 15 aa, Homo sapiens
VIWYDGSNKYYADSV

SEQ ID NO: 16 - protein, 16 aa, Homo sapiens
DPRGATLYYYYYGMDV

SEQ ID NO: 17 - protein, 11 aa, Homo sapiens
RASQSINSYLD

SEQ ID NO: 18 - protein, 7 aa, Homo sapiens
AASSLQS

SEQ ID NO: 19 - protein, 9 aa, Homo sapiens
QQYYSTPFT

SEQ ID NO: 20 - DNA, 1413 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatccgaggggagctaccctttactactactactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 21 - protein, 451 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - DNA, 714 bp, Homo sapiens
atggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattaacagctatttagattggtatcagcagaaaccagggaaagcccctaaactcctgatctatgctgcatccagtttgcaaagtggggtcccatcaaggttcagtggcagtggatctgggacagatttcactctcaccatcagcagtctgcaacctgaagattttgcaacttactactgtcaacagtattacagtactccattcactttcggccctgggaccaaagtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 23 - protein, 214 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 1392 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgtagcgtctggattcaccttcagtagccatggcatgcactgggtccgccaggctccaggcaaggggctggagtgggtggcagttatatggtatgatggaagaaataaatactatgcagactccgtgaagggccgattcaccatctccagagacaattccaagaacacgctgtttctgcaaatgaacagcctgagagccgaggacacggctgtgtattactgtgcgagaggaggtcacttcggtccttttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 25 - protein, 463 aa, Homo sapiens
MEFGLSWVFLVALLRGVQCQVQLVESGGGVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - DNA, 708 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtattagcagcagcttcttagcctggtaccagcagagacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 27 - protein, 235 aa, Homo sapiens
METPAQLLFLLLLWLPDTTGEIVLTQSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 167 aa, Homo sapiens
GVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQ

SEQ ID NO: 29 - protein, 141 aa, Homo sapiens
QSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIKRTVAAPSVFIFPPSDEQLKSGTASVVCLLNNFYPREAK

SEQ ID NO: 30 - protein, 10 aa, Homo sapiens
GFTFSSHGMH

SEQ ID NO: 31 - protein, 15 aa, Homo sapiens
VIWYDGRNKYYADSV

SEQ ID NO: 32 - protein, 9 aa, Homo sapiens
GGHFGPFDY

SEQ ID NO: 33 - protein, 12 aa, Homo sapiens
RASQSISSSFLA

SEQ ID NO: 34 - protein, 7 aa, Homo sapiens
GASSRAT

SEQ ID NO: 35 - protein, 10 aa, Homo sapiens
CQQYGTSPWT

SEQ ID NO: 36 - DNA, 1392 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtgtagcgtctggattcaccttcagtagccatggcatgcactgggtccgccaggctccaggcaaggggctggagtgggtggcagttatatggtatgatggaagaaataaatactatgcagactccgtgaagggccgattcaccatctccagagacaattccaagaacacgctgtttctgcaaatgaacagcctgagagccgaggacacggctgtgtattactgtgcgagaggaggtcacttcggtccttttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttccaaagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - protein, 463 aa, Homo sapiens
MEFGLSWVFLVALLRGVQCQVQLVESGGGVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 38 - DNA, 459 bp, Homo sapiens
cctgggaggtccctgagactctcctgtgcagcgtctggattcaccttcagtagtcatggcatccactgggtccgccaggctccaggcaaggggctggagtgggtggcagttatatggtatgatggaagaaataaagactatgcagactccgtgaagggccgattcaccatctccagagacaattccaagaacacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagc

SEQ ID NO: 39 - protein, 153 aa, Homo sapiens
PGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTS

SEQ ID NO: 40 - DNA, 440 bp, Homo sapiens
cagtctccaggcaccctgtctttgtctccaggggaaagagccaccctctcctgcagggccagtcagagtgtcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaggattttgcagtgtattactgtcaacagtatggtaggtcaccattcactttcggccctgggaccaaagtagatatcaagcgaactgtggctgcaccatctgtcttcatcttcccgccatctgatgagcagttgaaatctggaactgcctctgttgtgtgcctgctgaataacttctatcccagagaggccaaagtacagtggaaaggtggata

SEQ ID NO: 41 - protein, 146 aa, Homo sapiens
QSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQWKGG

SEQ ID NO: 42 - protein, 153 aa, Homo sapiens; identical to SEQ ID NO: 39

SEQ ID NO: 43 - protein, 146 aa, Homo sapiens; identical to SEQ ID NO: 41

SEQ ID NO: 44 - protein, 10 aa, Homo sapiens
GFTFSSHGIH

SEQ ID NO: 45 - protein, 15 aa, Homo sapiens
VIWYDGRNKDYADSV

SEQ ID NO: 46 - protein, 9 aa, Homo sapiens
VAPLGPLDY

SEQ ID NO: 47 - protein, 11 aa, Homo sapiens
RASQSVSSYLA

SEQ ID NO: 48 - protein, 7 aa, Homo sapiens
GASSRAT

SEQ ID NO: 49 - protein, 10 aa, Homo sapiens
CQQYGRSPFT

SEQ ID NO: 50 - DNA, 1395 bp, Homo sapiens
atggagtttgggctgagctgggttttcctcgttgctcttttaagaggtgtccagtgtcaggtgcagctggtggagtctgggggaggcgtggtccagcctgggaggtccctgagactctcctgtacagcgtctggattcaccttcagtaactatggcatgcactgggtccgccaggctccaggcaaggggctggagtgggtggcagttatatggtatgatggaagtaataaacactatggagactccgtgaagggccgattcaccatctccagtgacaattccaagaacacgctgtatctgcaaatgaacagcctgagagccgaggacacggctgtgtattactgtgcgagaggagagagactggggtcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 51 - protein, 464 aa, Homo sapiens
MEFGLSWVFLVALLRGVQCQVQLVESGGGVVQPGRSLRLSCTASGFTFSNYGMHWVRQAPGKGLEWVAVIWYDGSNKHYGDSVKGRFTISSDNSKNTLYLQMNSLRAEDTAVYYCARGERLGSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 52 - DNA, 702 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgcaggaccagtgttagcagcagttacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtctattactgtcagcagtatggcatctcacccttcactttcggcggagggaccaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 53 - protein, 233 aa, Homo sapiens
METPAQLLFLLLLWLPDTTGEIVLTQSPGTLSLSPGERATLSCRTSVSSSYLAWYQQKPGQAPRLLIYGASSRATGIPDRFSGSGSGTDFTLTISRLEPEDFAVYYCQQYGISPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 166 aa, Homo sapiens
GVVQPGRSLRLSCTASGFTFSNYGMHWVRQAPGKGLEWVAVIWYDGSNKHYGDSVKGRFTISSDNSKNTLYLQMNSLRAEDTAVYYCARGERLGSYFDYWGQGTLVTVSSASTKGPSVFPLAPCSRSTSESTAALGCLVKDYFPEPVTVSWNSGALTSGVHTFPAV

SEQ ID NO: 55 - protein, 141 aa, Homo sapiens
QSPGTLSLSPGERATLSCRTSVSSSYLAWYQQKPGQAPRLLIYGASSRATGIPDRFSGSGSGTDFTLTISRLEPEDFAVYYCQQYGISPFTFGGGTKVEIKRTVAAPSVFIFPPSDEQLKSGTASVVCLLNNFYPREAKVQ

SEQ ID NO: 56 - protein, 10 aa, Homo sapiens
GFTFSNYGMH

SEQ ID NO: 57 - protein, 15 aa, Homo sapiens
VIWYDGSNKHYGDSV

SEQ ID NO: 58 - protein, 10 aa, Homo sapiens
GERLGSYFDY

SEQ ID NO: 59 - protein, 10 aa, Homo sapiens
RTSVSSSYLA

SEQ ID NO: 60 - protein, 7 aa, Homo sapiens
GASSRAT

SEQ ID NO: 61 - protein, 10 aa, Homo sapiens
CQQYGISPFT

SEQ ID NO: 62 - DNA, 489 bp, Homo sapiens
cctgggaggtccctgagactctcctgtgcagcgtctggattcaccttcagtagtcatggcatccactgggtccgccaggctccaggcaaggggctggagtgggtggcagttatatggtatgatggaagaaataaagactatgcagactccgtgaagggccgattcaccatctccagagacaattccaagaagacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacag

SEQ ID NO: 63 - protein, 163 aa, Homo sapiens
PGRSLRLSCAASGFTFSSHGIHWVRQAPGKGLEWVAVIWYDGRNKDYADSVKGRFTISRDNSKKTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQ

SEQ ID NO: 64 - DNA, 417 bp, Homo sapiens
ggcaccctgtctttgtctccaggggaaagagccaccctctcctgcagggccagtcagagtgtcagcagctacttagcctggtaccagcagaaacctggccaggctcccagactcctcatctatggtgcatccagcagggccactggcatcccagacaggttcagtggcagtgggtctgggacagacttcactctcaccatcagcagactggagcctgaggattttgcagtgtattactgtcagcagtatggtaggtcaccattcactttcggccctgggaccaaagtggatatcaagcgaactgtggctgcaccatctgtcttcatcttcccgccatctgatgagcagttgaaatctggaactgcctctgttgtgtgcctgctgaataacttctatcccagagaggccaaagtacag

SEQ ID NO: 65 - protein, 139 aa, Homo sapiens
GTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQ

SEQ ID NO: 66 - protein, 163 aa, Homo sapiens; identical to SEQ ID NO: 63

SEQ ID NO: 67 - protein, 139 aa, Homo sapiens; identical to SEQ ID NO: 65

SEQ ID NO: 68 - protein, 10 aa, Homo sapiens
GFTFSSHGIH

SEQ ID NO: 69 - protein, 15 aa, Homo sapiens
VIWYDGRNKDYADSV

SEQ ID NO: 70 - protein, 9 aa, Homo sapiens
VAPLGPLDY

SEQ ID NO: 71 - protein, 11 aa, Homo sapiens
RASQSVSSYLA

SEQ ID NO: 72 - protein, 7 aa, Homo sapiens
GASSRAT

SEQ ID NO: 73 - protein, 10 aa, Homo sapiens
CQQYGRSPFT

SEQ ID NO: 74 - DNA, 1392 bp, Homo sapiens
atggagtttgggctgagctgggttttcctcgttgctcttttaagaggtgtccagtgtcaggtgcagctggtggagtctgggggaggcgtggtcgagcctgggaggtccctgagactctcctgtacagcgtctggattcaccttcagtagttatggcatgcactgggtccgccaggctccaggcaaggggctggagtgggtggcagttatatggtatgatggaagcaataaacactatgcagactccgcgaagggccgattcaccatctccagagacaattccaagaacacgctgtatctgcaaatgaacagcctgagagccgaggacacggctgtgtattactgtgcgagagccggactgctgggttactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 75 - protein, 463 aa, Homo sapiens
MEFGLSWVFLVALLRGVQCQVQLVESGGGVVEPGRSLRLSCTASGFTFSSYGMHWVRQAPGKGLEWVAVIWYDGSNKHYADSAKGRFTISRDNSKNTLYLQMNSLRAEDTAVYYCARAGLLGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 76 - DNA, 705 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgtagggccagtcaaagtgttagcagctacttagcctggtaccaacagaaacctggccaggctcccaggcccctcatctatggtgtatccagcagggccactggcatcccagacaggttcagtggcagtgggtctgggacagacttcactctcaccatcagcagactggagcctgaagattttgcagtgtattactgtcagcagtatggtatctcaccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 77 - protein, 234 aa, Homo sapiens
METPAQLLFLLLLWLPDTTGEIVLTQSPGTLSLSPGERATLSCRASQSVSSYLAWYQQKPGQAPRPLIYGVSSRATGIPDRFSGSGSGTDFTLTISRLEPEDFAVYYCQQYGISPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 138 aa, Homo sapiens
GGVVEPGRSLRLSCTASGFTFSSYGMHWVRQAPGKGLEWVAVIWYDGSNKHYADSAKGRFTISRDNSKNTLYLQMNSLRAEDTAVYYCARAGLLGYFDYWGQGTLVTVSSASTKGPSVFPLAPCSRSTSESTAALGCL

SEQ ID NO: 79 - protein, 142 aa, Homo sapiens
QSPGTLSLSPGERATLSCRASQSVSSYLAWYQQKPGQAPRPLIYGVSSRATGIPDRFSGSGSGTDFTLTISRLEPEDFAVYYCQQYGISPFTFGPGTKVDIKRTVAAPSVFIFPPSDEQLKSGTASVVCLLNNFYPREAKVQ

SEQ ID NO: 80 - protein, 10 aa, Homo sapiens
GFTFSSYGMH

SEQ ID NO: 81 - protein, 15 aa, Homo sapiens
VIWYDGSNKHYADSA

SEQ ID NO: 82 - protein, 9 aa, Homo sapiens
AGLLGYFDY

SEQ ID NO: 83 - protein, 11 aa, Homo sapiens
RASQSVSSYLA

SEQ ID NO: 84 - protein, 7 aa, Homo sapiens
GASSRAT

SEQ ID NO: 85 - protein, 10 aa, Homo sapiens
CQQYGISPFT

SEQ ID NO: 86 - DNA, 507 bp, Homo sapiens
ggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagaggggcccgtataataaccccttgtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacag

SEQ ID NO: 87 - protein, 169 aa, Homo sapiens
GVVQPGRSLRLSCAASGFTFSSYGMHWVRQAPGKGLEWVAVIWYDGSNKYYADSVKGRFTISRDNSKNTLYLQMNSLRAEDTAVYYCARGARIITPCMDVWGQGTTVTVSSASTKGPSVFPLAPCSRSTSESTAALGCLVKDYFPEPVTVSWNSGALTSGVHTFPAVLQ

SEQ ID NO: 88 - DNA, 458 bp, Homo sapiens
cagtctccatcctccctgtctgcatctgtaggagacagagtcaccatcacttgccgggcaagtcagagcattaacacctatttaatttggtatcagcagaaaccagggaaagcccctaacttcctgatctctgctacatccattttgcaaagtggggtcccatcaaggttccgtggcagtggctctgggacaaatttcactctcaccatcaacagtcttcatcctgaagattttgcaacttactactgtcaacagagttacagtaccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaa

SEQ ID NO: 89 - protein, 152 aa, Homo sapiens
QSPSSLSASVGDRVTITCRASQSINTYLIWYQQKPGKAPNFLISATSILQSGVPSRFRGSGSGTNFTLTINSLHPEDFATYYCQQSYSTPFTFGPGTKVDIKRTVAAPSVFIFPPSDEQLKSGTASVVCLLNNFYPREAKVQWKVDNALQSG

SEQ ID NO: 90 - protein, 169 aa, Homo sapiens; identical to SEQ ID NO: 87

SEQ ID NO: 91 - protein, 152 aa, Homo sapiens; identical to SEQ ID NO: 89

SEQ ID NO: 92 - protein, 10 aa, Homo sapiens
GFTFSSYGMH

SEQ ID NO: 93 - protein, 15 aa, Homo sapiens
VIWYDGSNKYYADSV

SEQ ID NO: 94 - protein, 11 aa, Homo sapiens
GARIITPCMDV

SEQ ID NO: 95 - protein, 11 aa, Homo sapiens
RASQSINTYLI

SEQ ID NO: 96 - protein, 7 aa, Homo sapiens
ATSILQS

SEQ ID NO: 97 - protein, 9 aa, Homo sapiens
QQSYSTPFT

SEQ ID NO: 98 - DNA, 501 bp, Homo sapiens
ggcgtggtccagcctgggaggtccctgagactctcctgtgtagcgtctggattcatcttcagtagtcatggcatccactgggtccgccaggctccaggcaaggggctggagtgggtggcagttatatggtatgatggaagaaataaagactatgcagactccgtgaagggccgattcaccatctccagagacaattccaagaacacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacag

SEQ ID NO: 99 - protein, 167 aa, Homo sapiens
GVVQPGRSLRLSCVASGFIFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQ

SEQ ID NO: 100 - DNA, 426 bp, Homo sapiens
tctccaggcaccctgtctttgtctccaggggaaagagccaccctctcctgcagggccagtcagagtattagcagcaatttcttagcctggtaccagcagaaacctggccaggctcccaggctcctcatctatcgtccatccagcagggccactggcatcccagacagtttcagtggcagtgggtctgggacagacttcactctcaccatcagcagactggagcctgaggattttgcattatattactgtcagcagtatggtacgtcaccattcactttcggccctgggaccaaagtggatatcaagcgaactgtggctgcaccatctgtcttcatcttcccgccatctgatgagcagttgaaatctggaactgcctctgttgtgtgcctgctgaataacttctatcccagagaggccaaagtacag

SEQ ID NO: 101 - protein, 142 aa, Homo sapiens
SPGTLSLSPGERATLSCRASQSISSNFLAWYQQKPGQAPRLLIYRPSSRATGIPDSFSGSGSGTDFTLTISRLEPEDFALYYCQQYGTSPFTFGPGTKVDIKRTVAAPSVFIFPPSDEQLKSGTASVVCLLNNFYPREAKVQ

SEQ ID NO: 102 - protein, 167 aa, Homo sapiens; identical to SEQ ID NO: 99

SEQ ID NO: 103 - protein, 142 aa, Homo sapiens; identical to SEQ ID NO: 101

SEQ ID NO: 104 - protein, 10 aa, Homo sapiens
GFIFSSHGIH

SEQ ID NO: 105 - protein, 15 aa, Homo sapiens
VIWYDGRNKDYADSV

SEQ ID NO: 106 - protein, 9 aa, Homo sapiens
VAPLGPLDY

SEQ ID NO: 107 - protein, 12 aa, Homo sapiens
RASQSISSNFLA

SEQ ID NO: 108 - protein, 7 aa, Homo sapiens
RPSSRAT

SEQ ID NO: 109 - protein, 10 aa, Homo sapiens
CQQYGTSPFT

SEQ ID NO: 110 - DNA, 516 bp, Homo sapiens
tcgggcccaggactggtgaagccttcacagatcctgtccctcacctgcactgtctctggtggctccatcagcagtggtggtcactactggagctggatccgccagcacccagggaagggcctggagtggattgggtacatctattacattgggaacacctactacaacccgtccctcaagagtcgagttaccatatcagtagacacgtctaagaaccagttctccctgaagctgagctctgtgactgccgcggacacggccgtgtattattgtgcgagagatagtggggactactacggtatagacgtctggggccaagggaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacaa

SEQ ID NO: 111 - protein, 172 aa, Homo sapiens
SGPGLVKPSQILSLTCTVSGGSISSGGHYWSWIRQHPGKGLEWIGYIYYIGNTYYNPSLKSRVTISVDTSKNQFSLKLSSVTAADTAVYYCARDSGDYYGIDVWGQGTTVTVSSASTKGPSVFPLAPCSRSTSESTAALGCLVKDYFPEPVTVSWNSGALTSGVHTFPAVLQ

SEQ ID NO: 112 - DNA, 465 bp, Homo sapiens
tctccagactttcagtctgtgactccaaaggagaaagtcaccatcacctgccgggccagtcagagcattggtagtagcttacattggtatcagcagaaaccagatcagtctccaaagctcctcatcaagtatgcttcccagtccttctctggggtcccctcgaggttcagtggcagtggatctgggacagatttcaccctcaccatcaatagcctggaagctgaagatgctgcaacgtattactgtcatcagagtagtagtttaccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggag

SEQ ID NO: 113 - protein, 155 aa, Homo sapiens
SPDFQSVTPKEKVTITCRASQSIGSSLHWYQQKPDQSPKLLIKYASQSFSGVPSRFSGSGSGTDFTLTINSLEAEDAATYYCHQSSSLPLTFGGGTKVEIKRTVAAPSVFIFPPSDEQLKSGTASVVCLLNNFYPREAKVQWKVDNALQSGNSQE

SEQ ID NO: 114 - protein, 172 aa, Homo sapiens; identical to SEQ ID NO: 111

SEQ ID NO: 115 - protein, 155 aa, Homo sapiens; identical to SEQ ID NO: 113

SEQ ID NO: 116 - protein, 8 aa, Homo sapiens
SSGGHYWS

SEQ ID NO: 117 - protein, 17 aa, Homo sapiens
GYIYYIGNTYYNPSLKS

SEQ ID NO: 118 - protein, 10 aa, Homo sapiens
DSGDYYGIDV

SEQ ID NO: 119 - protein, 12 aa, Homo sapiens
CRASQSIGSSLH

SEQ ID NO: 120 - protein, 7 aa, Homo sapiens
YASQSFS

SEQ ID NO: 121 - protein, 9 aa, Homo sapiens
HQSSSLPLT

SEQ ID NO: 122 - DNA, 451 bp, Homo sapiens
ggcgtggtccagcctgggaggtccctgagactctcctgtgcagcgtctggattcaccttcagtagctatggcatgcactgggtccgccaggctccaggcaaggggctggagtgggtggcagttatatggtatgatggaagtcataaatactatgcagactccgtgaagggccgattcaccatctccagagacaattccaagaacacgctgtatctgcaaatgaacagcctgagagccgaggacacggctgtgtattactgtgcgagaggcgctgtagtagtaccagctgctatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgt

SEQ ID NO: 123 - protein, 151 aa, Homo sapiens
GVVQPGRSLRLSCAASGFTFSSYGMHWVRQAPGKGLEWVAVIWYDGSHKYYADSVKGRFTISRDNSKNTLYLQMNSLRAEDTAVYYCARGAVVVPAAMDVWGQGTTVTVSSASTKGPSVFPLAPCSRSTSESTAALGCLVKDYFPEPVTVS

SEQ ID NO: 124 - DNA, 402 bp, Homo sapiens
  misc_feature (207)..(207): n is a, c, g, or t
acccagtctccatcctccctgtctgcatctgtaggagacagagtcaccatcacttgccgggcaagtcagaacattagcaggtatttaaattggtatcaacagaaaccagggaaagcccctaagttcctgatctatgttgcatctattttgcaaagtggggtcccatcagggttcagtgccagtggatctgggccagatttcactctnaccatcagcagtctgcaacctgaagattttgcaacttactactgtcaacagagttacagtaccccattcactttcggccctgggaccaaagtggatatcaaacgaactgtggctgcaccatctgtcttcatcttcccgccatctgatgagcagttgaaatctggaactgcctctgttgtgtgcctgctgaataac

SEQ ID NO: 125 - protein, 134 aa, Homo sapiens
TQSPSSLSASVGDRVTITCRASQNISRYLNWYQQKPGKAPKFLIYVASILQSGVPSGFSASGSGPDFTLTISSLQPEDFATYYCQQSYSTPFTFGPGTKVDIKRTVAAPSVFIFPPSDEQLKSGTASVVCLLNN

SEQ ID NO: 126 - protein, 150 aa, Homo sapiens
GVVQPGRSLRLSCAASGFTFSSYGMHWVRQAPGKGLEWVAVIWYDGSHKYYADSVKGRFTISRDNSKNTLYLQMNSLRAEDTAVYYCARGAVVVPAAMDVWGQGTTVTVSSASTKGPSVFPLAPCSRSTSESTAALGCLVKDYFPEPVTV

SEQ ID NO: 127 - protein, 134 aa, Homo sapiens; identical to SEQ ID NO: 125

SEQ ID NO: 128 - protein, 10 aa, Homo sapiens
GFTFSSYGMH

SEQ ID NO: 129 - protein, 15 aa, Homo sapiens
VIWYDGSHKYYADSV

SEQ ID NO: 130 - protein, 11 aa, Homo sapiens
GAVVVPAAMDV

SEQ ID NO: 131 - protein, 11 aa, Homo sapiens
RASQNISRYLN

SEQ ID NO: 132 - protein, 7 aa, Homo sapiens
VASILQS

SEQ ID NO: 133 - protein, 9 aa, Homo sapiens
QQSYSTPFT

SEQ ID NO: 134 - DNA, 438 bp, Homo sapiens
  misc_feature (64)..(64): n is a, c, g, or t
gtggtccagcctgggaggtccctgagactctcctgtgcagcgtctggattcaccttcagtagcngtggcatgcactgggtccgccaggctccaggcaaggggctggagtgggtggcagttatatggtctgatggaagtcataaatactatgcagactccgtgaagggccgattcaccatctccagagacaattccaagaacacgctgtatctgcaaatgaacagcctgagagccgaggacacggctgtgtattactgtgcgagaggaactatgatagtagtgggtacccttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccg

SEQ ID NO: 135 - protein, 145 aa, Homo sapiens
VVQPGRSLRLSCAASGFTFSSGMHWVRQAPGKGLEWVAVIWSDGSHKYYADSVKGRFTISRDNSKNTLYLQMNSLRAEDTAVYYCARGTMIVVGTLDYWGQGTLVTVSSASTKGPSVFPLAPCSRSTSESTAALGCLVKDYFPEP

SEQ ID NO: 136 - DNA, 451 bp, Homo sapiens
acccagtctccatcctccctgtctgcatctgtaggagacagagtcaccatcacttgccgggcaagtcagagcatttgcaactatttaaattggtatcagcagaaaccaggaaaagcccctagggtcctgatctatgctgcatccagtttgcaaggtggggtcccgtcaaggttcagtggcagtggatctgggacagattgcactctcaccatcagcagtctgcaacctgaagattttgcaacttactactgtcaacagagttacactaccccattcactttcggccctgggaccagagtggatatcgaacgaactgtggctgcaccatctgtcttcatcttcccgccatctgatgagcagttgaaatctggaactgcctctgttgtgtgcctgctgaataacttctatcccagagaggccaaagtacagtggaaggtggataacgcctatt

SEQ ID NO: 137 - protein, 150 aa, Homo sapiens
TQSPSSLSASVGDRVTITCRASQSICNYLNWYQQKPGKAPRVLIYAASSLQGGVPSRFSGSGSGIDCTLTISSLQPEDFATYYCQQSYITPFTFGPGTRVDIERTVAAPSVFIFPPSDEQLKSGTASVVCLLNNFYPREAKVQWKVDNAY

SEQ ID NO: 138 - protein, 146 aa, Homo sapiens
VVQPGRSLRLSCAASGFTFSSCGMHWVRQAPGKGLEWVAVIWSDGSHKYYADSVKGRFTISRDNSKNTLYLQMNSLRAEDTAVYYCARGTMIVVGTLDYWGQGTLVTVSSASTKGPSVFPLAPCSRSTSESTAALGCLVKDYFPEP

SEQ ID NO: 139 - protein, 150 aa, Homo sapiens; identical to SEQ ID NO: 137

SEQ ID NO: 140 - protein, 10 aa, Homo sapiens
GFTFSSCGMH

SEQ ID NO: 141 - protein, 15 aa, Homo sapiens
VIWSDGSHKYYADSV

SEQ ID NO: 142 - protein, 11 aa, Homo sapiens
GTMIVVGTLDY

SEQ ID NO: 143 - protein, 11 aa, Homo sapiens
RASQSICNYLN

SEQ ID NO: 144 - protein, 7 aa, Homo sapiens
AASSLQG

SEQ ID NO: 145 - protein, 9 aa, Homo sapiens
QQSYITPFT

SEQ ID NO: 146 - DNA, 562 bp, Homo sapiens
tcctgtgcagcgtctggattcaccttcagttactatggcgtctgggggaggcgtggtccagcctgggaggtccctgagactctcctgtgcagcgtctggattcaccttcagtagctatggcgtgcactgggtccgccaggctccaggcaaggggctggagtgggtggcagttatatggtatgatggaagtaataaatactatgcagactccgtgaagggccgattcaccatctccagagacaattccaagagcacgctgtatctgcaaatgaacagcctgagagccgaggacacggctgtgtattattgtgcgagagactcgtattacgatttttggagtggtcggggcggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtc

SEQ ID NO: 147 - protein, 174 aa, Homo sapiens
SGGGVVQPGRSLRLSCAASGFTFSSYGVHWVRQAPGKGLEWVAVIWYDGSNKYYADSVKGRFTISRDNSKSTLYLQMNSLRAEDTAVYYCARDSYYDFWSGRGGMDVWGQGTTVTVSSASTKGPSVFPLAPCSRSTSESTAALGCLVKDYFPEPVTVSWNSGALTSGVHTFPAV

SEQ ID NO: 148 - DNA, 419 bp, Homo sapiens
ccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaactgggactctggggtcccagacagattcagcggcagtgggtcaggcactgatttcacactgaaaatcagcagggtggaggctgaggatgttggggtttattactgcatgcaaggttcacactggcctccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccac

SEQ ID NO: 149 - protein, 139 aa, Homo sapiens
PLSLPVTLGQPASISCRSSQSLVYSDGNTYLNWFQQRPGQSPRRLIYKVSNWDSGVPDRFSGSGSGTDFTLKISRVEAEDVGVYYCMQGSHWPPTFGQGTKVEIKRTVAAPSVFIFPPSDEQLKSGTASVVCLLNNFYP

SEQ ID NO: 150 - protein, 171 aa, Homo sapiens
GVVQPGRSLRLSCAASGFTFSSYGVHWVRQAPGKGLEWVAVIWYDGSNKYYADSVKGRFTISRDNSKSTLYLQMNSLRAEDTAVYYCARDSYYDFWSGRGGMDVWGQGTTVTVSSASTKGPSVFPLAPCSRSTSESTAALGCLVKDYFPEPVTVSWNSGALTSGVHTFPAV

SEQ ID NO: 151 - protein, 139 aa, Homo sapiens; identical to SEQ ID NO: 149

SEQ ID NO: 152 - protein, 10 aa, Homo sapiens
GFTFSSYGVH

SEQ ID NO: 153 - protein, 15 aa, Homo sapiens
VIWYDGSNKYYADSV

SEQ ID NO: 154 - protein, 15 aa, Homo sapiens
DSYYDFWSGRGGMDV

SEQ ID NO: 155 - protein, 16 aa, Homo sapiens
RSSQSLVYSDGNTYLN

SEQ ID NO: 156 - protein, 7 aa, Homo sapiens
KVSNWDS

SEQ ID NO: 157 - protein, 9 aa, Homo sapiens
MQGSHWPPT

SEQ ID NO: 158 - DNA, 490 bp, Homo sapiens
gtccagcctgggaggtccctgagactctcctgtgcagcgtctggattcaccttcagtaactatgccatgcactgggtccgccaggctccaggcaaggggctggagtgggtggtagttatttggcatgatggaaataataaatactatgcagagtccgtgaagggccgattcaccatctccagagacaattccaagaacacgctgtatctgcaaatgaacagcctgagagccgaggacacggctgtatattactgtgcgagagatcagggcactggctggtacggaggctttgacttctggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcc

SEQ ID NO: 159 - protein, 163 aa, Homo sapiens
VQPGRSLRLSCAASGFTFSNYAMHWVRQAPGKGLEWVVVIWHDGNNKYYAESVKGRFTISRDNSKNTLYLQMNSLRAEDTAVYYCARDQGTGWYGGFDFWGQGTLVTVSSASTKGPSVFPLAPCSRSTSESTAALGCLVKDYFPEPVTVSWNSGALTSGVHTF

SEQ ID NO: 160 - DNA, 419 bp, Homo sapiens
cctggagagccggcttccatctcttgcaggtctagtcagagcctcctgcatagtaatggatacaactatttggattggtacctgcagaagccaggacagtctccacagctcctgatctatttgggttctaatcgggcctccggggtccctgacaggttcagtggcagtggatcaggcacagattttacactgaaactcagcagagtggaggctgaggatgttggggtttattactgcatgcaagctctacaaactcctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagaraggccaaagtacattccat

SEQ ID NO: 161 - protein, 133 aa, Homo sapiens
PGEPASISCRSSQSLLHSNGYNYLDWYLQKPGQSPQLLIYLGSNRASGVPDRFSGSGSGTDFTLKLSRVEAEDVGVYYCMQALQTPLTFGGGTKVEIKRTVAAPSVFIFPPSDEQLKSGTASVVCLLNNFYPR

SEQ ID NO: 162 - protein, 163 aa, Homo sapiens; identical to SEQ ID NO: 159

SEQ ID NO: 163 - protein, 133 aa, Homo sapiens; identical to SEQ ID NO: 161

SEQ ID NO: 164 - protein, 10 aa, Homo sapiens
GFTFSNYAMH

SEQ ID NO: 165 - protein, 15 aa, Homo sapiens
VIWHDGNNKYYAESV

SEQ ID NO: 166 - protein, 12 aa, Homo sapiens
DQGTGWYGGFDF

SEQ ID NO: 167 - protein, 16 aa, Homo sapiens
RSSQSLLHSNGYNYLD

SEQ ID NO: 168 - protein, 7 aa, Homo sapiens
LGSNRAS

SEQ ID NO: 169 - protein, 9 aa, Homo sapiens
MQALQTPLT

SEQ ID NO: 170 - DNA, 1612 bp, Mus musculus
tcaagcttttggaccctcgtacagaagctaatacgactcactatagggaaataagagagaaaagaagagtaagaagaaatataagagccaccatggaaacggacactctcctgctgtgggtactgttgctttgggtgccaggatctacgggcgaggcgaagctccaggaaagcgggccggtactggtcaaacccggcgctagcgtgaaaatgagttgcaaggccagcggttatactttcaccgactattacatgaattgggtaaagcagagccacgggaagtcattggaatggattggagtaatcaacccctacaacggggacaccagttataaccaaaagttcaagggaaaggcgacactcactgtggataaaagttcaagcacagcctacatggaacttaattccttgacatctgaggacagcgcagtatactattgtgctagatattacggtagttggttcgcctactgggggcaaggaacgctaatcactgtatcaaccgcaaaaactacagcaccctctgtataccctctcgcacctgtgtgcggagacacaactggcagctcagtaaccctcgggtgcctggttaaggggtatttcccagagccagtgactctcacctggaattctggaagccttagttcaggagtacatactttccccgctgtgctacaatcagatctgtacaccctgagttcaagcgttaccgttacgagtagtacttggccttctcagtccataacctgtaacgtcgcccatcccgcctcttctaccaaggttgacaagaagattgaaccgagaggacccaccattaaaccctgccctccttgcaagtgtcctgctccaaacctcttgggaggcccgtccgtcttcatctttcccccaaagatcaaagacgtgctcatgatttcgctgtcaccaatcgtaacgtgcgttgtggtggacgtctccgaggacgacccagatgtccagatatcctggttcgtaaacaatgtggaggtccatacagcccagacgcagacccaccgcgaggactataattctaccctcagggttgtctctgccctgccaattcagcatcaggactggatgagcgggaaagagttcaagtgtaaagtgaataataaagatctgcctgcccccatcgagagaactatcagtaaacctaaaggatccgtgcgcgctccccaggtctacgtcctgccccccccagaagaggaaatgactaagaagcaggtcacacttacctgcatggtgacggattttatgccagaagacatttatgtggaatggacaaataatggaaagacagagctcaattataagaacacagagccagtgttagattcagatggcagttattttatgtacagcaaactgcgcgtagaaaaaaaaaattgggtcgagcgcaatagctactcctgcagcgtcgtgcatgaagggctacacaatcaccacaccacgaagagcttttcccggacacctgggaaatgataataggctggagcctcggtggccatgcttcttgccccttgggcctccccccagcccctcctccccttcctgcacccgtacccccgtggtctttgaataaagtctgagtgggcggc

SEQ ID NO: 171 - protein, 467 aa, Mus musculus
METDTLLLWVLLLWVPGSTGEAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 172 - DNA, 1401 bp, Mus musculus
atggaaacggacactctcctgctgtgggtactgttgctttgggtgccaggatctacgggcgaggcgaagctccaggaaagcgggccggtactggtcaaacccggcgctagcgtgaaaatgagttgcaaggccagcggttatactttcaccgactattacatgaattgggtaaagcagagccacgggaagtcattggaatggattggagtaatcaacccctacaacggggacaccagttataaccaaaagttcaagggaaaggcgacactcactgtggataaaagttcaagcacagcctacatggaacttaattccttgacatctgaggacagcgcagtatactattgtgctagatattacggtagttggttcgcctactgggggcaaggaacgctaatcactgtatcaaccgcaaaaactacagcaccctctgtataccctctcgcacctgtgtgcggagacacaactggcagctcagtaaccctcgggtgcctggttaaggggtatttcccagagccagtgactctcacctggaattctggaagccttagttcaggagtacatactttccccgctgtgctacaatcagatctgtacaccctgagttcaagcgttaccgttacgagtagtacttggccttctcagtccataacctgtaacgtcgcccatcccgcctcttctaccaaggttgacaagaagattgaaccgagaggacccaccattaaaccctgccctccttgcaagtgtcctgctccaaacctcttgggaggcccgtccgtcttcatctttcccccaaagatcaaagacgtgctcatgatttcgctgtcaccaatcgtaacgtgcgttgtggtggacgtctccgaggacgacccagatgtccagatatcctggttcgtaaacaatgtggaggtccatacagcccagacgcagacccaccgcgaggactataattctaccctcagggttgtctctgccctgccaattcagcatcaggactggatgagcgggaaagagttcaagtgtaaagtgaataataaagatctgcctgcccccatcgagagaactatcagtaaacctaaaggatccgtgcgcgctccccaggtctacgtcctgccccccccagaagaggaaatgactaagaagcaggtcacacttacctgcatggtgacggattttatgccagaagacatttatgtggaatggacaaataatggaaagacagagctcaattataagaacacagagccagtgttagattcagatggcagttattttatgtacagcaaactgcgcgtagaaaaaaaaaattgggtcgagcgcaatagctactcctgcagcgtcgtgcatgaagggctacacaatcaccacaccacgaagagcttttcccggacacctgggaaa

SEQ ID NO: 173 - DNA, 1671 bp, Mus musculus
ggaaataagagagaaaagaagagtaagaagaaatataagagccaccatggaaacggacactctcctgctgtgggtactgttgctttgggtgccaggatctacgggcgaggcgaagctccaggaaagcgggccggtactggtcaaacccggcgctagcgtgaaaatgagttgcaaggccagcggttatactttcaccgactattacatgaattgggtaaagcagagccacgggaagtcattggaatggattggagtaatcaacccctacaacggggacaccagttataaccaaaagttcaagggaaaggcgacactcactgtggataaaagttcaagcacagcctacatggaacttaattccttgacatctgaggacagcgcagtatactattgtgctagatattacggtagttggttcgcctactgggggcaaggaacgctaatcactgtatcaaccgcaaaaactacagcaccctctgtataccctctcgcacctgtgtgcggagacacaactggcagctcagtaaccctcgggtgcctggttaaggggtatttcccagagccagtgactctcacctggaattctggaagccttagttcaggagtacatactttccccgctgtgctacaatcagatctgtacaccctgagttcaagcgttaccgttacgagtagtacttggccttctcagtccataacctgtaacgtcgcccatcccgcctcttctaccaaggttgacaagaagattgaaccgagaggacccaccattaaaccctgccctccttgcaagtgtcctgctccaaacctcttgggaggcccgtccgtcttcatctttcccccaaagatcaaagacgtgctcatgatttcgctgtcaccaatcgtaacgtgcgttgtggtggacgtctccgaggacgacccagatgtccagatatcctggttcgtaaacaatgtggaggtccatacagcccagacgcagacccaccgcgaggactataattctaccctcagggttgtctctgccctgccaattcagcatcaggactggatgagcgggaaagagttcaagtgtaaagtgaataataaagatctgcctgcccccatcgagagaactatcagtaaacctaaaggatccgtgcgcgctccccaggtctacgtcctgccccccccagaagaggaaatgactaagaagcaggtcacacttacctgcatggtgacggattttatgccagaagacatttatgtggaatggacaaataatggaaagacagagctcaattataagaacacagagccagtgttagattcagatggcagttattttatgtacagcaaactgcgcgtagaaaaaaaaaattgggtcgagcgcaatagctactcctgcagcgtcgtgcatgaagggctacacaatcaccacaccacgaagagcttttcccggacacctgggaaatgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 174 - DNA, 1630 bp, Mus musculus
tcaagcttttggaccctcgtacagaagctaatacgactcactatagggaaataagagagaaaagaagagtaagaagaaatataagagccaccatggaaacggacactctcctgctgtgggtactgttgctttgggtgccaggatctacgggcgaggcgaagctccaggaaagcgggccggtactggtcaaacccggcgctagcgtgaaaatgagttgcaaggccagcggttatactttcaccgactattacatgaattgggtaaagcagagccacgggaagtcattggaatggattggagtaatcaacccctacaacggggacaccagttataaccaaaagttcaagggaaaggcgacactcactgtggataaaagttcaagcacagcctacatggaacttaattccttgacatctgaggacagcgcagtatactattgtgctagatattacggtagttggttcgcctactgggggcaaggaacgctaatcactgtatcaaccgccaaaaccacaccgccttccgtgtaccctctagcgcccggttgtggtgacactacaggatcgtctgtgactttgggctgccttgtgaaaggatatttccctgaatctgttactgtgacctggaactctggctcgctgagtagttcagtgcacacattccccgccctgcttcagagcggtttgtacaccatgtcttcttcagttaccgtcccctcatcaacgtggccttcacagactgtcacgtgttctgttgcccacccagcaagctccaccaccgtggataaaaaactggagccaagcgggccgatctcaaccatcaatccgtgtcctccatgcaaggagtgccacaagtgtcctgccccaaatctggagggaggcccgtccgtctttatttttccacccaatattaaagacgtcctgatgataagcttgaccccaaaagtaacttgcgttgtggtggatgtctcggaggatgaccccgatgttcagatcagctggttcgtgaacaacgtcgaggtacatactgcgcagacccaaactcaccgagaagattataacagtactattcgagtggtgtctacccttcccatccagcaccaggactggatgtccggaaaggaatttaaatgcaaggtgaataataaagatctaccctcaccaattgaacgaacgataagtaagatcaaaggactggtgagggccccacaggtgtatattctcccacctcctgccgagcagctgtcccgcaaagacgtctccttaacatgtttagtcgtgggattcaaccctggagatattagcgtcgagtggaccagtaacggtcacacggaagagaactacaaagacacggcgcccgtgctcgacagtgacggcagttacttcatctactccaaacttaatatgaagaccagtaaatgggagaagacagactccttttcttgcaatgtgcgccacgagggcctgaagaactactacctgaaaaagacaatcagcagaagccctggcaagtgataataggctggagcctcggtggccatgcttcttgccccttgggcctccccccagcccctcctccccttcctgcacccgtacccccgtggtctttgaataaagtctgagtgggcggc

SEQ ID NO: 175 - protein, 473 aa, Mus musculus
METDTLLLWVLLLWVPGSTGEAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 176 - DNA, 1419 bp, Mus musculus
atggaaacggacactctcctgctgtgggtactgttgctttgggtgccaggatctacgggcgaggcgaagctccaggaaagcgggccggtactggtcaaacccggcgctagcgtgaaaatgagttgcaaggccagcggttatactttcaccgactattacatgaattgggtaaagcagagccacgggaagtcattggaatggattggagtaatcaacccctacaacggggacaccagttataaccaaaagttcaagggaaaggcgacactcactgtggataaaagttcaagcacagcctacatggaacttaattccttgacatctgaggacagcgcagtatactattgtgctagatattacggtagttggttcgcctactgggggcaaggaacgctaatcactgtatcaaccgccaaaaccacaccgccttccgtgtaccctctagcgcccggttgtggtgacactacaggatcgtctgtgactttgggctgccttgtgaaaggatatttccctgaatctgttactgtgacctggaactctggctcgctgagtagttcagtgcacacattccccgccctgcttcagagcggtttgtacaccatgtcttcttcagttaccgtcccctcatcaacgtggccttcacagactgtcacgtgttctgttgcccacccagcaagctccaccaccgtggataaaaaactggagccaagcgggccgatctcaaccatcaatccgtgtcctccatgcaaggagtgccacaagtgtcctgccccaaatctggagggaggcccgtccgtctttatttttccacccaatattaaagacgtcctgatgataagcttgaccccaaaagtaacttgcgttgtggtggatgtctcggaggatgaccccgatgttcagatcagctggttcgtgaacaacgtcgaggtacatactgcgcagacccaaactcaccgagaagattataacagtactattcgagtggtgtctacccttcccatccagcaccaggactggatgtccggaaaggaatttaaatgcaaggtgaataataaagatctaccctcaccaattgaacgaacgataagtaagatcaaaggactggtgagggccccacaggtgtatattctcccacctcctgccgagcagctgtcccgcaaagacgtctccttaacatgtttagtcgtgggattcaaccctggagatattagcgtcgagtggaccagtaacggtcacacggaagagaactacaaagacacggcgcccgtgctcgacagtgacggcagttacttcatctactccaaacttaatatgaagaccagtaaatgggagaagacagactccttttcttgcaatgtgcgccacgagggcctgaagaactactacctgaaaaagacaatcagcagaagccctggcaag

SEQ ID NO: 177 - DNA, 1689 bp, Mus musculus
ggaaataagagagaaaagaagagtaagaagaaatataagagccaccatggaaacggacactctcctgctgtgggtactgttgctttgggtgccaggatctacgggcgaggcgaagctccaggaaagcgggccggtactggtcaaacccggcgctagcgtgaaaatgagttgcaaggccagcggttatactttcaccgactattacatgaattgggtaaagcagagccacgggaagtcattggaatggattggagtaatcaacccctacaacggggacaccagttataaccaaaagttcaagggaaaggcgacactcactgtggataaaagttcaagcacagcctacatggaacttaattccttgacatctgaggacagcgcagtatactattgtgctagatattacggtagttggttcgcctactgggggcaaggaacgctaatcactgtatcaaccgccaaaaccacaccgccttccgtgtaccctctagcgcccggttgtggtgacactacaggatcgtctgtgactttgggctgccttgtgaaaggatatttccctgaatctgttactgtgacctggaactctggctcgctgagtagttcagtgcacacattccccgccctgcttcagagcggtttgtacaccatgtcttcttcagttaccgtcccctcatcaacgtggccttcacagactgtcacgtgttctgttgcccacccagcaagctccaccaccgtggataaaaaactggagccaagcgggccgatctcaaccatcaatccgtgtcctccatgcaaggagtgccacaagtgtcctgccccaaatctggagggaggcccgtccgtctttatttttccacccaatattaaagacgtcctgatgataagcttgaccccaaaagtaacttgcgttgtggtggatgtctcggaggatgaccccgatgttcagatcagctggttcgtgaacaacgtcgaggtacatactgcgcagacccaaactcaccgagaagattataacagtactattcgagtggtgtctacccttcccatccagcaccaggactggatgtccggaaaggaatttaaatgcaaggtgaataataaagatctaccctcaccaattgaacgaacgataagtaagatcaaaggactggtgagggccccacaggtgtatattctcccacctcctgccgagcagctgtcccgcaaagacgtctccttaacatgtttagtcgtgggattcaaccctggagatattagcgtcgagtggaccagtaacggtcacacggaagagaactacaaagacacggcgcccgtgctcgacagtgacggcagttacttcatctactccaaacttaatatgaagaccagtaaatgggagaagacagactccttttcttgcaatgtgcgccacgagggcctgaagaactactacctgaaaaagacaatcagcagaagccctggcaagtgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 178 - DNA, 928 bp, Mus musculus
tcaagcttttggaccctcgtacagaagctaatacgactcactatagggaaataagagagaaaagaagagtaagaagaaatataagagccaccatggaaacggacactctcctgctgtgggtactgttgctttgggtgccaggatctacgggcgatattgtgatgactcaaaccacactgagcctcccggtgagcctgggggatcaagcctccattagttgccgaagctcccaaagtatcgtgcactccaacggcaacacctatctggagtggtatttacagaagcccggacagagcccaaagctgctcatctacaaagtgtccaatcgcttctcgggcgtgccagacagattctcaggtagtgggtctgggacagattttaccctgaagatatctcgagtggaggcggaggatttaggagtctactattgtttccagggtagtcatgtgccttacactttcggtggggggacaaaattagaaatcaaaagggccgacgcagccccaacggtctctattttccctccatcttccgagcagctgacgagtggcggggcctcagtggtctgcttcctgaataatttctaccccaaggacataaacgtgaaatggaaaattgatggatcagaaaggcagaatggcgttctgaactcctggaccgaccaggatagcaaggactccacctactcgatgtccagtacactcactctgactaaggacgaatatgaaagacataattcatacacctgtgaggctacacataaaacatccacttccccaatcgtcaaatccttcaatcgaaatgagtgctgataataggctggagcctcggtggccatgcttcttgccccttgggcctccccccagcccctcctccccttcctgcacccgtacccccgtggtctttgaataaagtctgagtgggcggc

SEQ ID NO: 179 - protein, 239 aa, Mus musculus
METDTLLLWVLLLWVPGSTGDIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 180 - DNA, 717 bp, Mus musculus
atggaaacggacactctcctgctgtgggtactgttgctttgggtgccaggatctacgggcgatattgtgatgactcaaaccacactgagcctcccggtgagcctgggggatcaagcctccattagttgccgaagctcccaaagtatcgtgcactccaacggcaacacctatctggagtggtatttacagaagcccggacagagcccaaagctgctcatctacaaagtgtccaatcgcttctcgggcgtgccagacagattctcaggtagtgggtctgggacagattttaccctgaagatatctcgagtggaggcggaggatttaggagtctactattgtttccagggtagtcatgtgccttacactttcggtggggggacaaaattagaaatcaaaagggccgacgcagccccaacggtctctattttccctccatcttccgagcagctgacgagtggcggggcctcagtggtctgcttcctgaataatttctaccccaaggacataaacgtgaaatggaaaattgatggatcagaaaggcagaatggcgttctgaactcctggaccgaccaggatagcaaggactccacctactcgatgtccagtacactcactctgactaaggacgaatatgaaagacataattcatacacctgtgaggctacacataaaacatccacttccccaatcgtcaaatccttcaatcgaaatgagtgc

SEQ ID NO: 181 - DNA, 987 bp, Mus musculus
ggaaataagagagaaaagaagagtaagaagaaatataagagccaccatggaaacggacactctcctgctgtgggtactgttgctttgggtgccaggatctacgggcgatattgtgatgactcaaaccacactgagcctcccggtgagcctgggggatcaagcctccattagttgccgaagctcccaaagtatcgtgcactccaacggcaacacctatctggagtggtatttacagaagcccggacagagcccaaagctgctcatctacaaagtgtccaatcgcttctcgggcgtgccagacagattctcaggtagtgggtctgggacagattttaccctgaagatatctcgagtggaggcggaggatttaggagtctactattgtttccagggtagtcatgtgccttacactttcggtggggggacaaaattagaaatcaaaagggccgacgcagccccaacggtctctattttccctccatcttccgagcagctgacgagtggcggggcctcagtggtctgcttcctgaataatttctaccccaaggacataaacgtgaaatggaaaattgatggatcagaaaggcagaatggcgttctgaactcctggaccgaccaggatagcaaggactccacctactcgatgtccagtacactcactctgactaaggacgaatatgaaagacataattcatacacctgtgaggctacacataaaacatccacttccccaatcgtcaaatccttcaatcgaaatgagtgctgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 182 - DNA, 355 bp, Homo sapiens
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcag

SEQ ID NO: 183 - protein, 118 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 184 - DNA, 325 bp, Homo sapiens
gaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaac

SEQ ID NO: 185 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 186 - protein, 5 aa, Homo sapiens
SYTMH

SEQ ID NO: 187 - protein, 17 aa, Homo sapiens
FISYDGNNKYYADSVKG

SEQ ID NO: 188 - protein, 9 aa, Homo sapiens
TGWLGPFDY

SEQ ID NO: 189 - protein, 12 aa, Homo sapiens
RASQSVGSSYLA

SEQ ID NO: 190 - protein, 7 aa, Homo sapiens
GAFSRAT

SEQ ID NO: 191 - protein, 9 aa, Homo sapiens
QQYGSSPWT

SEQ ID NO: 192 - RNA, 85 nt, Homo sapiens
ccuuagcagagcuguggagugugacaaugguguuugugucuaaacuaucaaacgccauuaucacacuaaauagcuacugcuaggc

SEQ ID NO: 193 - RNA, 22 nt, Homo sapiens
aacgccauuaucacacuaaaua

SEQ ID NO: 194 - RNA, 22 nt, Homo sapiens
uauuuagugugauaauggcguu

SEQ ID NO: 195 - RNA, 22 nt, Homo sapiens
uggagugugacaaugguguuug

SEQ ID NO: 196 - RNA, 22 nt, Homo sapiens
caaacaccauugucacacucca

SEQ ID NO: 197 - RNA, 47 nt, Homo sapiens
gggaaauaagagagaaaagaagaguaagaagaaauauaagagccacc

SEQ ID NO: 198 - RNA, 47 nt, Homo sapiens
gggagaucagagagaaaagaagaguaagaagaaauauaagagccacc

SEQ ID NO: 199 - RNA, 145 nt, Homo sapiens
ggaauaaaagucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagcaac

SEQ ID NO: 200 - RNA, 42 nt, Homo sapiens
gggagacaagcuuggcauuccgguacuguugguaaagccacc

SEQ ID NO: 201 - RNA, 47 nt, Homo sapiens; identical to SEQ ID NO: 198

SEQ ID NO: 202 - RNA, 145 nt, Homo sapiens; identical to SEQ ID NO: 199

SEQ ID NO: 203 - RNA, 42 nt, Homo sapiens; identical to SEQ ID NO: 200

SEQ ID NO: 204 - RNA, 47 nt, Homo sapiens
gggaauuaacagagaaaagaagaguaagaagaaauauaagagccacc

SEQ ID NO: 205 - RNA, 47 nt, Homo sapiens
gggaaauuagacagaaaagaagaguaagaagaaauauaagagccacc

SEQ ID NO: 206 - RNA, 47 nt, Homo sapiens
gggaaauaagagaguaaagaacaguaagaagaaauauaagagccacc

SEQ ID NO: 207 - RNA, 47 nt, Homo sapiens
gggaaaaaagagagaaaagaagacuaagaagaaauauaagagccacc

SEQ ID NO: 208 - RNA, 47 nt, Homo sapiens
gggaaauaagagagaaaagaagaguaagaagauauauaagagccacc

SEQ ID NO: 209 - RNA, 47 nt, Homo sapiens
gggaaauaagagacaaaacaagaguaagaagaaauauaagagccacc

SEQ ID NO: 210 - RNA, 47 nt, Homo sapiens
gggaaauuagagaguaaagaacaguaaguagaauuaaaagagccacc

SEQ ID NO: 211 - RNA, 47 nt, Homo sapiens
gggaaauaagagagaauagaagaguaagaagaaauauaagagccacc

SEQ ID NO: 212 - RNA, 47 nt, Homo sapiens
gggaaauaagagagaaaagaagaguaagaagaaaauuaagagccacc

SEQ ID NO: 213 - RNA, 47 nt, Homo sapiens
gggaaauaagagagaaaagaagaguaagaagaaauuuaagagccacc

SEQ ID NO: 214 - RNA, 47 nt, Homo sapiens; identical to SEQ ID NO: 197

SEQ ID NO: 215 - RNA, 371 nt, Homo sapiens
gcgccugcccaccugccaccgacugcuggaacccagccagugggagggccuggcccaccagaguccugcucccucacuccucgccccgcccccugucccagagucccaccugggggcucucuccacccuucucagaguuccaguuucaaccagaguuccaaccaaugggcuccauccucuggauucuggccaaugaaauaucucccuggcaggguccucuucuuuucccagagcuccaccccaaccaggagcucuaguuaauggagagcucccagcacacucggagcuugugcuuugucuccacgcaaagcgauaaauaaaagcauugguggccuuuggucuuugaauaaagccugaguaggaagucuaga

SEQ ID NO: 216 - RNA, 568 nt, Homo sapiens
gccccugccgcucccacccccacccaucugggccccggguucaagagagagcggggucugaucucguguagccauauagaguuugcuucugagugucugcuuuguuuaguagaggugggcaggaggagcugaggggcuggggcugggguguugaaguuggcuuugcaugcccagcgaugcgccucccugugggaugucaucacccugggaaccgggaguggcccuuggcucacuguguucugcaugguuuggaucugaauuaauuguccuuucuucuaaaucccaaccgaacuucuuccaaccuccaaacuggcuguaaccccaaauccaagccauuaacuacaccugacaguagcaauugucugauuaaucacuggccccuugaagacagcagaaugucccuuugcaaugaggaggagaucugggcugggcgggccagcuggggaagcauuugacuaucuggaacuugugugugccuccucagguauggcagugacucaccugguuuuaauaaaacaaccugcaacaucucauggucuuugaauaaagccugaguaggaagucuaga

SEQ ID NO: 217 - RNA, 289 nt, Homo sapiens
acacacuccaccuccagcacgcgacuucucaggacgacgaaucuucucaaugggggggcggcugagcuccagccaccccgcagucacuuucuuuguaacaacuuccguugcugccaucguaaacugacacaguguuuauaacguguacauacauuaacuuauuaccucauuuuguuauuuuucgaaacaaagcccuguggaagaaaauggaaaacuugaagaagcauuaaagucauucuguuaagcugcguaaauggucuuugaauaaagccugaguaggaagucuaga

SEQ ID NO: 218 - RNA, 379 nt, Homo sapiens
caucacauuuaaaagcaucucagccuaccaugagaauaagagaaagaaaaugaagaucaaaagcuuauucaucuguuuuucuuuuucguugguguaaagccaacacccugucuaaaaaacauaaauuucuuuaaucauuuugccucuuuucucugugcuucaauuaauaaaaaauggaaagaaucuaauagagugguacagcacuguuauuuuucaaagauguguugcuauccugaaaauucuguagguucuguggaaguuccaguguucucucuuauuccacuucgguagaggauuucuaguuucuugugggcuaauuaaauaaaucauuaauacucuucuaauggucuuugaauaaagccugaguaggaagucuaga

SEQ ID NO: 219 - RNA, 118 nt, Homo sapiens
gcugccuucugcggggcuugccuucuggccaugcccuucuucucucccuugcaccuguaccucuuggucuuugaauaaagccugaguaggaaggcggccgcucgagcaugcaucuaga

SEQ ID NO: 220 - RNA, 908 nt, Homo sapiens
gccaagcccuccccaucccauguauuuaucucuauuuaauauuuaugucuauuuaagccucauauuuaaagacagggaagagcagaacggagccccaggccucuguguccuucccugcauuucugaguuucauucuccugccuguagcagugagaaaaagcuccuguccucccauccccuggacugggagguagauagguaaauaccaaguauuuauuacuaugacugcuccccagcccuggcucugcaaugggcacugggaugagccgcugugagccccugguccugaggguccccaccugggacccuugagaguaucaggucucccacgugggagacaagaaaucccuguuuaauauuuaaacagcaguguuccccaucuggguccuugcaccccucacucuggccucagccgacugcacagcggccccugcauccccuuggcugugaggccccuggacaagcagagguggccagagcugggaggcauggcccuggggucccacgaauuugcuggggaaucucguuuuucuucuuaagacuuuugggacaugguuugacucccgaacaucaccgacgcgucuccuguuuuucuggguggccucgggacaccugcccugcccccacgagggucaggacugugacucuuuuuagggccaggcaggugccuggacauuugccuugcuggacggggacuggggaugugggagggagcagacaggaggaaucaugucaggccugugugugaaaggaagcuccacugucacccuccaccucuucaccccccacucaccaguguccccuccacugucacauuguaacugaacuucaggauaauaaaguguuugccuccauggucuuugaauaaagccugaguaggaaggcggccgcucgagcaugcaucuaga

SEQ ID NO: 221 - RNA, 835 nt, Homo sapiens
acucaaucuaaauuaaaaaagaaagaaauuugaaaaaacuuucucuuugccauuucuucuucuucuuuuuuaacugaaagcugaauccuuccauuucuucugcacaucuacuugcuuaaauugugggcaaaagagaaaaagaaggauugaucagagcauugugcaauacaguuucauuaacuccuucccccgcucccccaaaaauuugaauuuuuuuuucaacacucuuacaccuguuauggaaaaugucaaccuuuguaagaaaaccaaaauaaaaauugaaaaauaaaaaccauaaacauuugcaccacuuguggcuuuugaauaucuuccacagagggaaguuuaaaacccaaacuuccaaagguuuaaacuaccucaaaacacuuucccaugagugugauccacauuguuaggugcugaccuagacagagaugaacugagguccuuguuuuguuuuguucauaauacaaaggugcuaauuaauaguauuucagauacuugaagaauguugauggugcuagaagaauuugagaagaaauacuccuguauugaguuguaucgugugguguauuuuuuaaaaaauuugauuuagcauucauauuuuccaucuuauucccaauuaaaaguaugcagauuauuugcccaaaucuucuucagauucagcauuuguucuuugccagucucauuuucaucuucuuccaugguuccacagaagcuuuguuucuugggcaagcagaaaaauuaaauuguaccuauuuuguauaugugagauguuuaaauaaauugugaaaaaaaugaaauaaagcauguuugguuuuccaaaagaacauau

SEQ ID NO: 222 - RNA, 297 nt, Homo sapiens
cgccgccgcccgggccccgcagucgagggucgugagcccaccccguccauggugcuaagcgggcccgggucccacacggccagcaccgcugcucacucggacgacgcccugggccugcaccucuccagcuccucccacgggguccccguagccccggcccccgcccagccccaggucuccccaggcccuccgcaggcugcccggccucccucccccugcagccaucccaaggcuccugaccuaccuggccccugagcucuggagcaagcccugacccaauaaaggcuuugaacccau

SEQ ID NO: 223 - RNA, 602 nt, Homo sapiens
ggggcuagagcccucuccgcacagcguggagacggggcaaggagggggguuauuaggauuggugguuuuguuuugcuuuguuuaaagccgugggaaaauggcacaacuuuaccucugugggagaugcaacacugagagccaaggggugggaguugggauaauuuuuauauaaaagaaguuuuuccacuuugaauugcuaaaaguggcauuuuuccuaugugcagucacuccucucauuucuaaaauagggacguggccaggcacgguggcucaugccuguaaucccagcacuuugggaggccgaggcaggcggcucacgaggucaggagaucgagacuauccuggcuaacacgguaaaacccugucucuacuaaaaguacaaaaaauuagcugggcgugguggugggcaccuguagucccagcuacucgggaggcugaggcaggagaaaggcaugaauccaagaggcagagcuugcagugagcugagaucacgccauugcacuccagccugggcaacaguguuaagacucugucucaaauauaaauaaauaaauaaauaaauaaauaaauaaauaaaaauaaagcgagauguugcccucaaa

SEQ ID NO: 224 - RNA, 785 nt, Homo sapiens
ggcccugccccgucggacugcccccagaaagccuccugcccccugccagugaaguccuucagugagccccuccccagccagcccuucccuggccccgccggauguauaaauguaaaaaugaaggaauuacauuuuauaugugagcgagcaagccggcaagcgagcacaguauuauuucuccauccccucccugccugcuccuuggcacccccaugcugccuucagggagacaggcagggagggcuuggggcugcaccuccuacccucccaccagaacgcaccccacugggagagcugguggugcagccuuccccucccuguauaagacacuuugccaaggcucuccccucucgccccaucccugcuugcccgcucccacagcuuccugagggcuaauucugggaagggagaguucuuugcugccccugucuggaagacguggcucugggugagguaggcgggaaaggauggaguguuuuaguucuugggggaggccaccccaaaccccagccccaacuccaggggcaccuaugagauggccaugcucaaccccccucccagacaggcccucccugucuccagggcccccaccgagguucccagggcuggagacuuccucugguaaacauuccuccagccuccccuccccuggggacgccaaggaggugggccacacccaggaagggaaagcgggcagccccguuuuggggacgugaacguuuuaauaauuuuugcugaauuccuuuacaacuaaauaacacagauauuguuauaaauaaaauugu

SEQ ID NO: 225 - RNA, 3001 nt, Homo sapiens
auauuaaggaucaagcuguuagcuaauaaugccaccucugcaguuuugggaacaggcaaauaaaguaucaguauacauggugauguacaucuguagcaaagcucuuggagaaaaugaagacugaagaaagcaaagcaaaaacuguauagagagauuuuucaaaagcaguaaucccucaauuuuaaaaaaggauugaaaauucuaaaugucuuucugugcauauuuuuuguguuaggaaucaaaaguauuuuauaaaaggagaaagaacagccucauuuuagauguaguccuguuggauuuuuuaugccuccucaguaaccagaaauguuuuaaaaaacuaaguguuuaggauuucaagacaacauuauacauggcucugaaauaucugacacaauguaaacauugcaggcaccugcauuuuauguuuuuuuuuucaacaaaugugacuaauuugaaacuuuuaugaacuucugagcuguccccuugcaauucaaccgcaguuugaauuaaucauaucaaaucaguuuuaauuuuuuaaauuguacuucagagucuauauuucaagggcacauuuucucacuacuauuuuaauacauuaaaggacuaaauaaucuuucagagaugcuggaaacaaaucauuugcuuuauauguuucauuagaauaccaaugaaacauacaacuugaaaauuaguaauaguauuuuugaagaucccauuucuaauuggagaucucuuuaauuucgaucaacuuauaauguguaguacuauauuaagugcacuugaguggaauucaacauuugacuaauaaaaugaguucaucauguuggcaagugauguggcaauuaucucuggugacaaaagaguaaaaucaaauauuucugccuguuacaaauaucaaggaagaccugcuacuaugaaauagaugacauuaaucugucuucacuguuuauaauacggauggauuuuuuuucaaaucaguguguguuuugaggucuuauguaauugaugacauuugagagaaaugguggcuuuuuuuagcuaccucuuuguucauuuaagcaccaguaaagaucaugucuuuuuauagaaguguagauuuucuuugugacuuugcuaucgugccuaaagcucuaaauauaggugaaugugugaugaauacucagauuauuugucucucuauauaauuaguuugguacuaaguuucucaaaaaauuauuaacacaugaaagacaaucucuaaaccagaaaaagaaguaguacaaauuuuguuacuguaaugcucgcguuuagugaguuuaaaacacacaguaucuuuugguuuuauaaucaguuucuauuuugcugugccugagauuaagaucuguguaugugugugugugugugugugcguuuguguguuaaagcagaaaagacuuuuuuaaaaguuuuaagugauaaaugcaauuuguuaauugaucuuagaucacuaguaaacucagggcugaauuauaccauguauauucuauuagaagaaaguaaacaccaucuuuauuccugcccuuuuucuucucucaaaguaguuguaguuauaucuagaaagaagcaauuuugauuucuugaaaagguaguuccugcacucaguuuaaacuaaaaauaaucauacuuggauuuuauuuauuuuugucauaguaaaaauuuuaauuuauauauauuuuuauuuaguauuaucuuauucuuugcuauuugccaauccuuugucaucaauuguguuaaaugaauugaaaauucaugcccuguucauuuuauuuuacuuuauugguuaggauauuuaaaggauuuuuguauauauaauuucuuaaauuaauauuccaaaagguuaguggacuuagauuauaaauuauggcaaaaaucuaaaaacaacaaaaaugauuuuuauacauucuauuucauuauuccucuuuuuccaauaagucauacaauugguagauaugacuuauuuuauuuuuguauuauucacuauaucuuuaugauauuuaaguauaaauaauuaaaaaaauuuauuguaccuuauagucugucaccaaaaaaaaaaaauuaucuguagguagugaaaugcuaauguugauuugucuuuaagggcuuguuaacuauccuuuauuuucucauuugucuuaaauuaggaguuuguguuuaaauuacucaucuaagcaaaaaauguauauaaaucccauuacuggguauauacccaaaggauuauaaaucaugcugcuauaaagacacaugcacacguauguuuauugcagcacuauucacaauagcaaagacuuggaaccaacccaaauguccaucaaugauagacuugauuaagaaaaugugcacauauacaccauggaauacuaugcagccauaaaaaaggaugaguucauguccuuuguagggacauggauaaagcuggaaaccaucauucugagcaaacuauugcaaggacagaaaaccaaacacugcauguucucacucauaggugggaauugaacaaugagaacacuuggacacaagguggggaacaccacacaccagggccugucaugggguggggggaguggggagggauagcauuaggagauauaccuaauguaaaugaugaguuaaugggugcagcacaccaacauggcacauguauacauauguagcaaaccugcacguugugcacauguacccuagaacuuaaaguauaauuaaaaaaaaaaagaaaacagaagcuauuuauaaagaaguuauuugcugaaauaaaugugaucuuucccauuaaaaaaauaaagaaauuuugggguaaaaaaacacaauauauuguauucuugaaaaauucuaagagaguggaugugaaguguucucaccacaaaagugauaacuaauugagguaaugcacauauuaauuagaaagauuuugucauuccacaauguauauauacuuaaaaauauguuauacacaauaaauacauacauuaaaaaauaaguaaaugua

SEQ ID NO: 226 - RNA, 1037 nt, Homo sapiens
cccacccugcacgccggcaccaaacccuguccucccaccccuccccacucaucacuaaacagaguaaaaugugaugcgaauuuucccgaccaaccugauucgcuagauuuuuuuuaaggaaaagcuuggaaagccaggacacaacgcugcugccugcuuugugcaggguccuccggggcucagcccugaguuggcaucaccugcgcagggcccucuggggcucagcccugagcuagugucaccugcacagggcccucugaggcucagcccugagcuggcgucaccugugcagggcccucuggggcucagcccugagcuggccucaccuggguuccccaccccgggcucuccugcccugcccuccugcccgcccucccuccugccugcgcagcuccuucccuaggcaccucugugcugcaucccaccagccugagcaagacgcccucucggggccugugccgcacuagccucccucuccucuguccccauagcugguuuuucccaccaauccucaccuaacaguuacuuuacaauuaaacucaaagcaagcucuucuccucagcuuggggcagccauuggccucugucucguuuugggaaaccaaggucaggaggccguugcagacauaaaucucggcgacucggccccgucuccugaggguccugcuggugaccggccuggaccuuggcccuacagcccuggaggccgcugcugaccagcacugaccccgaccucagagaguacucgcaggggcgcuggcugcacucaagacccucgagauuaacggugcuaaccccgucugcuccucccucccgcagagacuggggccuggacuggacaugagagccccuuggugccacagagggcugugucuuacuagaaacaacgcaaaccucuccuuccucagaauagugauguguucgacguuuuaucaaaggcccccuuucuauguucauguuaguuuugcuccuucuguguuuuuuucugaaccauauccauguugcugacuuuuccaaauaaagguuuucacuccucuc

SEQ ID NO: 227 - RNA, 577 nt, Homo sapiens
agaggccugccuccagggcuggacugaggccugagcgcuccugccgcagagcuggccgcgccaaauaaugucucugugagacucgagaacuuucauuuuuuuccaggcugguucggauuugggguggauuuugguuuuguuccccuccuccacucucccccacccccuccccgcccuuuuuuuuuuuuuuuuuuaaacugguauuuuaucuuugauucuccuucagcccucaccccugguucucaucuuucuugaucaacaucuuuucuugccucuguccccuucucucaucucuuagcuccccuccaaccuggggggcagugguguggagaagccacaggccugagauuucaucugcucuccuuccuggagcccagaggagggcagcagaaggggguggugucuccaaccccccagcacugaggaagaacggggcucuucucauuucaccccucccuuucuccccugcccccaggacugggccacuucuggguggggcagugggucccagauuggcucacacugagaauguaagaacuacaaacaaaauuucuauuaaauuaaauuuugugucucc

SEQ ID NO: 228 - RNA, 2212 nt, Homo sapiens
cucccuccaucccaaccuggcucccucccacccaaccaacuuuccccccaacccggaaacagacaagcaacccaaacugaacccccucaaaagccaaaaaaugggagacaauuucacauggacuuuggaaaauauuuuuuuccuuugcauucaucucucaaacuuaguuuuuaucuuugaccaaccgaacaugaccaaaaaccaaaagugcauucaaccuuaccaaaaaaaaaaaaaaaaaaagaauaaauaaauaacuuuuuaaaaaaggaagcuugguccacuugcuugaagacccaugcggggguaagucccuuucugcccguugggcuuaugaaaccccaaugcugcccuuucugcuccuuucuccacaccccccuuggggccuccccuccacuccuucccaaaucugucuccccagaagacacaggaaacaauguauugucugcccagcaaucaaaggcaaugcucaaacacccaaguggcccccacccucagcccgcuccugcccgcccagcacccccaggcccugggggaccugggguucucagacugccaaagaagccuugccaucuggcgcucccauggcucuugcaacaucuccccuucguuuuugagggggucaugccgggggagccaccagccccucacuggguucggaggagagucaggaagggccacgacaaagcagaaacaucggauuuggggaacgcgugucaaucccuugugccgcagggcugggcgggagagacuguucuguuccuuguguaacuguguugcugaaagacuaccucguucuugucuugaugugucaccggggcaacugccugggggcggggaugggggcaggguggaagcggcuccccauuuuauaccaaaggugcuacaucuaugugaugggugggguggggagggaaucacuggugcuauagaaauugagaugcccccccaggccagcaaauguuccuuuuuguucaaagucuauuuuuauuccuugauauuuuucuuuuuuuuuuuuuuuuuuuguggauggggacuugugaauuuuucuaaaggugcuauuuaacaugggaggagagcgugugcggcuccagcccagcccgcugcucacuuuccacccucucuccaccugccucuggcuucucaggccucugcucuccgaccucucuccucugaaacccuccuccacagcugcagcccauccucccggcucccuccuagucuguccugcguccucuguccccggguuucagagacaacuucccaaagcacaaagcaguuuuucccccuaggggugggaggaagcaaaagacucuguaccuauuuuguauguguauaauaauuugagauguuuuuaauuauuuugauugcuggaauaaagcauguggaaaugacccaaacauaauccgcaguggccuccuaauuuccuucuuuggaguugggggagggguagacauggggaaggggcuuuggggugaugggcuugccuuccauuccugcccuuucccuccccacuauucucuucuagaucccuccauaaccccacuccccuuucucucacccuucuuauaccgcaaaccuuucuacuuccucuuucauuuucuauucuugcaauuuccuugcaccuuuuccaaauccucuucuccccugcaauaccauacaggcaauccacgugcacaacacacacacacacucuucacaucugggguuguccaaaccucauacccacuccccuucaagcccauccacucuccacccccuggaugcccugcacuugguggcggugggaugcucauggauacugggagggugaggggaguggaacccgugaggaggaccugggggccucuccuugaacugacaugaagggucaucuggccucugcucccuucucacccacgcugaccuccugccgaaggagcaacgcaacaggagaggggucugcugagccuggcgagggucugggagggaccaggaggaaggcgugcucccugcucgcuguccuggcccugggggagugagggagacagacaccugggagagcuguggggaaggcacucgcaccgugcucuugggaaggaaggagaccuggcccugcucaccacggacugggugccucgaccuccugaauccccagaacacaaccccccugggcugggguggucuggggaaccaucgugcccccgccucccgccuacuccuuuuuaagcuu

SEQ ID NO: 229 - RNA, 729 nt, Homo sapiens
uuggccaggccugacccucuuggaccuuucuucuuugccgacaaccacugcccagcagccucugggaccucggggucccagggaacccaguccagccuccuggcuguugacuucccauugcucuuggagccaccaaucaaagagauucaaagagauuccugcaggccagaggcggaacacaccuuuauggcuggggcucuccgugguguucuggacccagccccuggagacaccauucacuuuuacugcuuuguagugacucgugcucuccaaccugucuuccugaaaaaccaaggcccccuucccccaccucuuccauggggugagacuugagcagaacaggggcuuccccaaguugcccagaaagacugucugggugagaagccauggccagagcuucucccaggcacagguguugcaccagggacuucugcuucaaguuuugggguaaagacaccuggaucagacuccaagggcugcccugagucugggacuucugccuccauggcuggucaugagagcaaaccguaguccccuggagacagcgacuccagagaaccucuugggagacagaagaggcaucugugcacagcucgaucuucuacuugccuguggggaggggagugacagguccacacaccacacugggucacccuguccuggaugccucugaagagagggacagaccgucagaaacuggagaguuucuauuaaaggucauuuaaacca

SEQ ID NO: 230 - RNA, 847 nt, Homo sapiens
uccuccgggaccccagcccucaggauuccugaugcuccaaggcgacugaugggcgcuggaugaaguggcacagucagcuucccugggggcuggugucauguugggcuccuggggcgggggcacggccuggcauuucacgcauugcugccaccccagguccaccugucuccacuuucacagccuccaagucuguggcucuucccuucuguccuccgaggggcuugccuucucucguguccagugaggugcucagugaucggcuuaacuuagagaagcccgcccccuccccuucuccgucugucccaagagggucugcucugagccugcguuccuagguggcucggccucagcugccuggguuguggccgcccuagcauccuguaugcccacagcuacuggaauccccgcugcugcuccgggccaagcuucugguugauuaaugagggcaugggguggucccucaagaccuuccccuaccuuuuguggaaccagugaugccucaaagacaguguccccuccacagcugggugccaggggcaggggauccucaguauagccggugaacccugauaccaggagccugggccucccugaaccccuggcuuccagccaucucaucgccagccuccuccuggaccucuuggcccccagccccuuccccacacagccccagaagggucccagagcugaccccacuccaggaccuaggcccagccccucagccucaucuggagccccugaagaccagucccacccaccuuucuggccucaucugacacugcuccgcauccugcuguguguccuguuccauguuccgguuccauccaaauacacuuucuggaacaaa

SEQ ID NO: 231 - RNA, 110 nt, Homo sapiens
gcuggagccucgguggccaugcuucuugccccuugggccuccccccagccccuccuccccuuccugcacccguacccccguggucuuugaauaaagucugagugggcggc

SEQ ID NO: 232 - RNA, 116 nt, Homo sapiens
uaauaggcuggagccucgguggccaugcuucuugccccuugggccuccccccagccccuccuccccuuccugcacccguacccccguggucuuugaauaaagucugagugggcggc

SEQ ID NO: 233 - DNA, 18 bp, Homo sapiens
attgggcacccgtaaggg

SEQ ID NO: 234 - protein, 20 aa, Homo sapiens
METPAQLLFLLLLWLPDTTG

SEQ ID NO: 235 - protein, 471 aa, Homo sapiens
METPAQLLFLLLLWLPDTTGQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 236 - protein, 234 aa, Homo sapiens
METPAQLLFLLLLWLPDTTGDIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - DNA, 325 bp, Homo sapiens
gaaattgtgttgacgcagtctccaggcaccctgtctttgtctccaggggaaagagccaccctctcctgcagggccagtcagagtgttagcagcagcttcttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaac

SEQ ID NO: 238 - protein, 108 aa, Homo sapiens
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIK

SEQ ID NO: 239 - DNA, 355 bp, Homo sapiens
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaagcaataaacactacgcagactccgtgaagggccgattcaccgtctccagagacaattccaagaacacgctgtatctgcaaatgaacagcctgagagctgaggacacggctatatattactgtgcgaggaccggctggctggggccctttgactactggggccagggaaccctggtcaccgtctcctcag

SEQ ID NO: 240 - protein, 118 aa, Homo sapiens
QVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSS

SEQ ID NO: 241 - DNA, 322 bp, Homo sapiens
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctccgacgttcggccaagggaccaaggtggaaatcaaac

SEQ ID NO: 242 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKVEIK

SEQ ID NO: 243 - DNA, 358 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgttttactgtgcgagagctcccaattatattggtgcttttgatgtctggggccaagggacaatggtcaccgtctcttcag

SEQ ID NO: 244 - protein, 119 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVFYCARAPNYIGAFDVWGQGTMVTVSS

SEQ ID NO: 245 - protein, 12 aa, Homo sapiens
RASQSVSSSFLA

SEQ ID NO: 246 - protein, 7 aa, Homo sapiens
GASSRAT

SEQ ID NO: 247 - protein, 9 aa, Homo sapiens
QQYGSSPWT

SEQ ID NO: 248 - protein, 5 aa, Homo sapiens
SYTMH

SEQ ID NO: 249 - protein, 17 aa, Homo sapiens
FISYDGSNKHYADSVKG

SEQ ID NO: 250 - protein, 9 aa, Homo sapiens
TGWLGPFDY

SEQ ID NO: 251 - DNA, 1637 bp, Homo sapiens
tcaagcttttggaccctcgtacagaagctaatacgactcactatagggaaataagagagaaaagaagagtaagaagaaatataagagccaccatggagacccccgcccagctgctgtttctgctgctgctgtggctgcccgacaccaccggccaggtgcagctggtggagagcggcggcggcgtggtgcagcccggcagaagcctgagactgagctgcgccgccagcggctttaccttcagcagctacaccatgcactgggtgcggcaggcccctggcaagggactggagtgggtgaccttcataagctacgacggcaataacaagtattacgccgacagcgtgaagggcagattcaccatcagcagagacaacagcaagaataccctgtacctgcagatgaatagcctgagagccgaggacaccgccatctactactgcgccagaacaggctggctgggcccattcgactactggggccagggcacactggtgacagtctccagcgctagcactaagggtcccagcgtgttccccctggccccgagcagcaaaagcaccagcggcggcaccgccgccctgggctgcctggtcaaggactacttccccgagcccgtgaccgtgtcttggaactccggggccctgaccagcggggtacacaccttccccgccgtgctgcagagcagcggcctgtattccctgagctccgtggtgaccgtccctagctccagcctgggcacccagacctacatctgcaacgtgaaccacaagccctccaacaccaaagtggacaagcgcgtggagcccaagagttgcgacaaaacccacacctgtcctccctgcccggctcctgagctgttgggcggacccagcgtcttcctgttccccccaaagcccaaggacaccctgatgatcagccggacccccgaggtgacttgcgtggtggtcgacgtgagccacgaggacccagaggtgaagttcaactggtacgtggacggcgtcgaggtgcacaacgccaagaccaagcctagagaggagcagtacaacagcacctacagggtggtgagcgtgctgaccgtgctgcatcaggactggctgaacggcaaagagtacaagtgcaaggtgtcaaataaggccctgcccgcacccattgagaagactatcagcaaggccaagggccagcccagagagccacaggtgtacaccctgccccccagcagagaggagatgaccaagaaccaggtgagcctcacctgcctggtcaagggcttttaccccagcgacatcgccgttgagtgggagagcaatggccagcccgagaacaactacaagaccacccccccggtgctggactccgacggcagctttttcctgtacagcaagctgaccgtggacaagagcagatggcagcagggcaacgtgttcagctgcagcgtgatgcacgaggccctgcacaaccactacacccagaagtctctgagcctgagccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 252 - protein, 468 aa, Homo sapiens
METPAQLLFLLLLWLPDTTGQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - DNA, 1404 bp, Homo sapiens
atggagacccccgcccagctgctgtttctgctgctgctgtggctgcccgacaccaccggccaggtgcagctggtggagagcggcggcggcgtggtgcagcccggcagaagcctgagactgagctgcgccgccagcggctttaccttcagcagctacaccatgcactgggtgcggcaggcccctggcaagggactggagtgggtgaccttcataagctacgacggcaataacaagtattacgccgacagcgtgaagggcagattcaccatcagcagagacaacagcaagaataccctgtacctgcagatgaatagcctgagagccgaggacaccgccatctactactgcgccagaacaggctggctgggcccattcgactactggggccagggcacactggtgacagtctccagcgctagcactaagggtcccagcgtgttccccctggccccgagcagcaaaagcaccagcggcggcaccgccgccctgggctgcctggtcaaggactacttccccgagcccgtgaccgtgtcttggaactccggggccctgaccagcggggtacacaccttccccgccgtgctgcagagcagcggcctgtattccctgagctccgtggtgaccgtccctagctccagcctgggcacccagacctacatctgcaacgtgaaccacaagccctccaacaccaaagtggacaagcgcgtggagcccaagagttgcgacaaaacccacacctgtcctccctgcccggctcctgagctgttgggcggacccagcgtcttcctgttccccccaaagcccaaggacaccctgatgatcagccggacccccgaggtgacttgcgtggtggtcgacgtgagccacgaggacccagaggtgaagttcaactggtacgtggacggcgtcgaggtgcacaacgccaagaccaagcctagagaggagcagtacaacagcacctacagggtggtgagcgtgctgaccgtgctgcatcaggactggctgaacggcaaagagtacaagtgcaaggtgtcaaataaggccctgcccgcacccattgagaagactatcagcaaggccaagggccagcccagagagccacaggtgtacaccctgccccccagcagagaggagatgaccaagaaccaggtgagcctcacctgcctggtcaagggcttttaccccagcgacatcgccgttgagtgggagagcaatggccagcccgagaacaactacaagaccacccccccggtgctggactccgacggcagctttttcctgtacagcaagctgaccgtggacaagagcagatggcagcagggcaacgtgttcagctgcagcgtgatgcacgaggccctgcacaaccactacacccagaagtctctgagcctgagccccggcaag

SEQ ID NO: 254 - DNA, 1696 bp, Homo sapiens
ggaaataagagagaaaagaagagtaagaagaaatataagagccaccatggagacccccgcccagctgctgtttctgctgctgctgtggctgcccgacaccaccggccaggtgcagctggtggagagcggcggcggcgtggtgcagcccggcagaagcctgagactgagctgcgccgccagcggctttaccttcagcagctacaccatgcactgggtgcggcaggcccctggcaagggactggagtgggtgaccttcataagctacgacggcaataacaagtattacgccgacagcgtgaagggcagattcaccatcagcagagacaacagcaagaataccctgtacctgcagatgaatagcctgagagccgaggacaccgccatctactactgcgccagaacaggctggctgggcccattcgactactggggccagggcacactggtgacagtctccagcgctagcactaagggtcccagcgtgttccccctggccccgagcagcaaaagcaccagcggcggcaccgccgccctgggctgcctggtcaaggactacttccccgagcccgtgaccgtgtcttggaactccggggccctgaccagcggggtacacaccttccccgccgtgctgcagagcagcggcctgtattccctgagctccgtggtgaccgtccctagctccagcctgggcacccagacctacatctgcaacgtgaaccacaagccctccaacaccaaagtggacaagcgcgtggagcccaagagttgcgacaaaacccacacctgtcctccctgcccggctcctgagctgttgggcggacccagcgtcttcctgttccccccaaagcccaaggacaccctgatgatcagccggacccccgaggtgacttgcgtggtggtcgacgtgagccacgaggacccagaggtgaagttcaactggtacgtggacggcgtcgaggtgcacaacgccaagaccaagcctagagaggagcagtacaacagcacctacagggtggtgagcgtgctgaccgtgctgcatcaggactggctgaacggcaaagagtacaagtgcaaggtgtcaaataaggccctgcccgcacccattgagaagactatcagcaaggccaagggccagcccagagagccacaggtgtacaccctgccccccagcagagaggagatgaccaagaaccaggtgagcctcacctgcctggtcaagggcttttaccccagcgacatcgccgttgagtgggagagcaatggccagcccgagaacaactacaagaccacccccccggtgctggactccgacggcagctttttcctgtacagcaagctgaccgtggacaagagcagatggcagcagggcaacgtgttcagctgcagcgtgatgcacgaggccctgcacaaccactacacccagaagtctctgagcctgagccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 255 - protein, 448 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - DNA, 1637 bp, Homo sapiens
tcaagcttttggaccctcgtacagaagctaatacgactcactatagggaaataagagagaaaagaagagtaagaagaaatataagagccaccatggagacccccgcacagctgctcttcctgctgctgctgtggctgcccgacaccaccggacaggtgcagctggtagagagcggcggcggcgtagtgcagcccggcagaagcctgaggctgtcctgcgccgccagtggctttaccttcagcagctacaccatgcactgggtgagacaggctccaggcaaaggcctggagtgggtgactttcatcagctatgacggaaacaacaagtactacgccgacagcgtgaagggcaggttcaccatctccagagacaactcgaaaaacaccctgtacctgcagatgaacagcctgagagccgaggataccgccatctactactgcgcccgcacaggctggctgggcccttttgactactggggccaaggcacactggttacagtgagcagcgcctcaaccaagggccccagtgtgttccccctggcaccctcgagcaagagcacctccggcggcaccgccgccctgggctgtctcgtgaaggactacttccccgagcccgtcaccgtgagctggaacagcggcgccctgacctcaggcgtgcacaccttccccgccgtgctccagtctagcggcctgtactccctgtcaagcgttgtgacagtgcccagcagcagcctgggcacccagacatacatctgcaatgtgaaccacaaaccctccaacaccaaggtggacaagagggtggagccaaagtcttgcgacaagacccacacctgccctccctgccccgcccccgagctgctgggcggccccagcgtgttcctgtttccccccaagcctaaggacacactgatgatcagcagaacccctgaggtgacctgtgtggtggtggacgtgagccacgaggaccccgaggtgaagtttaactggtacgtggacggcgtggaggtgcacaacgccaagaccaagcccagagaagagcagtacaacagcacctacagagtggtaagcgtgctgaccgtgctgcaccaggactggctgaacggcaaggagtacaagtgcaaggtgtccaataaggcccttcccgcccccatcgagaagaccatctccaaggccaagggccagcctagagagccccaagtgtacaccctgccccccagcagggaggagatgaccaagaatcaggtgagcctgacctgcctggtgaagggattttaccccagcgacatcgccgtggagtgggagagcaacggccagcccgagaacaactacaagaccaccccccccgtgctggacagcgacggctctttcttcctgtacagcaagctgaccgtggacaagtccaggtggcagcagggcaacgtgttcagctgcagcgtgatgcacgaggccctgcacaaccactacacccagaagagcttgtccctgagccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 257 - protein, 468 aa, Homo sapiens; identical to SEQ ID NO: 252

SEQ ID NO: 258 - DNA, 1404 bp, Homo sapiens
atggagacccccgcacagctgctcttcctgctgctgctgtggctgcccgacaccaccggacaggtgcagctggtagagagcggcggcggcgtagtgcagcccggcagaagcctgaggctgtcctgcgccgccagtggctttaccttcagcagctacaccatgcactgggtgagacaggctccaggcaaaggcctggagtgggtgactttcatcagctatgacggaaacaacaagtactacgccgacagcgtgaagggcaggttcaccatctccagagacaactcgaaaaacaccctgtacctgcagatgaacagcctgagagccgaggataccgccatctactactgcgcccgcacaggctggctgggcccttttgactactggggccaaggcacactggttacagtgagcagcgcctcaaccaagggccccagtgtgttccccctggcaccctcgagcaagagcacctccggcggcaccgccgccctgggctgtctcgtgaaggactacttccccgagcccgtcaccgtgagctggaacagcggcgccctgacctcaggcgtgcacaccttccccgccgtgctccagtctagcggcctgtactccctgtcaagcgttgtgacagtgcccagcagcagcctgggcacccagacatacatctgcaatgtgaaccacaaaccctccaacaccaaggtggacaagagggtggagccaaagtcttgcgacaagacccacacctgccctccctgccccgcccccgagctgctgggcggccccagcgtgttcctgtttccccccaagcctaaggacacactgatgatcagcagaacccctgaggtgacctgtgtggtggtggacgtgagccacgaggaccccgaggtgaagtttaactggtacgtggacggcgtggaggtgcacaacgccaagaccaagcccagagaagagcagtacaacagcacctacagagtggtaagcgtgctgaccgtgctgcaccaggactggctgaacggcaaggagtacaagtgcaaggtgtccaataaggcccttcccgcccccatcgagaagaccatctccaaggccaagggccagcctagagagccccaagtgtacaccctgccccccagcagggaggagatgaccaagaatcaggtgagcctgacctgcctggtgaagggattttaccccagcgacatcgccgtggagtgggagagcaacggccagcccgagaacaactacaagaccaccccccccgtgctggacagcgacggctctttcttcctgtacagcaagctgaccgtggacaagtccaggtggcagcagggcaacgtgttcagctgcagcgtgatgcacgaggccctgcacaaccactacacccagaagagcttgtccctgagccccggcaag

SEQ ID NO: 259 - DNA, 1696 bp, Homo sapiens
ggaaataagagagaaaagaagagtaagaagaaatataagagccaccatggagacccccgcacagctgctcttcctgctgctgctgtggctgcccgacaccaccggacaggtgcagctggtagagagcggcggcggcgtagtgcagcccggcagaagcctgaggctgtcctgcgccgccagtggctttaccttcagcagctacaccatgcactgggtgagacaggctccaggcaaaggcctggagtgggtgactttcatcagctatgacggaaacaacaagtactacgccgacagcgtgaagggcaggttcaccatctccagagacaactcgaaaaacaccctgtacctgcagatgaacagcctgagagccgaggataccgccatctactactgcgcccgcacaggctggctgggcccttttgactactggggccaaggcacactggttacagtgagcagcgcctcaaccaagggccccagtgtgttccccctggcaccctcgagcaagagcacctccggcggcaccgccgccctgggctgtctcgtgaaggactacttccccgagcccgtcaccgtgagctggaacagcggcgccctgacctcaggcgtgcacaccttccccgccgtgctccagtctagcggcctgtactccctgtcaagcgttgtgacagtgcccagcagcagcctgggcacccagacatacatctgcaatgtgaaccacaaaccctccaacaccaaggtggacaagagggtggagccaaagtcttgcgacaagacccacacctgccctccctgccccgcccccgagctgctgggcggccccagcgtgttcctgtttccccccaagcctaaggacacactgatgatcagcagaacccctgaggtgacctgtgtggtggtggacgtgagccacgaggaccccgaggtgaagtttaactggtacgtggacggcgtggaggtgcacaacgccaagaccaagcccagagaagagcagtacaacagcacctacagagtggtaagcgtgctgaccgtgctgcaccaggactggctgaacggcaaggagtacaagtgcaaggtgtccaataaggcccttcccgcccccatcgagaagaccatctccaaggccaagggccagcctagagagccccaagtgtacaccctgccccccagcagggaggagatgaccaagaatcaggtgagcctgacctgcctggtgaagggattttaccccagcgacatcgccgtggagtgggagagcaacggccagcccgagaacaactacaagaccaccccccccgtgctggacagcgacggctctttcttcctgtacagcaagctgaccgtggacaagtccaggtggcagcagggcaacgtgttcagctgcagcgtgatgcacgaggccctgcacaaccactacacccagaagagcttgtccctgagccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 260 - protein, 448 aa, Homo sapiens; identical to SEQ ID NO: 255

SEQ ID NO: 261 - DNA, 1637 bp, Homo sapiens
tcaagcttttggaccctcgtacagaagctaatacgactcactatagggaaataagagagaaaagaagagtaagaagaaatataagagccaccatggagacacccgcccagctgctcttcctgctgctgttgtggctgcccgacaccaccggacaggttcagctggtggaaagcggcggcggcgtggtgcagccaggcaggagcctgagactgtcctgcgccgcaagcggcttcaccttctccagctataccatgcactgggtgaggcaggcccccggcaaaggcctggagtgggtgacgttcatctcctacgacggtaacaataagtactacgccgacagcgtgaaaggcaggttcaccatcagcagagacaacagcaagaacactctgtacctgcagatgaactccctccgtgccgaggacaccgccatctactactgcgccaggaccggctggctgggccccttcgactactgggggcagggcaccctggtcaccgtgagtagcgccagcaccaagggacctagcgtgttccccctggcccccagcagcaagtccaccagcggcggcaccgccgccctggggtgcctggtgaaggactacttccccgaacccgtgaccgtgtcttggaacagcggggccctgacctccggcgtgcacaccttccccgccgtgctgcagagcagcggcctgtacagcctgagcagcgtggtgaccgtgcctagctctagcctcggcacccagacttacatctgcaacgtgaaccataagcccagcaacacaaaggtcgataagcgagtggagcccaagagctgcgacaagacccacacctgccccccctgccccgcccccgagctgctgggcggacccagcgtgttcctgttcccccctaaacccaaagacaccctgatgatcagcagaacccccgaggtgacctgtgtggtggtggacgtcagccacgaggaccccgaggtgaagttcaactggtacgtggacggcgttgaggtgcacaacgccaagaccaaacccagggaggagcagtacaatagcacctatagagtggtgagcgtgctgaccgtgctgcaccaggactggctgaatggcaaggagtacaagtgcaaggtgagcaacaaggccttacccgcccccatcgagaagaccatctccaaggccaagggccagcccagagagccccaggtgtacaccctgcctcccagcagagaggagatgactaagaaccaggtcagccttacctgcctggtgaagggcttttacccctcagacatcgccgtggaatgggagagcaacggccagcccgaaaataactacaagacaacccctcccgtgctggacagcgacgggagcttcttcttgtacagcaagctcaccgtggacaagagcagatggcagcagggcaacgtgttcagctgcagcgtgatgcatgaggccctgcacaaccactatacacagaagagcctgagcctgagccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 262 - protein, 468 aa, Homo sapiens; identical to SEQ ID NO: 252

SEQ ID NO: 263 - DNA, 1404 bp, Homo sapiens
atggagacacccgcccagctgctcttcctgctgctgttgtggctgcccgacaccaccggacaggttcagctggtggaaagcggcggcggcgtggtgcagccaggcaggagcctgagactgtcctgcgccgcaagcggcttcaccttctccagctataccatgcactgggtgaggcaggcccccggcaaaggcctggagtgggtgacgttcatctcctacgacggtaacaataagtactacgccgacagcgtgaaaggcaggttcaccatcagcagagacaacagcaagaacactctgtacctgcagatgaactccctccgtgccgaggacaccgccatctactactgcgccaggaccggctggctgggccccttcgactactgggggcagggcaccctggtcaccgtgagtagcgccagcaccaagggacctagcgtgttccccctggcccccagcagcaagtccaccagcggcggcaccgccgccctggggtgcctggtgaaggactacttccccgaacccgtgaccgtgtcttggaacagcggggccctgacctccggcgtgcacaccttccccgccgtgctgcagagcagcggcctgtacagcctgagcagcgtggtgaccgtgcctagctctagcctcggcacccagacttacatctgcaacgtgaaccataagcccagcaacacaaaggtcgataagcgagtggagcccaagagctgcgacaagacccacacctgccccccctgccccgcccccgagctgctgggcggacccagcgtgttcctgttcccccctaaacccaaagacaccctgatgatcagcagaacccccgaggtgacctgtgtggtggtggacgtcagccacgaggaccccgaggtgaagttcaactggtacgtggacggcgttgaggtgcacaacgccaagaccaaacccagggaggagcagtacaatagcacctatagagtggtgagcgtgctgaccgtgctgcaccaggactggctgaatggcaaggagtacaagtgcaaggtgagcaacaaggccttacccgcccccatcgagaagaccatctccaaggccaagggccagcccagagagccccaggtgtacaccctgcctcccagcagagaggagatgactaagaaccaggtcagccttacctgcctggtgaagggcttttacccctcagacatcgccgtggaatgggagagcaacggccagcccgaaaataactacaagacaacccctcccgtgctggacagcgacgggagcttcttcttgtacagcaagctcaccgtggacaagagcagatggcagcagggcaacgtgttcagctgcagcgtgatgcatgaggccctgcacaaccactatacacagaagagcctgagcctgagccccggcaag

SEQ ID NO: 264 - DNA, 1696 bp, Homo sapiens
ggaaataagagagaaaagaagagtaagaagaaatataagagccaccatggagacacccgcccagctgctcttcctgctgctgttgtggctgcccgacaccaccggacaggttcagctggtggaaagcggcggcggcgtggtgcagccaggcaggagcctgagactgtcctgcgccgcaagcggcttcaccttctccagctataccatgcactgggtgaggcaggcccccggcaaaggcctggagtgggtgacgttcatctcctacgacggtaacaataagtactacgccgacagcgtgaaaggcaggttcaccatcagcagagacaacagcaagaacactctgtacctgcagatgaactccctccgtgccgaggacaccgccatctactactgcgccaggaccggctggctgggccccttcgactactgggggcagggcaccctggtcaccgtgagtagcgccagcaccaagggacctagcgtgttccccctggcccccagcagcaagtccaccagcggcggcaccgccgccctggggtgcctggtgaaggactacttccccgaacccgtgaccgtgtcttggaacagcggggccctgacctccggcgtgcacaccttccccgccgtgctgcagagcagcggcctgtacagcctgagcagcgtggtgaccgtgcctagctctagcctcggcacccagacttacatctgcaacgtgaaccataagcccagcaacacaaaggtcgataagcgagtggagcccaagagctgcgacaagacccacacctgccccccctgccccgcccccgagctgctgggcggacccagcgtgttcctgttcccccctaaacccaaagacaccctgatgatcagcagaacccccgaggtgacctgtgtggtggtggacgtcagccacgaggaccccgaggtgaagttcaactggtacgtggacggcgttgaggtgcacaacgccaagaccaaacccagggaggagcagtacaatagcacctatagagtggtgagcgtgctgaccgtgctgcaccaggactggctgaatggcaaggagtacaagtgcaaggtgagcaacaaggccttacccgcccccatcgagaagaccatctccaaggccaagggccagcccagagagccccaggtgtacaccctgcctcccagcagagaggagatgactaagaaccaggtcagccttacctgcctggtgaagggcttttacccctcagacatcgccgtggaatgggagagcaacggccagcccgaaaataactacaagacaacccctcccgtgctggacagcgacgggagcttcttcttgtacagcaagctcaccgtggacaagagcagatggcagcagggcaacgtgttcagctgcagcgtgatgcatgaggccctgcacaaccactatacacagaagagcctgagcctgagccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 265 - protein, 448 aa, Homo sapiens; identical to SEQ ID NO: 255

SEQ ID NO: 266 - DNA, 938 bp, Homo sapiens
tcaagcttttggaccctcgtacagaagctaatacgactcactatagggaaataagagagaaaagaagagtaagaagaaatataagagccaccatggagacccccgcccagctgctgttcctgctgctgctgtggctgcccgacaccactggcgagatcgtgctgacccagtctcccggcacactgtctctgtctcccggcgagcgcgcaactctgagctgcagggcgagccagagcgtgggcagcagctacctggcctggtaccagcagaagccaggccaggcccctaggctgctgatctacggcgccttctcccgggcgaccgggatccccgacagattcagcggcagcggctccggcaccgacttcaccctgacaatcagcagactggagcccgaggacttcgccgtgtactactgccagcagtacggcagcagcccctggacatttggccagggcactaaggtggagatcaagagaaccgtggccgcccccagcgtgttcatcttcccccccagcgatgagcagctgaagagcggcaccgcctccgtggtctgcctgctgaacaacttctaccccagagaggccaaggttcagtggaaggtggacaacgccctgcagagcggcaacagccaggagtctgtgactgaacaggacagcaaggacagcacatacagcctgagcagcaccctcaccctgtccaaggccgattacgagaagcacaaggtgtacgcctgcgaggtgacccaccaaggcctgagcagccccgtgaccaagagcttcaacagaggcgagtgctgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 267 - protein, 235 aa, Homo sapiens
METPAQLLFLLLLWLPDTTGEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - DNA, 705 bp, Homo sapiens
atggagacccccgcccagctgctgttcctgctgctgctgtggctgcccgacaccactggcgagatcgtgctgacccagtctcccggcacactgtctctgtctcccggcgagcgcgcaactctgagctgcagggcgagccagagcgtgggcagcagctacctggcctggtaccagcagaagccaggccaggcccctaggctgctgatctacggcgccttctcccgggcgaccgggatccccgacagattcagcggcagcggctccggcaccgacttcaccctgacaatcagcagactggagcccgaggacttcgccgtgtactactgccagcagtacggcagcagcccctggacatttggccagggcactaaggtggagatcaagagaaccgtggccgcccccagcgtgttcatcttcccccccagcgatgagcagctgaagagcggcaccgcctccgtggtctgcctgctgaacaacttctaccccagagaggccaaggttcagtggaaggtggacaacgccctgcagagcggcaacagccaggagtctgtgactgaacaggacagcaaggacagcacatacagcctgagcagcaccctcaccctgtccaaggccgattacgagaagcacaaggtgtacgcctgcgaggtgacccaccaaggcctgagcagccccgtgaccaagagcttcaacagaggcgagtgc

SEQ ID NO: 269 - DNA, 997 bp, Homo sapiens
ggaaataagagagaaaagaagagtaagaagaaatataagagccaccatggagacccccgcccagctgctgttcctgctgctgctgtggctgcccgacaccactggcgagatcgtgctgacccagtctcccggcacactgtctctgtctcccggcgagcgcgcaactctgagctgcagggcgagccagagcgtgggcagcagctacctggcctggtaccagcagaagccaggccaggcccctaggctgctgatctacggcgccttctcccgggcgaccgggatccccgacagattcagcggcagcggctccggcaccgacttcaccctgacaatcagcagactggagcccgaggacttcgccgtgtactactgccagcagtacggcagcagcccctggacatttggccagggcactaaggtggagatcaagagaaccgtggccgcccccagcgtgttcatcttcccccccagcgatgagcagctgaagagcggcaccgcctccgtggtctgcctgctgaacaacttctaccccagagaggccaaggttcagtggaaggtggacaacgccctgcagagcggcaacagccaggagtctgtgactgaacaggacagcaaggacagcacatacagcctgagcagcaccctcaccctgtccaaggccgattacgagaagcacaaggtgtacgcctgcgaggtgacccaccaaggcctgagcagccccgtgaccaagagcttcaacagaggcgagtgctgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 270 - protein, 215 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - DNA, 938 bp, Homo sapiens
tcaagcttttggaccctcgtacagaagctaatacgactcactatagggaaataagagagaaaagaagagtaagaagaaatataagagccaccatggagacccccgcccagctgctcttcctgctgctgctgtggctgcctgacaccaccggcgagatcgtgctgacccaaagccccggtaccctgtccctgagccccggggagagagccaccctgagctgccgggccagccagagcgtgggctctagctatctggcctggtaccagcagaaacctggccaggcccccagactgctgatctatggcgcctttagcagagccaccggcattcccgacagattcagcggaagcggcagcggcaccgacttcaccctgacgatcagcagactggaacccgaggatttcgccgtgtactactgccagcagtacggcagcagcccctggaccttcgggcagggcaccaaggtggagattaagagaaccgtggccgcccccagcgtgttcatctttcccccctctgacgagcagctgaagagcggcaccgccagcgtggtgtgcctcctgaacaacttctacccccgggaggctaaggtgcagtggaaggtggacaacgccctgcagagcggcaactcacaggagagcgtgacagagcaggactcaaaggacagcacctacagcctgtccagcaccctgaccctgtcaaaggccgactacgagaagcacaaggtgtacgcctgcgaggtcacccaccagggactgagctcacccgttaccaagagcttcaaccgcggcgagtgctgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 272 - protein, 235 aa, Homo sapiens; identical to SEQ ID NO: 267

SEQ ID NO: 273 - DNA, 705 bp, Homo sapiens
atggagacccccgcccagctgctcttcctgctgctgctgtggctgcctgacaccaccggcgagatcgtgctgacccaaagccccggtaccctgtccctgagccccggggagagagccaccctgagctgccgggccagccagagcgtgggctctagctatctggcctggtaccagcagaaacctggccaggcccccagactgctgatctatggcgcctttagcagagccaccggcattcccgacagattcagcggaagcggcagcggcaccgacttcaccctgacgatcagcagactggaacccgaggatttcgccgtgtactactgccagcagtacggcagcagcccctggaccttcgggcagggcaccaaggtggagattaagagaaccgtggccgcccccagcgtgttcatctttcccccctctgacgagcagctgaagagcggcaccgccagcgtggtgtgcctcctgaacaacttctacccccgggaggctaaggtgcagtggaaggtggacaacgccctgcagagcggcaactcacaggagagcgtgacagagcaggactcaaaggacagcacctacagcctgtccagcaccctgaccctgtcaaaggccgactacgagaagcacaaggtgtacgcctgcgaggtcacccaccagggactgagctcacccgttaccaagagcttcaaccgcggcgagtgc

SEQ ID NO: 274 - DNA, 997 bp, Homo sapiens
ggaaataagagagaaaagaagagtaagaagaaatataagagccaccatggagacccccgcccagctgctcttcctgctgctgctgtggctgcctgacaccaccggcgagatcgtgctgacccaaagccccggtaccctgtccctgagccccggggagagagccaccctgagctgccgggccagccagagcgtgggctctagctatctggcctggtaccagcagaaacctggccaggcccccagactgctgatctatggcgcctttagcagagccaccggcattcccgacagattcagcggaagcggcagcggcaccgacttcaccctgacgatcagcagactggaacccgaggatttcgccgtgtactactgccagcagtacggcagcagcccctggaccttcgggcagggcaccaaggtggagattaagagaaccgtggccgcccccagcgtgttcatctttcccccctctgacgagcagctgaagagcggcaccgccagcgtggtgtgcctcctgaacaacttctacccccgggaggctaaggtgcagtggaaggtggacaacgccctgcagagcggcaactcacaggagagcgtgacagagcaggactcaaaggacagcacctacagcctgtccagcaccctgaccctgtcaaaggccgactacgagaagcacaaggtgtacgcctgcgaggtcacccaccagggactgagctcacccgttaccaagagcttcaaccgcggcgagtgctgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 275 - protein, 215 aa, Homo sapiens; identical to SEQ ID NO: 270

SEQ ID NO: 276 - DNA, 938 bp, Homo sapiens
tcaagcttttggaccctcgtacagaagctaatacgactcactatagggaaataagagagaaaagaagagtaagaagaaatataagagccaccatggagacacccgcccagttactgttcctcctgctgctgtggctgcctgacaccaccggcgagatcgtgctgacccagagccccggcaccctgagcctgtcccccggcgagagagccaccctgagctgccgcgcctcacagagcgtgggcagctcatacctggcctggtaccagcagaagcccggacaggctcccagactgctgatctacggcgccttttccagagccaccggcatccccgaccggttcagcggctctggaagcggcaccgacttcaccctgaccatcagccggttggagcccgaggatttcgctgtgtactactgtcagcaatacggaagcagcccctggaccttcggccagggcaccaaggtggagatcaagagaaccgtggccgcccccagcgtgttcatctttccccccagcgacgaacagctgaagagcggcaccgcctcagtggtctgcctgctcaataacttctacccccgggaggccaaagtgcagtggaaggtggacaacgccctgcagagcggcaacagccaggagagcgtgaccgagcaggacagcaaagactcaacatacagcctgagcagtaccctgactctgagcaaggccgactacgagaagcataaggtgtatgcctgcgaggtgacccaccagggcctgagcagccccgtgaccaagtcattcaaccgaggcgagtgctgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 277 - protein, 235 aa, Homo sapiens; identical to SEQ ID NO: 267

SEQ ID NO: 278 - DNA, 705 bp, Homo sapiens
atggagacacccgcccagttactgttcctcctgctgctgtggctgcctgacaccaccggcgagatcgtgctgacccagagccccggcaccctgagcctgtcccccggcgagagagccaccctgagctgccgcgcctcacagagcgtgggcagctcatacctggcctggtaccagcagaagcccggacaggctcccagactgctgatctacggcgccttttccagagccaccggcatccccgaccggttcagcggctctggaagcggcaccgacttcaccctgaccatcagccggttggagcccgaggatttcgctgtgtactactgtcagcaatacggaagcagcccctggaccttcggccagggcaccaaggtggagatcaagagaaccgtggccgcccccagcgtgttcatctttccccccagcgacgaacagctgaagagcggcaccgcctcagtggtctgcctgctcaataacttctacccccgggaggccaaagtgcagtggaaggtggacaacgccctgcagagcggcaacagccaggagagcgtgaccgagcaggacagcaaagactcaacatacagcctgagcagtaccctgactctgagcaaggccgactacgagaagcataaggtgtatgcctgcgaggtgacccaccagggcctgagcagccccgtgaccaagtcattcaaccgaggcgagtgc

SEQ ID NO: 279 - DNA, 997 bp, Homo sapiens
ggaaataagagagaaaagaagagtaagaagaaatataagagccaccatggagacacccgcccagttactgttcctcctgctgctgtggctgcctgacaccaccggcgagatcgtgctgacccagagccccggcaccctgagcctgtcccccggcgagagagccaccctgagctgccgcgcctcacagagcgtgggcagctcatacctggcctggtaccagcagaagcccggacaggctcccagactgctgatctacggcgccttttccagagccaccggcatccccgaccggttcagcggctctggaagcggcaccgacttcaccctgaccatcagccggttggagcccgaggatttcgctgtgtactactgtcagcaatacggaagcagcccctggaccttcggccagggcaccaaggtggagatcaagagaaccgtggccgcccccagcgtgttcatctttccccccagcgacgaacagctgaagagcggcaccgcctcagtggtctgcctgctcaataacttctacccccgggaggccaaagtgcagtggaaggtggacaacgccctgcagagcggcaacagccaggagagcgtgaccgagcaggacagcaaagactcaacatacagcctgagcagtaccctgactctgagcaaggccgactacgagaagcataaggtgtatgcctgcgaggtgacccaccagggcctgagcagccccgtgaccaagtcattcaaccgaggcgagtgctgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 280 - protein, 215 aa, Homo sapiens; identical to SEQ ID NO: 270

SEQ ID NO: 281 - DNA, 1658 bp, Homo sapiens
tcaagcttttggaccctcgtacagaagctaatacgactcactatagggaaataagagagaaaagaagagtaagaagaaatataagagccaccatggagacgcccgctcagctgctgttcctgctgctgctgtggctgcccgatacaactgggcaggtgcagttggtggaaagcggcggcggagtggtgcagccaggacggagcctgagactgagctgcgccgcctccggcttcactttctccagctatggcatgcattgggtgcggcaggcacccggcaagggcctggagtgggtggccgtcatctggtacgacggcagcaataagtactacgccgactccgtgaagggcaggttcaccatctccagggataacagcaagaatacactgtacctgcagatgaatagcctgagggctgaggacaccgccgtgtactactgtgcccgggaccccaggggcgccaccctgtactactactactacggaatggacgtgtggggccagggcaccacagtcacagtctctagcgcctccaccaagggccctagcgtgttccctctggcccccagcagcaagtctaccagcggcggcaccgccgccctgggctgcctggtgaaggactactttcccgagcccgtgaccgtcagctggaacagcggcgccctgaccagcggcgtccacaccttccccgccgtgctgcagagcagcggcctgtacagtctgagctccgtggtgacagttccctcaagcagcctgggcacacagacctatatctgcaacgtgaaccacaagcccagcaacacaaaggtggacaagagagtggagcccaagagctgcgacaagacccacacctgtcccccctgccccgccccggagctgctgggggggcccagcgtgttcctgttcccccccaaacccaaggacaccctgatgattagcaggactccagaagtcacctgcgtggtggtggacgtgtcgcacgaggaccccgaggtgaagttcaactggtatgtggacggcgtggaggtacacaatgccaagaccaagcctagggaggagcagtacaactccacctacagagtggtgagcgtgctgaccgtgctgcaccaggactggctgaacggcaaggagtacaagtgcaaagtgagcaataaggcactgcccgctcccatcgagaagaccatcagcaaggcaaaggggcagccccgcgagccccaggtgtacaccctgcccccctccagggaagagatgactaaaaatcaggtgagcctgacatgcctggtgaagggcttctacccaagcgatatcgccgtggagtgggagagcaacggccaacccgaaaacaactacaaaacgaccccaccggtcctggattcggatggcagcttcttcctgtatagcaagctgacagtggacaagtccagatggcagcagggaaacgtgttcagctgcagcgtcatgcacgaggccctgcacaaccactacacccagaagagcctcagcctgtcccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 282 - protein, 475 aa, Homo sapiens
METPAQLLFLLLLWLPDTTGQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 283 - DNA, 1425 bp, Homo sapiens
atggagacgcccgctcagctgctgttcctgctgctgctgtggctgcccgatacaactgggcaggtgcagttggtggaaagcggcggcggagtggtgcagccaggacggagcctgagactgagctgcgccgcctccggcttcactttctccagctatggcatgcattgggtgcggcaggcacccggcaagggcctggagtgggtggccgtcatctggtacgacggcagcaataagtactacgccgactccgtgaagggcaggttcaccatctccagggataacagcaagaatacactgtacctgcagatgaatagcctgagggctgaggacaccgccgtgtactactgtgcccgggaccccaggggcgccaccctgtactactactactacggaatggacgtgtggggccagggcaccacagtcacagtctctagcgcctccaccaagggccctagcgtgttccctctggcccccagcagcaagtctaccagcggcggcaccgccgccctgggctgcctggtgaaggactactttcccgagcccgtgaccgtcagctggaacagcggcgccctgaccagcggcgtccacaccttccccgccgtgctgcagagcagcggcctgtacagtctgagctccgtggtgacagttccctcaagcagcctgggcacacagacctatatctgcaacgtgaaccacaagcccagcaacacaaaggtggacaagagagtggagcccaagagctgcgacaagacccacacctgtcccccctgccccgccccggagctgctgggggggcccagcgtgttcctgttcccccccaaacccaaggacaccctgatgattagcaggactccagaagtcacctgcgtggtggtggacgtgtcgcacgaggaccccgaggtgaagttcaactggtatgtggacggcgtggaggtacacaatgccaagaccaagcctagggaggagcagtacaactccacctacagagtggtgagcgtgctgaccgtgctgcaccaggactggctgaacggcaaggagtacaagtgcaaagtgagcaataaggcactgcccgctcccatcgagaagaccatcagcaaggcaaaggggcagccccgcgagccccaggtgtacaccctgcccccctccagggaagagatgactaaaaatcaggtgagcctgacatgcctggtgaagggcttctacccaagcgatatcgccgtggagtgggagagcaacggccaacccgaaaacaactacaaaacgaccccaccggtcctggattcggatggcagcttcttcctgtatagcaagctgacagtggacaagtccagatggcagcagggaaacgtgttcagctgcagcgtcatgcacgaggccctgcacaaccactacacccagaagagcctcagcctgtcccccggcaag

SEQ ID NO: 284 - DNA, 1717 bp, Homo sapiens
ggaaataagagagaaaagaagagtaagaagaaatataagagccaccatggagacgcccgctcagctgctgttcctgctgctgctgtggctgcccgatacaactgggcaggtgcagttggtggaaagcggcggcggagtggtgcagccaggacggagcctgagactgagctgcgccgcctccggcttcactttctccagctatggcatgcattgggtgcggcaggcacccggcaagggcctggagtgggtggccgtcatctggtacgacggcagcaataagtactacgccgactccgtgaagggcaggttcaccatctccagggataacagcaagaatacactgtacctgcagatgaatagcctgagggctgaggacaccgccgtgtactactgtgcccgggaccccaggggcgccaccctgtactactactactacggaatggacgtgtggggccagggcaccacagtcacagtctctagcgcctccaccaagggccctagcgtgttccctctggcccccagcagcaagtctaccagcggcggcaccgccgccctgggctgcctggtgaaggactactttcccgagcccgtgaccgtcagctggaacagcggcgccctgaccagcggcgtccacaccttccccgccgtgctgcagagcagcggcctgtacagtctgagctccgtggtgacagttccctcaagcagcctgggcacacagacctatatctgcaacgtgaaccacaagcccagcaacacaaaggtggacaagagagtggagcccaagagctgcgacaagacccacacctgtcccccctgccccgccccggagctgctgggggggcccagcgtgttcctgttcccccccaaacccaaggacaccctgatgattagcaggactccagaagtcacctgcgtggtggtggacgtgtcgcacgaggaccccgaggtgaagttcaactggtatgtggacggcgtggaggtacacaatgccaagaccaagcctagggaggagcagtacaactccacctacagagtggtgagcgtgctgaccgtgctgcaccaggactggctgaacggcaaggagtacaagtgcaaagtgagcaataaggcactgcccgctcccatcgagaagaccatcagcaaggcaaaggggcagccccgcgagccccaggtgtacaccctgcccccctccagggaagagatgactaaaaatcaggtgagcctgacatgcctggtgaagggcttctacccaagcgatatcgccgtggagtgggagagcaacggccaacccgaaaacaactacaaaacgaccccaccggtcctggattcggatggcagcttcttcctgtatagcaagctgacagtggacaagtccagatggcagcagggaaacgtgttcagctgcagcgtcatgcacgaggccctgcacaaccactacacccagaagagcctcagcctgtcccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 285 - protein, 455 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - DNA, 1658 bp, Homo sapiens
tcaagcttttggaccctcgtacagaagctaatacgactcactatagggaaataagagagaaaagaagagtaagaagaaatataagagccaccatggaaaccccagcccagctcctgttcctgctgctgctgtggctgcccgacaccaccggccaggtgcagctggtggagtcaggcggcggcgtggtgcaaccaggccgcagcctgaggctcagctgcgccgcctccggcttcaccttcagttcctacggcatgcactgggtgagacaggcccccggcaagggcctggagtgggtggccgtgatctggtacgacggcagcaacaaatactatgctgacagcgtgaagggccggttcaccatctccagggacaatagcaaaaacaccctgtacctgcaaatgaatagcctgagggccgaggacaccgctgtgtactactgcgccagggaccctaggggcgccaccctttactactactactacggcatggacgtgtggggccagggcactaccgtcaccgtgtccagcgccagcaccaagggccccagcgtgttcccactggcacccagctctaagagcaccagcggcggcaccgccgccctgggctgcctggtgaaggactacttccccgagcccgtgaccgtgagctggaacagcggcgccctgacttccggcgtccacacattccccgccgtgctgcagagcagcgggctgtacagcctgtcatccgtggtgaccgtgcctagcagcagtctgggcactcagacctatatttgcaacgtgaaccacaagccctccaacaccaaagtggataagagagtggagcctaagagctgtgataagacccacacctgccctccttgcccagctccggagctgctgggcggccccagcgtgttcctgttccccccgaagcccaaggacacactgatgatcagcagaacccccgaagttacttgcgtggtggtggacgtcagccacgaagaccccgaggtcaagttcaattggtatgtggacggcgtggaggtgcacaacgccaagacaaagccgagagaggagcagtacaattccacctatagagtggtgagcgtgctgacagtcctccaccaggactggctgaacggcaaggagtacaagtgcaaggtcagcaacaaagccctgcctgctcccatcgagaagaccatctccaaggctaaaggccagcctagagagccccaggtgtacaccctgcccccctccagagaggagatgaccaagaaccaggtgagcctgacatgcctggtgaagggcttctaccccagcgacatcgctgtggagtgggagagcaatggccagcccgagaataactacaagaccaccccccccgtgctcgacagcgacggcagcttcttcctgtacagcaagctgaccgtggataagagcagatggcaacagggtaatgtgtttagctgctccgtgatgcacgaggccctccacaaccattacacccagaaaagcctgtccctgagccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 287 - protein, 475 aa, Homo sapiens; identical to SEQ ID NO: 282

SEQ ID NO: 288 - DNA, 1425 bp, Homo sapiens
atggaaaccccagcccagctcctgttcctgctgctgctgtggctgcccgacaccaccggccaggtgcagctggtggagtcaggcggcggcgtggtgcaaccaggccgcagcctgaggctcagctgcgccgcctccggcttcaccttcagttcctacggcatgcactgggtgagacaggcccccggcaagggcctggagtgggtggccgtgatctggtacgacggcagcaacaaatactatgctgacagcgtgaagggccggttcaccatctccagggacaatagcaaaaacaccctgtacctgcaaatgaatagcctgagggccgaggacaccgctgtgtactactgcgccagggaccctaggggcgccaccctttactactactactacggcatggacgtgtggggccagggcactaccgtcaccgtgtccagcgccagcaccaagggccccagcgtgttcccactggcacccagctctaagagcaccagcggcggcaccgccgccctgggctgcctggtgaaggactacttccccgagcccgtgaccgtgagctggaacagcggcgccctgacttccggcgtccacacattccccgccgtgctgcagagcagcgggctgtacagcctgtcatccgtggtgaccgtgcctagcagcagtctgggcactcagacctatatttgcaacgtgaaccacaagccctccaacaccaaagtggataagagagtggagcctaagagctgtgataagacccacacctgccctccttgcccagctccggagctgctgggcggccccagcgtgttcctgttccccccgaagcccaaggacacactgatgatcagcagaacccccgaagttacttgcgtggtggtggacgtcagccacgaagaccccgaggtcaagttcaattggtatgtggacggcgtggaggtgcacaacgccaagacaaagccgagagaggagcagtacaattccacctatagagtggtgagcgtgctgacagtcctccaccaggactggctgaacggcaaggagtacaagtgcaaggtcagcaacaaagccctgcctgctcccatcgagaagaccatctccaaggctaaaggccagcctagagagccccaggtgtacaccctgcccccctccagagaggagatgaccaagaaccaggtgagcctgacatgcctggtgaagggcttctaccccagcgacatcgctgtggagtgggagagcaatggccagcccgagaataactacaagaccaccccccccgtgctcgacagcgacggcagcttcttcctgtacagcaagctgaccgtggataagagcagatggcaacagggtaatgtgtttagctgctccgtgatgcacgaggccctccacaaccattacacccagaaaagcctgtccctgagccccggcaag

SEQ ID NO: 289 - DNA, 1717 bp, Homo sapiens
ggaaataagagagaaaagaagagtaagaagaaatataagagccaccatggaaaccccagcccagctcctgttcctgctgctgctgtggctgcccgacaccaccggccaggtgcagctggtggagtcaggcggcggcgtggtgcaaccaggccgcagcctgaggctcagctgcgccgcctccggcttcaccttcagttcctacggcatgcactgggtgagacaggcccccggcaagggcctggagtgggtggccgtgatctggtacgacggcagcaacaaatactatgctgacagcgtgaagggccggttcaccatctccagggacaatagcaaaaacaccctgtacctgcaaatgaatagcctgagggccgaggacaccgctgtgtactactgcgccagggaccctaggggcgccaccctttactactactactacggcatggacgtgtggggccagggcactaccgtcaccgtgtccagcgccagcaccaagggccccagcgtgttcccactggcacccagctctaagagcaccagcggcggcaccgccgccctgggctgcctggtgaaggactacttccccgagcccgtgaccgtgagctggaacagcggcgccctgacttccggcgtccacacattccccgccgtgctgcagagcagcgggctgtacagcctgtcatccgtggtgaccgtgcctagcagcagtctgggcactcagacctatatttgcaacgtgaaccacaagccctccaacaccaaagtggataagagagtggagcctaagagctgtgataagacccacacctgccctccttgcccagctccggagctgctgggcggccccagcgtgttcctgttccccccgaagcccaaggacacactgatgatcagcagaacccccgaagttacttgcgtggtggtggacgtcagccacgaagaccccgaggtcaagttcaattggtatgtggacggcgtggaggtgcacaacgccaagacaaagccgagagaggagcagtacaattccacctatagagtggtgagcgtgctgacagtcctccaccaggactggctgaacggcaaggagtacaagtgcaaggtcagcaacaaagccctgcctgctcccatcgagaagaccatctccaaggctaaaggccagcctagagagccccaggtgtacaccctgcccccctccagagaggagatgaccaagaaccaggtgagcctgacatgcctggtgaagggcttctaccccagcgacatcgctgtggagtgggagagcaatggccagcccgagaataactacaagaccaccccccccgtgctcgacagcgacggcagcttcttcctgtacagcaagctgaccgtggataagagcagatggcaacagggtaatgtgtttagctgctccgtgatgcacgaggccctccacaaccattacacccagaaaagcctgtccctgagccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 290 - protein, 455 aa, Homo sapiens; identical to SEQ ID NO: 285

SEQ ID NO: 291 - DNA, 1658 bp, Homo sapiens
tcaagcttttggaccctcgtacagaagctaatacgactcactatagggaaataagagagaaaagaagagtaagaagaaatataagagccaccatggaaacccccgctcagctgctgtttctgcttctgctgtggctccctgacactaccggacaggtgcagctcgtggagagcggcggcggcgtggtgcagcccggcaggagcctgagactgagctgcgccgcctctggcttcacattcagcagctacggcatgcactgggtgcggcaggcccccggcaagggactggagtgggtggccgtgatctggtacgacggatcaaacaagtactacgccgactcggtgaagggcaggttcaccatcagccgcgacaacagcaagaacaccctgtacctgcagatgaacagcctgagagccgaggacaccgcggtgtactactgcgcccgggatcccaggggcgccaccctgtactactactactacggcatggacgtgtggggccagggcaccacagtgacagtgagcagcgccagcaccaagggccctagcgtgtttcctctggccccaagctcaaagagcaccagtggcggaaccgccgccctgggctgcctggtgaaggactactttcccgagcccgtgaccgtgtcctggaacagcggcgccctgacaagcggagtgcacaccttcccagcggttctgcagtcatcagggctgtacagcctgagctccgtggtgaccgtgcccagctccagcctgggtacccagacctacatctgcaatgtgaaccacaagcccagcaacaccaaggtggacaagcgcgtcgagcctaaaagctgcgacaagacccacacctgccccccctgccccgcccccgagctgctggggggccccagcgtcttcctattcccccctaagcccaaggacaccctcatgatcagcagaacccccgaggtgacctgcgtggtggtggacgtgagccatgaggaccccgaggtgaagttcaattggtacgtggacggggtggaagtgcataacgccaagaccaagccaagagaggagcagtacaacagcacctaccgcgtggtgtccgtgctgaccgtgttgcatcaggattggcttaacggcaaggagtataagtgtaaagtgtctaacaaggctctccccgcccccattgagaagaccatctccaaggccaagggccagcccagagagccccaggtgtatacactgccccccagcagagaggagatgaccaagaaccaggtgagcctgacctgtctggtgaagggcttctaccctagcgacattgccgtggagtgggagtccaacggccagcccgaaaataactacaagaccactcctcccgtgcttgacagcgacgggagcttcttcctgtacagcaagctgaccgtggacaagagcaggtggcagcagggcaacgtgttctcttgcagcgtgatgcacgaggcgctccacaatcactacacccagaagagcctgtccctgtctcccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 292 - protein, 475 aa, Homo sapiens; identical to SEQ ID NO: 282

SEQ ID NO: 293 - DNA, 1425 bp, Homo sapiens
atggaaacccccgctcagctgctgtttctgcttctgctgtggctccctgacactaccggacaggtgcagctcgtggagagcggcggcggcgtggtgcagcccggcaggagcctgagactgagctgcgccgcctctggcttcacattcagcagctacggcatgcactgggtgcggcaggcccccggcaagggactggagtgggtggccgtgatctggtacgacggatcaaacaagtactacgccgactcggtgaagggcaggttcaccatcagccgcgacaacagcaagaacaccctgtacctgcagatgaacagcctgagagccgaggacaccgcggtgtactactgcgcccgggatcccaggggcgccaccctgtactactactactacggcatggacgtgtggggccagggcaccacagtgacagtgagcagcgccagcaccaagggccctagcgtgtttcctctggccccaagctcaaagagcaccagtggcggaaccgccgccctgggctgcctggtgaaggactactttcccgagcccgtgaccgtgtcctggaacagcggcgccctgacaagcggagtgcacaccttcccagcggttctgcagtcatcagggctgtacagcctgagctccgtggtgaccgtgcccagctccagcctgggtacccagacctacatctgcaatgtgaaccacaagcccagcaacaccaaggtggacaagcgcgtcgagcctaaaagctgcgacaagacccacacctgccccccctgccccgcccccgagctgctggggggccccagcgtcttcctattcccccctaagcccaaggacaccctcatgatcagcagaacccccgaggtgacctgcgtggtggtggacgtgagccatgaggaccccgaggtgaagttcaattggtacgtggacggggtggaagtgcataacgccaagaccaagccaagagaggagcagtacaacagcacctaccgcgtggtgtccgtgctgaccgtgttgcatcaggattggcttaacggcaaggagtataagtgtaaagtgtctaacaaggctctccccgcccccattgagaagaccatctccaaggccaagggccagcccagagagccccaggtgtatacactgccccccagcagagaggagatgaccaagaaccaggtgagcctgacctgtctggtgaagggcttctaccctagcgacattgccgtggagtgggagtccaacggccagcccgaaaataactacaagaccactcctcccgtgcttgacagcgacgggagcttcttcctgtacagcaagctgaccgtggacaagagcaggtggcagcagggcaacgtgttctcttgcagcgtgatgcacgaggcgctccacaatcactacacccagaagagcctgtccctgtctcccggcaag

SEQ ID NO: 294 - DNA, 1717 bp, Homo sapiens
ggaaataagagagaaaagaagagtaagaagaaatataagagccaccatggaaacccccgctcagctgctgtttctgcttctgctgtggctccctgacactaccggacaggtgcagctcgtggagagcggcggcggcgtggtgcagcccggcaggagcctgagactgagctgcgccgcctctggcttcacattcagcagctacggcatgcactgggtgcggcaggcccccggcaagggactggagtgggtggccgtgatctggtacgacggatcaaacaagtactacgccgactcggtgaagggcaggttcaccatcagccgcgacaacagcaagaacaccctgtacctgcagatgaacagcctgagagccgaggacaccgcggtgtactactgcgcccgggatcccaggggcgccaccctgtactactactactacggcatggacgtgtggggccagggcaccacagtgacagtgagcagcgccagcaccaagggccctagcgtgtttcctctggccccaagctcaaagagcaccagtggcggaaccgccgccctgggctgcctggtgaaggactactttcccgagcccgtgaccgtgtcctggaacagcggcgccctgacaagcggagtgcacaccttcccagcggttctgcagtcatcagggctgtacagcctgagctccgtggtgaccgtgcccagctccagcctgggtacccagacctacatctgcaatgtgaaccacaagcccagcaacaccaaggtggacaagcgcgtcgagcctaaaagctgcgacaagacccacacctgccccccctgccccgcccccgagctgctggggggccccagcgtcttcctattcccccctaagcccaaggacaccctcatgatcagcagaacccccgaggtgacctgcgtggtggtggacgtgagccatgaggaccccgaggtgaagttcaattggtacgtggacggggtggaagtgcataacgccaagaccaagccaagagaggagcagtacaacagcacctaccgcgtggtgtccgtgctgaccgtgttgcatcaggattggcttaacggcaaggagtataagtgtaaagtgtctaacaaggctctccccgcccccattgagaagaccatctccaaggccaagggccagcccagagagccccaggtgtatacactgccccccagcagagaggagatgaccaagaaccaggtgagcctgacctgtctggtgaagggcttctaccctagcgacattgccgtggagtgggagtccaacggccagcccgaaaataactacaagaccactcctcccgtgcttgacagcgacgggagcttcttcctgtacagcaagctgaccgtggacaagagcaggtggcagcagggcaacgtgttctcttgcagcgtgatgcacgaggcgctccacaatcactacacccagaagagcctgtccctgtctcccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 295 - protein, 455 aa, Homo sapiens; identical to SEQ ID NO: 285

SEQ ID NO: 296 - DNA, 1646 bp, Homo sapiens
tcaagcttttggaccctcgtacagaagctaatacgactcactatagggaaataagagagaaaagaagagtaagaagaaatataagagccaccatggagacccccgcccagctgctgttcctgctgctgctgtggctgcccgataccaccggccaggtccagctcgtggagagcggcggcggcgtggttcagcccggcagaagcctgagactgagctgcgccgccagcggcttcaccttctcaagctacggcatgcactgggtgagacaggccccgggcaagggcctcgaatgggtggccgtgatctggtacgacggaagcaacaagtactacgccgatagtgtgaagggcaggttcactatcagccgagataacagcaaaaacaccctgtatctgcagatgaactcactgcgtgccgaggataccgctgtgtactattgcgccagggaccctagaggagccacgctgtactactactactacggcatggacgtttggggccagggaaccaccgtcaccgtgagcagcgcctccaccaaaggccccagcgtgttccccctggccccttgctccaggagtaccagcgagagcaccgccgccctgggctgcctggtgaaggactatttccccgagcccgtgaccgtgtcctggaactctggcgccctgaccagcggggtgcacaccttccccgccgtcctgcagagtagcggactgtacagcctgagcagcgtagtgactgtgccctccagcaacttcggcactcagacctatacttgtaacgtcgaccacaagccttccaacacaaaggtggacaaaaccgtggagaggaagtgttgcgttgagtgccccccctgtccggccccgcccgtggcggggcctagcgtgttcctcttccctcccaagccaaaggacaccctgatgatcagcaggacccccgaggtgacctgcgtggtggtggatgtgagccatgaggaccccgaggtgcagtttaactggtacgtggacggagtggaggtgcataacgccaagaccaagcctagggaggagcagttcaacagcaccttcagagtggttagcgtgctgaccgtggtgcaccaggattggctgaacgggaaggagtacaagtgcaaggtgagcaacaagggcctgcccgccccaatcgagaagaccatctcaaagaccaaggggcagcctagagagccccaggtgtacaccctgccccctagcagagaggagatgaccaagaaccaggtgagcctcacctgcctcgtgaagggcttctaccccagcgacatcgctgtggagtgggagtccaacggccagcccgagaacaactacaaaaccacacccccaatgctggactccgacggcagcttcttcctgtacagcaagctgacagtggacaaaagcagatggcagcagggcaacgtgttcagctgctctgtgatgcacgaggccctgcacaatcattatactcagaagagcctgagcctgtcccccggaaagtgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 297 - protein, 471 aa, Homo sapiens; identical to SEQ ID NO: 235

SEQ ID NO: 298 - DNA, 1413 bp, Homo sapiens
atggagacccccgcccagctgctgttcctgctgctgctgtggctgcccgataccaccggccaggtccagctcgtggagagcggcggcggcgtggttcagcccggcagaagcctgagactgagctgcgccgccagcggcttcaccttctcaagctacggcatgcactgggtgagacaggccccgggcaagggcctcgaatgggtggccgtgatctggtacgacggaagcaacaagtactacgccgatagtgtgaagggcaggttcactatcagccgagataacagcaaaaacaccctgtatctgcagatgaactcactgcgtgccgaggataccgctgtgtactattgcgccagggaccctagaggagccacgctgtactactactactacggcatggacgtttggggccagggaaccaccgtcaccgtgagcagcgcctccaccaaaggccccagcgtgttccccctggccccttgctccaggagtaccagcgagagcaccgccgccctgggctgcctggtgaaggactatttccccgagcccgtgaccgtgtcctggaactctggcgccctgaccagcggggtgcacaccttccccgccgtcctgcagagtagcggactgtacagcctgagcagcgtagtgactgtgccctccagcaacttcggcactcagacctatacttgtaacgtcgaccacaagccttccaacacaaaggtggacaaaaccgtggagaggaagtgttgcgttgagtgccccccctgtccggccccgcccgtggcggggcctagcgtgttcctcttccctcccaagccaaaggacaccctgatgatcagcaggacccccgaggtgacctgcgtggtggtggatgtgagccatgaggaccccgaggtgcagtttaactggtacgtggacggagtggaggtgcataacgccaagaccaagcctagggaggagcagttcaacagcaccttcagagtggttagcgtgctgaccgtggtgcaccaggattggctgaacgggaaggagtacaagtgcaaggtgagcaacaagggcctgcccgccccaatcgagaagaccatctcaaagaccaaggggcagcctagagagccccaggtgtacaccctgccccctagcagagaggagatgaccaagaaccaggtgagcctcacctgcctcgtgaagggcttctaccccagcgacatcgctgtggagtgggagtccaacggccagcccgagaacaactacaaaaccacacccccaatgctggactccgacggcagcttcttcctgtacagcaagctgacagtggacaaaagcagatggcagcagggcaacgtgttcagctgctctgtgatgcacgaggccctgcacaatcattatactcagaagagcctgagcctgtcccccggaaag

SEQ ID NO: 299 - DNA, 1705 bp, Homo sapiens
ggaaataagagagaaaagaagagtaagaagaaatataagagccaccatggagacccccgcccagctgctgttcctgctgctgctgtggctgcccgataccaccggccaggtccagctcgtggagagcggcggcggcgtggttcagcccggcagaagcctgagactgagctgcgccgccagcggcttcaccttctcaagctacggcatgcactgggtgagacaggccccgggcaagggcctcgaatgggtggccgtgatctggtacgacggaagcaacaagtactacgccgatagtgtgaagggcaggttcactatcagccgagataacagcaaaaacaccctgtatctgcagatgaactcactgcgtgccgaggataccgctgtgtactattgcgccagggaccctagaggagccacgctgtactactactactacggcatggacgtttggggccagggaaccaccgtcaccgtgagcagcgcctccaccaaaggccccagcgtgttccccctggccccttgctccaggagtaccagcgagagcaccgccgccctgggctgcctggtgaaggactatttccccgagcccgtgaccgtgtcctggaactctggcgccctgaccagcggggtgcacaccttccccgccgtcctgcagagtagcggactgtacagcctgagcagcgtagtgactgtgccctccagcaacttcggcactcagacctatacttgtaacgtcgaccacaagccttccaacacaaaggtggacaaaaccgtggagaggaagtgttgcgttgagtgccccccctgtccggccccgcccgtggcggggcctagcgtgttcctcttccctcccaagccaaaggacaccctgatgatcagcaggacccccgaggtgacctgcgtggtggtggatgtgagccatgaggaccccgaggtgcagtttaactggtacgtggacggagtggaggtgcataacgccaagaccaagcctagggaggagcagttcaacagcaccttcagagtggttagcgtgctgaccgtggtgcaccaggattggctgaacgggaaggagtacaagtgcaaggtgagcaacaagggcctgcccgccccaatcgagaagaccatctcaaagaccaaggggcagcctagagagccccaggtgtacaccctgccccctagcagagaggagatgaccaagaaccaggtgagcctcacctgcctcgtgaagggcttctaccccagcgacatcgctgtggagtgggagtccaacggccagcccgagaacaactacaaaaccacacccccaatgctggactccgacggcagcttcttcctgtacagcaagctgacagtggacaaaagcagatggcagcagggcaacgtgttcagctgctctgtgatgcacgaggccctgcacaatcattatactcagaagagcctgagcctgtcccccggaaagtgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 300 - protein, 451 aa, Homo sapiens; identical to SEQ ID NO: 21

SEQ ID NO: 301 - DNA, 1646 bp, Homo sapiens
tcaagcttttggaccctcgtacagaagctaatacgactcactatagggaaataagagagaaaagaagagtaagaagaaatataagagccaccatggagaccccagcccagctgctgttcctgcttctgctgtggctgccagataccaccggccaggtgcagctggttgagtcaggcggcggcgtggtgcagcccggcagaagcctgcggctgagctgcgccgccagcggcttcaccttcagcagctacggcatgcactgggtgagacaggcccctggcaaggggctggagtgggtggccgtgatctggtatgacggaagtaacaagtactacgccgacagcgtgaagggcagattcacgatcagcagagacaactccaagaacacgctgtacttgcagatgaacagcctgagagccgaggacacagccgtgtattactgcgccagagaccccagaggcgccaccctgtactactactactacggcatggacgtgtggggccagggcaccaccgtgaccgtgagcagtgccagcaccaagggccccagcgtgttccccctggccccctgcagtcggagcaccagcgaaagcaccgccgccctgggctgcctggtgaaagactatttccccgagcctgtgacagtgagctggaacagcggggccctcacctctggcgtgcacacgttcccagccgtgctgcagagcagcgggctgtacagcctgtcctctgtggtgaccgtgcccagcagcaatttcggcacccagacctacacgtgcaatgtggatcacaagcccagcaacacaaaagtggacaaaaccgtggagagaaagtgttgcgtggagtgccccccttgccccgccccccccgtggctggcccgtcagtgttcctgttccctcctaagcccaaggacaccctcatgatcagcagaacccccgaggtgacctgcgtggtcgtggacgtgagtcacgaggaccccgaggtccagttcaactggtacgtggatggagtggaggtgcacaacgccaagaccaagccccgggaggagcagtttaatagcacctttagggtggtgagcgtgctgaccgttgtacaccaggactggctgaacggcaaagagtacaaatgcaaggtttcaaataagggcctgcccgccccaattgagaagacaatcagcaagactaagggccagcctagggaaccccaggtctacactctgcccccctctcgggaggagatgaccaagaaccaggtgagcctgacctgcctggtgaagggcttctacccttctgacatcgctgtggagtgggagagcaatggacaacccgagaacaactacaagaccacccccccaatgctggactctgatggcagcttctttctgtacagcaagctgacagtggataaaagcaggtggcagcagggtaatgtgttcagctgcagcgtgatgcacgaggcactgcacaatcactacacccagaagagcctgtccctgtcccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 302 - protein, 471 aa, Homo sapiens; identical to SEQ ID NO: 235

SEQ ID NO: 303 - DNA, 1413 bp, Homo sapiens
atggagaccccagcccagctgctgttcctgcttctgctgtggctgccagataccaccggccaggtgcagctggttgagtcaggcggcggcgtggtgcagcccggcagaagcctgcggctgagctgcgccgccagcggcttcaccttcagcagctacggcatgcactgggtgagacaggcccctggcaaggggctggagtgggtggccgtgatctggtatgacggaagtaacaagtactacgccgacagcgtgaagggcagattcacgatcagcagagacaactccaagaacacgctgtacttgcagatgaacagcctgagagccgaggacacagccgtgtattactgcgccagagaccccagaggcgccaccctgtactactactactacggcatggacgtgtggggccagggcaccaccgtgaccgtgagcagtgccagcaccaagggccccagcgtgttccccctggccccctgcagtcggagcaccagcgaaagcaccgccgccctgggctgcctggtgaaagactatttccccgagcctgtgacagtgagctggaacagcggggccctcacctctggcgtgcacacgttcccagccgtgctgcagagcagcgggctgtacagcctgtcctctgtggtgaccgtgcccagcagcaatttcggcacccagacctacacgtgcaatgtggatcacaagcccagcaacacaaaagtggacaaaaccgtggagagaaagtgttgcgtggagtgccccccttgccccgccccccccgtggctggcccgtcagtgttcctgttccctcctaagcccaaggacaccctcatgatcagcagaacccccgaggtgacctgcgtggtcgtggacgtgagtcacgaggaccccgaggtccagttcaactggtacgtggatggagtggaggtgcacaacgccaagaccaagccccgggaggagcagtttaatagcacctttagggtggtgagcgtgctgaccgttgtacaccaggactggctgaacggcaaagagtacaaatgcaaggtttcaaataagggcctgcccgccccaattgagaagacaatcagcaagactaagggccagcctagggaaccccaggtctacactctgcccccctctcgggaggagatgaccaagaaccaggtgagcctgacctgcctggtgaagggcttctacccttctgacatcgctgtggagtgggagagcaatggacaacccgagaacaactacaagaccacccccccaatgctggactctgatggcagcttctttctgtacagcaagctgacagtggataaaagcaggtggcagcagggtaatgtgttcagctgcagcgtgatgcacgaggcactgcacaatcactacacccagaagagcctgtccctgtcccccggcaag

SEQ ID NO: 304 - DNA, 1705 bp, Homo sapiens
ggaaataagagagaaaagaagagtaagaagaaatataagagccaccatggagaccccagcccagctgctgttcctgcttctgctgtggctgccagataccaccggccaggtgcagctggttgagtcaggcggcggcgtggtgcagcccggcagaagcctgcggctgagctgcgccgccagcggcttcaccttcagcagctacggcatgcactgggtgagacaggcccctggcaaggggctggagtgggtggccgtgatctggtatgacggaagtaacaagtactacgccgacagcgtgaagggcagattcacgatcagcagagacaactccaagaacacgctgtacttgcagatgaacagcctgagagccgaggacacagccgtgtattactgcgccagagaccccagaggcgccaccctgtactactactactacggcatggacgtgtggggccagggcaccaccgtgaccgtgagcagtgccagcaccaagggccccagcgtgttccccctggccccctgcagtcggagcaccagcgaaagcaccgccgccctgggctgcctggtgaaagactatttccccgagcctgtgacagtgagctggaacagcggggccctcacctctggcgtgcacacgttcccagccgtgctgcagagcagcgggctgtacagcctgtcctctgtggtgaccgtgcccagcagcaatttcggcacccagacctacacgtgcaatgtggatcacaagcccagcaacacaaaagtggacaaaaccgtggagagaaagtgttgcgtggagtgccccccttgccccgccccccccgtggctggcccgtcagtgttcctgttccctcctaagcccaaggacaccctcatgatcagcagaacccccgaggtgacctgcgtggtcgtggacgtgagtcacgaggaccccgaggtccagttcaactggtacgtggatggagtggaggtgcacaacgccaagaccaagccccgggaggagcagtttaatagcacctttagggtggtgagcgtgctgaccgttgtacaccaggactggctgaacggcaaagagtacaaatgcaaggtttcaaataagggcctgcccgccccaattgagaagacaatcagcaagactaagggccagcctagggaaccccaggtctacactctgcccccctctcgggaggagatgaccaagaaccaggtgagcctgacctgcctggtgaagggcttctacccttctgacatcgctgtggagtgggagagcaatggacaacccgagaacaactacaagaccacccccccaatgctggactctgatggcagcttctttctgtacagcaagctgacagtggataaaagcaggtggcagcagggtaatgtgttcagctgcagcgtgatgcacgaggcactgcacaatcactacacccagaagagcctgtccctgtcccccggcaagtgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 305 - protein, 451 aa, Homo sapiens; identical to SEQ ID NO: 21

SEQ ID NO: 306 - DNA, 1646 bp, Homo sapiens
tcaagcttttggaccctcgtacagaagctaatacgactcactatagggaaataagagagaaaagaagagtaagaagaaatataagagccaccatggaaacccccgctcagctgctgttcctgttgctgctgtggctgcccgacactaccgggcaagtgcagctggtggagagcggcggcggcgtggtgcagcccggcagaagccttaggctgagctgcgccgccagtggcttcaccttcagcagctacggcatgcactgggtgagacaggcccccggcaagggcctggagtgggtggctgtgatctggtacgacggcagcaacaagtactacgcagacagcgtgaagggaagattcaccatcagccgcgacaacagcaagaacaccctgtacctgcagatgaacagcctgagagctgaggacaccgcagtgtactactgcgccagagaccccaggggcgccaccctgtactactattattacggcatggatgtgtggggccagggcaccaccgtgaccgtgtccagcgccagcaccaagggcccaagcgtgttcccacttgccccatgcagcagaagcaccagcgagtcaaccgccgccctggggtgcttggtgaaagactacttccccgagcccgtgaccgtgagctggaacagcggagcactgaccagcggtgtgcacaccttccccgccgtgctgcagagcagcggactgtactccctgtccagcgtggtcaccgtgcccagcagcaacttcgggactcaaacttacacttgcaacgtggaccacaaaccctccaataccaaggtggataaaaccgtggagcggaagtgttgcgtggagtgccctccctgccccgcgcctcccgtggccggccccagcgtgttcctgttcccacccaagcctaaggatacactgatgatcagcagaacccccgaggtcacttgcgtggtggtggacgtgagccacgaggacccggaggtgcagtttaactggtacgttgacggggtggaggtgcacaacgctaagaccaagcccagagaagaacagttcaacagcaccttcagggtagtgagcgtgcttaccgtggtgcaccaggactggctgaacggcaaggagtacaagtgcaaggtgagcaacaagggattgcccgccccgatcgagaagacaatctctaagacgaagggccagcccagagagccccaggtgtacaccctgccccccagcagagaggagatgaccaagaatcaggtgagcctgacatgcttggtgaagggcttctaccccagcgacatcgccgtcgagtgggagagcaacggacagcccgagaacaactacaaaaccaccccacctatgctggacagcgacggcagcttcttcctgtactccaaactgaccgtggataagagcagatggcagcagggcaacgtgtttagctgcagcgtgatgcacgaggccctgcacaaccactacacccagaagagcctgtctttgagccccgggaagtgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 307 - protein, 471 aa, Homo sapiens; identical to SEQ ID NO: 235

SEQ ID NO: 308 - DNA, 1413 bp, Homo sapiens
atggaaacccccgctcagctgctgttcctgttgctgctgtggctgcccgacactaccgggcaagtgcagctggtggagagcggcggcggcgtggtgcagcccggcagaagccttaggctgagctgcgccgccagtggcttcaccttcagcagctacggcatgcactgggtgagacaggcccccggcaagggcctggagtgggtggctgtgatctggtacgacggcagcaacaagtactacgcagacagcgtgaagggaagattcaccatcagccgcgacaacagcaagaacaccctgtacctgcagatgaacagcctgagagctgaggacaccgcagtgtactactgcgccagagaccccaggggcgccaccctgtactactattattacggcatggatgtgtggggccagggcaccaccgtgaccgtgtccagcgccagcaccaagggcccaagcgtgttcccacttgccccatgcagcagaagcaccagcgagtcaaccgccgccctggggtgcttggtgaaagactacttccccgagcccgtgaccgtgagctggaacagcggagcactgaccagcggtgtgcacaccttccccgccgtgctgcagagcagcggactgtactccctgtccagcgtggtcaccgtgcccagcagcaacttcgggactcaaacttacacttgcaacgtggaccacaaaccctccaataccaaggtggataaaaccgtggagcggaagtgttgcgtggagtgccctccctgccccgcgcctcccgtggccggccccagcgtgttcctgttcccacccaagcctaaggatacactgatgatcagcagaacccccgaggtcacttgcgtggtggtggacgtgagccacgaggacccggaggtgcagtttaactggtacgttgacggggtggaggtgcacaacgctaagaccaagcccagagaagaacagttcaacagcaccttcagggtagtgagcgtgcttaccgtggtgcaccaggactggctgaacggcaaggagtacaagtgcaaggtgagcaacaagggattgcccgccccgatcgagaagacaatctctaagacgaagggccagcccagagagccccaggtgtacaccctgccccccagcagagaggagatgaccaagaatcaggtgagcctgacatgcttggtgaagggcttctaccccagcgacatcgccgtcgagtgggagagcaacggacagcccgagaacaactacaaaaccaccccacctatgctggacagcgacggcagcttcttcctgtactccaaactgaccgtggataagagcagatggcagcagggcaacgtgtttagctgcagcgtgatgcacgaggccctgcacaaccactacacccagaagagcctgtctttgagccccgggaag

SEQ ID NO: 309 - DNA, 1705 bp, Homo sapiens
ggaaataagagagaaaagaagagtaagaagaaatataagagccaccatggaaacccccgctcagctgctgttcctgttgctgctgtggctgcccgacactaccgggcaagtgcagctggtggagagcggcggcggcgtggtgcagcccggcagaagccttaggctgagctgcgccgccagtggcttcaccttcagcagctacggcatgcactgggtgagacaggcccccggcaagggcctggagtgggtggctgtgatctggtacgacggcagcaacaagtactacgcagacagcgtgaagggaagattcaccatcagccgcgacaacagcaagaacaccctgtacctgcagatgaacagcctgagagctgaggacaccgcagtgtactactgcgccagagaccccaggggcgccaccctgtactactattattacggcatggatgtgtggggccagggcaccaccgtgaccgtgtccagcgccagcaccaagggcccaagcgtgttcccacttgccccatgcagcagaagcaccagcgagtcaaccgccgccctggggtgcttggtgaaagactacttccccgagcccgtgaccgtgagctggaacagcggagcactgaccagcggtgtgcacaccttccccgccgtgctgcagagcagcggactgtactccctgtccagcgtggtcaccgtgcccagcagcaacttcgggactcaaacttacacttgcaacgtggaccacaaaccctccaataccaaggtggataaaaccgtggagcggaagtgttgcgtggagtgccctccctgccccgcgcctcccgtggccggccccagcgtgttcctgttcccacccaagcctaaggatacactgatgatcagcagaacccccgaggtcacttgcgtggtggtggacgtgagccacgaggacccggaggtgcagtttaactggtacgttgacggggtggaggtgcacaacgctaagaccaagcccagagaagaacagttcaacagcaccttcagggtagtgagcgtgcttaccgtggtgcaccaggactggctgaacggcaaggagtacaagtgcaaggtgagcaacaagggattgcccgccccgatcgagaagacaatctctaagacgaagggccagcccagagagccccaggtgtacaccctgccccccagcagagaggagatgaccaagaatcaggtgagcctgacatgcttggtgaagggcttctaccccagcgacatcgccgtcgagtgggagagcaacggacagcccgagaacaactacaaaaccaccccacctatgctggacagcgacggcagcttcttcctgtactccaaactgaccgtggataagagcagatggcagcagggcaacgtgtttagctgcagcgtgatgcacgaggccctgcacaaccactacacccagaagagcctgtctttgagccccgggaagtgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 310 - protein, 451 aa, Homo sapiens; identical to SEQ ID NO: 21

SEQ ID NO: 311 - DNA, 935 bp, Homo sapiens
tcaagcttttggaccctcgtacagaagctaatacgactcactatagggaaataagagagaaaagaagagtaagaagaaatataagagccaccatggagacccctgcacagctgctgttcctgctgctgctgtggctgcccgacaccaccggcgacatccagatgacccagagccccagctccctgagcgccagcgtgggcgacagagtgaccatcacctgtagggccagccagagcatcaatagctacctggactggtaccagcagaagcctggcaaggcccccaagctgctgatctacgccgccagcagcctgcaaagcggcgtgcccagcagattcagcgggagcggcagcggcacagactttacactgaccatcagcagcctgcagcccgaggacttcgccacctactactgccaacagtattactccaccccctttaccttcggccccggaaccaaggtggagatcaagagaaccgtggccgcccccagcgtgttcatcttccccccctctgatgagcagctgaagagcggcaccgccagcgtggtgtgcctgctgaacaacttctatcccagagaggccaaggtgcagtggaaggtggacaacgccctgcagagcggcaacagccaggagagcgtgactgagcaggacagcaaggacagcacctacagcctgagcagcaccctgacactgtcaaaggccgattacgagaagcacaaagtgtacgcctgcgaggtgacccaccagggcctgagcagccccgtcaccaagagcttcaacaggggcgagtgctgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 312 - protein, 234 aa, Homo sapiens; identical to SEQ ID NO: 236

SEQ ID NO: 313 - DNA, 702 bp, Homo sapiens
atggagacccctgcacagctgctgttcctgctgctgctgtggctgcccgacaccaccggcgacatccagatgacccagagccccagctccctgagcgccagcgtgggcgacagagtgaccatcacctgtagggccagccagagcatcaatagctacctggactggtaccagcagaagcctggcaaggcccccaagctgctgatctacgccgccagcagcctgcaaagcggcgtgcccagcagattcagcgggagcggcagcggcacagactttacactgaccatcagcagcctgcagcccgaggacttcgccacctactactgccaacagtattactccaccccctttaccttcggccccggaaccaaggtggagatcaagagaaccgtggccgcccccagcgtgttcatcttccccccctctgatgagcagctgaagagcggcaccgccagcgtggtgtgcctgctgaacaacttctatcccagagaggccaaggtgcagtggaaggtggacaacgccctgcagagcggcaacagccaggagagcgtgactgagcaggacagcaaggacagcacctacagcctgagcagcaccctgacactgtcaaaggccgattacgagaagcacaaagtgtacgcctgcgaggtgacccaccagggcctgagcagccccgtcaccaagagcttcaacaggggcgagtgc

SEQ ID NO: 314 - DNA, 994 bp, Homo sapiens
ggaaataagagagaaaagaagagtaagaagaaatataagagccaccatggagacccctgcacagctgctgttcctgctgctgctgtggctgcccgacaccaccggcgacatccagatgacccagagccccagctccctgagcgccagcgtgggcgacagagtgaccatcacctgtagggccagccagagcatcaatagctacctggactggtaccagcagaagcctggcaaggcccccaagctgctgatctacgccgccagcagcctgcaaagcggcgtgcccagcagattcagcgggagcggcagcggcacagactttacactgaccatcagcagcctgcagcccgaggacttcgccacctactactgccaacagtattactccaccccctttaccttcggccccggaaccaaggtggagatcaagagaaccgtggccgcccccagcgtgttcatcttccccccctctgatgagcagctgaagagcggcaccgccagcgtggtgtgcctgctgaacaacttctatcccagagaggccaaggtgcagtggaaggtggacaacgccctgcagagcggcaacagccaggagagcgtgactgagcaggacagcaaggacagcacctacagcctgagcagcaccctgacactgtcaaaggccgattacgagaagcacaaagtgtacgcctgcgaggtgacccaccagggcctgagcagccccgtcaccaagagcttcaacaggggcgagtgctgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 315 - protein, 214 aa, Homo sapiens; identical to SEQ ID NO: 23

SEQ ID NO: 316 - DNA, 935 bp, Homo sapiens
tcaagcttttggaccctcgtacagaagctaatacgactcactatagggaaataagagagaaaagaagagtaagaagaaatataagagccaccatggagacccccgcccagctgctgttcctgctgctgctgtggctgcccgacacaaccggcgacatccagatgacccagagccctagcagcctgagcgccagcgtgggcgacagagtgaccatcacctgcagagccagccagtccatcaacagctacctggactggtaccagcagaagcccggcaaggcccccaaactgctgatctatgctgccagctcactgcagagcggcgtgcctagcagattcagcggctctggcagcggaaccgacttcactctgaccatcagcagcctgcagcccgaggacttcgccacctactactgccagcagtactacagcacccccttcaccttcgggcccggcactaaggtggagatcaagagaaccgtggccgccccaagcgtgttcatcttccccccctccgacgagcagctgaagagcggcaccgccagcgtggtgtgcctgctcaacaacttctaccccagagaggccaaggtgcagtggaaggtggacaacgccctgcagtcgggcaacagccaggagagcgtgaccgagcaggacagcaaggacagcacctacagcctgagcagcactctgaccctgagcaaggctgattacgagaagcacaaggtctacgcctgcgaggtgacccaccagggcctcagcagcccagtgaccaagagcttcaacagaggagagtgctgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 317 - protein, 234 aa, Homo sapiens; identical to SEQ ID NO: 236

SEQ ID NO: 318 - DNA, 702 bp, Homo sapiens
atggagacccccgcccagctgctgttcctgctgctgctgtggctgcccgacacaaccggcgacatccagatgacccagagccctagcagcctgagcgccagcgtgggcgacagagtgaccatcacctgcagagccagccagtccatcaacagctacctggactggtaccagcagaagcccggcaaggcccccaaactgctgatctatgctgccagctcactgcagagcggcgtgcctagcagattcagcggctctggcagcggaaccgacttcactctgaccatcagcagcctgcagcccgaggacttcgccacctactactgccagcagtactacagcacccccttcaccttcgggcccggcactaaggtggagatcaagagaaccgtggccgccccaagcgtgttcatcttccccccctccgacgagcagctgaagagcggcaccgccagcgtggtgtgcctgctcaacaacttctaccccagagaggccaaggtgcagtggaaggtggacaacgccctgcagtcgggcaacagccaggagagcgtgaccgagcaggacagcaaggacagcacctacagcctgagcagcactctgaccctgagcaaggctgattacgagaagcacaaggtctacgcctgcgaggtgacccaccagggcctcagcagcccagtgaccaagagcttcaacagaggagagtgc

SEQ ID NO: 319 - DNA, 994 bp, Homo sapiens
ggaaataagagagaaaagaagagtaagaagaaatataagagccaccatggagacccccgcccagctgctgttcctgctgctgctgtggctgcccgacacaaccggcgacatccagatgacccagagccctagcagcctgagcgccagcgtgggcgacagagtgaccatcacctgcagagccagccagtccatcaacagctacctggactggtaccagcagaagcccggcaaggcccccaaactgctgatctatgctgccagctcactgcagagcggcgtgcctagcagattcagcggctctggcagcggaaccgacttcactctgaccatcagcagcctgcagcccgaggacttcgccacctactactgccagcagtactacagcacccccttcaccttcgggcccggcactaaggtggagatcaagagaaccgtggccgccccaagcgtgttcatcttccccccctccgacgagcagctgaagagcggcaccgccagcgtggtgtgcctgctcaacaacttctaccccagagaggccaaggtgcagtggaaggtggacaacgccctgcagtcgggcaacagccaggagagcgtgaccgagcaggacagcaaggacagcacctacagcctgagcagcactctgaccctgagcaaggctgattacgagaagcacaaggtctacgcctgcgaggtgacccaccagggcctcagcagcccagtgaccaagagcttcaacagaggagagtgctgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 320 - protein, 214 aa, Homo sapiens; identical to SEQ ID NO: 23

SEQ ID NO: 321 - DNA, 935 bp, Homo sapiens
tcaagcttttggaccctcgtacagaagctaatacgactcactatagggaaataagagagaaaagaagagtaagaagaaatataagagccaccatggagactcccgcccagctgctgttcctgttgctgctgtggctgcccgacaccacaggggacatccagatgactcagagtcccagcagcctgagcgccagcgtcggcgacagagttaccatcacctgccgggccagccagagcattaacagctacctggactggtaccagcagaagcctggaaaggcccccaagctgctgatctacgccgccagcagcctgcagagcggcgtgcctagcagattcagcggcagcggaagcggcaccgacttcaccctaaccatcagcagtctgcagccagaggacttcgcgacctactactgccagcagtactactcaacacccttcaccttcggccccggcaccaaggtggagatcaagagaaccgtggccgccccctctgtgttcatcttcccccccagcgacgagcagctgaagtccggcaccgcaagcgtggtgtgcctgctgaacaacttctaccccagagaggccaaggtgcagtggaaggtggacaacgccctgcagtccggcaacagccaggagagcgtgaccgagcaggacagcaaggactccacctacagcctcagcagcacactgaccctgagcaaggccgactacgagaagcacaaggtgtacgcctgcgaggtgacccaccagggcctgagcagccccgtgaccaagagcttcaacagaggagagtgctgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 322 - protein, 234 aa, Homo sapiens; identical to SEQ ID NO: 236

SEQ ID NO: 323 - DNA, 702 bp, Homo sapiens
atggagactcccgcccagctgctgttcctgttgctgctgtggctgcccgacaccacaggggacatccagatgactcagagtcccagcagcctgagcgccagcgtcggcgacagagttaccatcacctgccgggccagccagagcattaacagctacctggactggtaccagcagaagcctggaaaggcccccaagctgctgatctacgccgccagcagcctgcagagcggcgtgcctagcagattcagcggcagcggaagcggcaccgacttcaccctaaccatcagcagtctgcagccagaggacttcgcgacctactactgccagcagtactactcaacacccttcaccttcggccccggcaccaaggtggagatcaagagaaccgtggccgccccctctgtgttcatcttcccccccagcgacgagcagctgaagtccggcaccgcaagcgtggtgtgcctgctgaacaacttctaccccagagaggccaaggtgcagtggaaggtggacaacgccctgcagtccggcaacagccaggagagcgtgaccgagcaggacagcaaggactccacctacagcctcagcagcacactgaccctgagcaaggccgactacgagaagcacaaggtgtacgcctgcgaggtgacccaccagggcctgagcagccccgtgaccaagagcttcaacagaggagagtgc

SEQ ID NO: 324 - DNA, 994 bp, Homo sapiens
ggaaataagagagaaaagaagagtaagaagaaatataagagccaccatggagactcccgcccagctgctgttcctgttgctgctgtggctgcccgacaccacaggggacatccagatgactcagagtcccagcagcctgagcgccagcgtcggcgacagagttaccatcacctgccgggccagccagagcattaacagctacctggactggtaccagcagaagcctggaaaggcccccaagctgctgatctacgccgccagcagcctgcagagcggcgtgcctagcagattcagcggcagcggaagcggcaccgacttcaccctaaccatcagcagtctgcagccagaggacttcgcgacctactactgccagcagtactactcaacacccttcaccttcggccccggcaccaaggtggagatcaagagaaccgtggccgccccctctgtgttcatcttcccccccagcgacgagcagctgaagtccggcaccgcaagcgtggtgtgcctgctgaacaacttctaccccagagaggccaaggtgcagtggaaggtggacaacgccctgcagtccggcaacagccaggagagcgtgaccgagcaggacagcaaggactccacctacagcctcagcagcacactgaccctgagcaaggccgactacgagaagcacaaggtgtacgcctgcgaggtgacccaccagggcctgagcagccccgtgaccaagagcttcaacagaggagagtgctgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 325 - protein, 214 aa, Homo sapiens; identical to SEQ ID NO: 23

(19) United States
(12) Patent Application Publication
(10) Pub. No.: US 2019/0241658 A1
(43) Pub. Date: Aug. 8, 2019

(54) Therapeutic mRNAs encoding anti CTLA-4 antibodies

(71) Applicant: ModernaTX, Inc., Cambridge, MA (US)

(72) Inventor: Joshua Frederick, Boston, MA (US)

(21) Appl. No.: 16/068,787

(22) PCT Filed: Jan. 10, 2017
(86) PCT No.: PCT/US2017/012880

§ 371 Date: Jul. 9, 2018

Related U.S. Application Data
(60) Provisional application No. 62/276,958, filed on Jan. 10, 2016.
(60) Provisional application No. 62/276,959, filed on Jan. 10, 2016.
(60) Provisional application No. 62/290,436, filed on Feb. 2, 2016.
(60) Provisional application No. 62/290,435, filed on Feb. 2, 2016.

(51) Int. Cl. C07K 16/28; A61P 35/00

(52) U.S. Cl. CPC C07K 16/2818; A61K 2039/505; A61P 35/00

(57) ABSTRACT

The invention relates to compositions and methods for their preparation, manufacture and therapeutic use wherein those composition comprise of one or more polynucleotides (e.g., mRNAs) encoding a binding molecule (e.g., an antibody or an antigen binding portion thereof) which specifically binds to CTLA-4 (cytotoxic T-lymphocyte-associated protein 4). The therapeutic methods disclosed herein comprise, e.g., the administration of one or more mRNAs encoding an anti-CTLA-4 antibody or an antigen-binding portion thereof for the treatment of cancer, e.g., by reducing the size of a tumor or inhibiting the growth of a tumor, in a subject in need thereof. In some aspects, the disclosed mRNA or mRNAs encoding the anti-CTLA-4 antibody or an antigen-binding portion thereof are administered intratumorally. When several mRNAs are used to express the disclosed anti-CTLA-4 antibodies or antigen-binding portions thereof, the ratio of mRNAs can be the same or different.

### REFERENCE TO SEQUENCE LISTING SUBMITTED ELECTRONICALLY

The content of the electronically submitted sequence listing in ASCII text file (Name 3529_061PC06_ST25_SQLST.txt, Size: 386,079 bytes; and Date of Creation: Jan. 10, 2017) filed with the application is incorporated herein by reference in its entirety.

### BACKGROUND

Antibodies against the immune checkpoint inhibitor Cytotoxic T-lymphocyte-associated antigen 4 (CLTA4) have demonstrated significant therapeutic efficacy in animal models of cancer, reducing tumor burden and enhancing survival. CTLA-4 (also known as CD152) is the prototypical inhibitory checkpoint receptor. Checkpoint blockade, e.g., by anti-CTLA-4 antibodies, is a transformative therapeutic approach to a broad spectrum of malignancies because it harnesses the power of antitumor immunity to obtain durable responses.

In both preclinical and early clinical trials, CTLA-4 blockade has been shown to release inhibitory controls on T cell activation and proliferation, thereby inducing antitumor immunity. In early preclinical studies, blockade of CTLA-4 by antagonistic CTLA-4 antibodies led to an approximately 2-fold increase in T-cell proliferation and a 6-fold increase in production of the immunocytokine, interleukin-2 (Krummel and Allison, J Exp Med. 183:2533-2540 (1996)). Moreover, anti-CTLA-4 treatment in preclinical studies in a relevant mouse model not only resulted in rejection of tumors but also in immunity to a secondary exposure to tumor cells without additional CTLA-4 blockade, thereby establishing the development of immune memory (Leach et al., Science 271:1734-1736 (1996)).

More recently, CTLA-4 blockade has been shown to promote T-cell activation and to deplete intratumoral Tregs in a process dependent on the presence of Fcγ receptor-expressing macrophages within the tumor microenvironment (Peggs et al., J Exp Med. 206:1717-1725 (2009); Simpson et al., J Exp Med. 210:1695-1710 (2013)).

Based on these preclinical findings, at least two antagonistic anti-CTLA-4 antibodies are advancing in clinical development for use as human therapeutics. Ipilimumab (trade name YERVOY, formerly known as MDX-010 and MDX-101), is a fully human IgG1 antibody developed by Bristol-Myers Squibb and Medarex that works to activate the immune system by targeting CTLA-4. Ipilimumab is approved for the treatment of melanoma (FDA approved for melanoma in 2011) and is undergoing clinical trials for the treatment of non-small cell lung carcinoma (NSCLC), small cell lung cancer (SCLC), bladder cancer and metastatic hormone-refractory prostate cancer. Ipilimumab is the first approved immune checkpoint blockade therapy and has generated considerable excitement over this class of antagonistic antibodies as powerful cancer therapeutics, despite reports of adverse events and undesirable side effects (Beer et al., J. Clin. Oncol. ASCO Ann. Meeting Proceedings, 26 (15S): 5004 (2008); Pardol, Nat. Rev. Cancer 12 (4): 252-64 (2012); Wolchok et al., Ann. Oncol. 24(8): 2174-2180 (2013)).

Tremelimumab (formerly ticilimumab, CP-675,206) is a fully human IgG2 monoclonal antibody produced by Pfizer and in development by AstraZeneca in conjunction with AstraZeneca's biologics research and development arm, MedImmune. Tremelimumab is reported to have therapeutic benefits over ipilimumab including a longer half-life and cause less antibody-dependent cellular toxicity as compared to ipilimumab. AstraZeneca, with MedImmune, has developed tremelimumab for treatment of cancer and the drug is presently undergoing human clinical trials for treatment of several metastatic cancers, either alone or in combination with other immunotherapeutic approaches, e.g., MedImmune's anti-PD-L1 antibody (Ribas et al, The Oncologist 12 (7): 873-883 (2007); Ribas et al., J. Clin. Oncol. 31(5): 616-622 (2013); Camacho et al., J Clin Oncol. 27 (7):1075-81 (2009)).

### BRIEF SUMMARY

The present invention relates to compositions and methods for treating cancer in a subject in need thereof comprising administering mRNAs encoding CTLA-4 antibodies or antigen-binding portions thereof.

The present disclosure provides a method of reducing the size of a tumor or inhibiting the growth of a tumor in a subject in need thereof comprising administering to said subject one or more mRNAs encoding an antibody or an antigen binding portion thereof which specifically binds to CTLA-4 (cytotoxic T-lymphocyte-associated protein 4). Also provided is a method of reducing the size of a tumor or inhibiting the growth of a tumor in a subject in need thereof comprising administering to said subject one or more mRNAs encoding an antibody or an antigen binding portion thereof which specifically binds to the same CTLA-4 epitope as (i) an antibody or antigen-binding portion thereof comprising a heavy chain variable region (VH) of SEQ ID NO: 9, 28, or 39, and a light chain variable region (VL) of SEQ ID NO: 11, 29 or 41; or, (ii) an antibody or antigen-binding portion comprising a VH of SEQ ID NO: 183 or SEQ ID NO: 240 and a VL of SEQ ID NO: 185 or SEQ ID NO: 238.

Also provided is a method of reducing the size of a tumor or inhibiting the growth of a tumor in a subject in need thereof comprising administering to said subject one or more mRNAs encoding an antibody or an antigen binding portion thereof which specifically binds to CTLA-4 and competitively inhibits CTLA-4 binding by (i) an antibody or antigen-binding portion thereof comprising a VH of SEQ ID NO: 9, 28, or 39 and a VL of SEQ ID NO: 11, 29 or 41; or, (ii) an antibody or antigen-binding portion thereof comprising a VH of SEQ ID NO: 183 or SEQ ID NO: 240 and a VL of SEQ ID NO: 185 or SEQ ID NO: 238. Also provided is a method of reducing the size of a tumor or inhibiting the growth of a tumor in a subject in need thereof comprising administering to said subject one or more mRNAs encoding (i) an antibody or an antigen binding portion thereof which specifically binds to CTLA-4 comprising a VH of SEQ ID NO: 9, 28, or 39 and a VL of SEQ ID NO: 11, 29 or 41, or (ii) an antibody or antigen-binding portion thereof which specifically binds to CTLA-4 comprising a VH of SEQ ID NO: 183 or SEQ ID NO: 240 and a VL of SEQ ID NO: 185 or SEQ ID NO: 238.

The present disclosure also provides a method of reducing the size of a tumor or inhibiting the growth of a tumor in a subject in need thereof comprising administering to said subject one or more mRNAs encoding an antibody or an antigen binding portion thereof which specifically binds to CTLA-4, wherein the antibody or an antigen binding portion thereof comprises (i) a VL complementarity determining region 1 (VL-CDR1) amino acid sequence identical to, or identical except for four, three, two or one amino acid substitutions to SEQ ID NO: 17, 33, or 47 or SEQ ID NO: 189 or SEQ ID NO: 245; (ii) a VL complementarity determining region 1 (VL-CDR2) amino acid sequence identical to, or identical except for four, three, two or one amino acid substitutions to SEQ ID NO: 18, 34 or 48 or SEQ ID NO: 190 or SEQ ID NO: 246; a VL complementarity determining region 1 (VL-CDR3) amino acid sequence identical to, or identical except for four, three, two or one amino acid substitutions to SEQ ID NO: 19, 35 or 49 or SEQ ID NO: 191 or SEQ ID NO: 247; (iv) a VH complementarity determining region I (VH-CDR1) amino acid sequence identical to, or identical except for four, three, two or one amino acid substitutions to SEQ ID NO: 14, 30 or 44 or SEQ ID NO: 186 or SEQ ID NO: 248; (v) a VH complementarity deter mining region 1 (VH-CDR2) amino acid sequence identical to, or identical except for four, three, two or one amino acid substitutions to SEQ ID NO:15, 31 or 45 or SEQ ID NO: 187 or SEQ ID NO: 249; and (vi) a VH complementarity determining region 1 (VH-CDR3) amino acid sequence identical to, or identical except for four, three, two or one amino acid substitutions to SEQ ID NO:16, 32 or 46 or SEQ ID NO:188 or SEQ ID NO: 250.

Also provided is a method of reducing the size of a tumor or inhibiting the growth of a tumor in a subject in need thereof comprising administering to said subject one or more mRNAs encoding an antibody or an antigen binding portion thereof which specifically binds to CTLA-4, and which comprises (i) a VL, wherein the VL comprises VL-CDR1, VL-CDR2, and VL-CDR3 amino acid sequences identical to, or identical except for four, three, two, or one amino acid substitutions in one or more of the VL-CDRS to: SEQ ID NOs: 17, 18 and 19, SEQ ID NOs: 33, 34, and 35, SEQ ID NOs: 47, 48, and 49; SEQ ID NOs: 189, 190, and 191, or SEQ ID NOs: 245, 246, or 247 respectively; and (ii) a VH, wherein the VH comprises VH-CDR1, VH-CDR2, and VH-CDR3 amino acid sequences identical to, or identical except for four, three, two, or one amino acid substitutions in one or more of the VH-CDRS to: SEQ ID NOs:14, 15 and 16, SEQ ID NOs: 30, 31 and 32, SEQ ID NOs: 44, 45, and 46; SEQ ID NOs: 186, 187, and 188, or SEQ ID NOs: 248, 249, and 250respectively.

The present disclosure also provides a method of reducing the size of a tumor or inhibiting the growth of a tumor in a subject in need thereof comprising administering to said subject one or more mRNAs encoding an antibody or an antigen binding portion thereof which specifically binds to CTLA-4, and which comprises a VL and a VH comprising VL-CDR1, VL-CDR2, VL-CDR3, VH-CDR1, VH-CDR2, and VH-CDR3 amino acid sequences identical or identical except for four, three, two, or one amino acid substitutions in one or more CDRs to: (i) SEQ ID NOs: 17, 18, 19, 14, 15, and 16; (ii) SEQ ID NOs: 33, 34, 35, 30, 31, and 32; (iii) SEQ ID NOs: 47, 48, 39, 44, 45, and 46; (iv) SEQ ID NOs: 189, 190, 191, 186, 187 and 188; or (v) SEQ ID NOs: 245, 246, 247, 248, 249, and 250. Also provided is a method of reducing the size of a tumor or inhibiting the growth of a tumor in a subject in need thereof comprising administering to said subject one or more mRNAs encoding an antibody or an antigen binding portion thereof which specifically binds to CTLA-4, and which comprises a VL and a VH, wherein the VL comprises an amino acid sequence having at least 80%, at least 85%, at least 90%, at least 95%, at least 96%, at least 97%, at least 98%, at least 99% sequence identity to SEQ ID NO: 11, SEQ ID NO: 29, SEQ ID NO:41. or SEQ ID NO: 185 or SEQ ID NO: 238.

The present disclosure also provides a method of reducing the size of a tumor or inhibiting the growth of a tumor in a subject in need thereof comprising administering to said subject one or more mRNAs encoding an antibody or an antigen binding portion thereof which specifically binds to CTLA-4, and which comprises a VL and a VH, wherein the VH comprises an amino acid sequence having at least 80%, at least 85%, at least 90%, at least 95%, at least 96%, at least 97%, at least 98%, at least 99% sequence identity to SEQ ID NO: 14, SEQ ID NO: 30, SEQ ID NO:44 or SEQ ID NO: 186 or SEQ ID NO: 240. Also provided is a method of reducing the size of a tumor or inhibiting the growth of a tumor in a subject in need thereof comprising administering to said subject one or more mRNAs encoding an antibody or an antigen binding portion thereof which specifically binds to CTLA-4, and which comprises a VL and a VH, wherein (i) the VL comprises an amino acid sequence having at least 80%, at least 85%, at least 90%, at least 95%, at least 96%, at least 97%, at least 98%, at least 99% sequence identity to SEQ ID NO: 11, SEQ ID NO: 29, or SEQ ID NO:41, and the VH comprises an amino acid sequence having at least 80%, at least 85%, at least 90%, at least 95%, at least 96%, at least 97%, at least 98%, at least 99% sequence identity to SEQ ID NO: 9, SEQ ID NO: 28, or SEQ ID NO:39; or, (ii) the VL comprises an amino acid sequence having at least 80%, at least 85%, at least 90%, at least 95%, at least 96%, at least 97%, at least 98%, at least 99% sequence identity to SEQ ID NO: 185 or SEQ ID NO: 238, and the VH comprises an amino acid sequence having at least 80%, at least 85%, at least 90%, at least 95%, at least 96%, at least 97%, at least 98%, at least 99% sequence identity to SEQ ID NO: 183 or SEQ ID NO: 240.

The present disclosure also provides a method of reducing the size of a tumor or inhibiting the growth of a tumor in a subject in need thereof comprising administering to said subject one or more mRNAs encoding an antibody or an antigen binding portion thereof which specifically binds to CTLA-4, and which comprises a VL and a VH, wherein (i) the VL consists or consists essentially of the amino acid sequence of SEQ ID NO: 11, SEQ ID NO: 29, or SEQ ID NO:41, and the VH consists or consists essentially of the amino acid sequence of SEQ ID NO: SEQ ID NO: 9, SEQ ID NO: 28, or SEQ ID NO:39; or, (ii) the VL consists or consists essentially of the amino acid sequence of SEQ ID NO: 185 or SEQ ID NO: 238, wherein the VH consists or consists essentially of the amino acid sequence of SEQ ID NO: 183 or SEQ ID NO: 240.

In some aspects of the methods disclosed above, at least one mRNA further encodes a heavy chain constant region or fragment thereof. In some aspects, the heavy chain constant region or fragment thereof is an IgG constant region. In some aspects, the IgG constant region is selected from an IgG1 constant region, an IgG2 constant region, an IgG3 constant region and an IgG4 constant region. In some aspects, the IgG constant region comprises a CHI domain, a CH2 domain, a CH3 domain, or a combination thereof. In some aspects of the methods disclosed above, at least one mRNA further encodes a light chain constant region or fragment thereof. In some aspects, the light chain constant region is selected from the group consisting of a kappa constant region and a lambda constant region.

The present disclosure also provides a method of reducing the size of a tumor or inhibiting the growth of a tumor in a subject in need thereof comprising administering to said subject one or more mRNAs encoding an antibody or an antigen binding portion thereof which specifically binds to CTLA-4, and which comprises a heavy chain (HC) a light chain (LC), wherein the HC comprises, consists, or consists essentially of the amino acid sequence of SEQ ID NO: 20 or SEQ ID NO:24, and the LC comprises, consists, or consists essentially of the amino acid sequence of SEQ ID NO: 22 or SEQ ID NO: 26.

In some aspects of the methods disclosed above, the antibody or antigen binding portion thereof which specifically binds to CTLA-4 is a complete antibody, an antibody variant, an antibody fragment, or a combination thereof. In some aspects, the antibody or antigen binding portion thereof which specifically binds to CTLA-4 comprises, consists, or consists essentially of the antibody fragment, and wherein the antibody fragment is an scFv, Fv, Fab, F(ab′)2, Fab′, dsFv, or sc(Fv)2. In some aspects, the antibody or antigen binding portion thereof which specifically binds to CTLA-4 is an intrabody, a bicistronic antibody, a pseudobicistronic antibody, a single domain antibody, or a bispecific antibody. In some aspects, the CTLA-4 is human CTLA-4.

In some aspects of the methods disclosed above, binding of the antibody or antigen binding portion thereof to CTLA-4 (i) reduces the size of the tumor; (ii) inhibits the growth of the tumor; (iii) reduces tumor cell proliferation in the subject; (iv) increases survival rate; or, (v) a combination thereof.

In some aspects, the tumor is selected from the group consisting of melanoma tumor, lung cancer tumor, bladder cancer tumor, colon cancer tumor, and prostate cancer tumor. In some aspects, the tumor is a melanoma tumor and wherein the melanoma tumor is a metastatic melanoma tumor. In some aspects, the tumor is lung cancer tumor and wherein the lung cancer tumor is a non-small cell lung carcinoma (NSCLC) tumor or a small cell lung cancer (SCLC) tumor. In some aspects, the tumor is a prostate cancer tumor and wherein the prostate cancer tumor is a metastatic hormone-refractory prostate cancer tumor. In some aspects, the subject is human.

In some aspects, the antibody or antigen binding portion thereof which specifically binds to CTLA-4 is encoded by a polynucleotide sequence at least 60%, at least 70%, at least 80%, at least 85%, at least 90%, at least 95%, at least 96%, at least 97%, at least 98%, at least 99%, or 100% identical to SEQ ID NO: 20, SEQ ID NO:24, SEQ ID NO:22, or SEQ ID NO:26, a subsequence thereof, or a combination thereof. In some aspects, said polynucleotide sequence at least 60%, at least 70%, at least 80%, at least 85%, at least 90%, at least 95%, at least 96%, at least 97%, at least 98%, at least 99%, or 100% identical to at least one of SEQ ID NO: 20, SEQ ID NO:22, SEQ ID NO:24 or SEQ ID NO:26 or to a subsequence thereof encodes (i) one, two or three VH-CDRs; (ii) one, two or three VL-CDRs; (iii) a VH; (iv) a VL; (v) a HC; (vi) a LC; (vii) a fragment thereof; or, (viii) a combination thereof, wherein the antibody or antigen binding portion thereof which specifically binds to CTLA-4 encoded by said polynucleotide sequence binds to at least one CTIA-4 molecule.

In some aspects of the methods disclosed above, (i) at least one of the one or more mRNAs encoding an antibody or an antigen binding portion thereof which specifically binds to CTLA-4 comprises at least one chemically modified nucleoside; or, (ii) each one of the one or more mRNAs encoding an antibody or an antigen binding portion thereof which specifically binds to CTIA-4 comprises at least one chemically modified nucleoside. In some aspects, the at least one chemically modified nucleoside is selected from the group consisting of pseudouridine (ψ), N1-methylpseudouridine (m1ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine , 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof. In some aspects, the at least one chemically modified nucleoside is selected from the group consisting of pseudouridine (ψ), N1-methylpseudouridine 5-methylcytosine, 5-methoxyuridine, and a combination thereof.

In some aspects, the one or more mRNAs encoding an antibody or an antigen binding portion thereof which specifically binds to CTLA-4 comprise at least 10%, at least 20%, at least 30%, at least 40%, at least 50%, at least 60%, at least 70%, at least 80%, at least 90%, at least 95%, at least 99%, or 100% chemically modified nucleosides. In some aspects, the chemically modified nucleosides in the one or more mRNAs are selected from the group consisting of uridine nucleosides, adenine nucleosides, cytosine nucleosides, guanine nucleosides, and any combination thereof. In some aspects, the uridine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%. In some aspects, the adenine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%. In some aspects, the cytosine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%. In some aspects, the guanine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%.

In some aspects of the methods disclosed above, one or more mRNAs encoding an antibody or an antigen binding portion thereof which specifically binds to CTLA-4 further comprise a 5′ UTR. In some aspects, the 5′ UTR comprises a nucleic acid sequence at least 90%, at least 95%, at least 96%, at least 97%, at least 98%, at least 99%, or 100% identical to a sequence selected from SEQ ID NOS: 197-214. In some aspects, the 5′ UTR is codon optimized. In some aspects, one or more mRNAs encoding an antibody or an antigen binding portion thereof which specifically binds to CTLA-4 further comprise a 3′ UTR. In some aspects, the 3′ UTR comprises a nucleic acid sequence at least 90%, 95%, 96%, 97%, 98%, 99%, or 100% identical to a sequence selected from SEQ ID NO: 215-232. In some aspects, the 3′ UTR is codon optimized. In some aspects, one or more mRNAs encoding an antibody or an antigen binding portion thereof which specifically binds to CTLA-4 further comprise a 5′ terminal cap. In some aspects, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, guanosine, 2-azidoguanosine, Cap2, Cap4, 5′ methylG cap, or an analog thereof. In some aspects, one or more mRNAs encoding an antibody or an antigen binding portion thereof which specifically binds to CTLA-4 further comprise a 3′ polyA tail.

In some aspects of the methods disclosed above, one or more mRNAs encoding an antibody or an antigen binding portion thereof which specifically binds to CTLA-4 are in vitro transcribed (IVT). In some aspects, one or more mRNAs encoding an antibody or an antigen binding portion thereof which specifically binds to CTLA-4 are chimeric. In some aspects, one or more mRNAs encoding an antibody or an antigen binding portion thereof which specifically binds to CTLA-4 are circular. In some aspects, one or more mRNAs are purified by strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE). In some aspects, the one or more mRNAs encoding an antibody or an antigen binding portion thereof which specifically binds to CTLA-4 are formulated with a delivery agent. In some aspects, the delivery agent comprises a lipidoid, a liposome, a lipoplex, a lipid nanoparticle, a polymeric compound, a peptide, a protein, a cell, a nanoparticle mimic, a nanotube, or a conjugate. In some aspects, the delivery agent is a lipid nanoparticle. In some aspects, the lipid nanoparticle comprises the lipid selected from the group consisting of DLin-DMA, DLin-K-DMA, 98N12-5, C12-200, DLin-MC3-DMA, DLin-KC2-DMA, DODMA, PLGA, PEG, PEG-DMG, PEGylated lipids, amino alcohol lipids, KL22, and combinations thereof. In some aspects, the one or more mRNAs encoding an antibody or an antigen binding portion thereof which specifically binds to CTLA-4 are formulated for in vivo delivery. In some aspects, the one or more mRNAs encoding an antibody or an antigen binding portion thereof which specifically binds to CTLA-4 are formulated for subcutaneous, intravenous, intraperitoneal, intramuscular, intra-articular, intra-synovial, intrasternal, intrathecal, intrahepatic, intralesional, intracranial, intraventricular, oral, inhalation spray, topical, rectal, nasal, buccal, vaginal, intratumoral, or implanted reservoir intramuscular, subcutaneous, intratumoral, or intradermal in vivo delivery. In some aspects, a host cell comprises the one or more mRNAs encoding an antibody or an antigen binding portion thereof which specifically binds to CTLA-4.

The present disclosure, in addition to methods of use (e.g., methods of therapeutic use of mRNA encoding a therapeutic antibody or an antigen binding portion thereof that specifically binds to CTIA-4) also provides, e.g., compositions of matter. Accordingly, the present disclosure provides a polynucleotide comprising one or more mRNAs which encode an antibody or an antigen binding portion thereof which specifically binds to CTLA-4 and which specifically binds to the same CTLA-4 epitope as (i) an antibody or antigen-binding portion thereof comprising a heavy chain variable region (VH) of SEQ ID NO: 9, 28, or 39, and a light chain variable region (VL) of SEQ ID NO: 11, 29 or 41, or (ii) an antibody or antigen-binding portion comprising a VH of SEQ ID NO: 183 and a VL of SEQ ID NO: 185.

Also provided is a polynucleotide comprising one or more mRNAs which encode an antibody or an antigen binding portion thereof which specifically binds to CTLA-4 and competitively inhibits CTLA-4 binding by (i) an antibody or antigen-binding portion thereof comprising a VH of SEQ ID NO: 9, 28, or 39 and a VL of SEQ ID NO: 11, 29 or 41; or, (ii) an antibody or antigen-binding portion thereof comprising a VH of SEQ ID NO: 183 or SEQ ID NO: 240 and a VL of SEQ ID NO: 185 or SEQ ID NO: 238. Also provided is a polynucleotide comprising one or more mRNAs which encodes (i) an antibody or an antigen binding portion thereof which specifically binds to CTLA-4 comprising a VH of SEQ ID NO: 9, 28, or 39 and a VL of SEQ ID NO: 11, 29 or 41; or, (ii) an antibody or antigen-binding portion thereof comprising a VH of SEQ ID NO: 183 or SEQ ID NO: 240 and a VL of SEQ ID NO: 185 or SEQ ID NO: 238. Also provided is a polynucleotide comprising one or more mRNAs which encode an antibody or an antigen binding portion thereof which specifically binds to CTLA-4, wherein the antibody or an antigen binding portion thereof comprises (i) a VL complementarity determining region 1 (VL-CDR1) amino acid sequence identical to, or identical except for four, three, two or one amino acid substitutions to SEQ ID NO: 17, 33, or 47 or SEQ ID NO: 189 or SEQ ID NO: 245; (ii) a VL complementarity determining region 1 (VL-CDR2) amino acid sequence identical to, or identical except for four, three, two or one amino acid substitutions to SEQ ID NO: 18, 34 or 48 or SEQ ID NO: 190 or SEQ ID NO: 246; (iii) a VL complementarity determining region 1 (VL-CDR3) amino acid sequence identical to, or identical except for four, three, two or one amino acid substitutions to SEQ ID NO: 19, 35 or 49 or SEQ ID NO: 191 or SEQ ID NO: 247; (iv) a VH complementarity determining region 1 (VH-CDR1) amino acid sequence identical to, or identical except for four, three, two or one amino acid substitutions to SEQ ID NO: 14, 30 or 44 or SEQ ID NO: 186 or SEQ ID NO: 248; (v) a VH complementarity determining region 1 (VH-CDR2) amino acid sequence identical to, or identical except for four, three, two or one amino acid substitutions to SEQ ID NO:15, 31 or 45 or SEQ ID NO: 187 or SEQ ID NO: 249; and, (vi) a VH complementarity determining region 1 (VH-CDR3) amino acid sequence identical to, or identical except for four, three, two or one amino acid substitutions to SEQ ID NO:16, 32 or 46 or SEQ ID NO:188 or SEQ ID NO: 250.

The present disclosure also provides a polynucleotide comprising one or more mRNAs which encode an antibody or an antigen binding portion thereof which specifically binds to CTLA-4, and which comprises (i) a VL, wherein the VL comprises VL-CDR1, VL-CDR2, and VL-CDR3 amino acid sequences identical to, or identical except for four, three, two, or one amino acid substitutions in one or more of the VL-CDRS to: SEQ ID NOs: 17, 18 and 19, SEQ ID NOs: 33, 34, and 35, SEQ ID NOs: 47, 48, and 49, SEQ ID NOs: 189, 190, and 191, SEQ ID NOs: 245, 246, and 247, respectively; and (ii) a VH, wherein the VH comprises VH-CDR1, VH-CDR2, and VH-CDR3 amino acid sequences identical to, or identical except for four, three, two, or one amino acid substitutions in one or more of the VH-CDRS to: SEQ ID NOs:14,15 and 16, SEQ ID NOs: 30, 31 and 32, SEQ ID NOs: 44, 45, and 46, SEQ ID NOs: 186, 187, and 188, SEQ ID NOs: 248, 249, and 250, respectively. Also provides is a polynucleotide comprising one or more mRNAs which encode an antibody or an antigen binding portion thereof which specifically binds to CTLA-4, and which comprises a VL and a VH comprising VL-CDR1, VL-CDR2, VL-CDR3, VH-CDR1, VH-CDR2, and VH-CDR3 amino acid sequences identical or identical except for four, three, two, or one amino acid substitutions in one or more CDRs to: (i) SEQ ID NOs: 17, 18, 19, 14, 15, and 16; (ii) SEQ ID NOs: 33, 34, 35, 30, 31, and 32; (iii) SEQ ID NOs: 47, 48, 39, 44, 45, and 46; (iv) SEQ ID NOs: 189, 190, 191, 186, 187 and 188, or (v) SEQ ID NOs: 245, 246, 247, 248, 249, or 250. Also provided is a polynucleotide comprising one or more mRNAs which encode an antibody or an antigen binding portion thereof which specifically binds to CTLA-4, and which comprises a VL and a VH, wherein the VL comprises an amino acid sequence having at least 80%, at least 85%, at least 90%, at least 95%, at least 96%, at least 97%, at least 98%, at least 99% sequence identity to SEQ ID NO: 11, SEQ ID NO: 29, SEQ ID NO:41 or SEQ ID NO: 185 or 238.

The present disclosure also provides a polynucleotide comprising one or more mRNAs which encode an antibody or an antigen binding portion thereof which specifically binds to CTLA-4, and which comprises a VL and a VH, wherein the VH comprises an amino acid sequence having at least 80%, at least 85%, at least 90%, at least 95%, at least 96%, at least 97%, at least 98%, at least 99% sequence identity to SEQ ID NO: 14, SEQ ID NO: 30, SEQ ID NO:44 or SEQ ID NO: 183 or 240, wherein at least one nucleoside in the polynucleotide is a chemically modified nucleoside. Also provided is a polynucleotide comprising one or more mRNAs which encode an antibody or an antigen binding portion thereof which specifically binds to CTLA-4, and which comprises a VL and a VH, wherein (i) the VL comprises an amino acid sequence having at least 80%, at least 85%, at least 90%, at least 95%, at least 96%, at least 97%, at least 98%, at least 99% sequence identity to SEQ ID NO: 11, SEQ ID NO: 29, or SEQ ID NO:41, and the VH comprises an amino acid sequence having at least 80%, at least 85%, at least 90%, at least 95%, at least 96%, at least 97%, at least 98%, at least 99% sequence identity to SEQ ID NO: 9, SEQ ID NO: 28, or SEQ ID NO:39; or, (ii) the VL comprises an amino acid sequence having at least 80%, at least 85%, at least 90%, at least 95%, at least 96%, at least 97%, at least 98%, at least 99% sequence identity to SEQ ID NO: 185 or 238, and the VH comprises an amino acid sequence having at least 80%, at least 85%, at least 90%, at least 95%, at least 96%, at least 97%, at least 98%, at least 99% sequence identity to SEQ ID NO: 183 or 240. Also provided is a polynucleotide comprising one or more mRNAs which encode an antibody or an antigen binding portion thereof which specifically binds to CTLA-4, and which comprises a VL and a VH, wherein (i) the VL consists or consists essentially of the amino acid sequence of SEQ ID NO: 11, SEQ ID NO: 29, or SEQ ID NO:41, and the VH consists or consists essentially of the amino acid sequence of SEQ ID NO: SEQ ID NO: 9, SEQ ID NO: 28, or SEQ ID NO:39; or, (ii) the VL consists or consists essentially of the amino acid sequence of SEQ ID NO: 185 or 238, wherein the VH consists or consists essentially of the amino acid sequence of SEQ ID NO: 183 or 240.

In some aspects of the polynucleotides disclosed above, at least one mRNA encoding an antibody or an antigen binding portion thereof which specifically binds to CTLA-4 further encodes a heavy chain constant region or fragment thereof. In some aspects, the heavy chain constant region or fragment thereof is an IgG constant region. In some aspects, the IgG constant region is selected from an IgG1 constant region, an IgG2 constant region, an IgG3 constant region and an IgG4 constant region. In some aspects, the IgG constant region comprises a CH1 domain, a CH2 domain, a CH3 domain, or a combination thereof. In some aspects, at least one mRNA encoding an antibody or an antigen binding portion thereof which specifically binds to CTLA-4 further encodes a light chain constant region or fragment thereof. In some aspects, the light chain constant region is selected from the group consisting of a kappa constant region and a lambda constant region.

The present disclosure also provides a polynucleotide comprising one or more mRNAs which encode an antibody or an antigen binding portion thereof which specifically binds to CTLA-4, and which comprises a heavy chain (HC) a light chain (LC), wherein the HC comprises, consists, or consists essentially of the amino acid sequence of SEQ ID NO: 20 or SEQ ID NO:24, and the LC comprises, consists, or consists essentially of the amino acid sequence of SEQ ID NO: 22 or SEQ ID NO: 26. In some aspects, the antibody or antigen binding portion thereof which specifically binds to CTLA-4 is a complete antibody, an antibody variant, an antibody fragment, or a combination thereof. In some aspects, the antibody or antigen binding portion thereof which specifically binds to CTLA-4 comprises, consists, or consists essentially of the antibody fragment, and wherein the antibody fragment is an scFv, Fv, Fab, F(ab′)2, Fab′, dsFv, or sc(Fv)2. In some aspects, the antibody or antigen binding portion thereof which specifically binds to CTLA-4 is an intrabody, a bicistronic antibody, a pseudobicistronic antibody, a single domain antibody, or a bispecific antibody. In some aspects, the CTLA-4 is human CTLA-4. In some aspects, binding of the antibody or antigen binding portion thereof to CTLA-4 (i) reduces the size of the tumor; (ii) inhibits the growth of the tumor; (iii) reduces tumor cell proliferation in the subject; (iv) increases survival rate; or, (v) a combination thereof. In some aspects, the tumor is selected from the group consisting of melanoma tumor, lung cancer tumor, bladder cancer tumor, colon cancer tumor, and prostate cancer tumor. In some aspects, the tumor is a melanoma tumor and wherein the melanoma tumor is a metastatic melanoma tumor. In some aspects, the tumor is lung cancer tumor and wherein the lung cancer tumor is a non-small cell lung carcinoma (NSCLC) tumor or a small cell lung cancer (SCLC) tumor. In some aspects, the tumor is a prostate cancer tumor and wherein the prostate cancer tumor is a metastatic hormone-refractory prostate cancer tumor. In some aspects, the subject is human. In some aspects, the antibody or antigen binding portion thereof which specifically binds to CTLA-4 is encoded by a polynucleotide sequence at least 60%, at least 70%, at least 80%, at least 85%, at least 90%, at least 95%, at least 96%, at least 97%, at least 98%, at least 99%, or 100% identical to SEQ ID NO: 20, SEQ ID NO:24, SEQ ID NO:22, or SEQ ID NO:26, a subsequence thereof, or a combination thereof. In some aspects, said polynucleotide sequence at least 60%, at least 70%, at least 80%, at least 85%, at least 90%, at least 95%, at least 96%, at least 97%, at least 98%, at least 99%, or 100% identical to at least one of SEQ ID NO: 20, SEQ ID NO:22, SEQ ID NO:24 or SEQ ID NO:26 or to a subsequence thereof encodes (i) one, two or three VH-CDRs; (ii) one, two or three VL-CDRs; (iii) a VH; (iv) a VL; (v) a HC; (vi) a LC; (vii) a fragment thereof; or, (viii) a combination thereof, wherein the antibody or antigen binding portion thereof which specifically binds to CTLA-4 encoded by said polynucleotide sequence binds to at least one CTLA-4 molecule.

In some aspects of the polynucleotides disclosed above (i) at least one of the one or more mRNAs encoding an antibody or an antigen binding portion thereof which specifically binds to CTLA-4 comprises at least one chemically modified nucleoside; or, (ii) each one of the one or more mRNAs encoding an antibody or an antigen binding portion thereof which specifically binds to CTLA-4 comprises at least one chemically modified nucleoside. In some aspects, the at least one chemically modified nucleoside is selected from the group consisting of pseudouridine (ψ), N1-methylpseudouridine (m1ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof. In some aspects, the at least one chemically modified nucleoside is selected from the group consisting of pseudouridine (ψ), N1-methylpseudouridine (m1ψ), 5-methylcytosine, 5-methoxyuridine, and a combination thereof. In some aspects, the one or more mRNAs encoding an antibody or an antigen binding portion thereof which specifically binds to CTLA-4 comprise at least 10%, at least 20%, at least 30%, at least 40%, at least 50%, at least 60%, at least 70%, at least 80%, at least 90%, at least 95%, at least 99%, or 100% chemically modified nucleosides. In some aspects, the chemically modified nucleosides in the one or more mRNAs are selected from the group consisting of uridine nucleosides, adenine nucleosides, cytosine nucleosides, guanine nucleosides, and any combination thereof. In some aspects, the uridine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%. In some aspects, the adenine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%. In some aspects, the cytosine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%. In some aspects, the guanine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%.

In some aspects of the polynucleotides disclosed above, one or more mRNAs encoding an antibody or an antigen binding portion thereof which specifically binds to CTLA-4 further comprise a 5′ UTR. In some aspects, the 5′ UTR comprises a nucleic acid sequence at least 90%, at least 95%, at least 96%, at least 97%, at least 98%, at least 99%, or 100% identical to a sequence selected from SEQ ID NOS: 197-214. In some aspects, the 5′ UTR is codon optimized. In some aspects, one or more mRNAs encoding an antibody or an antigen binding portion thereof which specifically binds to CTLA-4 further comprise a 3′ UTR. In some aspects, the 3′ UTR comprises a nucleic acid sequence at least 90%, 95%, 96%, 97%, 98%, 99%, or 100% identical to a sequence selected from SEQ ID NO: 215-232. In some aspects, the 3′ UTR is codon optimized. In some aspects, one or more mRNAs encoding an antibody or an antigen binding portion thereof which specifically binds to CTLA-4 further comprise a 5′ terminal cap. In some aspects, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof. In some aspects, one or more mRNAs encoding an antibody or an antigen binding portion thereof which specifically binds to CTLA-4 further comprise a 3′ polyA tail. In some aspects, one or more mRNAs encoding an antibody or an antigen binding portion thereof which specifically binds to CTLA-4 are in vitro transcribed (IVT). In some aspects, one or more mRNAs encoding an antibody or an antigen binding portion thereof which specifically binds to CTLA-4 are chimeric. In some aspects, one or more mRNAs encoding an antibody or an antigen binding portion thereof which specifically binds to CTLA-4 are circular. In some aspects, one or more mRNAs encoding an antibody or an antigen binding portion thereof which specifically binds to CTLA-4 are purified by strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE). In some aspects, the one or more mRNAs encoding an antibody or an antigen binding portion thereof which specifically binds to CTLA-4 comprise a leader sequence comprising, consisting, or consisting essentially of the sequence of SEQ ID NO: 234.

The present disclosure also provides a composition comprising any of the polynucleotides disclosed above and a delivery agent. In some aspects, the delivery agent comprises a lipidoid, a liposome, a lipoplex, a lipid nanoparticle, a polymeric compound, a peptide, a protein, a cell, a nanoparticle mimic, a nanotube, or a conjugate. In some aspects, the delivery agent is a lipid nanoparticle. In some aspects, the lipid nanoparticle comprises the lipid selected from the group consisting of DLin-DMA, DLin-K-DMA, 98N12-5, C12-200, DLin-MC3-DMA, DLin-KC2-DMA, DODMA, PLGA, PEG, PEG-DMG, PEGylated lipids, amino alcohol lipids, KL22, and combinations thereof. In some aspects, the composition is formulated for in vivo delivery. In some aspects the composition is formulated for subcutaneous, intravenous, intraperitoneal, intramuscular, intra-articular, intra-synovial, intrasternal, intrathecal, intrahepatic, intralesional, intracranial, intraventricular, oral, inhalation spray, topical, rectal, nasal, buccal, vaginal, intratumoral, or implanted reservoir intramuscular, subcutaneous, intratumoral, or intradermal in viva delivery.

The present disclosure also provides a host cell comprising any of the polynucleotide disclosed above. Also provided is method of making a polynucleotide disclosed above comprising enzymatically or chemically synthesizing the polynucleotide. Also provided is a method of producing an antibody in a cell, tissue, or organism, comprising contacting said cell, tissue, or organism with the polynucleotide disclosed above, or a composition disclosed above.

The present disclosure also provides a method of expressing in viva an active anti CTLA-4 antibody in a subject in need thereof comprising administering to the subject an effective amount of a polynucleotide disclosed herein, a composition disclosed herein, or a cell disclosed herein.

Also provided is a method of treating cancer in a subject in a subject in need thereof, the method comprising administering to the subject an effective amount of the polynucleotide disclosed herein, a composition disclosed herein, or a cell disclosed herein.

The present disclosure also provides a method of treating cancer in a subject in need thereof, the method comprising administering to the subject an effective amount of (a) an mRNA encoding SEQ ID NO: 21 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 20 or to the subsequence thereof encoding said CTLA-4-binding portion; (b) an mRNA encoding SEQ ID NO: 23 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 22 or to the subsequence thereof encoding said CTLA-4-binding portion; (c) an mRNA encoding SEQ ID NO: 25 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 24 or to the subsequence thereof encoding said CTLA-4-binding portion; (d) an mRNA encoding SEQ ID NO: 27 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 26 or to the subsequence thereof encoding said CTLA-4-binding portion; (e) the mRNA according to (a) and the mRNA according to (b); (f) the mRNA according to (c) and the mRNA according to (d); (g) an mRNA comprising (a) and (b); or (h) an mRNA comprising (c) and (d). In some aspects, the mRNA comprises at least one chemically modified nucleoside at least one chemically modified nucleoside is selected from the group consisting of pseudouridine (ψ), N1-methylpseudouridine (m1ψ), 5-methylcytosine, 5-methoxyuridine, and a combination thereof. In some aspects, the mRNA sequence or subsequence is a codon optimized sequence.

The present disclosure also provides a method of reducing the size of a tumor or inhibiting the growth of a tumor in a subject in need thereof comprising administering to said subject at least one effective dose of the polynucleotide or the pharmaceutical composition disclosed above comprising said polynucleotide, wherein the at least one effective dose is such that (i) a plasma level of the antibody or an antigen binding portion thereof which specifically binds to CTLA-4 of at least 3 μg/mL is reached within 24 hours after administration of the dose; (ii) a plasma level of the antibody or an antigen binding portion thereof which specifically binds to CTLA-4 of at least 2 μg/mL is reached within 48 hours after administration of the dose; (iii) a plasma level of the antibody or an antigen binding portion thereof which specifically binds to CTLA-4 of at least 1 μg/mL is reached within 72 hours after administration of the dose; or, (iv) a plasma level of the antibody or an antigen binding portion thereof which specifically binds to CTLA-4 of at least 0.5 μg/mL is reached within 7 days after administration of the dose. In some aspects, the polynucleotide comprises. consists, or consists essentially of (a) an mRNA encoding SEQ ID NO: 21 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 20 or to the subsequence thereof encoding said CTLA-4-binding portion; (b) an mRNA encoding SEQ ID NO: 23 or a CTA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 22 or to the subsequence thereof encoding said CTLA-4-binding portion; (c) an mRNA encoding SEQ ID NO: 25 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 24 or to the subsequence thereof encoding said CTLA-4-binding portion; (d) an mRNA encoding SEQ ID NO: 27 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 26 or to the subsequence thereof encoding said CTLA-4-binding portion; (e) the mRNA according to (a) and the mRNA according to (b); (f) the mRNA according to (c) and the mRNA according to (d); (g) an mRNA comprising (a) and (b); or (h) an mRNA comprising (c) and (d). In some aspects, the at least one effective dose comprises one, two, or three doses. In some aspects, the doses are administered within at least 3 day intervals. In some aspects, the polynucleotide is administered at 0.5 mg/kg/dose. In some aspects, the administration of the at least one effective dose results in a remission rate of at least 60%. in some aspects, the administration of the at least one effective dose results in (i) reduction of the size of a tumor by at least 50%, (ii) inhibition of the growth of the tumor by at least 50%, or (iii) a combination thereof.

### BRIEF DESCRIPTION OF THE DRAWINGS/FIGURES

FIG. 1 shows total IgG (mIgG) versus active CTLA-4 binding (mCTLA4 binding) in HeLa cells expressing mRNA-encoded 9D9 antibodies. The 9D9 antibodies are HC-2Aa:LC (9D9 IgG2a antibody) and HC-2B:LC (9D9 IgG2b antibody).

FIGS. 2A-2C show positive/negative efficacy control data in CT26 carcinoma model from 3 doses of 5 mg/kg anti-CTLA-4 protein (FIG. 2B) or 0.5 mg/kg NST FIX (FIG. 2C) administered at days 3, 6 and 9 as compared to untreated animals (FIG. 2A). NST FIX is a negative control mRNA (mRNA encoding non-translated (“non-start”) Factor IX).

FIGS. 3A-3F show treatment with 1, 2 or 3 doses of mRNA encoding anti-CTLA-4 (9D9 2b) antibody. FIGS. 3A and 3B show data corresponding to control sample NST FIX and 9D9 2b anti-CTLA-4 antibody, respectively, administered at day 3. FIGS. 3C and 3D show data corresponding to control sample NST FIX and 9D9 2b anti-CTLA-4 antibody, respectively, administered at day 3 and day 9. FIGS. 3E and 3F show data corresponding to control sample NST FIX and 9D9 2b anti-CTLA-4 antibody, respectively, administered at day 3, day 6 and day 9.

FIGS. 4A-4F show treatment with 1, 2 or 3 doses of mRNA encoding anti-CTLA-4 (9D9 2a). FIG. 4A and 4B show data corresponding to control sample NST FIX and 9D9 2a anti-CTLA-4 antibody, respectively, administered at day 3. FIG. 4C and 4D show data corresponding to control sample NST FIX and 9D9 2a anti-CTLA-4 antibody, respectively, administered at day 3 and day 9. FIG. 4E and 4F show data corresponding to control sample NST FIX and 9D9 2a anti-CTLA-4 antibody, respectively, administered at day 3, day 6 and day 9.

FIGS. 5A-5B show the efficacy of 9D9 2b anti-CTLA antibody protein in the CT26 carcinoma model (FIG. 5B) compared to untreated controls (FIG. 5A). The 9D9 2b anti-CTLA-4 antibody was administered at days 3, 6 and 9 after tumor implantation.

FIG. 6 shows senim levels of anti-CTL A-4 9D9 antibody after administration of a 5 mg/kg dose (observed at 24 hours (left bar), 48 hours (middle bar), and 72 hours (right bar) timepoints) or after administration of 0.5 mg/kg of mRNA encoding the 9D9 2b antibody, mRNA encoding the 9D9 2a antibody, or controls (NST FIX or DPBS) (observed at 24 hours, 48 hours, 72 hours, and 7 days timepoints, from left to right respectively).

FIGS. 7A-7B show individual tumor growth curves for untreated animals (FIG. 7A) and control animals treated with NST FIX/LNP (FIG. 7B). mRNA encoding the negative control NST FIX was administered at 0.5 mg RNA/kg at day 3, at day 6, and at day 9 after tumor implantation.

FIG. 8 shows individual tumor growth curves for animals treated with 3 doses of 5 mg/kg anti-CTLA-4 9D9 antibody protein administered at day 3, at day 6, and at day 9 after tumor implantation.

FIG. 9 shows individual tumor growth curves for animals treated with 3 doses of mRNA designed to express anti-CTLA-4 9D9 2b antibody. 0.5 mg mRNA/kg doses were administered at day 3, at day 6, and at day 9 after tumor implantation.

FIG. 10 shows individual tumor growth curves for animals treated with 3 doses of mRNA designed to express anti-CTLA-4 9D9 2a antibody. 0.5 mg mRNA/kg doses were administered at day 3, at day 6, and at day 9 after tumor implantation.

FIGS. 11A-11B show individual tumor growth curves for animals treated with NST FIX mRNA control (FIG. 11A) and animals treated with mRNA designed to expressed anti-CTLA-4 9D9 2a (FIG. 11B). mRNAs were administered at 0.5 mg RNA/kg at day 3 and at day 9 after tumor implantation.

FIGS. 12A-12B shows individual tumor growth curves for animals treated with NST FIX mRNA control (FIG. 12A) and animals treated with mRNA designed to expressed anti-CTLA-4 9D9 2a (FIG. 12B). mRNAs were administered at 0.5 mg RNA/kg at day 3 after tumor implantation.

FIG. 13 shows the survival benefit from treatment with mRNAs encoding CTLA-4 antibodies.

### DETAILED DESCRIPTION

The present application is directed to methods treating cancer in a subject in need thereof comprising administering to the subject a polynucleotide comprising one or more mRNAs encoding a binding molecule (e.g., an antibody or an antigen binding portion thereof) which specifically hinds to CTLA-4. Following in vivo expression, the polypeptide encoded by the polynucleotides described herein can reduce or decrease a size of a tumor or inhibit a tumor growth in a subject in need thereof by providing a polynucleotide comprising one or more mRNAs which encodes a binding molecule (e.g., an antibody or an antigen binding portion thereof) which specifically binds to CTLA-4.

The studies presented herein demonstrate the therapeutic efficacy of mRNAs encoding two variants of the anti-CTLA-4 antibodies against lethal challenge with tumors in a model system. Therapeutic administration of LNP-encapsulated mRNA encoding an anti-CTLA-4 antibody showed inhibition in tumor burden and significant inhibition of tumor growth. Thus, expression of mRNA encoding anti-CTLA-4 antibodies can provide significant therapeutic benefit in combatting tumor growth. These data also demonstrate that in vivo administration of mRNA encoding anti-CTLA-4 antibodies results in the rejection of tumors, indicating that blockade of the inhibitory effects of CTLA-4 using mRNA-encoded antagonistic antibodies provide for effective immune responses against tumor cells.

The headings provided herein are not limitations of the various aspects or aspects of the disclosure, which can be defined by reference to the specification as a whole. Accordingly, the terms defined immediately below are more fully defined by reference to the specification in its entirety. Before describing the present invention in detail, it is to be understood that this invention is not limited to specific compositions or process steps, as such can vary.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The invention includes embodiments in which exactly one member of the group is present in, employed in, or otherwise relevant to a given product or process. The invention includes embodiments in which more than one, or all of the group members are present in, employed in, or otherwise relevant to a given product or process.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

About: The term “about” as used in connection with a numerical value throughout the specification and the claims denotes an interval of accuracy, familiar and acceptable to a person skilled in the art. In general, such interval of accuracy is ±10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

Amino acid substitution: The term “amino acid substitution” refers to replacing an amino acid residue present in a parent sequence (e.g., a consensus sequence) with another amino acid residue. An amino acid can be substituted in a parent sequence, for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position X, and Y and Z are alternative substituting amino acid residue, i.e., In the context of the present disclosure, substitutions (even when they referred to as amino acid substitution) are conducted at the nucleic acid level, i.e., substituting an amino acid residue with an alternative amino acid residue is conducted by substituting the codon encoding the first amino acid with a codon encoding the second amino acid.

Codon substitution: The terms “codon substitution” or “codon replacement” in the context of codon optimization refer to replacing a codon present in a candidate nucleotide sequence (e.g., an mRNA encoding the heavy chain or light chain of an antibody or a fragment thereof) with another codon. A codon can be substituted in a candidate nucleic acid sequence, for example, via chemical peptide synthesis or through recombinant methods known in the art. Accordingly, references to a “substitution” or “replacement” at a certain location in a nucleic acid sequence (e.g., an mRNA) or within a certain region or subsequence of a nucleic acid sequence (e.g., an mRNA) refer to the substitution of a codon at such location or region with an alternative codon. A candidate nucleic acid sequence can be codon-optimized by replacing all or part of its codons according to a substitution table map. As used herein, the terms “candidate nucleic acid sequence” and “candidate nucleotide sequence” refer to a nucleotide sequence (e.g., a nucleotide sequence encoding an antibody or a functional fragment thereof) that can be codon-optimized, for example, to improve its translation efficacy. In some aspects, the candidate nucleotide sequence is optimized for improved translation efficacy after in vivo administration.

Conservative amino acid substitution: A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitution: Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can be readily identified by workers of ordinary skill. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats a tumor, an effective amount of an agent is, for example, an amount sufficient to reduce or decrease a size of a tumor or to inhibit a tumor growth, as compared to the response obtained without administration of the agent. The term “effective amount” can be used interchangeably with “effective dose,” “therapeutically effective amount,” or “therapeutically effective dose.”

Expression: As used herein, “expression” of a nucleic acid sequence refers to one or more of the following events: (1) production of an mRNA template from a DNA sequence (e.g., by transcription); (2) processing of an mRNA transcript (e.g., by splicing, editing, 5′ cap formation, and/or 3′ end processing); (3) translation of an mRNA into a polypeptide or protein; and (4) post-translational modification of a polypeptide or protein.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity “%ID” of a first amino acid sequence (or nucleic acid sequence) to a second amino acid sequence (or nucleic acid sequence) is calculated as %ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

Immune response: The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

Isolated: As used herein, the term “isolated” refers to a substance or entity that has been separated from at least some of the components with which it was associated (whether in nature or in an experimental setting). Isolated substances (e.g., nucleotide sequence or protein sequence) can have varying levels of purity in reference to the substances from which they have been associated. Isolated substances and/or entities can be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was for riled or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof.

A polynucleotide, vector, polypeptide, cell, or any composition disclosed herein which is “isolated” is a polynucleotide, vector, polypeptide, cell, or composition which is in a form not found in nature. Isolated polynucleotides, vectors, polypeptides, or compositions include those which have been purified to a degree that they are no longer in a form in which they are found in nature. In some aspects, a polynucleotide, vector, polypeptide, or composition which is isolated is substantially pure.

Nucleic acid sequence: The terms “nucleic acid sequence,” “nucleotide sequence,” or “polynucleotide” are used interchangeably and refer to a contiguous nucleic acid sequence. The sequence can be either single stranded or double stranded DNA or RNA, e.g., an mRNA.

The phrase “nucleotide sequence encoding” and variants thereof refers to the nucleic acid (e.g., an mRNA or DNA molecule) coding sequence that comprises a nucleotide sequence which encodes a polypeptide or functional fragment thereof as set forth herein. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding mRNA sequences (comprising U) are considered codon-optimized nucleotide sequence of the present invention. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ΨΨC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am Chem Soc. 1 14:3675-3683 and Switzer et al., supra.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid.

Polynucleotide of the present disclosure: The terms “polynucleotide of the present disclosure,” “polynucleotide of the present invention,” and grammatical variants thereof are used interchangeably throughout this disclosure and refer to one or more polynucleotides that would be required to express in vivo a functional binding molecule (e.g., an antibody or an antigen-binding portion thereof) that specifically binds to CTLA-4. Accordingly, e.g., each one of (i) a polynucleotide encoding the heavy chain of an antibody, (ii) a polynucleotide encoding the light chain of an antibody, (ii) a set of polynucleotides encoding the heavy chain and the light of an antibody, respectively, or (iv) a polynucleotide encoding both the heavy chain and the light chain of an antibody, would he considered a “polynucleotide of the present disclosure.”

Prophylaxis: As used herein, a “prophylaxis” refers to a measure taken to maintain health and prevent the spread of disease. An “immune prophylaxis” refers to a measure to produce active or passive immunity to prevent the spread of disease.

Pseudouridine: As used herein, pseudouridine (ψ) refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m¹ψ), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine (acp³ψ), and 2′-O-methyl-pseudouridine (ψm).

Signal transduction pathway: A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and the transmission of such a signal across the plasma membrane of a cell. An example of a “cell surface receptor” of the present invention is the CTLA-4 receptor.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of a cancer treatment.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Treating, treatment, therapy: As used herein, the term “treating” or “treatment” or “therapy” refers to partially or completely alleviating, ameliorating, improving, relieving, delaying onset of, inhibiting progression of, reducing severity of, and/or reducing incidence of one or more symptoms or features of a hyper-proliferative disease, e.g., cancer. For example, “treating” cancer can refer to inhibiting survival, growth, and/or spread of a tumor. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified can, but does not always, refer to the wild type or native form of a biomolecule. Molecules can undergo a series of modifications whereby each modified molecule can serve as the “unmodified” starting molecule for a subsequent modification.

Antibody: The terms “antibody” or “immunoglobulin,” are used interchangeably herein, and include whole antibodies and any antigen binding portion or single chains thereof. A typical antibody comprises at least two heavy (H) chains and two light (L) chains interconnected by disulfide bonds. Each heavy chain is comprised of a heavy chain variable region (abbreviated herein as VH or VH) and a heavy chain constant region. The heavy chain constant region is comprised of three domains, CH1, CH2, and CH3. Each light chain is comprised of a light chain variable region (abbreviated herein as VL or VL) and a light chain constant region. The light chain constant region is comprised of one domain, CL. The VH and VL regions can be further subdivided into regions of hypervariability, termed Complementarity Determining Regions (CDR), interspersed with regions that are more conserved, termed framework regions (FW). Each VH and VL is composed of three CDRs and four FWs, arranged from amino-terminus to carboxy-terminus in the following order: FW1, CDR1, FW2, CDR2, FW3, CDR3, and FW4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antibody” encompasses any immunoglobulin molecules that recognize and specifically bind to a target, e.g., CTLA-4, through at least one antigen recognition site within the variable region of the immunoglobulin molecule. The term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations.

The term antibody also encompasses molecules comprising an immunoglobulin domain from an antibody (e.g., a VH, CL, CL, CH1, CH2 or CH3 domain) fused to other molecules, i.e., fusion proteins. In some aspects, such fusion protein comprises an antigen-binding moiety (e.g., an scFv). The antibody moiety of a fusion protein comprising g an antigen-binding moiety can be used to direct a therapeutic agent (e.g., a cytotoxin) to a desired cellular or tissue location determined by the specificity of the antigen-binding moiety.

Therapeutic antibody: The term “therapeutic antibody” is used in a broad sense, and encompasses any antibody or a functional fragment thereof that functions to deplete target cells in a patient. Such target cells include, e.g., tumor cells. The therapeutic antibodies can, for instance, mediate a cytotoxic effect or cell lysis, particularly by antibody-dependent cell-mediated cytotoxicity (ADCC). Therapeutic antibodies according to the disclosure can be directed to epitopes of surface which are overexpressed by cancer cells.

Blocking antibody: In some aspects, the therapeutic antibody is a blocking antibody. The teens “blocking antibody” or “antagonist antibody” refer to an antibody which inhibits or reduces the biological activity of the antigen it binds. In a certain aspect blocking antibodies or antagonist antibodies substantially or completely inhibit the biological activity of the antigen. In some aspects, the biological activity is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%,at least 85%, at least 90%, at least 95%, or even 100%.

Antigen binding portion thereof. The term “antigen binding portion” as used herein, when used in reference to an antibody disclosed herein, is intended to refer to a portion of the antibody which is capable of specifically binding an antigen that is specifically bound by the antibody. The term antigen binding portion also refers to a construct derived from an antibody that functions as a blocking or a targeting antibody, e.g., an scFv. Whether a binding portion is still capable to specifically binding to its antigen can be determined using binding assays known in the art (e.g., BIACORE).

Variant: The term “variant” as used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleotide sequence or the complement thereof; (iv) a nucleotide sequence that hybridizes under stringent conditions to the referenced nucleotide sequence, complement thereof, or a sequence substantially identical thereto, or (v) a nucleotide sequence comprising one or more substitutions and encodes a polypeptide retaining at least one biological activity (e.g., antigen binding) of the polypeptide encoded by the referenced nucleotide sequence. “Variant” with respect to a polypeptide refers to a polypeptide that differs in amino acid sequence by the insertion, deletion, or conservative substitution of amino acids, but retains at least one biological activity of a reference polypeptide sequence (e.g., antigen binding).

Variable region: :A “variable region” of an antibody refers to the variable region of the antibody light chain or the variable region of the antibody heavy chain, either alone or in combination. The variable regions of the heavy and light chain each consist of four FW regions connected by three CDR regions. The CDRs in each chain are held together in close proximity by the FW regions and, with the CDRs from the other chain, contribute to the formation of the antigen-binding site of antibodies. There are several techniques for determining the location of CDRs. The Kabat numbering system is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The boundaries of the antibody structural elements presented in this disclosure, namely, CDR1, CDR2, and CDR3 of a VH or VL domain; VH and VL domain; and constant domains CL, CH1, CH2, and CH3 can be identified according to methods know in the art. For example, the boundaries between structural elements in an antibody can be identified from sequence data alone by using the Paratome tool available at URL tools.immuneepitope.org/paratome/. See, Kunik et al. (2012) PLoS Comput. Biol. 8:2; Kunik et al. (2012). Nucleic Acids Res. 40(Web Server issue):W521-4.

Epitope: The term “epitope” as used herein refers to an antigenic protein determinant capable of binding to an antibody or antigen-binding portion thereof disclosed herein. Epitopes usually consist of chemically active surface groupings of molecules such as amino acids or sugar side chains and usually have specific three dimensional structural characteristics, as well as specific charge characteristics. The part of an antibody or binding molecule that recognizes the epitope is called a paratope. The epitopes of protein antigens are divided into two categories, conformational epitopes and linear epitopes, based on their structure and interaction with the paratope. A conformational epitope is composed of discontinuous sections of the antigen's amino acid sequence. These epitopes interact with the paratope based on the 3-D surface features and shape or tertiary structure of the antigen. By contrast, linear epitopes interact with the paratope based on their primary structure. A linear epitope is formed by a continuous sequence of amino acids from the antigen.

### II. Methods of Use

CTLA-4 is a member of the CD28-B7 immunoglobulin superfamily of immune regulatory molecules. CTLA-4 is expressed on the surface of T cells, where it is able to suppress T cell activation downstream of T-cell receptor (TCR) signaling. (Schwartz, Cell 71:1065-1068 (1992); Krummel and Allison, J Exp. Med. 182:459-465 (1995)). CTLA-4 is also believed to outcompete the T cell costimulatory CD28 for the B7 ligands, CD80 and CD86, on the surface of antigen-presenting cells (APCs) by binding them with higher affinity and avidity (Linsley et al., J Exp Med. 174:561-569 (1991)). The physiologic role of CTLA-4 is not only to suppress effector T cells (Teffs), but also to increase the function of immunosuppressive, regulatory T cells (Tregs) (Wing et al., Science 322:271-5 (2008)).

The canonical sequence of human CTLA-4 (SEQ ID NO:1), isoform 1, is 223 amino acids long. It contains a signal peptide at positions 1-35. The mature form comprises residues 36 to 223 (SEQ ID NO:3), of which residues 36-161 are the extracellular domain (SEQ ID NO: 2), residues 162-182 are a transmembrane helix, and residues 183-223 are the intracellular domain. Four additional isoforms have been described in the literature. Isoform 2 (SEQ ID NO:4), also known as ss-CTLA-4, is 56 amino acids long and lacks the region from residue 38 to residue 204. Isoform 3 (SEQ ID NO:5) is 58 amino acids long and also lacks the region from residue 38 to residue 204. In addition, isoform 3 has an alternative sequence between residues 205 and 223. Isoform 4 (SEQ ID NO:6) is 79 amino acids long, lacks the region from residue 59 to residue 204, has an alternative sequence from residue 205 to residue 223, and contains a C58S point mutation. Isoform 5 (SEQ ID NO:7) is 174 amino acids long, lacks the sequence from residue 175 to residue 223, and contains an alternative sequence from residue 153 to residue 174.

The methods of the present invention provide for the use of a polynucleotide comprising an mRNA (or more than one mRNA) encoding a binding molecule that can specifically bind to CTLA-4, e.g., an anti-CTLA-4 antibody or an antigen binding portion thereof.

In some aspects, the CTLA-4 binding molecule is a oligomer, e.g., an IgG antibody, in which case the CTLA-4 binding molecule can be encoded by a single mRNA molecule comprising an open reading frame (ORF) encoding the heavy chain of the antibody, and an ORF encoding the light of the antibody, or alternatively, the antibody can be encoded by two or more mRNA molecules, each of then comprising at least one ORF (e.g., an mRNA encoding the antibody heavy chains and a second mRNA encoding the antibody light chains).

In some aspects, when the CTLA-4 binding molecule (e.g., an antibody) is encoded by more than one mRNA, the mRNAs can be administered at equimolar ratios or at different ratios. In some aspects, when the CTLA-4 binding molecule (e.g., an antibody) is encoded by more than one mRNA, the mRNAs (e.g., an mRNA encoding the light chain and an mRNA encoding the heavy chain) can be administered in a single composition or in separate formulations. In addition, when the CTLA-4 binding molecule (e.g., an antibody) is encoded by more than one mRNA, the mRNAs (e.g., an mRNA encoding the light chain and an mRNA encoding the heavy chain) can be administered concurrently or sequentially.

In some aspects, the polynucleotides of the present invention (e.g., one or more mRNAs encoding a binding molecule that can specifically bind to CTLA-4 such as an anti-CTLA-4 antibody) can be used to prevent and/or treat cancers, i.e., it can have prophylactic as well as therapeutic uses. Accordingly, the present disclosure provides method to prevent and/or treat cancers, and in particular tumors associated, for example, with melanoma, lung cancer, bladder cancer, or prostate cancer.

The present disclosure provides methods of reducing the size of a tumor or inhibiting the growth of a tumor in a subject in need thereof comprising administering to said subject one or more mRNAs encoding an antibody or an antigen binding portion thereof which specifically binds to CTLA-4. The present disclosure also provides a method of activating T cells in a subject in need thereof comprising administering to the subject one or more mRNAs encoding an antibody or an antigen binding portion thereof which specifically binds to CTLA-4. In one aspect, the activation of T cells in the subject is directed to an anti-tumor immune response in the subject. In another aspect, the activated T cells in the subject reduce or decrease the size of a tumor or inhibit the growth of a tumor in the subject. Activation of T cells can be measured using applications in the art such as measuring T cell proliferation; measuring cytokine production with enzyme-linked immunosorbant assays (ELISA) or enzyme-linked immunospot assays (ELISPOT); or detection of cell-surface markers associated with T cell activation (e.g., CD69, CD40L, CD137, CD25, CD71, CD26, CD27, CD28, CD30, CD154, and CD134) with techniques such as flow cytometry.

In some embodiments, the present invention provides a method of inducing T cell proliferation in a subject in need thereof comprising administering to the subject one or more mRNAs encoding an antibody or an antigen binding portion thereof which specifically binds to CTLA-4. In one aspect, the T cell proliferation in the subject is directed to an anti-tumor immune response in the subject. In another aspect, the T cell proliferation in the subject reduces or decreases the size of a tumor or inhibits the growth of a tumor in the subject. T cell proliferation can be measured using applications in the art such as cell counting, viability staining, optical density assays, or detection of cell-surface markers associated with T cell activation (e.g., CD69, CD40L, CD137, CD25, CD71, CD26, CD27, CD28, CD30, CD154, and CD134) with techniques such as flow cytometry.

In other embodiments, the present invention provides a method of inducing a memory T cell response in a subject in need thereof comprising administering to the subject one or more mRNAs encoding an antibody or an antigen binding portion thereof which specifically binds to CTLA-4. In one aspect, the memory T cell response in the subject is directed to an anti-tumor immune response in the subject. In another aspect, the memory T cell response in the subject reduces or decreases the size of a tumor or inhibits the growth of a tumor in the subject. A memory T cell response can be measured using applications in the art such as measuring T cell markers associated with memor T cells, measuring local cytokine production related to memory immune response, or detecting memory T cell-surface markers with techniques such as flow cytometry.

The present disclosure further provides a method of increasing immunocytokine (e.g., interleukin-2) production in a subject in need thereof comprising administering to the subject one or more mRNAs encoding an antibody or an antigen binding portion thereof which specifically binds to CTLA-4. In one aspect, the increase in immunokine production in the subject is directed to an anti-tumor immune response in the subject. In one embodiment, the increase in immunokine production is at least about two-fold, at least about three-fold, at least about four-fold, at least about five-fold, or at least about six-fold higher than a control (e.g., PBS treated). The IL-2 expression can be measured using any available techniques, such as ELISA or ELISPOT assays.

The polynucleotide (e.g., mRNA) of the present disclosure can be administered in any route available, including, but not limited to, intratumoral, enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraperitoneal (into the peritoneum), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In other embodiments, the mRNA of the present disclosure is administered parenterally (e.g., includes subcutaneous, intravenous, intraperitoneal, intratumoral, intramuscular, intra-articular, intra-synovial, intrasternal, intrathecal, intrahepatic, intralesional and intracranial injection or infusion techniques), intraventricularly, orally, by inhalation spray, topically, rectally, nasally, buccally, vaginally or via an implanted reservoir.

The present disclosure also includes a method of inducing a memory T cells response in a subject in need thereof comprising administering, e.g., administering intratumorally, one or more mRNAs encoding an antibody or an antigen binding portion thereof which specifically binds to CTLA-4. In certain embodiments, the intratumoral administration of the present mRNA can increase the efficacy of the anti-tumor effect (e.g., memory T cell response) compared to other routes of administration.

The present disclosure further provides a method of increasing immunocytokine (e.g., interleukin-2) production in a subject in need thereof comprising administering, e.g., administering intratumorally, one or more mRNAs encoding an antibody or an antigen binding portion thereof which specifically binds to CTLA-4 to the subject. In one aspect, the increase in immunocytokine (e.g., interleukin-2) production in the subject is directed to an anti-tumor immune response in the subject.

The present disclosure also includes a method of activating T cells in a subject in need thereof comprising administering, e.g., administering intratumorally, one or more mRNAs encoding an antibody or an antigen binding portion thereof which specifically binds to CTLA-4 to the subject. In certain embodiments, the intratumoral administration of the one or more mRNAs can increase the efficacy of the anti-tumor effect (e.g., T cell activation) compared to other routes of administration.

The present disclosure also includes a method of inducing T cell proliferation in a subject in need thereof comprising administering, e.g., administering intratumorally, one or more mRNAs encoding an antibody or an antigen binding portion thereof which specifically binds to CTLA-4 to the subject. In certain embodiments, the intratumoral administration of the one or more mRNAs can increase the efficacy of the anti-tumor effect (e.g., T cell proliferation) compared to other routes of administration.

The present disclosure further includes a method of reducing the size of a tumor or inhibiting the growth of a tumor in a subject in need thereof comprising administering (e.g., intratumorally, intraperitoneally, intramuscularly, intradermally or intravenously) one or more mRNAs encoding an antibody or an antigen binding portion thereof which specifically binds to CTLA-4 as a monotherapy, i.e., without any other anti-cancer agent in combination. In some embodiments, the methods disclosed above also comprise administering (e.g., intratumorally, intraperitoneally, intramuscularly, intradermally or intravenously) one or more mRNAs encoding an antibody or an antigen binding portion thereof which specifically binds to CTLA-4 as a monotherapy.

In some aspects of the methods disclosed herein, the size of a tumor can be reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%, with respect to the original size of the tumor prior to treatment with one or more mRNAs encoding an antibody or an antigen binding portion thereof which specifically binds to CTLA-4.

In some aspects of the methods disclosed herein, the growth of a tumor can be inhibited by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%, with respect to the original growth rate of the tumor prior to treatment with one or more mRNAs encoding an antibody or an antigen binding portion thereof which specifically binds to CTLA-4.

In some aspects of the methods disclosed herein, the survival rate can be increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100%, with respect to the survival rate of a population of subjects which have not been treated with one or more mRNAs encoding an antibody or an antigen binding portion thereof which specifically binds to CTLA-4. In some aspects of the methods disclosed herein, the survival rate in a subject treated with one or more mRNAs encoding an antibody or an antigen binding portion thereof which specifically binds to CTLA-4 can be at least 2-fold, at least 3-fold, at least 4-fold, at least 5-fold, at least 6-fold, at least 7-fold, at least 8-fold, at least 9-fold or at least 10-fold higher than the survival rate of a population of subjects which have not been treated with one or more mRNAs encoding an antibody or an antigen binding portion thereof which specifically binds to CTLA-4.

In some aspects of the methods disclosed herein, the survival rate in a population of subjects in need of treatment which have been treated with one or more mRNAs encoding an antibody or an antigen binding portion thereof which specifically binds to CTLA-4 can be at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%.

In some aspects, response rate (e.g., partial response or complete response) in a population of subjects in need of treatment which have been treated with one or more mRNAs encoding an antibody or an antigen binding portion thereof which specifically binds to CTLA-4 can be at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%.

In some aspects, the complete response or complete remission rate in a population of subjects in need of treatment which have been treated with one or more mRNAs encoding an antibody or an antigen binding portion thereof which specifically binds to CTLA-4 can be at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%. In some specific aspects, the complete remission rate for subjects treated with one or more mRNAs encoding an antibody or an antigen binding portion thereof which specifically binds to CTLA-4 disclosed herein is about 100% when the one or more mRNAs are administered in one, two, or three doses, wherein the doses are about 0.5 mg mRNA/kg, and wherein such doses result in plasma levels of the antibody or an antigen binding portion thereof between 0.5 μg/mL and 5 μg/mL.

In some aspects, the inhibition of tumor growth following the administration of one or more mRNAs encoding an antibody or an antigen binding portion thereof which specifically binds to CTLA-4 can result in complete remission (i.e., complete response), improvement in response or partial response (e.g., increase in time survival, size of tumors, etc.), lowering of tumor burden, or a combination thereof. In one aspect, the administration of one or more mRNAs encoding an antibody or an antigen binding portion thereof which specifically binds to CTLA-4 results in complete remission.

In some aspects, the plasma concentration of an antibody or an antigen binding portion thereof which specifically binds to CTLA-4 encoded by one or more mRNAs following administration to a subject in need thereof is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, at least about 0.1 μg/mL, at least about 0.2 μg/mL, at least about 0.3 μg/mL, at least about 0.4 μg/mL, at least about 0.5 μg/mL, at least about 0.6 μg/mL, at least about 0.7 μg/mL, at least about 0.8 μg/mL, at least about 0.9 μg/mL, at least about 1 μg/mL, at least about 2 μg/mL, at least about 3 μg/mL, at least about 4 μg/mL, at least about 5 μg/mL, at least about 6 μg/mL, at least about 7 μg/mL, at least about 8 μg/mL, at least about 9 μg/mL, at least about 10 μg/mL, at least about 15 μg/mL, at least about 20 μg/mL, at least about 25 μg/mL, at least about 30 μg/mL, at least about 35 μg/mL, at least about 40 μg/mL, at least about 45 μg/mL, or at least about 50 μg/mL.

In certain embodiments, the methods disclosed herein comprise administering (e.g., intratumorally, intraperitoneally, intramuscularly, intradermally or intravenously) one or more mRNAs encoding an antibody or an antigen binding portion thereof which specifically binds to CTLA-4 in combination with one or more anti-cancer agents to the subject. In certain embodiments, the one or more anti-cancer agents are an mRNA encoding a tumor antigen. In other embodiments, the one or more anti-cancer agents are not a tumor antigen or an mRNA encoding a tumor antigen. In some embodiments, the one or more anti-cancer agents is an approved agent by the United States Food and Drug Administration. In other embodiments, the one or more anti-cancer agents is a pre-approved agent by the United States Food and Drug Administration.

In some aspects, the subject for the present methods has been treated with one or more standard of care therapies. In other aspects, the subject for the present methods has not been responsive to one or more standard of care therapies or anti-cancer therapies.

In some aspects, the subject has been treated with a CTLA-4 antagonist prior to the treatment with a polynucleotide of the present disclosure. In another aspect, the subject has been previously treated with a monoclonal antibody that binds to CTLA-4 prior to the treatment with one or more mRNAs encoding an antibody or an antigen binding portion thereof which specifically binds to CTLA-4. In another aspect, the subject has been treated with an anti-CTLA-4 monoclonal antibody prior to the treatment with a polynucleotide of the present invention.

The administration of polynucleotides, e.g., mRNA, as referred to in the present disclosure is not in the form of a dendritic cell comprising the polynucleotide, e.g., polynucleotides encoding one or more mRNAs encoding an antibody or an antigen binding portion thereof which specifically binds to CTLA-4. Rather, the administration in the present disclosure is a direct administration of one or more polynucleotides, e.g., mRNAs, encoding an antibody or an antigen binding portion thereof which specifically binds to CTLA-4 to the subject (e.g., to a tumor in a subject).

The present disclosure provides a method of reducing the size of a tumor or inhibiting the growth of a tumor in a subject in need thereof comprising administering to said subject one or more mRNAs (e.g., two, three or more mRNAs) encoding an antibody or an antigen binding portion thereof which specifically binds to the same CTLA-4 epitope as:

(i) an antibody or antigen-binding portion thereof comprising a heavy chain variable region (VH) of SEQ ID NO: 9, 28, or 39, and a light chain variable region (VL) of SEQ ID NO: 11, 29 or 41; or,

(ii) an antibody or antigen-binding portion comprising a VH of SEQ ID NO: 183 and a VL of SEQ ID NO: 185.

Also provided is method of reducing the size of a tumor or inhibiting the growth of a tumor in a subject in need thereof comprising administering to said subject one or more mRNAs (e.g., two, three or more mRNAs) encoding an antibody or an antigen binding portion thereof which specifically binds to CTLA-4 and competitively inhibits CTLA-4 binding by:

(i) an antibody or antigen-binding portion thereof comprising a VH of SEQ ID NO: 9, 28, or 39 and a VL of SEQ ID NO: 11, 29 or 41; or,

(ii) an antibody or antigen-binding portion thereof comprising a VH of SEQ ID NO: 183 and a VL of SEQ ID NO: 185.

Also provided is a method of reducing the size of a tumor or inhibiting the growth of a tumor in a subject in need thereof comprising administering to said subject one or more mRNAs (e.g., two, three or more mRNAs) encoding:

(i) an antibody or an antigen binding portion thereof which specifically binds to CTLA-4 comprising a VH of SEQ ID NO: 9, 28, or 39 and a VL of SEQ ID NO: 11, 29 or 41; or,

(ii) an antibody or antigen-binding portion thereof which specifically binds to CTLA-4 comprising a VH of SEQ ID NO: 183 and a VL of SEQ ID NO: 185.

Also provided is a method of reducing the size of a tumor or inhibiting the growth of a tumor in a subject in need thereof comprising administering to said subject one or more mRNAs encoding an antibody or an antigen binding portion thereof which specifically binds to CTLA-4, wherein the antibody or an antigen binding portion thereof comprises:

(i) a VL complementarity determining region 1 (VL-CDR1) amino acid sequence identical to, or identical except for four, three, two or one amino acid substitutions to SEQ ID NO: 17, 33, or 47, or SEQ ID NO: 189;

(ii) a VL complementarity determining region 1 (VL-CDR2) amino acid sequence identical to, or identical except for four, three, two or one amino acid substitutions to SEQ ID NO: 18, 34 or 48, or SEQ ID NO: 190;

(iii) a VL complementarity determining region 1 (VL-CDR3) amino acid sequence identical to, or identical except for four, three, two or one amino acid substitutions to SEQ ID NO: 19, 35 or 49, or SEQ ID NO: 191;

(iv) a VH complementarity determining region 1 (VH-CDR1) amino acid sequence identical to, or identical except for four, three, two or one amino acid substitutions to SEQ ID NO: 14, 30 or 44, or SEQ ID NO: 186;

(v) a VH complementarity determining region 1 (VH-CDR2) amino acid sequence identical to, or identical except for four, three, two or one amino acid substitutions to SEQ ID NO:15, 31 or 45, or SEQ ID NO: 187;

(vi) a VH complementarity determining region 1 (VH-CDR3) amino acid sequence identical to, or identical except for four, three, two or one amino acid substitutions to SEQ ID NO:16, 32 or 46 or SEQ ID NO:188;

(vii) a combination thereof.

The present disclosure also provides a method of reducing the size of a tumor or inhibiting the growth of a tumor in a subject in need thereof comprising administering to said subject one or more mRNAs (e.g., two, three or more mRNAs) encoding an antibody or an antigen binding portion thereof which specifically binds to CTLA-4, and which comprises:

(i) a VL, wherein the VL comprises VL-CDR1, VL-CDR2, and VL-CDR3 amino acid sequences identical to, or identical except for four, three, two, or one amino acid substitutions in one or more of the VL-CDRS to: SEQ ID NOs: 17, 18 and 19, SEQ ID NOs: 33, 34, and 35, SEQ ID NOs: 47, 48, and 49, or SEQ ID NOs: 189, 190, and 191, respectively;

(ii) a VH, wherein the VH comprises VH-CDR1, VH-CDR2, and. VH-CDR3 amino acid sequences identical to, or identical except for four, three, two, or one amino acid substitutions in one or more of the VH-CDRS to: SEQ ID NOs:14, 15 and 16, SEQ ID NOs: 30, 31 and 32, SEQ ID NOs: 44, 45, and 46, or SEQ D NOs: 186, 187, and 188, respectively; or,

(iii) a combination thereof.

Also provided is method of reducing the size of a tumor or inhibiting the growth of a tumor in a subject in need thereof comprising administering to said subject one or more mRNAs (e.g., two, three or more mRNAs) encoding an antibody or an antigen binding portion thereof which specifically binds to CTLA-4, and which comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 17, 18, 19, 14, 15, and 16, or SEQ ID NOs: 33, 34, 35, 30, 31, and 32, or SEQ ID NOs: 47, 48, 39, 44, 45, 46, or SEQ ID NOs: 189, 190, 191, 186, 187 and 188, respectively.

Also provided is a method of reducing the size of a tumor or inhibiting the growth of a tumor in a subject in need thereof comprising administering to said subject one or more mRNAs (e.g., two, three or more mRNAs) encoding an antibody or an antigen binding portion thereof which specifically binds to CTLA-4, and which comprises a VL and a VH, wherein the VL comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%,at least 95%, at least 96%, at least 97%, at least 98%, at least 99% sequence identity, or 100% sequence identity to SEQ ID NO: 11, SEQ ID NO: 29, SEQ ID NO:41 or SEQ ID NO: 185.

The present disclosure also provides a method of reducing the size of a tumor or inhibiting the growth of a tumor in a subject in need thereof comprising administering to said subject one or more mRNAs (e.g., two, three or more mRNAs) encoding an antibody or an antigen binding portion thereof which specifically binds to CTLA-4, and which comprises a VL and a VH, wherein the VH comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14, SEQ ID NO: 30, SEQ ID NO:44 , or SEQ ID NO: 186.

Also provided is a method of reducing the size of a tumor or inhibiting the growth of a tumor in a subject in need thereof comprising administering to said subject one or more mRNAs (e.g., two, three or more mRNAs) encoding an antibody or an antigen binding portion thereof which specifically binds to CTLA-4, and which comprises a VL and a VH wherein

(i) the VL comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11, SEQ ID NO: 29, or SEQ ID NO:41, and the VH comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9, SEQ ID NO: 28, or SEQ ID NO:39; or,

(ii) the VL comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 185, and the VH comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 183.

Also provided is a method of reducing the size of a tumor or inhibiting the growth of a tumor in a subject in need thereof comprising administering to said subject one or more mRNAs (e.g., two, three or more mRNAs) encoding an antibody or an antigen binding portion thereof which specifically binds to CTLA-4, and which comprises a VL and a VH, wherein

(i) the VL consists or consists essentially of the amino acid sequence of SEQ ID NO: 11, SEQ ID NO: 29, or SEQ ID NO:41, and the VH consists or consists essentially of the amino acid sequence of SEQ ID NO: SEQ ID NO: 9, SEQ ID NO: 28, or SEQ ID NO:39; or,

(ii) the VL consists or consists essentially of the amino acid sequence of SEQ ID NO: 185, wherein the VH consists or consists essentially of the amino acid sequence of SEQ ID NO: 183.

In some aspects of the methods disclosed herein, at least one mRNA encoding an antibody or an antigen binding portion thereof which specifically binds to CTLA-4 further encodes a heavy chain constant region or fragment thereof. In some aspects, the heavy chain constant region or fragment thereof is an IgG constant region. In some aspects, the IgG heavy chain constant region is selected from an IgG1 constant region, an IgG2 constant region, an IgG3 constant region and an IgG4 constant region. In some aspects, the IgG heavy chain constant region comprises a CH1 domain, a CH2 domain, a CH3 domain, or a combination thereof.

In some aspects of the methods disclosed herein, at least one mRNA encoding an antibody or an antigen binding portion thereof which specifically binds to CTLA-4 further encodes a light chain constant region or fragment thereof. In some aspects, the light chain constant region is selected from the group consisting of a kappa constant region and a lambda constant region.

The present disclosure also provides a method of reducing the size of a tumor or inhibiting the growth of a tumor in a subject in need thereof comprising administering to said subject one or more mRNAs (e.g., two, three or more mRNAs) encoding an antibody or an antigen binding portion thereof which specifically binds to CTLA-4, and which comprises a heavy chain (HC) a light chain (LC), wherein the HC comprises, consists, or consists essentially of the amino acid sequence of SEQ ID NO: 20 or SEQ ID NO:24, and the LC comprises, consists, or consists essentially of the amino acid sequence of SEQ ID NO: 22 or SEQ ID NO: 26. In some aspects, the one or more mRNAs are administered in one, two, or three doses. In some aspects, the one or more mRNAs are administered at a dose of about 0.5 mg/kg. In some aspects, the survival rate after administration of the one or more mRNAs is about 100%. In some aspects, administration of the one or more mRNAs results in partial or complete tumor remission. In some aspects, the plasma concentration of the antibody or antigen binding portion thereof which specifically binds to CTLA-4 encoded by the one or more mRNAs, is between 0.1 μg/mL and 5 μg/mL. In some aspects, the plasma concentration is between 0.1 μg/mL and 0.5 μg/mL.

In some aspects of the methods disclosed herein, the antibody or antigen binding portion thereof which specifically binds to CTLA-4 is a complete antibody, an antibody variant, an antibody fragment, or a combination thereof. In some aspects, the antibody or antigen binding portion thereof which specifically binds to CTLA-4 comprises, consists, or consists essentially of the antibody fragment, and wherein the antibody fragment is an scFv, Fv, Fab, F(ab′)2, Fab′, dsFv, or sc(Fv)2. In some aspects, the antibody or antigen binding portion thereof which specifically binds to CTLA-4 is an intrabody, a bicistronic antibody, a pseudobicistronic antibody, a single domain antibody, or a bispecific antibody.

In some aspects of the methods disclosed herein, the CTLA-4 to which the antibody or antigen binding portion thereof encoded by one or more mRNAs of the present invention specifically binds is human CTLA-4. In some aspects of the methods disclosed herein, the binding of an antibody or antigen binding portion thereof encoded by one or more mRNAs of the present invention to CTLA-4, e.g., human CTLA-4 (i) reduces the size of the tumor; (ii) inhibits the growth of the tumor; (iii) reduces and/or inhibits tumor cell proliferation in the subject; (iv) increases the subjects survival rate; or, (v) a combination thereof.

In some aspects of the methods disclosed herein, the tumor is selected from the group consisting of melanoma tumor, lung cancer tumor, bladder cancer tumor, colon cancer tumor, and prostate cancer tumor. In some aspects, the melanoma tumor is a metastatic melanoma tumor. In some other aspects, the lung cancer tumor is a non-small cell lung carcinoma (NSCLC) tumor or a small cell lung cancer (SCLC) tumor. In yet other aspects, the prostate cancer tumor is a metastatic hormone-refractory prostate cancer tumor.

In some aspects, the subject in need of treatment with one or more mRNAs encoding an antibody or an antigen binding portion thereof which specifically binds to CTLA-4 is human.

In some aspects of the methods disclosed herein, the antibody or antigen binding portion thereof which specifically binds to CTLA-4 is encoded by a polynucleotide sequence (e.g., an mRNA) at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 20, SEQ ID NO:24, SEQ ID NO:22, or SEQ ID NO:26, a subsequence thereof, or a combination thereof.

In some aspects of the methods disclosed herein, the antibody or antigen binding portion thereof which specifically binds to CTLA-4 is encoded by a polynucleotide sequence (e.g., an mRNA) at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to at least one of SEQ ID NO: 20, SEQ ID NO:22, SEQ ID NO:24 or SEQ ID NO:26 or to a subsequence or combination of subsequences thereof which encode:
(i) one, two or three VH-CDRs;
(ii) one, two or three VL-CDR.s;
(iii) a VH;
(iv) a VL;
(v) a HC;
(vi) a LC;
(vii) a fragment thereof; or,
(viii) a combination thereof,
   wherein the antibody or antigen binding portion thereof which specifically binds to CTLA-4 encoded by said polynucleotide sequence binds to at least one CTLA-4 molecule.

In some aspects of the methods disclosed herein (i) at least one of the one or more mRNAs encoding an antibody or an antigen binding portion thereof which specifically binds to CTLA-4 comprises at least one chemically modified nucleoside; or, (ii) each one of the one or more mRNAs encoding an antibody or an antigen binding portion thereof which specifically binds to CTLA-4 comprises at least one chemically modified nucleoside. In some aspects, at least one chemically modified nucleoside is selected from the group consisting of pseudouridine (ψ), N1-methylpseudouridine (m1ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine , 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (mH), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof. In some specific aspects, at least one chemically modified nucleoside is selected from the group consisting of pseudouridine (ψ), N1-methylpseudouridine (m1ψ), 5-methylcytosine, 5-methoxyuridine, and a combination thereof.

In some aspects, the one or more rnRNAs encoding an antibody or an antigen binding portion thereof which specifically binds to CTLA-4 comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% chemically modified nucleosides.

In some aspects, the chemically modified nucleosides in the one or more mRNAs are selected from the group consisting of uridine nucleosides, adenine nucleosides, cytosine nucleosides, guanine nucleosides, and any combination thereof. In some aspects, the uridine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%. In some aspects, the adenine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%. In some aspects, the cytosine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%. In some aspects, the guanine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%.

In some aspects of the methods disclosed herein, an mRNA encoding an antibody or an antigen binding portion thereof which specifically binds to CTLA-4 can further comprise a 5′ UTR. In some aspects, the 5′ UTR comprises a nucleic acid sequence at least 90%, at least 95%, at least 96%, at least 97%, at least 98%, at least 99%, or 100% identical to a sequence selected from SEQ ID NOS: 197-214. In some aspects, the 5′ UTR is codon optimized.

In some aspects of the methods disclosed herein, an mRNA encoding an antibody or an antigen binding portion thereof which specifically binds to CTLA-4 can further comprise a 3′ UTR. In some aspects, the 3′ UTR comprises a nucleic acid sequence at least 90%, 95%, 96%, 97%, 98%, 99%, or 100% identical to a sequence selected from SEQ ID NO: 215-232. In some aspects, the 3′ UTR is codon optimized.

In some aspects of the methods disclosed herein, an mRNA encoding an antibody or an antigen binding portion thereof which specifically binds to CTLA-4 can further comprise a 5′ terminal cap. In some aspects, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In some aspects of the methods disclosed herein, an mRNA encoding an antibody or an antigen binding portion thereof which specifically binds to CTLA-4 can further comprise a 3′ polyA tail.

In some aspects of the methods disclosed herein, the one or more mRNAs encoding an antibody or an antigen binding portion thereof which specifically binds to CTLA-4 are in vitro transcribed (IVT). In some aspects, one or more mRNAs are chimeric. In some aspects, one or more mRNAs are circular. In some aspects of the methods disclosed herein, mRNAs are purified by strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

In some aspects of the methods disclosed herein, the one or more mRNAs encoding an antibody or an antigen binding portion thereof which specifically binds to CTLA-4 are formulated with a delivery agent. In some aspects, the delivery agent comprises a lipidoid, a liposome, a lipoplex, a lipid nanoparticle, a polymeric compound, a peptide, a protein, a cell, a nanoparticle mimic, a nanotube, or a conjugate. in some aspects, the delivery agent is a lipid nanoparticle. In some aspects, the lipid nanoparticle comprises the lipid selected from the group consisting of DLin-DMA, DLin-K-DMA, 98N12-5, C12-200, DLin-MC3-DMA, DLin-KC2-DMA, DODMA, PLGA, PEG, PEG-DMG, PEGylated lipids, amino alcohol lipids, KL22, and combinations thereof.

In some aspects of the methods disclosed herein, the one or more mRNAs encoding an antibody or an antigen binding portion thereof which specifically binds to CTLA-4 are formulated for in vivo delivery. In some aspects, the one or more RNAs are formulated for subcutaneous, intravenous, intraperitoneal, intramuscular, intra-articular, intra-synovial, intrasternal, intrathecal, intrahepatic, intralesional, intracranial, intraventricular, oral, inhalation spray, topical, rectal, nasal, buccal, vaginal, intratumoral, or implanted reservoir intramuscular, subcutaneous, intratumoral, or intradermal in vivo delivery.

In some aspects, prior to and during treatment with the polynucleotides described herein a subject may be tested for to determine liver and thyroid function. In some aspects, the polynucleotides encoding an antibody or an antigen binding portion thereof which specifically binds to CTLA-4 disclosed herein have a half-life of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days. In some aspects, the polynucleotides encoding an antibody or an antigen binding portion thereof which specifically binds to CTLA-4 disclosed herein have a half-life of at least 4 week, at least 5 weeks, at least 6 weeks, at least 7 weeks, at least 8 weeks, at least 9 weeks, at least 10 weeks, at least 11 weeks, or at least 12 weeks. In some aspects, the dose of the polynucleotides encoding an antibody or an antigen binding portion thereof which specifically binds to CTLA-4 disclosed herein can be between 0.01 and 50 mg/kg (mg of polynucleotide per kg of body weight), including, but not limited to, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg and 50 mg/kg.

In some aspects, the polynucleotides encoding an antibody or an antigen binding portion thereof which specifically binds to CTLA-4 disclosed herein are administered a single time or multiple times (e.g, one time, two times, three times, four times, five times, six times, seven times, eight times, nine times or ten times). In some aspects, the doses are administered 1 day, 2 day, 3 days, 4 days, 5 days, 6 days or 7 days apart. In some aspects, the doses are administered weekly. In some aspects, the doses are administered biweekly. In some aspects, the doses are administered monthly.

The present disclosure also provides a method of treating cancer in a subject in need thereof, the method comprising administering to the subject an effective amount of:
(a) an mRNA encoding SEQ ID NO: 21 or a CTLA-4-binding portion thereof, wherein the mRNA comprises a nucleotide sequence at least 60%, at least 65%, at least 70%, at least 75%, at least 80%, at least 85%, at least 90%, at least 95%, at least 96%, at least 97%, at least of 98%, at least 99%, or 100% identical to SEQ ID NO: 20 or to the subsequence thereof encoding said CTLA-4-binding portion;
(b) an mRNA encoding SEQ ID NO: 23 or a CTLA-4-binding portion thereof, wherein the mRNA comprises a nucleotide sequence at least 60%, at least 65%, at least 70%, at least 75%, at least 80%, at least 85%, at least 90%, at least 95%, at least 96%, at least 97%, at least of 98%, at least 99%, or 100% identical to SEQ ID NO: 22 or to the subsequence thereof encoding said CTLA-4-binding portion;
(c) an mRNA encoding SEQ ID NO: 25 or a CTLA-4-binding portion thereof, wherein the mRNA comprises a nucleotide sequence at least 60%, at least 65%, at least 70%, at least 75%, at least 80%, at least 85%, at least 90%, at least 95%, at least 96%, at least 97%, at least of 98%, at least 99%, or 100% identical to SEQ ID NO: 24 or to the subsequence thereof encoding said. CTLA-4-binding portion;
(d) an mRNA encoding SEQ ID NO: 27 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 26 or to the subsequence thereof encoding said CTLA-4-binding portion;
(e) the mRNA according to (a) and the mRNA according to (b);
(f) the mRNA according to (c) and the mRNA according to (d);
(g) an mRNA comprising (a) and (b);
(h) an mRNA comprising (c) and (d); or,
(h) a combination thereof.

The present disclosure also provides a method of reducing the size of a tumor or inhibiting the growth of a tumor in a subject in need thereof comprising administering to said subject at least one effective dose of the polynucleotide or the pharmaceutical composition disclosed above comprising said polynucleotide, wherein the at least one effective dose is such that

(i) a plasma level of the antibody or an antigen binding portion thereof which specifically binds to CTLA-4 of at least 3 μg/mL is reached within 24 hours after administration of the dose;

(ii) a plasma level of the antibody or an antigen binding portion thereof which specifically binds to CTLA-4 of at least 2 μg/mL is reached within 48 hours after administration of the dose;

(iii) a plasma level of the antibody or an antigen binding portion thereof which specifically binds to CTLA-4 of at least 1 μg/mL is reached within 72 hours after administration of the dose; or,

(iv) a plasma level of the antibody or an antigen binding portion thereof which specifically binds to CTLA-4 of at least 0.5 μg/mL is reached within 7 days after administration of the dose.

In some aspects, the plasma level after the administration of the effective dose of the mRNA is at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, at least about 0.1 μg/mL, at least about 0.2 μg/mL, at least about 0.3 μg/mL, at least about 0.4 μg/mL, at least about 0.5 μg/mL, at least about 0.6 μg/mL, at least about 0.7 μg/mL, at least about 0.8 μg/mL, at least about 0.9 μg/mL, at least about 1 μg/mL, at least about 2 μg/mL, at least about 3 μg/mL, at least about 4 μg/mL, at least about 5 μg/mL, at least about 6 μg/mL, at least about 7 μg/mL, at least about 8 μg/mL, at least about 9 μg/mL, at least about 10 μg/mL, at least about 15 μg/mL, at least about 20 μg/mL, at least about 25 μg/mL, at least about 30 μg/mL, at least about 35 μg/mL, at least about 40 μg/mL, at least about 45 μg/mL, or about 50 μg/mL.

In some aspects, the plasma level after the administration of the effective dose of the mRNA is below 0.1 μg/mL, below 0.2 μg/mL, below 0.3 μg/mL, below 0.4 μg/mL, below 0.5 μg/mL, below 0.6 μg/mL, below 0.7 μg/mL, below 0.8 μg/mL, below 0.9 μg/mL, below 1 μg/mL, below 2 μg/mL, below 3 μg/mL, below 4 μg/mL, below 5 μg/mL, below 6 μg/mL, below 7 μg/mL, below 8 μg/mL, below 9 μg/mL, below 10 μg/mL, below 15 μg/mL, below 20 μg/mL, below 25 μg/mL, below 30 μg/mL, below 35 μg/mL, below 40 μg/mL, below 45 μg/mL, or below 50 μg/mL, wherein the effective dose is capable of reducing the size of a tumor or inhibiting the growth of a tumor.

In some aspects, the plasma level of the antibody or an antigen binding portion thereof which specifically binds to CTLA-4 is below 10 μg/mL within 24 hours after administration of the effective dose (e.g., a 0.5 mg/kg mRNA dose, wherein the effective dose is capable of reducing the size of a tumor or inhibiting the growth of a tumor).

In some aspects, the plasma level of the antibody or an antigen binding portion thereof which specifically binds to CTLA-4 is below 5 μg/mL within 48 hours after administration of the effective dose (e.g., a 0.5 mg/kg mRNA dose), wherein the effective dose is capable of reducing the size of a tumor or inhibiting the growth of a tumor.

In some aspects, the plasma level of the antibody or an antigen binding portion thereof which specifically binds to CTLA-4 is below 2 μg/mL within 72 hours after administration of the effective dose (e.g., a 0.5 mg/kg mRNA dose), wherein the effective dose is capable of reducing the size of a tumor or inhibiting the growth of a tumor.

In some aspects, the plasma level of the antibody or an antigen binding portion thereof which specifically binds to CTLA-4 is below 0.5 μg/mL within 7 days after administration of the effective dose (e.g., a 0.5 mg/kg mRNA dose), wherein the effective dose is capable of reducing the size of a tumor or inhibiting the growth of a tumor.

In some aspects, the plasma level of the antibody or an antigen binding portion thereof which specifically binds to CTLA-4 is below 50 μg/mL one, two, or three days after administration of the effective dose (e.g., a 0.5 mg/kg mRNA dose), wherein the effective dose is capable of reducing the size of a tumor or inhibiting the growth of a tumor.

In some aspects, the plasma level of the antibody or an antigen binding portion thereof which specifically binds to CTLA-4 is below 40 μg/mL one, two, or three days after administration of the effective dose (e.g., a 0.5 mg/kg mRNA dose), wherein the effective dose is capable of reducing the size of a tumor or inhibiting the growth of a tumor.

In some aspects, the plasma level of the antibody or an antigen binding portion thereof which specifically binds to CTLA-4 is below 30 μg/mL one, two, or three days after administration of the effective dose (e.g., a 0.5 mg/kg mRNA dose), wherein the effective dose is capable of reducing the size of a tumor or inhibiting the growth of a tumor.

In some aspects, the plasma level of the antibody or an antigen binding portion thereof which specifically binds to CTLA-4 is below 20 μg/mL one, two, or three days after administration of the effective dose (e.g., a 0.5 mg/kg mRNA dose), wherein the effective dose is capable of reducing the size of a tumor or inhibiting the growth of a tumor.

In some aspects, the plasma level of the antibody or an antigen binding portion thereof which specifically binds to CTLA-4 is below 10 μg/mL one, two, or three days after administration of the effective dose (e.g., a 0.5 mg/kg mRNA dose), wherein the effective dose is capable of reducing the size of a tumor or inhibiting the growth of a tumor.

In some aspects, the plasma level of the antibody or an antigen binding portion thereof which specifically binds to CTLA-4 is below 5 μg/mL one, two, or three days after administration of the effective dose (e.g., a 0.5 mg/kg mRNA dose), wherein the effective dose is capable of reducing the size of a tumor or inhibiting the growth of a tumor.

As used herein the terms “plasma level,” “plasma concentration,” “serum level,” or “serum concentration,” are used interchangeable. The term plasma levels refers to the concentration (e.g., in μg/mL) of an analyte (e.g., an antibody) in the plasma from blood obtained from a subject, for example, a human subject.

In some aspects, the polynucleotide used in the method of reducing the size of a tumor or inhibiting the growth of a tumor in a subject in need thereof comprises, consists, or consists essentially of (a) an mRNA encoding SEQ ID NO: 21 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 20 or to the subsequence thereof encoding said CTLA-4-binding portion; (b) an mRNA encoding SEQ ID NO: 23 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 22 or to the subsequence thereof encoding said CTLA-4-binding portion; (c) an mRNA encoding SEQ ID NO: 25 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 24 or to the subsequence thereof encoding said CTLA-4-binding portion; (d) an mRNA encoding SEQ ID NO: 27 or a CTLA-4-binding portion thereof, wherein the mRNA comprises a nucleotide sequence at least 60%, at least 70%, at least 80%, at least 85%, at least 90%, at least 95%, at least 96%, at least 97%, at least of 98%, at least 99%, or 100% identical to SEQ ID NO: 26 or to the subsequence thereof encoding said CTLA-4-binding portion; (e) the mRNA according to (a) and the mRNA according to (b); (f) the mRNA according to (c) and the mRNA according to (d); (g) an mRNA comprising (a) and (b); or (h) an mRNA comprising (c) and (d).

In some aspects, the at least one effective dose comprises one, two, three, or more doses, which can be administered at intervals of, for example, three days. Thus, if a first dose is administered at day 1, a second dose may be administered at day 4, a third dose may be administered at day 7, and so on. In a particular aspect, the polypeptide is administered in three doses at 3 day intervals.

In some aspects, the polynucleotide (or combination of polynucleotides) is administered at a dose of about 0.5 mg/kg of body weight of the subject. In other aspects, the polynucleotide (or combination of polynucleotides) is administered at a dose of about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg of body weight of the subject.

In some aspects, the administration of the at least one effective dose results in a remission rate (e.g., partial remission or complete remission) of at least 10%, at least 15%, at least 20%, at least 25%, at least 30%, at least 35%, at least 40%, at least 45%, at least 50%, at least 55%, at least 60%, at least 65%, at least 70%, at least 75%, about least 80%, at least 85%, at least 90%, at least 95%, or 100%.

In some aspects, the administration of the at least one effective dose results in (i) reduction of the size of a tumor by at least 10%, at least 15%, at least 20%, at least 25%, at least 30%, at least 35%, at least 40%, at least 45%, at least 50%, at least 55%, at least 60%, at least 65%, at least 70%, at least 75%, about least 80%, at least 85%, at least 90%, at least 95%, or 100% with respect to the initial size of the untreated tumor, or (ii) inhibition of the growth of the tumor by at least 10%, at least 15%, at least 20%, at least 25%, at least 30%, at least 35%, at least 40%, at least 45%, at least 50%, at least 55%, at least 60%, at least 65%, at least 70%, at least 75%, about least 80%, at least 85%, at least 90%, at least 95%, or 100%, with respect to the rate of growth of the untreated tumor, or (iii) a combination thereof.

In some embodiments, the mRNA sequences are codon optimized sequences.

In some aspects of the methods disclosed herein, the one or more mRNAs encoding an antibody or an antigen binding portion thereof which specifically binds to CTLA-4 comprise a leader sequence comprising, consisting, or consisting essentially of the sequence of SEQ ID NO: 234 (METPAQLLFLLLLWLPDTTG). Such leader sequence can be appended, for example, to any protein sequences listed in TABLE 1 or antigen binding portions thereof. Examples of constructs comprising such leader sequence are SEQ ID NOS: 235-236.

While exemplary aspects of the methods disclosed above are described in teens of mRNAs (e.g., mRNAs being administered in the therapeutic methods of the invention), the use of the term mRNA does not exclude other polynucleotides that may be used to practice the claimed invention. The skilled artisan will appreciate the acceptable use of the term polynucleotide in the described aspects.

III. Diseases, Disorders and/or Conditions

In some embodiments, the polynucleotides (e.g., one or more mRNAs) encoding a binding molecule (e.g., an antibody or antigen binding portion thereof) which specifically binds to CTLA-4 can be used to reduce or decrease a size of a tumor or inhibit a tumor growth in a subject in need thereof.

In some embodiments, the tumor is associated with a disease, disorder, and/or condition. In a particular embodiment, the disease, disorder, and/or condition is a cancer. Thous, in one aspect, the administration of the polynucleotides disclosed herein (e.g., one or mre mRNAs) encoding an antibody or an antigen binding portion thereof which specifically binds to CTLA-4 treats a cancer.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream.

A “cancer” or “cancer tissue” can include a tumor at various stages. In certain embodiments, the cancer or tumor is stage 0, such that, e.g., the cancer or tumor is very early in development and has not metastasized. In some embodiments, the cancer or tumor is stage I, such that, e.g., the cancer or tumor is relatively small in size, has not spread into nearby tissue, and has not metastasized. In other embodiments, the cancer or tumor is stage II or stage III, such that, e.g., the cancer or tumor is larger than in stage 0 or stage I, and it has grown into neighboring tissues but it has not metastasized, except potentially to the lymph nodes. In other embodiments, the cancer or tumor is stage IV, such that, e.g., the cancer or tumor has metastasized. Stage IV can also be referred to as advanced or metastatic cancer.

In some aspects, the cancer can include, but is not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In some aspects, the tumor is a solid tumor. A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidemoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be treated include, e.g., Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

### IV. Compositions

1. Polynucleotides Encoding Anti CTLA-4 Antibodies

The present disclosure provides polynucleotides (e.g., one or more mRNAs) encoding an antibody or antigen binding portion thereof which specifically binds to CTLA-4. In some aspects, the polynucleotides of the invention comprise one or more mRNAs (e.g., two, three, four, or more mRNAs) encoding a protein sequence listed in TABLE 1 or a portion thereof that specifically binds to CTLA-4.

In other aspects, the polynucleotides of the invention comprise one or more mRNAs corresponding to the full sequence or a subsequence of a DNA sequence listed in TABLE 1, wherein said subsequence encodes a polypeptide that specifically binds to CTLA-4.

TABLE 1
Anti-CTLA-4 Antibody Polypeptide and Polynucleotide Sequences

| SEQ ID | |
|---|---|
| NO | Description |
| 8 | VH DNA (CTLA-4-Ab_001) |
| 9 | VH protein (CTLA-4-Ab_001) |
| 10 | VL DNA (CTLA-4-Ab_001) |
| 11 | VL protein (CTLA-4-Ab_001) |
| 12 | VH protein (CTLA-4-Ab_001A) |
| 13 | VL protein (CTLA-4-Ab_001A) |
| 14 | VH-CDR1 protein (CTLA-4-Ab_001) |
| 15 | VH-CDR2 protein (CTLA-4-Ab_001) |
| 16 | VH-CDR3 protein (CTLA-4-Ab_001) |
| 17 | VL-CDR1 protein (CTLA-4-Ab_001) |
| 18 | VL-CDR2 protein (CTLA-4-Ab_001) |
| 19 | VL-CDR3 protein (CTLA-4-Ab_001) |
| 20 | HC DNA (CTLA-4-Ab_001, VH encoding portion differs from |
| | Sequence 8 by three nucleotides) |
| 21 | HC protein (CTLA-4-Ab_001, VH portion differs from |
| | Sequence 9 by two amino acids) |
| 22 | LC DNA (CTLA-4-Ab_001) |
| 23 | LC protein (CTLA-4-Ab_001) |
| 24 | HC DNA (CTLA-4-Ab_002) |
| 25 | HC protein (CTLA-4-Ab_002) |
| 26 | LC DNA (CTLA-4-Ab_002) |
| 27 | LC protein (CTLA-4-Ab_002) |
| 28 | VH protein (CTLA-4-Ab_002) |
| 29 | VL protein (CTLA-4-Ab_002) |
| 30 | VH-CDR1 protein (CTLA-4-Ab_002) |
| 31 | VH-CDR2 protein (CTLA-4-Ab_002) |
| 32 | VH-CDR3 protein (CTLA-4-Ab_002) |
| 33 | VL-CDR1 protein (CTLA-4-Ab_002) |
| 34 | VL-CDR2 protein (CTLA-4-Ab_002) |
| 35 | VL-CDR3 protein (CTLA-4-Ab_002) |
| 36 | HC DNA N294Q aglycosylated (CTLA-4-Ab_002) |
| 37 | HC protein N294Q aglycosylated (CTLA-4-Ab_002) |
| 38 | VH DNA (CTLA-4-Ab_003) |
| 39 | VH protein (CTLA-4-Ab_003) |
| 40 | VL DNA (CTLA-4-Ab_003) |
| 41 | VL protein (CTLA-4-Ab_003) |
| 42 | VH protein (CTLA-4-Ab_003A) |
| 43 | VL protein (CTLA-4-Ab_003A) |
| 44 | VH-CDR1 protein (CTLA-4-Ab_003) |
| 45 | VH-CDR2 protein (CTLA-4-Ab_003) |
| 46 | VH-CDR3 protein (CTLA-4-Ab_003) |
| 47 | VL-CDR1 protein (CTLA-4-Ab_003) |
| 48 | VL-CDR2 protein (CTLA-4-Ab_003) |
| 49 | VL-CDR3 protein (CTLA-4-Ab_003) |
| 50 | HC DNA (CTLA-4-Ab_004) |
| 51 | HC protein (CTLA-4-Ab_004) |
| 52 | LC DNA (CTLA-4-Ab_004) |
| 53 | LC protein (CTLA-4-Ab_004) |
| 54 | VH protein (CTLA-4-Ab_004) |
| 55 | VL protein (CTLA-4-Ab_004) |
| 56 | VH-CDR1 protein (CTLA-4-Ab_004) |
| 57 | VH-CDR2 protein (CTLA-4-Ab_004) |
| 58 | VH-CDR3 protein (CTLA-4-Ab_004) |
| 59 | VL-CDR1 protein (CTLA-4-Ab_004) |
| 60 | VL-CDR2 protein (CTLA-4-Ab_004) |
| 61 | VL-CDR3 protein (CTLA-4-Ab_004) |
| 62 | VH DNA (CTLA-4-Ab_005) |
| 63 | VH protein (CTLA-4-Ab_005) |
| 64 | VL DNA (CTLA-4-Ab_005) |
| 65 | VL protein (CTLA-4-Ab_005) |
| 66 | VH protein (CTLA-4-Ab_005A) |
| 67 | VL protein (CTLA-4-Ab_005A) |
| 68 | VH-CDR1 protein (CTLA-4-Ab_005) |
| 69 | VH-CDR2 protein (CTLA-4-Ab_005) |
| 70 | VH-CDR3 protein (CTLA-4-Ab_005) |
| 71 | VL-CDR1 protein (CTLA-4-Ab_005) |
| 72 | VL-CDR2 protein (CTLA-4-Ab_005) |
| 73 | VL-CDR3 protein (CTLA-4-Ab_005) |
| 74 | HC DNA (CTLA-4-Ab_006) |
| 75 | HC protein (CTLA-4-Ab_006) |
| 76 | LC DNA (CTLA-4-Ab_006) |
| 77 | LC protein (CTLA-4-Ab_006) |
| 78 | VH protein (CTLA-4-Ab_006) |
| 79 | VL protein (CTLA-4-Ab_006) |
| 80 | VH-CDR1 protein (CTLA-4-Ab_006) |
| 81 | VH-CDR2 protein (CTLA-4-Ab_006) |
| 82 | VH-CDR3 protein (CTLA-4-Ab_006) |
| 83 | VL-CDR1 protein (CTLA-4-Ab_006) |
| 84 | VL-CDR2 protein (CTLA-4-Ab_006) |
| 85 | VL-CDR3 protein (CTLA-4-Ab_006) |
| 86 | VH DNA (CTLA-4-Ab_007) |
| 87 | VH protein (CTLA-4-Ab_007) |
| 88 | VL DNA (CTLA-4-Ab_007) |
| 89 | VL protein (CTLA-4-Ab_007) |
| 90 | VH protein (CTLA-4-Ab_007A) |
| 91 | VL protein (CTLA-4-Ab_007A) |
| 92 | VH-CDR1 protein (CTLA-4-Ab_007) |
| 93 | VH-CDR2 protein (CTLA-4-Ab_007) |
| 94 | VH-CDR3 protein (CTLA-4-Ab_007) |
| 95 | VL-CDR1 protein (CTLA-4-Ab_007) |
| 96 | VL-CDR2 protein (CTLA-4-Ab_007) |
| 97 | VL-CDR3 protein (CTLA-4-Ab_007) |
| 98 | VH DNA (CTLA-4-Ab_008) |
| 99 | VH protein (CTLA-4-Ab_008) |
| 100 | VL DNA (CTLA-4-Ab_008) |
| 101 | VL protein (CTLA-4-Ab_008) |
| 102 | VH protein (CTLA-4-Ab_008A) |
| 103 | VL protein (CTLA-4-Ab_008A) |
| 104 | VH-CDR1 protein (CTLA-4-Ab_008) |
| 105 | VH-CDR2 protein (CTLA-4-Ab_008) |
| 106 | VH-CDR3 protein (CTLA-4-Ab_008) |
| 107 | VL-CDR1 protein (CTLA-4-Ab_008) |
| 108 | VL-CDR2 protein (CTLA-4-Ab_008) |
| 109 | VL-CDR3 protein (CTLA-4-Ab_008) |
| 110 | VH DNA (CTLA-4-Ab_009) |
| 111 | VH protein (CTLA-4-Ab_009) |
| 112 | VL DNA (CTLA-4-Ab_009) |
| 113 | VL protein (CTLA-4-Ab_009) |
| 114 | VH protein (CTLA-4-Ab_009) |
| 115 | VL protein (CTLA-4-Ab_009) |
| 116 | VH-CDR1 protein (CTLA-4-Ab_009) |
| 117 | VH-CDR2 protein (CTLA-4-Ab_009) |
| 118 | VH-CDR3 protein (CTLA-4-Ab_009) |
| 119 | VL-CDR1 protein (CTLA-4-Ab_009) |
| 120 | VL-CDR2 protein (CTLA-4-Ab_009) |
| 121 | VL-CDR3 protein (CTLA-4-Ab_009) |
| 122 | VH DNA (CTLA-4-Ab_010) |
| 123 | VH protein (CTLA-4-Ab_010) |
| 124 | VL DNA (CTLA-4-Ab_010) |
| 125 | VL protein (CTLA-4-Ab_010) |
| 126 | VH protein (CTLA-4-Ab_010) |
| 127 | VL protein (CTLA-4-Ab_010) |
| 128 | VH-CDR1 protein (CTLA-4-Ab_010) |
| 129 | VH-CDR2 protein (CTLA-4-Ab_010) |
| 130 | VH-CDR3 protein (CTLA-4-Ab_010) |
| 131 | VL-CDR1 protein (CTLA-4-Ab_010) |
| 132 | VL-CDR2 protein (CTLA-4-Ab_010) |
| 133 | VL-CDR3 protein (CTLA-4-Ab_010) |
| 134 | VH DNA (CTLA-4-Ab_011) |
| 135 | VH protein (CTLA-4-Ab_011) |
| 136 | VL DNA (CTLA-4-Ab_011) |
| 137 | VL protein (CTLA-4-Ab_011) |
| 138 | VH protein (CTLA-4-Ab_011) |
| 139 | VL protein (CTLA-4-Ab_011) |
| 140 | VH-CDR1 protein (CTLA-4-Ab_011) |
| 141 | VH-CDR2 protein (CTLA-4-Ab_011) |
| 142 | VH-CDR3 protein (CTLA-4-Ab_011) |
| 143 | VL-CDR1 protein (CTLA-4-Ab_011) |
| 144 | VL-CDR2 protein (CTLA-4-Ab_011) |
| 145 | VL-CDR3 protein (CTLA-4-Ab_011) |
| 146 | VH DNA (CTLA-4-Ab_012) |
| 147 | VH protein (CTLA-4-Ab_012) |
| 148 | VL DNA (CTLA-4-Ab_012) |
| 149 | VL protein (CTLA-4-Ab_012) |
| 150 | VH protein (CTLA-4-Ab_012) |
| 151 | VL protein (CTLA-4-Ab_0124) |
| 152 | VH-CDR1 protein (CTLA-4-Ab_012) |
| 153 | VH-CDR2 protein (CTLA-4-Ab_012) |
| 154 | VH-CDR3 protein (CTLA-4-Ab_012) |
| 155 | VL-CDR1 protein (CTLA-4-Ab_012) |
| 156 | VL-CDR2 protein (CTLA-4-Ab_012) |
| 157 | VL-CDR3 protein (CTLA-4-Ab_012) |
| 158 | VH DNA (CTLA-4-Ab_013) |
| 159 | VH protein (CTLA-4-Ab_013) |
| 160 | VL DNA (CTLA-4-Ab_013) |
| 161 | VL protein (CTLA-4-Ab_013) |
| 162 | VH protein (CTLA-4-Ab_013) |
| 163 | VL protein (CTLA-4-Ab_013) |
| 164 | VH-CDR1 protein (CTLA-4-Ab_013) |
| 165 | VH-CDR2 protein (CTLA-4-Ab_013) |
| 166 | VH-CDR3 protein (CTLA-4-Ab_013) |
| 167 | VL-CDR1 protein (CTLA-4-Ab_013) |
| 168 | VL-CDR2 protein (CTLA-4-Ab_013) |
| 169 | VL-CDR3 protein (CTLA-4-Ab_013) |
| 170 | 9D9 VH mIgG2Aa; Construct Sequence, RNA (5′ UTR, |
| | ORF, 3′ UTR) |
| 171 | 9D9 VH mIgG2Aa; ORF Sequence, protein |
| 172 | 9D9 VH mIgG2Aa; ORF Sequence, RNA |
| 173 | 9D9 VH mIgG2Aa; mRNA Sequence (assumes T100 tail) |
| 174 | 9D9 VH mIgG2B; Construct Sequence, RNA (5′ UTR, |
| | ORF, 3′ UTR) |
| 175 | 9D9 VH mIgG2B; ORF Sequence, protein |
| 176 | 9D9 VH mIgG2B; ORF Sequence, RNA |
| 177 | 9D9 VH mIgG2B; mRNA Sequence (assumes T100 tail) |
| 178 | 9D9 VL mIgK; Construct Sequence, RNA (5′ UTR, ORF, |
| | 3′ UTR) |
| 179 | 9D9 VL mIgKappa; ORF Sequence, protein |
| 180 | 9D9 VL mIgKappa; ORF Sequence, RNA |
| 181 | 9D9 VL mIgKappa; mRNA Sequence (assumes T100 tail) |
| 182 | VH DNA (CTLA-4-Ab_014) |
| 183 | VH protein (CTLA-4-Ab_014) |
| 184 | VL DNA (CTLA-4-Ab_014) |
| 185 | VL protein (CTLA-4-Ab_014) |
| 186 | VH-CDR1 protein (CTLA-4-Ab_014) |
| 187 | VH-CDR2 protein (CTLA-4-Ab_014) |
| 188 | VH-CDR3 protein (CTLA-4-Ab_014) |
| 189 | VL-CDR1 protein (CTLA-4-Ab_014) |
| 190 | VL-CDR2 protein (CTLA-4-Ab_014) |
| 191 | VL-CDR3 protein (CTLA-4-Ab_014) |
| 237 | VL DNA (CTLA-4-Ab_015) |
| 238 | VL protein (CTLA-4-Ab_015) |
| 239 | VH DNA (CTLA-4-Ab_015) |
| 240 | VH protein (CTLA-4-Ab_015) |
| 241 | VL DNA (CTLA-4-Ab_015A) |
| 242 | VL protein (CTLA-4-Ab_015A) |
| 243 | VH DNA (CTLA-4-Ab_015A) |
| 244 | VH protein (CTLA-4-Ab_015A) |
| 245 | VL-CDR1 (CTLA-4-Ab_015) |
| 246 | VL-CDR2 (CTLA-4-Ab_015) |
| 247 | VL-CDR3 (CTLA-4-Ab_015) |
| 248 | VH-CDR1 (CTLA-4-Ab_015) |
| 249 | VH-CDR2 (CTLA-4-Ab_015) |
| 250 | VH-CDR3 (CTLA-4-Ab_015) |

In addition to the sequences provided in TABLE 1, the methods disclosed herein can be practiced using any of the sequences of anti-CTLA-4 antibodies and antigen binding portions thereof disclosed in U.S. Pat. Nos. 8,017,114 and 6,984,720, International Publication Nos. WO2000037504 and WO2007113648, U.S. Patent Appl. Publ. No. US2014010591.4 (humanized anti CTLA-4), U.S. Pat. No. 8,697,845 (targeting soluble CTLA-4), U.S. Pat. No. 7,034,121, U.S. Pat. Nos. 8,883,984 and 7,824,679, U.S. Pat. No. 6,682,736, or International Publication No. WO 2014209804 (bispecific anti CTLA-4/PD-1), all of which are herein incorporated by reference in their entireties.

In some aspects, a polynucleotide of the present disclosure comprises one or more mRNAs (e.g., two, three, four, or more mRNAs) encoding an antibody or an antigen binding portion thereof which specifically binds to CTLA-4, e.g., a mammalian CTLA-4 polypeptide. In some aspects, the mammalian CTLA-4 polypeptide is a human CTLA-4 polypeptide. In some embodiments, the CTLA-4 polypeptide comprises the amino acid sequence set forth in SEQ ID NO: 1. In another embodiment, the CTLA-4 polypeptide comprises an amino acid sequence set forth in SEQ ID NOS: 2-7.

In some aspects, an mRNA encoding an antibody or an antigen binding portion thereof which specifically binds to CTLA-4 of the present invention comprises an amino acid sequence at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to an amino acid sequence listed in TABLE 1 or an amino acid sequence encoded by a nucleotide sequence listed in TABLE 1, wherein the protein encoded by said amino acid sequence is capable of specifically binding to CTLA-4.

In other aspects, an antibody or an antigen binding portion thereof which specifically binds to CTLA-4 useful for the disclosure comprises an amino acid sequence listed in TABLE 1 with one or more conservative substitutions, wherein the conservative substitutions do not affect the binding of the antibody or an antigen binding portion thereof to CTLA-4, i.e., the antibody or an antigen binding portion thereof binds to CTLA-4 after the substitutions. In some aspects, the amino acid sequences comprises at least one nonconservative substitution.

In other embodiments, a nucleotide sequence (i.e., an mRNA) encoding an antibody or an antigen binding portion thereof which specifically binds to CTLA-4 useful for the disclosure comprises a sequence at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to a nucleic acid sequence listed in TABLE 1 or a subsequence thereof.

In some embodiments, the mRNA comprises a codon optimized sequence encoding an antibody or an antigen binding portion thereof which specifically binds to CTLA-4 useful for the disclosure, e.g., a codon optimized nucleic acid sequence corresponding to a nucleic acid sequence or subsequence thereof from TABLE 1, or corresponding to a nucleic acid sequence encoding a polypeptide sequence from TABLE 1 or a combination thereof (e.g., a codon optimized sequence comprising several polynucleotide subsequences encoding a combination of CDRs disclosed in TABLE 1).

The present disclosure also provides a polynucleotide comprising one or more mRNAs (e.g., two, three or more mRNAs) which encode an antibody or an antigen binding portion thereof which specifically binds to CTLA-4 and which specifically binds to the same CTLA-4 epitope as: (i) an antibody or antigen-binding portion thereof comprising a heavy chain variable region (VH) of SEQ ID NO: 9, 28, or 39, and a light chain variable region (VL) of SEQ ID NO: 11, 29 or 41, or (ii) an antibody or antigen-binding portion comprising a VH of SEQ ID NO: 183 and a VL of SEQ ID NO: 185.

The present disclosure also provides polynucleotide comprising one or more mRNAs (e.g., two, three or more mRNAs) which encode an antibody or an antigen binding portion thereof which specifically binds to CTLA-4 and competitively inhibits CTLA-4 binding by: (i) an antibody or antigen-binding portion thereof comprising a VH of SEQ ID NO: 9, 28, or 39 and a VL of SEQ ID NO: 11, 29 or 41; or, (ii) an antibody or antigen-binding portion thereof comprising a VH of SEQ ID NO: 183 and a VL of SEQ ID NO: 185.

Also provided is a polynucleotide comprising one or more mRNAs (e.g., two, three or more mRNAs) which encode: (i) an antibody or an antigen binding portion thereof which specifically binds to CTLA-4 comprising a VH of SEQ ID NO: 9, 28, or 39 and a VL of SEQ ID NO: 11, 29 or 41; or, (ii) an antibody or antigen-binding portion thereof comprising a VH of SEQ ID NO: 183 and a VL of SEQ ID NO: 185.

The present disclosure also provides a polynucleotide comprising one or more mRNAs (e.g., two, three or more mRNAs) which encode an antibody or an antigen binding portion thereof which specifically binds to CTLA-4, wherein the antibody or an antigen binding portion thereof comprises: (i) a VL complementarity determining region 1 (VL-CDR1) amino acid sequence identical to, or identical except for four, three, two or one amino acid substitutions to SEQ ID NO: 17, 33, or 47 or SEQ ID NO: 189; (ii) a VL complementarity determining region 1 (VL-CDR2) amino acid sequence identical to, or identical except for four, three, two or one amino acid substitutions to SEQ ID NO: 18, 34 or 48 or SEQ ID NO: 190; (iii) a VL complementarity determining region 1 (VL-CDR3) amino acid sequence identical to, or identical except for four, three, two or one amino acid substitutions to SEQ ID NO: 19, 35 or 49 or SEQ ID NO: 191; (iv) a VH complementarity determining region 1 (VH-CDR1) amino acid sequence identical to, or identical except for four, three, two or one amino acid substitutions to SEQ ID NO: 14, 30 or 44 or SEQ ID NO: 186; (v) a VH complementarity determining region 1 (VH-CDR2) amino acid sequence identical to, or identical except for four, three, two or one amino acid substitutions to SEQ ID NO:15, 31 or 45 or SEQ ID NO: 187; (vi) a VH complementarity determining region 1 (VH-CDR3) amino acid sequence identical to, or identical except for four, three, two or one amino acid substitutions to SEQ ID NO:16, 32 or 46 or SEQ ID NO:188; (vii) a combination thereof. In some aspects, the substitutions are conservative substitutions. In other aspects, the substitutions are non-conservative substitutions.

The present disclosure also provides a polynucleotide comprising one or more mRNAs (e.g., two, three or more mRNAs) which encode an antibody or an antigen binding portion thereof which specifically binds to CTLA-4, and which comprises (i) a VL, wherein the VL comprises VL-CDR1, VL-CDR2, and VL-CDR3 amino acid sequences identical to, or identical except for four, three, two, or one amino acid substitutions in one or more of the VL-CDRS to: SEQ ID NOs: 17, 18 and 19, SEQ ID NOs: 33, 34, and 35, SEQ ID NOs: 47, 48, and 49 or SEQ ID NOs: 189, 190, and 191, respectively; (ii) a VH, wherein the VH comprises VH-CDR1, VH-CDR2, and VH-CDR3 amino acid sequences identical to, or identical except for four, three, two, or one amino acid substitutions in one or more of the VH-CDRS to: SEQ ID NOs:14,15 and 16, SEQ ID NOs: 30, 31 and 32, SEQ ID NOs: 44, 45, and 46 or SEQ ID NOs: 186, 187, and 188, respectively; or, (iii) a combination thereof. In some aspects, the substitutions are conservative substitutions. In other aspects, the substitutions are non-conservative substitutions.

The present disclosure also provides a polynucleotide comprising one or more mRNAs (e.g., two, three, four, or more mRNAs) which encode an antibody or an antigen binding portion thereof which specifically binds to CTLA-4, and which comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 17, 18, 19, 14, 15, and 16, or SEQ ID NOs: 33, 34, 35, 30, 31, and 32, or SEQ ID NOs: 47, 48, 39, 44, 45, 46, or SEQ ID NOs: 189, 190, 191, 186, 187 and 188, respectively. In some aspects, the substitutions are conservative substitutions. In other aspects, the substitutions are non-conservative substitutions.

Also provided is a polynucleotide comprising one or more mRNAs (e.g., two, three, four, or more mRNAs) which encode an antibody or an antigen binding portion thereof which specifically binds to CTLA-4, and which comprises a VL and a VH, wherein the VL comprises an amino acid sequence having at least 80%, at least 85%, at least 90%, at least 95%, at least 96%, at least 97%, at least 98%, at least 99%, or 100% sequence identity to SEQ ID NO: 11, SEQ ID NO: 29, SEQ ID NO:41 or SEQ ID NO: 185.

The present disclosure also provides a polynucleotide comprising one or more mRNAs (e.g., two, three, four, or more mRNAs) which encode an antibody or an antigen binding portion thereof which specifically binds to CTLA-4, and which comprises a VL and a VH, wherein the VH comprises an amino acid sequence having at least 80%, at least 85%, at least 90%, at least 95%, at least 96%, at least 97%, at least 98%, at least 99%, or 100% sequence identity to SEQ ID NO: 14, SEQ ID NO: 30, SEQ ID NO:44 or SEQ ID NO: 186, wherein at least one nucleoside in the polynucleotide is a chemically modified nucleoside.

Also provided is a polynucleotide comprising one or more mRNAs (e.g., two, three, four, or more mRNAs) which encode an antibody or an antigen binding portion thereof which specifically binds to CTLA-4, and which comprises a VL and a VH, wherein

(i) the VL comprises an amino acid sequence having at least 80%, at least 85%, at least 90%, at least 95%, at least 96%, at least 97%, at least 98%, at least 99%, or 100% sequence identity to SEQ ID NO: 11, SEQ ID NO: 29, or SEQ ID NO:41, and the VH comprises an amino acid sequence having at least 80%, at least 85%, at least 90%, at least 95%, at least 96%, at least 97%, at least 98%, at least 99%, or 100% sequence identity to SEQ ID NO: 9, SEQ ID NO: 28, or SEQ ID NO:39; or,

(ii) the VL comprises an amino acid sequence having at least 80%, at least 85%, at least 90%, at least 95%, at least 96%, at least 97%, at least 98%, at least 99% sequence identity to SEQ ID NO: 185, and the VH comprises an amino acid sequence having at least 80%, at least 85%, at least 90%, at least 95%, at least 96%, at least 97%, at least 98%, at least 99% sequence identity to SEQ ID NO: 183.

The present disclosure also provides a polynucleotide comprising one or more mRNAs (e.g., two, three, four or more mRNAs) which encode an antibody or an antigen binding portion thereof which specifically binds to CTLA-4, and which comprises a VL and a VH, wherein

(i) the VL consists or consists essentially of the amino acid sequence of SEQ ID NO: 11, SEQ ID NO: 29, or SEQ ID NO:41, and the VH consists or consists essentially of the amino acid sequence of SEQ ID NO: SEQ ID NO: 9, SEQ ID NO: 28, or SEQ ID NO:39; or,

(ii) the VL consists or consists essentially of the amino acid sequence of SEQ ID NO: 185, wherein the VH consists or consists essentially of the amino acid sequence of SEQ ID NO: 183.

In some aspects, at least one mRNA encoding an antibody or an antigen binding portion thereof which specifically binds to CTLA-4 further encodes a heavy chain constant region or fragment thereof. In some aspects, the heavy chain constant region or fragment thereof is an IgG constant region. In some aspects, the heavy chain IgG constant region is selected from an IgG1 constant region, an IgG2 constant region, an IgG3 constant region and an IgG4 constant region. In some aspects, the IgG constant region comprises a CH1 domain, a CH2 domain, a CH3 domain, or a combination thereof.

In some aspects, at least one mRNA encoding an antibody or an antigen binding portion thereof which specifically binds to CTLA-4 further encodes a light chain constant region or fragment thereof. In some aspects, the light chain constant region is selected from the group consisting of a kappa constant region and a lambda constant region.

The present disclosure also provides a polynucleotide comprising one or more mRNAs (e.g., two, three, four or more) which encode an antibody or an antigen binding portion thereof which specifically binds to CTLA-4, and which comprises a heavy chain (HC) a light chain (LC), wherein the HC comprises, consists, or consists essentially of the amino acid sequence of SEQ ID NO: 20 or SEQ ID NO:24, and the LC comprises, consists, or consists essentially of the amino acid sequence of SEQ ID NO: 22 or SEQ ID NO: 26.

In some aspects, the antibody or antigen binding portion thereof which specifically binds to CTLA-4 is a complete antibody, an antibody variant, an antibody fragment, or a combination thereof. In other aspects, the antibody or antigen binding portion thereof which specifically binds to CTLA-4 comprises, consists, or consists essentially of the antibody fragment, and wherein the antibody fragment is an scFv, Fab, F(ab′)2, Fab′, dsFv, or sc(Fv)2. In some aspects, the antibody or antigen binding portion thereof which specifically binds to CTLA-4 is an intrabody, a bicistronic antibody, a pseudobicistronic antibody, a single domain antibody, or a bispecific antibody. In some aspects, the CTLA-4 is human CTLA-4.

In some aspects, binding of the antibody or antigen binding portion thereof to CTLA-4: (i) reduces the size of the tumor; (ii) inhibits the growth of the tumor; (iii) reduces tumor cell proliferation in the subject; (iv) increases survival rate; or, (v) a combination thereof. In some aspects, the tumor is selected from the group consisting of melanoma tumor, lung cancer tumor, bladder cancer tumor, colon cancer tumor, and prostate cancer tumor. In some aspects, the tumor is a melanoma tumor and wherein the melanoma tumor is a metastatic melanoma tumor. In some aspects, the tumor is lung cancer tumor and wherein the lung cancer tumor is a non-small cell lung carcinoma (NSCLC) tumor or a small cell lung cancer (SCLC) tumor. In some aspect, the tumor is a prostate cancer tumor and wherein the prostate cancer tumor is a metastatic hormone-refractory prostate cancer tumor. In some aspects, the subject is human.

In some aspects, the antibody or antigen binding portion thereof which specifically binds to CTLA-4 is encoded by a polynucleotide sequence at least 60%, at least 65%, at least 70%, at least 75%, at least 80%, at least 85%, at least 90%, at least 95%, at least 96%, at least 97%, at least 98%, at least 99%, or 100% identical to SEQ ID NO: 20, SEQ ID NO:24, SEQ ID NO:22, or SEQ ID NO:26, a subsequence thereof, or a combination thereof. In some aspects, the polynucleotide sequence at least 60%, at least 65%, at least 70%, at least 75%, at least 80%, at least 85%, at least 90%, at least 95%, at least 96%, at least 97%, at least 98%, at least 99%, or 100% identical to at least one of SEQ ID NO: 20, SEQ ID NO:22, SEQ ID NO:24 or SEQ ID NO:26 or to a subsequence thereof that encodes (i) one, two or three VH-CDRs; (ii) one, two or three VL-CDRs; (iii) a VH; (iv) a VL; (v) a HC; (vi) a LC; (vii) a fragment thereof; or, (ix) a combination thereof, wherein the antibody or antigen binding portion thereof which specifically binds to CTLA-4 encoded by said polynucleotide sequence binds to at least one CTLA-4 molecule.

In some aspects, at least one of the one or more mRNAs encoding an antibody or an antigen binding portion thereof which specifically binds to CTLA-4 comprises at least one chemically modified nucleoside; or, (ii) each one of the one or more mRNAs encoding an antibody or an antigen binding portion thereof which specifically binds to CTLA-4 comprises at least one chemically modified nucleoside.

In some aspects, the polypeptide that encodes a polypeptide of the present invention (e.g., an mRNA) comprises at least one chemically modified nucleoside is selected from the group consisting of pseudouridine (ψ), N1-methylpseudouridine (m1ψ) 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof.

In some aspects, at least one chemically modified nucleoside is selected from the group consisting of pseudouridine (ψ), N1-methylpseudouridine (m1ψ), 5-methylcytosine, 5-methoxyuridine, and a combination thereof. In other aspects, the one or more mRNAs encoding an antibody or an antigen binding portion thereof which specifically binds to CTLA-4 comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% chemically modified nucleosides. In some aspects, the chemically modified nucleosides in the one or more mRNAs are selected from the group consisting of uridine nucleosides, adenine nucleosides, cytosine nucleosides, guanine nucleosides, and any combination thereof.

In some aspects, the uridine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%.

In some aspects, the adenine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%.

In some aspects, the cytosine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%.

In some aspects, the guanine nucleosides in the one or more mRNAs are chemically modified by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%.

In some aspects, one or more mRNAs encoding an antibody or an antigen binding portion thereof which specifically binds to CTLA-4 further comprise a 5′ UTR. In some aspects, the 5′ UTR comprises a nucleic acid sequence at least 90%, at least 95%, at least 96%, at least 97%, at least 98%, at least 99%, or 100% identical to a sequence selected from SEQ ID NOS: 197-214. In some aspects, the 5′ UTR is codon optimized. In some aspects, one or more mRNAs encoding an antibody or an antigen binding portion thereof which specifically binds to CTLA-4 further comprise a 3′ UTR. In some aspects, the 3′ UTR comprises a nucleic acid sequence at least 90%, 95%, 96%, 97%, 98%, 99%, or 100% identical to a sequence selected from SEQ ID NO: 215-232. In some aspects, the 3′ UTR is codon optimized. In some aspects, one or more mRNAs encoding an antibody or an antigen binding portion thereof which specifically binds to CTLA-4 further comprise a 5′ terminal cap. In some aspects, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof. In some aspects, one or more mRNAs encoding an antibody or an antigen binding portion thereof which specifically binds to CTLA-4 further comprise a 3′ poly A tail.

In some aspects, one or more mRNAs encoding an antibody or an antigen binding portion thereof which specifically binds to CTLA-4 are in vitro transcribed (IVT). In some aspects, one or more mRNAs encoding an antibody or an antigen binding portion thereof which specifically binds to CTLA-4 are chimeric. In some aspects, one or more mRNAs encoding an antibody or an antigen binding portion thereof which specifically binds to CTLA-4 are circular.

In some aspects, one or more mRNAs encoding an antibody or an antigen binding portion thereof which specifically binds to CTLA-4 are purified by strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

In some aspects, the one or more mRNAs encoding an antibody or an antigen binding portion thereof which specifically binds to CTLA-4 comprise a leader sequence comprising, consisting, or consisting essentially of the sequence of SEQ ID NO: 234 (METPAQLLFLLLLWLPDTTG). Such leader sequence can be appended, for example, to any protein sequences listed in TABLE 1 or antigen binding portions thereof. Examples of constructs comprising such leader sequence are SEQ ID NOS: 235-236.

Also provided is a composition comprising any of the polypeptides of the invention and a delivery agent, e.g., a lipidoid, a liposome, a lipoplex, a lipid nanoparticle, a polymeric compound, a peptide, a protein, a cell, a nanoparticle mimic, a nanotube, or a conjugate. In some aspects, the delivery agent is a lipid nanoparticle. In some aspects, the lipid nanoparticle comprises the lipid selected from the group consisting of DLin-DMA, DLin-K-DMA, 98N12-5, C12-200, DLin-MC3-DMA, DLin-KC2-DMA, DODMA, PEG, PEG-DMG, PEGylated lipids, amino alcohol lipids, KL22, and combinations thereof. In some aspects, the compositions disclosed herein are formulated for in vivo delivery. For example, the compositions can be formulated for subcutaneous, intravenous, intraperitoneal, intramuscular, intra-articular, intra-synovial, intrasternal, intrathecal, intrahepatic, intralesional, intracranial, intraventricular, oral, inhalation spray, topical, rectal, nasal, buccal, vaginal, intratumoral, or implanted reservoir intramuscular, subcutaneous, intratumoral, or intradermal in vivo delivery.

The present disclosure also provides a host cell comprising a polynucleotide of the invention. In some aspects, the host cell is an autologous cell (i.e., a cell obtained from the subject). In other aspects, the host cell is a heterologous cell (e.g., a cell obtained from another subject).

The present disclosure also provides a method of making a polynucleotide of the invention comprising enzymatically or chemically synthesizing the polynucleotide. Also provided is a method of producing an antibody in a cell, tissue, or organism, comprising contacting said cell, tissue, or organism with a polynucleotide of the invention or a composition comprising a polynucleotide of the invention and a delivery agent.

Also provided is method of expressing in vivo an active anti CTLA-4 antibody in a subject in need thereof comprising administering to the subject an effective amount of a polynucleotide of the invention, a composition comprising a polynucleotide of the invention and a delivery agent, or the cell disclosed above.

In some embodiments, the polynucleotide (e.g., mRNA) of the present invention is structurally modified or chemically modified. As used herein, a “structural” modification is one in which two or more linked nucleosides are inserted, deleted, duplicated, inverted or randomized in a polynucleotide without significant chemical modification to the mRNA themselves. Because chemical bonds will necessarily be broken and reformed to effect a structural modification, structural modifications are of a chemical nature and hence are chemical modifications. However, structural modifications will result in a different sequence of nucleotides. For example, the mRNA “AUCG” can be chemically modified to “AU-5meC-G”. The same mRNA can be structurally modified from “AUCG” to “AUCCCG”. Here, the dinucleotide “CC” has been inserted, resulting in a structural modification to the polynucleotide.

In some embodiments, the polynucleotide (e.g., an mRNA) of the present invention, can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine or 5-methoxyuridine. In another embodiment, the polynucleotide (e.g., mRNA) encoding an antibody or antigen binding portion thereof which specifically binds to CTLA-4 can have a uniform chemical modification of two, three, or four of the same nucleoside type throughout the entire polynucleotide (e.g., mRNA) (such as all uridines and all cytosines, etc. are modified in the same way).

When polynucleotides of the present disclosure (e.g., one or more mRNAs) encoding an antibody or antigen binding portion thereof which specifically binds to CTLA-4 are chemically and/or structurally modified the mRNA can be referred to as “modified mRNA.” Non-limiting examples of chemical modifications are described elsewhere herein.

While exemplary aspects of the compositions and methods disclosed above are described in terms of mRNAs (e.g., mRNAs being administered in the therapeutic methods of the invention), the use of the term mRNA does not exclude other polynucleotides that may be used to practice the claimed invention. The skilled artisan will appreciate the acceptable use of the term polynucleotide in the described aspects.

2. MicroRNA Binding Sites

The polynucleotides of the present disclosure (e.g., one or more mRNAs) encoding an antibody or antigen binding portion thereof which specifically binds to CTLA-4 can further comprise one or more microRNA binding sites.

microRNAs (or miRNA) are 19-25 nucleotides long noncoding RNAs that bind to the 3′UTR of nucleic acid molecules and down-regulate gene expression either by reducing nucleic acid molecule stability or by inhibiting translation.

By engineering microRNA target sequences into the polynucleotides (e.g., in a 3′UTR like region or other region) of the invention, one can target the molecule for degradation or reduced translation, provided the microRNA in question is available. In one embodiment, the miRNA binding site (e.g., miR-122 binding site) binds to the corresponding mature miRNA that is part of an active RNA-induced silencing complex (RISC) containing Dicer. In another embodiment, binding of the miRNA binding site to the corresponding miRNA in RISC degrades the mRNA containing the miRNA binding site or prevents the mRNA from being translated.

As used herein, the term “microRNA binding site” refers to a microRNA target site or a microRNA recognition site, or any nucleotide sequence to which a microRNA binds or associates. It should be understood that “binding” can follow traditional Watson-Crick hybridization rules or can reflect any stable association of the microRNA with the target sequence at or adjacent to the microRNA site.

Some microRNAs, e.g., miR-122, are abundant in normal tissue but are present n much lower levels in cancer or tumor tissue. Thus, engineering microRNA target sequences (i.e., microRNA binding site) into the polynucleotides encoding an antibody or an antigen binding portion thereof which specifically binds to CTLA-4 (e.g., in a 3′UTR like region or other region) can effectively target the molecule for degradation or reduced translation in normal tissue (where the microRNA is abundant) while providing high levels of translation in the cancer or tumor tissue (where the microRNA is present in much lower levels). This provides a tumor-targeting approach for the methods and compositions of the invention.

In some embodiments, the microRNA binding site (e.g., miR-122 binding site) is fully complementary to miRNA (e.g., miR-122), thereby degrading the mRNA fused to the miRNA binding site. In other embodiments, the miRNA binding site is not fully complementary to the corresponding miRNA. In certain embodiments, the miRNA binding site (e.g., miR-122 binding site) is the same length as the corresponding miRNA (e.g., miR-122). In other embodiments, the microRNA binding site (e.g., miR-122 binding site) is one nucleotide shorter than the corresponding microRNA (e.g., miR-122, which has 22 nts) at the 5′ terminus, the 3′ terminus, or both.

In still other embodiments, the microRNA binding site (e.g., miR-122 binding site) is two nucleotides shorter than the corresponding microRNA (e.g., miR-122) at the 5′ terminus, the 3′ terminus, or both. In yet other embodiments, the microRNA binding site miR-122 binding site) is three nucleotides shorter than the corresponding microRNA (e.g., miR-122) at the 5′ terminus, the 3′ terminus, or both. In some embodiments, the microRNA binding site (e.g., miR-122 binding site) is four nucleotides shorter than the corresponding microRNA (e.g., miR-122) at the 5′ terminus, the 3′ terminus, or both. In other embodiments, the microRNA binding site (e.g., miR-122 binding site) is five nucleotides shorter than the corresponding microRNA (e.g., miR-122) at the 5′ terminus, the 3′ terminus, or both.

In some embodiments, the microRNA binding site (e.g., miR-122 binding site) is six nucleotides shorter than the corresponding microRNA (e.g., miR-122) at the 5′ terminus, the 3′ terminus, or both. In other embodiments, the microRNA binding site (e.g., miR-122 binding site) is seven nucleotides shorter than the corresponding microRNA (e.g., miR-122) at the 5′ terminus or the 3′ terminus. In other embodiments, the microRNA binding site (e.g., miR-122 binding site) is eight nucleotides shorter than the corresponding microRNA (e.g., miR-122) at the 5′ terminus or the 3′ terminus. In other embodiments, the microRNA binding site (e.g., miR-122 binding site) is nine nucleotides shorter than the corresponding microRNA (e.g., miR-122) at the 5′ terminus or the 3′ terminus.

In other embodiments, the microRNA binding site (e.g., miR-122 binding site) is ten nucleotides shorter than the corresponding microRNA (e.g., miR-122) at the 5′ terminus or the 3′ terminus. In other embodiments, the microRNA binding site (e.g., miR-122 binding site) is eleven nucleotides shorter than the corresponding microRNA (e.g., miR-122) at the 5′ terminus or the 3′ terminus. In other embodiments, the microRNA binding site (e.g., miR-122 binding site) is twelve nucleotides shorter than the corresponding microRNA (e.g., miR-122) at the 5′ terminus or the 3′ terminus. The miRNA binding sites that are shorter than the corresponding miRNAs are still capable of degrading the mRNA incorporating one or more of the miRNA binding sites or preventing the mRNA from translation.

In some embodiments, the microRNA binding site (e.g., miR-122 binding site) has sufficient complementarity to miRNA (e.g., miR-122) so that a RISC complex comprising the miRNA (e.g., miR-122) cleaves the polynucleotide comprising the microRNA binding site.

In other embodiments, the microRNA binding site (e.g., miR-122 binding site) has imperfect complementarity so that a RISC complex comprising the miRNA (e.g., miR-122) induces instability in the polynucleotide comprising the microRNA binding site. In another embodiment, the microRNA binding site (e.g., miR-122 binding site) has imperfect complementarity so that a RISC complex comprising the miRNA (e.g., miR-122) represses transcription of the polynucleotide comprising the microRNA binding site.

In one embodiment, the miRNA binding site (e.g., miR-122 binding site) has one mismatch from the corresponding miRNA (e.g., miR-122). In another embodiment, the miRNA binding site has two mismatches from the corresponding miRNA. In other embodiments, the miRNA binding site has three mismatches from the corresponding miRNA. In other embodiments, the miRNA binding site has four mismatches from the corresponding miRNA. In some embodiments, the miRNA binding site has five mismatches from the corresponding miRNA. In other embodiments, the miRNA binding site has six mismatches from the corresponding miRNA. In certain embodiments, the miRNA binding site has seven mismatches from the corresponding miRNA. In other embodiments, the miRNA binding site has eight mismatches from the corresponding miRNA. In other embodiments, the miRNA binding site has nine mismatches from the corresponding miRNA. In other embodiments, the miRNA binding site has ten mismatches from the corresponding miRNA. In other embodiments, the miRNA binding site has eleven mismatches from the corresponding miRNA. In other embodiments, the miRNA binding site has twelve mismatches from the corresponding miRNA.

In certain embodiments, the miRNA binding site (e.g., miR-122 binding site) has at least about ten contiguous nucleotides complementary to at least about ten contiguous nucleotides of the corresponding miRNA (e.g., miR-122), at least about eleven contiguous nucleotides complementary to at least about eleven contiguous nucleotides of the corresponding miRNA, at least about twelve contiguous nucleotides complementary to at least about twelve contiguous nucleotides of the corresponding miRNA, at least about thirteen contiguous nucleotides complementary to at least about thirteen contiguous nucleotides of the corresponding miRNA, or at least about fourteen contiguous nucleotides complementary to at least about fourteen contiguous nucleotides of the corresponding miRNA.

In some embodiments, the miRNA binding sites have at least about fifteen contiguous nucleotides complementary to at least about fifteen contiguous nucleotides of the corresponding miRNA, at least about sixteen contiguous nucleotides complementary to at least about sixteen contiguous nucleotides of the corresponding miRNA, at least about seventeen contiguous nucleotides complementary to at least about seventeen contiguous nucleotides of the corresponding miRNA, at least about eighteen contiguous nucleotides complementary to at least about eighteen contiguous nucleotides of the corresponding miRNA, at least about nineteen contiguous nucleotides complementary to at least about nineteen contiguous nucleotides of the corresponding miRNA, at least about twenty contiguous nucleotides complementary to at least about twenty contiguous nucleotides of the corresponding miRNA, or at least about twenty one contiguous nucleotides complementary to at least about twenty one contiguous nucleotides of the corresponding miRNA.

In some embodiments, the polynucleotide of the invention comprises an mRNA encoding an antibody or antigen binding portion thereof which specifically binds to CTLA-4 and at least one miR122 binding site, at least two miR122 binding sites, at least three miR122 binding sites, at least four miR122 binding sites, or at least five miR122 binding sites.

In one aspect, the miRNA binding site binds miR-122 or is complementary to miR-122. In another aspect, the miRNA binding site binds to miR-122-3p or miR-122-5p. In a particular aspect, the miRNA binding site comprises a nucleotide sequence at least 80%, at least 85%, at least 90%, at least 95%, or 100% identical to SEQ ID NO: 194, wherein the miRNA binding site binds to miR-122. In another particular aspect, the miRNA binding site comprises a nucleotide sequence at least 80%, at least 85%, at least 90%, at least 95%, or 100% identical to SEQ ID NO: 196, wherein the miRNA binding site binds to miR-122. These sequences are shown below in TABLE 2.

TABLE 2
miR-122 and miR-122 binding sites

| SEQ ID | Descrip- | |
|---|---|---|
| NO. | tion | Sequence |
| SEQ ID | miR-122 | CCUUAGCAGAGCUGUGGAGUGUGACAAUGGU |
| NO: 192 | | GUUUGUGUCUAAACUAUCAAACGCCAUUAUC |
| | | ACACUAAAUAGCUACUGCUAGGC |
| SEQ ID | miR-122- | AACGCCAUUAUCACACUAAAUA |
| NO: 193 | 3p | |
| SEQ ID | miR-122- | UAUUUAGUGUGAUAAUGGCGUU |
| NO: 194 | 3p | |
| | binding | |
| | site | |
| SEQ ID | miR-122- | UGGAGUGUGACAAUGGUGUUUG |
| NO: 195 | 5p | |
| SEQ ID | miR-122- | CAAACACCAUUGUCACACUCCA |
| NO: 196 | 5p | |
| | binding | |
| | site | |

In some embodiments, a miRNA binding site (e.g., miR-122 binding site) is inserted in the polynucleotide of the invention in any position of the polynucleotide (e.g., 3′ UTR); the insertion site in the polynucleotide can be anywhere in the polynucleotide as long as the insertion of the miRNA binding site in the polynucleotide does not interfere with the translation of the functional polypeptide (e.g., an antibody or antigen binding portion thereof which specifically binds to CTLA-4 disclosed herein) in the absence of the corresponding miRNA (e.g., miR122); and in the presence of the miRNA (e.g., miR122), the insertion of the miRNA binding site in the polynucleotide and the binding of the miRNA binding site to the corresponding miRNA are capable of degrading the polynucleotide or preventing the translation of the polynucleotide. In one embodiment, a miRNA binding site is inserted in a 3′UTR of the polynucleotide.

In certain embodiments, a miRNA binding site is inserted in at least about 30 nucleotides downstream from the stop codon of an mRNA encoding an antibody or antigen binding portion thereof which specifically binds to CTLA-4 disclosed herein. In other embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of the polynucleotide, e.g., an mRNA encoding antibody or antigen binding portion thereof which specifically binds to CTLA-4 disclosed herein.

In other embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of the polynucleotide, e.g., an mRNA encoding antibody or antigen binding portion thereof which specifically binds to CTLA-4 disclosed herein.

3. IVT Polynucleotide Architecture

In some embodiments, the polynucleotide of the present invention comprising an mRNA (or set of mRNAs) encoding an antibody or antigen binding portion thereof with specifically binds to CTLA-4 is an IVT polynucleotide (or a set of IVT polynucleotides, e.g., a first IVT polynucleotide encoding the HC of the antibody and a second IVT polynucleotide encoding the LC of the antibody).

Traditionally, the basic components of an mRNA molecule include at least a coding region, a 5′UTR, a 3′UTR, a 5′ cap and a poly-A tail. The IVT polynucleotides of the present invention can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded antibody or antigen binding portion thereof with specifically binds to CTLA-4. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR.

The IVT polynucleotide encoding an antibody or antigen binding portion thereof with specifically binds to CTLA-4 can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap.

The second flanking region can comprise a region of linked nucleotides comprising one or more complete or incomplete 3′ UTRs which can encode the native 3′ UTR of an antibody or antigen binding portion thereof with specifically binds to CTLA-4 or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Bridging the 5′ terminus of the first region and the first flanking region is a first operational region. Traditionally, this operational region comprises a Start codon. The operational region can alternatively comprise any translation initiation sequence or signal including a Start codon.

Bridging the 3′ terminus of the first region and the second flanking region is a second operational region. Traditionally this operational region comprises a Stop codon. The operational region can alternatively comprise any translation initiation sequence or signal including a Stop codon. Multiple serial stop codons can also be used in the IVT polynucleotide. In some embodiments, the operation region of the present invention can comprise two stop codons. The first stop codon can be “TGA” or “UGA” and the second stop codon can be selected from the group consisting of “TAA,” “TGA,” “TAG,” “UAA,” “UGA” or “UAG.”

The IVT polynucleotide primary construct comprises a first region of linked nucleotides that is flanked by a first flanking region and a second flaking region. As used herein, the “first region” can be referred to as a “coding region” or “region encoding” or simply the “first region.” This first region can include, but is not limited to, the encoded polypeptide of interest. In one aspect, the first region can include, but is not limited to, the open reading frame encoding at least one polypeptide of interest. The open reading frame can be codon optimized in whole or in part. The flanking region can comprise a region of linked nucleotides comprising one or more complete or incomplete 5′ UTRs sequences which can be completely codon optimized or partially codon optimized. The flanking region can include at least one nucleic acid sequence including, but not limited to, miR sequences, TERZAK™ sequences and translation control sequences. The flanking region can also comprise a 5′ terminal cap 138. The 5′ terminal capping region can include a naturally occurring cap, a synthetic cap or an optimized cap. The second flanking region can comprise a region of linked nucleotides comprising one or more complete or incomplete 3′ UTRs. The second flanking region can be completely codon optimized or partially codon optimized. The flanking region can include at least one nucleic acid sequence including, but not limited to, miR sequences and translation control sequences. After the second flanking region the polynucleotide primary construct can comprise a 3′ tailing sequence. The 3′ tailing sequence can include a synthetic tailing region and/or a chain terminating nucleoside. Non-liming examples of a synthetic tailing region include a polyA sequence, a polyC sequence, a polyA-G quartet. Non-limiting examples of chain terminating nucleosides include 2′-O methyl, F and locked nucleic acids (LNA).

Bridging the 5′ terminus of the first region and the first flanking region is a first operational region. Traditionally this operational region comprises a Start codon. The operational region can alternatively comprise any translation initiation sequence or signal including a Start codon.

Bridging the 3′ terminus of the first region and the second flanking region is a second operational region. Traditionally this operational region comprises a Stop codon. The operational region can alternatively comprise any translation initiation sequence or signal including a Stop codon. According to the present invention, multiple serial stop codons can also be used.

In some embodiments, the first and second flanking regions of the IVT polynucleotide can range independently from 15-5,500 nucleotides in length (e.g., greater than 15, 20, 30, 40, 45, 50, 55, 60, 70, 80, 90, 100, 120, 140, 160, 180, 200, 250, 300, 350, 400, 450, 500, 600, 700, 800, 900, 1,000, 1,500, 2,000, 2,500, 3,000, 3,500, 4,000, 4,500, 5,000, or 5,500 nucleotides or at least 15, 20, 30, 40, 45, 50, 55, 60, 70, 80, 90, 100, 120, 140, 160, 180, 200, 250, 300, 350, 400, 450, 500, 600, 700, 800, 900, 1,000, 1,500, 2,000, 2,500, 3,000, 3,500, 4,000, 4,500, 5,000, or 5,500 nucleotides).

In some embodiments, the tailing sequence of the IVT polynucleotide can range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length can be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

In some embodiments, the capping region of the IVT polynucleotide can comprise a single cap or a series of nucleotides forming the cap. In this embodiment the capping region can be from 1 to 10, e.g. 2-9, 3-8, 4-7, 1-5, 5-10, or at least 2, or 10 or fewer nucleotides in length. In some embodiments, the cap is absent.

In some embodiments, the first and second operational regions of the IVT polynucleotide can range from 3 to 40, e.g., 5-30, 10-20, 15, or at least 4, or 30 or fewer nucleotides in length and can comprise, in addition to a Start and/or Stop codon, one or more signal and/or restriction sequences.

In some embodiments, the IVT polynucleotides can be structurally modified or chemically modified. When the IVT polynucleotides are chemically and/or structurally modified the polynucleotides can be referred to as “modified IVT polynucleotides.”

In some embodiments, if the IVT polynucleotides are chemically modified they can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine or 5-methoxyuridine. In another embodiment, the IVT polynucleotides can have a uniform chemical modification of two, three, or four of the same nucleoside type throughout the entire polynucleotide (such as all uridines and all cytosines, etc. are modified in the same way).

In some embodiments, the IVT polynucleotides can include a sequence encoding a self-cleaving peptide, described herein, such as but not limited to the 2A peptide. The polynucleotide sequence of the 2A peptide in the IVT polynucleotide can be modified or codon optimized by the methods described herein and/or are known in the art. In some embodiments, this sequence can be used to separate the coding region of two or more polypeptides of interest in the IVT polynucleotide.

4. Chimeric Polynucleotide Architecture

In some embodiments, the polynucleotide of the present invention is a chimeric polynucleotide. The chimeric polynucleotides or RNA constructs (e.g., mRNAs) disclosed herein maintain a modular organization similar to IVT polynucleotides, but the chimeric polynucleotides comprise one or more structural and/or chemical modifications or alterations which impart useful properties to the polynucleotide. As such, the chimeric polynucleotides which are modified mRNA molecules of the present invention are termed “chimeric modified mRNA” or “chimeric mRNA.”

Chimeric polynucleotides have portions or regions which differ in size and/or chemical modification pattern, chemical modification position, chemical modification percent or chemical modification population and combinations of the foregoing.

Examples of parts or regions, where the chimeric polynucleotide functions as an mRNA and encodes an antibody or an antigen binding portion thereof which specifically binds to CTLA-4 (e.g., the mRNA can encode both the HC and LC of the antibody, or only the HC or the LC), but is not limited to, untranslated regions (UTRs, such as the 5′ UTR or 3′ UTR), coding regions, cap regions, polyA tail regions, start regions, stop regions, signal sequence regions, and combinations thereof. Regions or parts that join or lie between other regions can also be designed to have subregions.

In some embodiments, the chimeric polynucleotides of the invention have a structure comprising Formula I.

5′ [An]x-L1-[Bo]y-L2-[Cp]z-L3 3′ Formula I

wherein:
each of A and B independently comprise a region of linked nucleosides;
either A or B or both A and B encode an antibody or an antigen binding portion thereof which specifically binds to CTLA-4 described elsewhere herein (e.g., A could encode an antibody HC and B could encode an antibody LC);
C is an optional region of linked nucleosides (e.g., C would encode a sequence targeting the antibody to a certain tissue, such a microRNA sequence);
at least one of regions A, B, or C is positionally modified, wherein said positionally modified region comprises at least two chemically modified nucleosides of one or more of the same nucleoside type of adenosine, thymidine, guanosine, cytidine, or uridine, and wherein at least two of the chemical modifications of nucleosides of the same type are different chemical modifications;
n, o and p are independently an integer between 15 and 3000;
x and y are independently an integer between 1 and 20;
z is an integer between 0 and 5;
L1 and L2 are independently optional linker moieties, said linker moieties being either nucleic acid based or non-nucleic acid based; and
L3 is an optional conjugate or an optional linker moiety, said linker moiety being either nucleic acid based or non-nucleic acid based.

In some embodiments, at least one of the regions of linked nucleosides of A comprises a sequence of linked nucleosides which can function as a 5′ untranslated region (UTR). The sequence of linked nucleosides can be a natural or synthetic 5′ UTR.

As a non-limiting example, the chimeric polynucleotide can encode an antibody or antigen binding portion thereof which specifically binds to CTLA-4, and the sequence of linked nucleosides of A can encode the native 5′ UTR of the oroginal antibody or antigen binding portion thereof which specifically binds to CTLA-4 or a non-heterologous 5′ UTR such as, but not limited to a synthetic UTR.

In another embodiment, at least one of the regions of linked nucleosides of A is a cap region. The cap region can be located 5′ to a region of linked nucleosides of A functioning as a 5′UTR. The cap region can comprise at least one cap such as, but not limited to, Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azido-guanosine, Cap2 and Cap4.

In some embodiments, the polynucleotide or polynucleotides of the invention comprise a Cap1 5′ UTR. In some embodiments, a polynucleotide of the invention comprising 5′ UTR sequence, e.g., Cap1, for encoding an antibody or antigen binding portion thereof which specifically binds to CTLA-4 disclosed herein increases expression of the antibody or antigen binding portion thereof compared to polynucleotides encoding the same antibody or antigen binding portion thereof comprising a different 5′UTR Cap0, ARCA, inosine, N1-methyl-guanosine, 2′fluoro-guanosine, 7-deaza-guanosine, 8-oxo-guanosine, 2-amino-guanosine, LNA-guanosine, 2-azido-guanosine, Cap2 or Cap4).

In some embodiments, a polynucleotide comprises the Cap1 5′UTR, wherein the polynucleotide encodes an antibody or antigen-binding portion thereof which specifically binds to CTLA-4. In some embodiments, polynucleotide comprising the Cap1 5′UTR, increases expression of an antibody or antigen-binding portion thereof which specifically binds to CTLA-4.

In some embodiments, at least one of the regions of linked nucleosides of B comprises at least one open reading frame of a nucleic acid sequence encoding an antibody or antigen-binding portion thereof which specifically binds to CTLA-4. The nucleic acid sequence can be codon optimized and/or comprise at least one modification.

In some embodiments, at least one of the regions of linked nucleosides of C comprises a sequence of linked nucleosides which can function as a 3′ UTR. The sequence of linked nucleosides can be a natural or synthetic 3′ UTR. As a non-limiting example, the chimeric polynucleotide can encode an antibody or antigen-binding portion thereof which specifically binds to CTLA-4 and the sequence of linked nucleosides of C can encode the native 3′ UTR of an antibody or antigen-binding portion thereof which specifically binds to CTLA-4 or a non-heterologous 3′ UTR such as, but not limited to a synthetic UTR.

In some embodiments, at least one of the regions of linked nucleosides of A comprises a sequence of linked nucleosides which functions as a 5′ UTR and at least one of the regions of linked nucleosides of C comprises a sequence of linked nucleosides which functions as a 3′ UTR. In some embodiments, the 5′ UTR and the 3′ UTR can be from the same or different species. In another embodiment, the 5′ UTR and the 3′ UTR can encode the native untranslated regions from different proteins from the same or different species.

Chimeric polynucleotides, including the parts or regions thereof, of the present invention can be classified as hemimers, gapmers, wingmers, or blockmers.

As used herein, a “hemimer” is a chimeric polynucleotide comprising a region or part which comprises half of one pattern, percent, position or population of a chemical modification(s) and half of a second pattern, percent, position or population of a chemical modification(s). Chimeric polynucleotides of the present invention can also comprise hemimer subregions. In some embodiments, a part or region is 50% of one and 50% of another.

In some embodiments, the entire chimeric polynucleotide is 50% of one and 50% of the other. Any region or part of any chimeric polynucleotide of the invention can be a hemimer. Types of hemimers include pattern hemimers, population hemimers or position hemimers. By definition, hemimers are 50:50 percent hemimers.

As used herein, a “gapmer” is a chimeric polynucleotide having at least three parts or regions with a gap between the parts or regions. The “gap” can comprise a region of linked nucleosides or a single nucleoside which differs from the chimeric nature of the two parts or regions flanking it. The two parts or regions of a gapmer can be the same or different from each other.

As used herein, a “wingmer” is a chimeric polynucleotide having at least three parts or regions with a gap between the parts or regions. Unlike a gapmer, the two flanking parts or regions surrounding the gap in a wingmer are the same in degree or kind. Such similarity can be in the length of number of units of different modifications or in the number of modifications. The wings of a wingmer can be longer or shorter than the gap. The wing parts or regions can be 20, 30, 40, 50, 60 70, 80, 90 or 95% greater or shorter in length than the region which comprises the gap.

As used herein, a “blockmer” is a patterned polynucleotide where parts or regions are of equivalent size or number and type of modifications. Regions or subregions in a blockmer can be 50, 51, 52, 53, 54, 55, 56, 57, 58, 59, 60, 61 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 310, 320, 330, 340, 350, 360, 370, 380, 390, 400, 410, 420, 430, 440, 450, 460, 470, 480, 490 or 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 nucleosides long.

Chimeric polynucleotides, including the parts or regions thereof, of the present invention having a chemical modification pattern are referred to as “pattern chimeras.” Pattern chimeras can also be referred to as blockmers. Pattern chimeras are those polynucleotides having a pattern of modifications within, across or among regions or parts.

Patterns of modifications within a part or region are those which start and stop within a defined region. Patterns of modifications across a part or region are those patterns which start in on part or region and end in another adjacent part or region. Patterns of modifications among parts or regions are those which begin and end in one part or region and are repeated in a different part or region, which is not necessarily adjacent to the first region or part.

The regions or subregions of pattern chimeras or blockmers can have simple alternating patterns such as ABAB[AB]n where each “A” and each “B” represent different chemical modifications (at least one of the base, sugar or backbone linker), different types of chemical modifications (e.g., naturally occurring and non-naturally occurring), different percentages of modifications or different populations of modifications. The pattern can repeat n number of times where n=3-1000. Further, each A or B can represent from 1-5000 units (e.g., nucleosides) in the pattern. Patterns can also be alternating multiples such as AABBAABB[AABB]n (an alternating double multiple) or AAABBBAAABBB[AAABBB]n (an alternating triple multiple) pattern. The pattern can repeat n number of times where n=3-300.

Different patterns can also be mixed together to form a second order pattern. For example, a single alternating pattern can be combined with a triple alternating pattern to form a second order alternating pattern A′B′. One example would be [ABABAB][AAABBBAAABBB][ABABAB][AAABBBAAABBB] [ABABAB][AAAB BBAAABBB], where [ABABAB] is A′ and [AAABBBAAABBB] is B′. In like fashion, these patterns can be repeated n number of times, where n=3-1000.

Patterns can include three or more different modifications to form an ABCABC[ABC]n pattern. These three component patterns can also be multiples, such as AABBCCAABBCC[AABBCC]n and can be designed as combinations with other patterns such as ABCABCAABBCCABCABCAABBCC, and can be higher order patterns.

Regions or subregions of position, percent, and population modifications need not reflect an equal contribution from each modification type. They can form series such as “1-2-3-4”, “1-2-4-8”, where each integer represents the number of units of a particular modification type. Alternatively, they can be odd only, such as “1-3-3-1-3-1-5” or even only “2-4-2-4-6-4-8” or a mixture of both odd and even number of units such as “1-3-4-2-5-7-3-3-4”.

Pattern chimeras can vary in their chemical modification by degree (such as those described above) or by kind (e.g., different modifications).

Chimeric polynucleotides, including the parts or regions thereof, of the present invention having at least one region with two or more different chemical modifications of two or more nucleoside members of the same nucleoside type (A, C, G, T, or U) are referred to as “positionally modified” chimeras. Positionally modified chimeras are also referred to herein as “selective placement” chimeras or “selective placement polynucleotides”. As the name implies, selective placement refers to the design of polynucleotides which, unlike polynucleotides in the art where the modification to any A, C, G, T or U is the same by virtue of the method of synthesis, can have different modifications to the individual As, Cs, Gs, Ts or Us in a polynucleotide or region thereof. For example, in a positionally modified chimeric polynucleotide, there can be two or more different chemical modifications to any of the nucleoside types of As, Cs, Gs, Ts, or Us. There can also be combinations of two or more to any two or more of the same nucleoside type. For example, a positionally modified or selective placement chimeric polynucleotide can comprise 3 different modifications to the population of adenines in the molecule and also have 3 different modifications to the population of cytosines in the construct all of which can have a unique, non-random, placement.

Chimeric polynucleotides, including the parts or regions thereof, of the present invention having a chemical modification percent are referred to as “percent chimeras.” Percent chimeras can have regions or parts which comprise at least 1%, at least 2%, at least 5%, at least 8%, at least 10%, at least 20%, at least 30%, at least 40%, at least 50%, at least 60%, at least 70%, at least 80%, at least 90%, at least 95%, or at least 99% positional, pattern or population of modifications. Alternatively, the percent chimera can be completely modified as to modification position, pattern, or population. The percent of modification of a percent chimera can be split between naturally occurring and non-naturally occurring modifications.

Chimeric polynucleotides, including the parts or regions thereof, of the present invention having a chemical modification population are referred to as “population chimeras.” A population chimera can comprise a region or part where nucleosides (their base, sugar or backbone linkage, or combination thereof) have a select population of modifications. Such modifications can be selected from functional populations such as modifications which induce, alter or modulate a phenotypic outcome. For example, a functional population can be a population or selection of chemical modifications which increase the level of a cytokine. Other functional populations can individually or collectively function to decrease the level of one or more cytokines. Use of a selection of these like-function modifications in a chimeric polynucleotide would therefore constitute a “functional population chimera.” As used herein, a “functional population chimera” can be one whose unique functional feature is defined by the population of modifications as described above or the term can apply to the overall function of the chimeric polynucleotide itself. For example, as a whole the chimeric polynucleotide can function in a different or superior way as compared to an unmodified or non-chimeric polynucleotide.

It should be noted that polynucleotides which have a uniform chemical modification of all of any of the same nucleoside type or a population of modifications produced by mere downward titration of the same starting modification in all of any of the same nucleoside type, or a measured percent of a chemical modification of all any of the same nucleoside type but with random incorporation, such as where all uridines are replaced by a uridine analog, e.g., pseudouridine or 5-methoxyuridine, are not considered chimeric polynucleotides. Likewise, polynucleotides having a uniform chemical modification of two, three, or four of the same nucleoside type throughout the entire polynucleotide (such as all uridines and all cytosines, etc. are modified in the same way) are not considered chimeric polynucleotides. One example of a polynucleotide which is not chimeric is the canonical pseudouridine/5-methyl cytosine modified polynucleotide. These uniform polynucleotides are arrived at entirely via in vitro transcription (IVT) enzymatic synthesis; and due to the limitations of the synthesizing enzymes, they contain only one kind of modification at the occurrence of each of the same nucleoside type, i.e., adenosine (A), thymidine (T), guanosine (G), cytidine (C) or uridine (U), found in the polynucleotide. Such polynucleotides can be characterized as “IVT polynucleotides.”

The chimeric polynucleotides of the present invention can be structurally modified or chemically modified. When the chimeric polynucleotides of the present invention are chemically and/or structurally modified the polynucleotides can be referred to as “modified chimeric polynucleotides.”

The regions or parts of the chimeric polynucleotides can be separated by a linker or spacer moiety. Such linkers or spaces can be nucleic acid based or non-nucleosidic.

In some embodiments, the chimeric polynucleotides can include a sequence encoding a self-cleaving peptide described herein, such as, but not limited to, a 2A peptide. The polynucleotide sequence of the 2A peptide in the chimeric polynucleotide can be modified or codon optimized by the methods described herein and/or are known in the art.

Notwithstanding the foregoing, the chimeric polynucleotides of the present invention can comprise a region or part which is not positionally modified or not chimeric as defined herein. For example, a region or part of a chimeric polynucleotide can be uniformly modified at one or more A, T, C, G, or U, but the polynucleotides will not be uniformly modified throughout the entire region or part.

Chimeric polynucleotides of the present invention can be completely positionally modified or partially positionally modified. They can also have subregions which can be of any pattern or design.

In some embodiments, regions or subregions of the polynucleotides can range from absent to 500 nucleotides in length (e.g., at least 60, 70, 80, 90, 100, 110, 120, 130, 140, 150, 160, 170, 180, 190, 200, 250, 300, 350, 400, 450, or 500 nucleotides). Where the region is a polyA tail, the length can be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that poly A tails of about 80 nucleotides to about 160 nucleotides are functional. The chimeric polynucleotides of the present invention which function as an mRNA need not comprise a polyA tail.

According to the present invention, chimeric polynucleotides which function as an mRNA can have a capping region. The capping region can comprise a single cap or a series of nucleotides forming the cap. In this embodiment the capping region can be from 1 to 10, e.g. 2-9, 3-8, 4-7, 1-5, 5-10, or at least 2, or 10 or fewer nucleotides in length. In some embodiments, the cap is absent.

The present invention contemplates chimeric polynucleotides which are circular or cyclic. As the name implies circular polynucleotides are circular in nature meaning that the termini are joined in some fashion, whether by ligation, covalent bond, common association with the same protein or other molecule or complex or by hybridization.

Chimeric polynucleotides, formulations and compositions comprising chimeric polynucleotides, and methods of making, using and administering chimeric polynucleotides are also described in International Patent Application Publ. No. WO2015034928.

In some embodiments, the chimeric polynucleotide encodes an antibody or an antigen binding portion thereof which specifically binds to CTLA-4. In some embodiments, the chimeric polynucleotides of the invention comprise any of the antibody heavy chain (HC) or light chain (LC) polynucleotide sequences of TABLE 1, or a subsequence thereof which encodes (i) one, two or three VH-CDRs; (ii) one, two or three VL-CDRs; (iii) a VH; (iv) a VL; (v) a HC; (vi) a LC; (vii) a fragment thereof; or, (viii) a combination thereof. In some embodiments, the chimeric polynucleotides of the invention comprise a subsequence of any of the antibody heavy chain (HC) or light chain (LC) polynucleotide sequences of TABLE 1, wherein the subsequence encodes any of the polypeptide sequences listed in TABLE 1.

5. Circular Polynucleotides

The polynucleotides of the present invention (e.g., one or more mRNAs encoding an antibody or an antigen binding portion thereof which specifically binds to CTLA-4) can be circular or cyclic. As used herein, “circular polynucleotides” or “circP” means a single stranded circular polynucleotide which acts substantially like, and has the properties of, an RNA. The term “circular” is also meant to encompass any secondary or tertiary configuration of the circP. Circular polynucleotides are circular in nature meaning that the termini are joined in some fashion, whether by ligation, covalent bond, common association with the same protein or other molecule or complex or by hybridization.

Circular polynucleotides, formulations and compositions comprising circular polynucleotides, and methods of making, using and administering circular polynucleotides are also disclosed in International Patent Application Publ. No. WO2015034925.

In some embodiments, the circular polynucleotide encodes an antibody or an antigen binding portion thereof which specifically binds to CTLA-4). In some embodiments, the circular polynucleotides of the invention comprise any one of the nucleic acid sequences listed in TABLE 1, any subsequences of said nucleic acid sequences encoding the polypeptide sequences listed in TABLE 1, or combinations thereof. In some embodiments, the circular polynucleotides of the invention encode any one of the polypeptides listed in TABLE 1 or combinations thereof. In some embodiments, the circular polynucleotide increases expression of the antibody or an antigen binding portion thereof which specifically binds to CTLA-4.

6. Multimers of Polynucleotides

In some embodiments, multiple distinct chimeric polynucleotides and/or IVT polynucleotides can be linked together through the 3′-end using nucleotides which are modified at the 3′-terminus. Chemical conjugation can be used to control the stoichiometry of delivery into cells. This can be controlled by chemically linking chimeric polynucleotides and/or IVT polynucleotides using a 3′-azido terminated nucleotide on one polynucleotides species and a C5-ethynyl or alkynyl-containing nucleotide on the opposite polynucleotide species. The modified nucleotide is added post-transcriptionally using terminal transferase (New England Biolabs, Ipswich, Mass.) according to the manufacturer's protocol. After the addition of the 3′-modified nucleotide, the two polynucleotides species can be combined in an aqueous solution, in the presence or absence of copper, to form a new covalent linkage via a click chemistry mechanism as described in the literature.

In another example, more than two chimeric polynucleotides and/or IVT polynucleotides can be linked together using a functionalized linker molecule. For example, a functionalized saccharide molecule can be chemically modified to contain multiple chemical reactive groups (SH—, NH2-, N3, etc.) to react with the cognate moiety on a 3′-functionalized mRNA molecule (i.e., a 3′-maleimide ester, 3′-NHS-ester, alkynyl). The number of reactive groups on the modified saccharide can be controlled in a stoichiometric fashion to directly control the stoichiometric ratio of conjugated chimeric polynucleotides and/or IVT polynucleotides.

In some embodiments, the chimeric polynucleotides and/or IVT polynucleotides can be linked together in a pattern. The pattern can be a simple alternating pattern such as CD[CD]x where each “C” and each “D” represent a chimeric polynucleotide, IVT polynucleotide, different chimeric polynucleotides or different IVT polynucleotides. The pattern can repeat x number of times, where x=1-1000. Patterns can also be alternating multiples such as CCDD[CCDD]x (an alternating double multiple) or CCCDDD[CCCDDD]x (an alternating triple multiple) pattern. The alternating double multiple or alternating triple multiple can repeat x number of times, where x=1-1000.

7. Conjugates and Combinations of Polynucleotides

The polynucleotides of the present invention (e.g., one or more mRNAs encoding an antibody or an antigen binding portion thereof which specifically binds to CTLA-4) can be designed to be conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases, chelators (e.g. EDTA), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins (e.g., glycoproteins, or peptides), molecules having a specific affinity for a co-ligand, or antibodies (e.g., an antibody that binds to a specified cell type such as a cancer cell, endothelial cell, or bone cell), hormones and hormone receptors, non-peptidic species (such as lipids, lectins, carbohydrates, vitamins, cofactors), or a drug.

Conjugation can result in increased stability and/or half-life (e.g., plasma half life) and can be particularly useful in targeting the polynucleotides to specific sites in the cell, tissue or organism.

8. Polynucleotides having Untranslated Regions (UTRs)

The polynucleotides of the present invention (e.g., one or more mRNAs encoding an antibody or an antigen binding portion thereof which specifically binds to CTLA-4) can further comprise a nucleotide sequence encoding one or more heterologous polypeptides. In one embodiment, the one or more heterologous polypeptides improve a pharmacokinetic property or pharmacodynamic property of the antibody or antigen binding portion thereof which specifically binds to CTLA-4. In another embodiment, the one or more heterologous polypeptides comprise a polypeptide that can extend a half-life (e.g., the plasma half life) of the antibody or antigen binding portion thereof which specifically binds to CTLA-4.

In one embodiment, an mRNA of the present invention encodes an antibody or an antigen binding portion thereof which specifically binds to CTLA-4 and one or more heterologous polypeptides.

In another embodiment, an mRNA of the present invention encodes an antibody or an antigen binding portion thereof which specifically binds to CTLA-4 and a heterologous polypeptide. In another embodiment, the mRNA encodes a fusion protein comprising an antibody or an antigen binding portion thereof which specifically binds to CTLA-4 and a polypeptide that can extend a half-life (e.g., plasma half life) of the antibody or antigen binding portion thereof which specifically binds to CTLA-4.

The polynucleotides of the present invention (e.g., one or more mRNAs encoding an antibody or an antigen binding portion thereof which specifically binds to CTLA-4) can further comprise one or more regions or parts which act or function as an untranslated region. By definition, wild type untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present invention to, among other things, enhance the stability of the molecule. The specific features can also be incorporated to ensure controlled down-regulation of the transcript in case they are misdirected to undesired organs sites.

TABLE 3 and TABLE 4 provide a listing of exemplary UTRs which can be utilized in the polynucleotides of the present invention.

9. 5′ UTR and Translation Initiation

In certain embodiments, the polynucleotide of the present invention (e.g., one or more mRNAs encoding an antibody or an antigen binding portion thereof which specifically binds to CTLA-4) polypeptide further comprises a 5′ UTR and/or a translation initiation sequence. Natural 5′UTRs bear features which play roles in translation initiation. They harbor signatures like Kozak sequences which are commonly known to be involved in the process by which the ribosome initiates translation of many genes. 5′UTR also have been known to form secondary structures which are involved in elongation factor binding.

By engineering the features typically found in abundantly expressed genes of specific target organs, one can enhance the stability and protein production of the polynucleotides of the invention. For example, introduction of 5′ UTR of mRNA known to be upregulated in cancers, such as c-myc, could be used to enhance expression of a nucleic acid molecule, such as a polynucleotides, in cancer cells. Untranslated regions useful in the design and manufacture of polynucleotides include, but are not limited, to those disclosed in International Patent Publication No. WO2014/164253.

Shown in TABLE 3 is a listing of a 5′-untranslated regions that can be utilized in the polynucleotides of the present invention (e.g., one or more mRNAs encoding an antibody or an antigen binding portion thereof which specifically binds to CTLA-4). Variants of 5′ UTRs can be utilized wherein one or more nucleotides are added or removed to the termini, including A, U, C or G.

TABLE 3
5′-Untranslated Regions

| | Name/ | | |
|---|---|---|---|
| 5′ UTR | De- | | |
| Identi- | scrip- | | SEQ ID |
| fier | tion | Sequence | NO. |
| 5UTR- | Up- | GGGAAAUAAGAGAGAAAAGAAGAGUAA | SEQ ID |
| 001 | stream | GAAGAAAUAUAAGAGCCACC | NO: 197 |
| | UTR | | |
| 5UTR- | Up- | GGGAGAUCAGAGAGAAAAGAAGAGUAA | SEQ ID |
| 002 | stream | GAAGAAAUAUAAGAGCCACC | NO: 198 |
| | UTR | | |
| 5UTR- | Up- | GGAAUAAAAGUCUCAACACAACAUAUA | SEQ ID |
| 003 | stream | CAAAACAAACGAAUCUCAAGCAAUCAA | NO: 199 |
| | UTR | GCAUUCUACUUCUAUUGCAGCAAUUUA | |
| | | AAUCAUUUCUUUUAAAGCAAAAGCAAU | |
| | | UUUCUGAAAAUUUUCACCAUUUACGAA | |
| | | CGAUAGCAAC | |
| 5UTR- | Up- | GGGAGACAAGCUUGGCAUUCCGGUACU | SEQ ID |
| 004 | stream | GUUGGUAAAGCCACC | NO: 200 |
| | UTR | | |
| 5UTR- | Up- | GGGAGAUCAGAGAGAAAAGAAGAGUAA | SEQ ID |
| 005 | stream | GAAGAAAUAUAAGAGCCACC | NO: 201 |
| | UTR | | |
| 5UTR- | Up- | GGAAUAAAAGUCUCAACACAACAUAUA | SEQ ID |
| 006 | stream | CAAAACAAACGAAUCUCAAGCAAUCAA | NO: 202 |
| | UTR | GCAUUCUACUUCUAUUGCAGCAAUUUA | |
| | | AAUCAUUUCUUUUAAAGCAAAAGCAAU | |
| | | UUUCUGAAAAUUUUCACCAUUUACGAA | |
| | | CGAUAGCAAC | |
| 5UTR- | Up- | GGGAGACAAGCUUGGCAUUCCGGUACU | SEQ ID |
| 007 | stream | GUUGGUAAAGCCACC | NO: 203 |
| | UTR | | |
| 5UTR- | Up- | GGGAAUUAACAGAGAAAAGAAGAGUAA | SEQ ID |
| 008 | stream | GAAGAAAUAUAAGAGCCACC | NO: 204 |
| | UTR | | |
| 5UTR- | Up- | GGGAAAUUAGACAGAAAAGAAGAGUAA | SEQ ID |
| 009 | stream | GAAGAAAUAUAAGAGCCACC | NO: 205 |
| | UTR | | |
| 5UTR- | Up- | GGGAAAUAAGAGAGUAAAGAACAGUAA | SEQ ID |
| 010 | stream | GAAGAAAUAUAAGAGCCACC | NO: 206 |
| | UTR | | |
| 5UTR- | Up- | GGGAAAAAAGAGAGAAAAGAAGACUAA | SEQ ID |
| 011 | stream | GAAGAAAUAUAAGAGCCACC | NO: 207 |
| | UTR | | |
| 5UTR- | Up- | GGGAAAUAAGAGAGAAAAGAAGAGUAA | SEQ ID |
| 012 | stream | GAAGAUAUAUAAGAGCCACC | NO: 208 |
| | UTR | | |
| 5UTR- | Up- | GGGAAAUAAGAGACAAAACAAGAGUAA | SEQ ID |
| 013 | stream | GAAGAAAUAUAAGAGCCACC | NO: 209 |
| | UTR | | |
| 5UTR- | Up- | GGGAAAUUAGAGAGUAAAGAACAGUAA | SEQ ID |
| 014 | stream | GUAGAAUUAAAAGAGCCACC | NO: 210 |
| | UTR | | |
| 5UTR- | Up- | GGGAAAUAAGAGAGAAUAGAAGAGUAA | SEQ ID |
| 015 | stream | GAAGAAAUAUAAGAGCCACC | NO: 211 |
| | UTR | | |
| 5UTR- | Up- | GGGAAAUAAGAGAGAAAAGAAGAGUAA | SEQ ID |
| 016 | stream | GAAGAAAAUUAAGAGCCACC | NO: 212 |
| | UTR | | |
| 5UTR- | Up- | GGGAAAUAAGAGAGAAAAGAAGAGUAA | SEQ ID |
| 017 | stream | GAAGAAAUUUAAGAGCCACC | NO: 213 |
| | UTR | | |
| 5UTR- | Up- | GGGAAAUAAGAGAGAAAAGAAGAGUAA | SEQ ID |
| 018 | stream | GAAGAAAUAUAAGAGCCACC | NO: 214 |
| | UTR | | |

Other non-UTR sequences can also be used as regions or subregions within the polynucleotides. For example, introns or portions of introns sequences can be incorporated into regions of the polynucleotides. Incorporation of intronic sequences can increase protein production as well as polynucleotide levels.

Combinations of features can be included in flanking regions and can be contained within other features. For example, the ORF can be flanked by a 5′ UTR which can contain a strong Kozak translational initiation signal and/or a 3′ UTR which can include an oligo(dT) sequence for templated addition of a poly-A tail. 5′UTR can comprise a first polynucleotide fragment and a second polynucleotide fragment from the same and/or different genes such as the 5′UTRs described in US Patent Application Publication No. US20100293625.

These UTRs or portions thereof can be placed in the same orientation as in the transcript from which they were selected or can be altered in orientation or location. Hence a 5′ or 3′ UTR can be inverted, shortened, lengthened, made with one or more other 5′ UTRs or 3′ UTRs.

In some embodiments, the UTR sequences can be changed in some way in relation to a reference sequence. For example, a 3′ or 5′ UTR can be altered relative to a wild type or native UTR by the change in orientation or location as taught above or can be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ or 3′ UTR can be used. As used herein, a “double” UTR is one in which two copies of the same UTR are encoded either in series or substantially in series. For example, a double beta-globin 3′ UTR can be used as described in U.S. Patent Application Publ. No. US20100129877.

In some embodiments, flanking regions can be heterologous. In some embodiments, the 5′ untranslated region can be derived from a different species than the 3′ untranslated region. The untranslated region can also include translation enhancer elements (TEE). As a non-limiting example, the TEE can include those described in U.S. Patent Application Publ. No. US20090226470.

10. 3′ UTR and the AU Rich Elements

In certain embodiments, a polynucleotide of the present invention (e.g., one or more mRNAs encoding an antibody or an antigen binding portion thereof which specifically binds to CTLA-4) further comprises a 3′ UTR.

3′-UTR is the section of mRNA that immediately follows the translation termination codon and often contains regulatory regions that post-transcriptionally influence gene expression. Regulatory regions within the 3′-UTR can influence polyadenylation, translation efficiency, localization, and stability of the mRNA. In one embodiment, the 3′-UTR useful for the invention comprises a binding site for regulatory proteins or microRNAs. In some embodiments, the 3′-UTR has a silencer region, which binds to repressor proteins and inhibits the expression of the mRNA. In other embodiments, the 3′-UTR comprises an AU-rich element. Proteins bind AREs to affect the stability or decay rate of transcripts in a localized manner or affect translation initiation. In other embodiments, the 3′-UTR comprises the sequence AAUAAA that directs addition of several hundred adenine residues called the poly(A) tail to the end of the mRNA transcript.

TABLE 4 shows a listing of 3′-untranslated regions useful for the mRNAs of the present invention (e.g., one or more mRNAs encoding an antibody or an antigen binding portion thereof which specifically hinds to CTLA-4). Variants of 3′ UTRs can be utilized wherein one or more nucleotides are added or removed to the termini, including A, U, C or G.

TABLE 4
Exemplary 3′-Untranslated Regions

| 3′ UTR | Name/ | | SEQ ID |
|---|---|---|---|
| Identifier | Description | Sequence | NO. |
| 3UTR-001 | Creatine | GCGCCUGCCCACCUGCCACCGACUGCUGGAACCCAGCCA | SEQ ID |
| | Kinase | GUGGGAGGGCCUGGCCCACCAGAGUCCUGCUCCCUCACU | NO: 215 |
| | | CCUCGCCCCGCCCCCUGUCCCAGAGUCCCACCUGGGGGC | |
| | | UCUCUCCACCCUUCUCAGAGUUCCAGUUUCAACCAGAGU | |
| | | UCCAACCAAUGGGCUCCAUCCUCUGGAUUCUGGCCAAUG | |
| | | AAAUAUCUCCCUGGCAGGGUCCUCUUCUUUUCCCAGAGC | |
| | | UCCACCCCAACCAGGAGCUCUAGUUAAUGGAGAGCUCCC | |
| | | AGCACACUCGGAGCUUGUGCUUUGUCUCCACGCAAAGCG | |
| | | AUAAAUAAAAGCAUUGGUGGCCUUUGGUCUUUGAAUAAA | |
| | | GCCUGAGUAGGAAGUCUAGA | |
| 3UTR-002 | Myoglobin | GCCCCUGCCGCUCCCACCCCCACCCAUCUGGGCCCCGGG | SEQ ID |
| | | UUCAAGAGAGAGCGGGGUCUGAUCUCGUGUAGCCAUAUA | NO: 216 |
| | | GAGUUUGCUUCUGAGUGUCUGCUUUGUUUAGUAGAGGUG | |
| | | GGCAGGAGGAGCUGAGGGGCUGGGGCUGGGGUGUUGAAG | |
| | | UUGGCUUUGCAUGCCCAGCGAUGCGCCUCCCUGUGGGAU | |
| | | GUCAUCACCCUGGGAACCGGGAGUGGCCCUUGGCUCACU | |
| | | GUGUUCUGCAUGGUUUGGAUCUGAAUUAAUUGUCCUUUC | |
| | | UUCUAAAUCCCAACCGAACUUCUUCCAACCUCCAAACUG | |
| | | GCUGUAACCCCAAAUCCAAGCCAUUAACUACACCUGACA | |
| | | GUAGCAAUUGUCUGAUUAAUCACUGGCCCCUUGAAGACA | |
| | | GCAGAAUGUCCCUUUGCAAUGAGGAGGAGAUCUGGGCUG | |
| | | GGCGGGCCAGCUGGGGAAGCAUUUGACUAUCUGGAACUU | |
| | | GUGUGUGCCUCCUCAGGUAUGGCAGUGACUCACCUGGUU | |
| | | UUAAUAAAACAACCUGCAACAUCUCAUGGUCUUUGAAUA | |
| | | AAGCCUGAGUAGGAAGUCUAGA | |
| 3UTR-003 | α-actin | ACACACUCCACCUCCAGCACGCGACUUCUCAGGACGACG | SEQ ID |
| | | AAUCUUCUCAAUGGGGGGGCGGCUGAGCUCCAGCCACCC | NO: 217 |
| | | CGCAGUCACUUUCUUUGUAACAACUUCCGUUGCUGCCAU | |
| | | CGUAAACUGACACAGUGUUUAUAACGUGUACAUACAUUA | |
| | | ACUUAUUACCUCAUUUUGUUAUUUUUCGAAACAAAGCCC | |
| | | UGUGGAAGAAAAUGGAAAACUUGAAGAAGCAUUAAAGUC | |
| | | AUUCUGUUAAGCUGCGUAAAUGGUCUUUGAAUAAAGCCU | |
| | | GAGUAGGAAGUCUAGA | |
| 3UTR-004 | Albumin | CAUCACAUUUAAAAGCAUCUCAGCCUACCAUGAGAAUAA | SEQ ID |
| | | GAGAAAGAAAAUGAAGAUCAAAAGCUUAUUCAUCUGUUU | NO: 218 |
| | | UUCUUUUUCGUUGGUGUAAAGCCAACACCCUGUCUAAAA | |
| | | AACAUAAAUUUCUUUAAUCAUUUUGCCUCUUUUCUCUGU | |
| | | GCUUCAAUUAAUAAAAAAUGGAAAGAAUCUAAUAGAGUG | |
| | | GUACAGCACUGUUAUUUUUCAAAGAUGUGUUGCUAUCCU | |
| | | GAAAAUUCUGUAGGUUCUGUGGAAGUUCCAGUGUUCUCU | |
| | | CUUAUUCCACUUCGGUAGAGGAUUUCUAGUUUCUUGUGG | |
| | | GCUAAUUAAAUAAAUCAUUAAUACUCUUCUAAUGGUCUU | |
| | | UGAAUAAAGCCUGAGUAGGAAGUCUAGA | |
| 3UTR-005 | α-globin | GCUGCCUUCUGCGGGGCUUGCCUUCUGGCCAUGCCCUUC | SEQ ID |
| | | UUCUCUCCCUUGCACCUGUACCUCUUGGUCUUUGAAUAA | NO: 219 |
| | | AGCCUGAGUAGGAAGGCGGCCGCUCGAGCAUGCAUCUAG | |
| | | A | |
| 3UTR-006 | G-CSF | GCCAAGCCCUCCCCAUCCCAUGUAUUUAUCUCUAUUUAA | SEQ ID |
| | | UAUUUAUGUCUAUUUAAGCCUCAUAUUUAAAGACAGGGA | NO: 220 |
| | | AGAGCAGAACGGAGCCCCAGGCCUCUGUGUCCUUCCCUG | |
| | | CAUUUCUGAGUUUCAUUCUCCUGCCUGUAGCAGUGAGAA | |
| | | AAAGCUCCUGUCCUCCCAUCCCCUGGACUGGGAGGUAGA | |
| | | UAGGUAAAUACCAAGUAUUUAUUACUAUGACUGCUCCCC | |
| | | AGCCCUGGCUCUGCAAUGGGCACUGGGAUGAGCCGCUGU | |
| | | GAGCCCCUGGUCCUGAGGGUCCCCACCUGGGACCCUUGA | |
| | | GAGUAUCAGGUCUCCCACGUGGGAGACAAGAAAUCCCUG | |
| | | UUUAAUAUUUAAACAGCAGUGUUCCCCAUCUGGGUCCUU | |
| | | GCACCCCUCACUCUGGCCUCAGCCGACUGCACAGCGGCC | |
| | | CCUGCAUCCCCUUGGCUGUGAGGCCCCUGGACAAGCAGA | |
| | | GGUGGCCAGAGCUGGGAGGCAUGGCCCUGGGGUCCCACG | |
| | | AAUUUGCUGGGGAAUCUCGUUUUUCUUCUUAAGACUUUU | |
| | | GGGACAUGGUUUGACUCCCGAACAUCACCGACGCGUCUC | |
| | | CUGUUUUUCUGGGUGGCCUCGGGACACCUGCCCUGCCCC | |
| | | CACGAGGGUCAGGACUGUGACUCUUUUUAGGGCCAGGCA | |
| | | GGUGCCUGGACAUUUGCCUUGCUGGACGGGGACUGGGGA | |
| | | UGUGGGAGGGAGCAGACAGGAGGAAUCAUGUCAGGCCUG | |
| | | UGUGUGAAAGGAAGCUCCACUGUCACCCUCCACCUCUUC | |
| | | ACCCCCCACUCACCAGUGUCCCCUCCACUGUCACAUUGU | |
| | | AACUGAACUUCAGGAUAAUAAAGUGUUUGCCUCCAUGGU | |
| | | CUUUGAAUAAAGCCUGAGUAGGAAGGCGGCCGCUCGAGC | |
| | | AUGCAUCUAGA | |
| 3UTR-007 | C0l1a2; | ACUCAAUCUAAAUUAAAAAAGAAAGAAAUUUGAAAAAAC | SEQ ID |
| | collagen, | UUUCUCUUUGCCAUUUCUUCUUCUUCUUUUUUAACUGAA | NO: 221 |
| | type I, | AGCUGAAUCCUUCCAUUUCUUCUGCACAUCUACUUGCUU | |
| | alpha 2 | AAAUUGUGGGCAAAAGAGAAAAAGAAGGAUUGAUCAGAG | |
| | | CAUUGUGCAAUACAGUUUCAUUAACUCCUUCCCCCGCUC | |
| | | CCCCAAAAAUUUGAAUUUUUUUUUCAACACUCUUACACC | |
| | | UGUUAUGGAAAAUGUCAACCUUUGUAAGAAAACCAAAAU | |
| | | AAAAAUUGAAAAAUAAAAACCAUAAACAUUUGCACCACU | |
| | | UGUGGCUUUUGAAUAUCUUCCACAGAGGGAAGUUUAAAA | |
| | | CCCAAACUUCCAAAGGUUUAAACUACCUCAAAACACUUU | |
| | | CCCAUGAGUGUGAUCCACAUUGUUAGGUGCUGACCUAGA | |
| | | CAGAGAUGAACUGAGGUCCUUGUUUUGUUUUGUUCAUAA | |
| | | UACAAAGGUGCUAAUUAAUAGUAUUUCAGAUACUUGAAG | |
| | | AAUGUUGAUGGUGCUAGAAGAAUUUGAGAAGAAAUACUC | |
| | | CUGUAUUGAGUUGUAUCGUGUGGUGUAUUUUUUAAAAAA | |
| | | UUUGAUUUAGCAUUCAUAUUUUCCAUCUUAUUCCCAAUU | |
| | | AAAAGUAUGCAGAUUAUUUGCCCAAAUCUUCUUCAGAUU | |
| | | CAGCAUUUGUUCUUUGCCAGUCUCAUUUUCAUCUUCUUC | |
| | | CAUGGUUCCACAGAAGCUUUGUUUCUUGGGCAAGCAGAA | |
| | | AAAUUAAAUUGUACCUAUUUUGUAUAUGUGAGAUGUUUA | |
| | | AAUAAAUUGUGAAAAAAAUGAAAUAAAGCAUGUUUGGUU | |
| | | UUCCAAAAGAACAUAU | |
| 3UTR-008 | Col6a2; | CGCCGCCGCCCGGGCCCCGCAGUCGAGGGUCGUGAGCCC | SEQ ID |
| | collagen, | ACCCCGUCCAUGGUGCUAAGCGGGCCCGGGUCCCACACG | NO: 222 |
| | type VI, | GCCAGCACCGCUGCUCACUCGGACGACGCCCUGGGCCUG | |
| | alpha 2 | CACCUCUCCAGCUCCUCCCACGGGGUCCCCGUAGCCCCG | |
| | | GCCCCCGCCCAGCCCCAGGUCUCCCCAGGCCCUCCGCAG | |
| | | GCUGCCCGGCCUCCCUCCCCCUGCAGCCAUCCCAAGGCU | |
| | | CCUGACCUACCUGGCCCCUGAGCUCUGGAGCAAGCCCUG | |
| | | ACCCAAUAAAGGCUUUGAACCCAU | |
| 3UTR-009 | RPN1; | GGGGCUAGAGCCCUCUCCGCACAGCGUGGAGACGGGGCA | SEQ ID |
| | ribophorin I | AGGAGGGGGGUUAUUAGGAUUGGUGGUUUUGUUUUGCUU | NO: 223 |
| | | UGUUUAAAGCCGUGGGAAAAUGGCACAACUUUACCUCUG | |
| | | UGGGAGAUGCAACACUGAGAGCCAAGGGGUGGGAGUUGG | |
| | | GAUAAUUUUUAUAUAAAAGAAGUUUUUCCACUUUGAAUU | |
| | | GCUAAAAGUGGCAUUUUUCCUAUGUGCAGUCACUCCUCU | |
| | | CAUUUCUAAAAUAGGGACGUGGCCAGGCACGGUGGCUCA | |
| | | UGCCUGUAAUCCCAGCACUUUGGGAGGCCGAGGCAGGCG | |
| | | GCUCACGAGGUCAGGAGAUCGAGACUAUCCUGGCUAACA | |
| | | CGGUAAAACCCUGUCUCUACUAAAAGUACAAAAAAUUAG | |
| | | CUGGGCGUGGUGGUGGGCACCUGUAGUCCCAGCUACUCG | |
| | | GGAGGCUGAGGCAGGAGAAAGGCAUGAAUCCAAGAGGCA | |
| | | GAGCUUGCAGUGAGCUGAGAUCACGCCAUUGCACUCCAG | |
| | | CCUGGGCAACAGUGUUAAGACUCUGUCUCAAAUAUAAAU | |
| | | AAAUAAAUAAAUAAAUAAAUAAAUAAAUAAAAAUAAAGC | |
| | | GAGAUGUUGCCCUCAAA | |
| 3UTR-010 | LRP1; low | GGCCCUGCCCCGUCGGACUGCCCCCAGAAAGCCUCCUGC | SEQ ID |
| | density | CCCCUGCCAGUGAAGUCCUUCAGUGAGCCCCUCCCCAGC | NO: 224 |
| | lipoprotein | CAGCCCUUCCCUGGCCCCGCCGGAUGUAUAAAUGUAAAA | |
| | receptor- | AUGAAGGAAUUACAUUUUAUAUGUGAGCGAGCAAGCCGG | |
| | related | CAAGCGAGCACAGUAUUAUUUCUCCAUCCCCUCCCUGCC | |
| | protein 1 | UGCUCCUUGGCACCCCCAUGCUGCCUUCAGGGAGACAGG | |
| | | CAGGGAGGGCUUGGGGCUGCACCUCCUACCCUCCCACCA | |
| | | GAACGCACCCCACUGGGAGAGCUGGUGGUGCAGCCUUCC | |
| | | CCUCCCUGUAUAAGACACUUUGCCAAGGCUCUCCCCUCU | |
| | | CGCCCCAUCCCUGCUUGCCCGCUCCCACAGCUUCCUGAG | |
| | | GGCUAAUUCUGGGAAGGGAGAGUUCUUUGCUGCCCCUGU | |
| | | CUGGAAGACGUGGCUCUGGGUGAGGUAGGCGGGAAAGGA | |
| | | UGGAGUGUUUUAGUUCUUGGGGGAGGCCACCCCAAACCC | |
| | | CAGCCCCAACUCCAGGGGCACCUAUGAGAUGGCCAUGCU | |
| | | CAACCCCCCUCCCAGACAGGCCCUCCCUGUCUCCAGGGC | |
| | | CCCCACCGAGGUUCCCAGGGCUGGAGACUUCCUCUGGUA | |
| | | AACAUUCCUCCAGCCUCCCCUCCCCUGGGGACGCCAAGG | |
| | | AGGUGGGCCACACCCAGGAAGGGPAAGCGGGCAGCCCCG | |
| | | UUUUGGGGACGUGAACGUUUUAAUAAUUUUUGCUGAAUU | |
| | | CCUUUACAACUAAAUAACACAGAUAUUGUUAUAAAUAAA | |
| | | AUUGU | |
| 3UTR-011 | Nnt1; | AUAUUAAGGAUCAAGCUGUUAGCUAAUAAUGCCACCUCU | SEQ ID |
| | cardiotrophin- | GCAGUUUUGGGAACAGGCAAAUAAAGUAUCAGUAUACAU | NO: 225 |
| | like | GGUGAUGUACAUCUGUAGCAAAGCUCUUGGAGAAAAUGA | |
| | cytokine | AGACUGAAGAAAGCAAAGCAAAAACUGUAUAGAGAGAUU | |
| | factor 1 | UUUCAAAAGCAGUAAUCCCUCAAUUUUAAAAAAGGAUUG | |
| | | AAAAUUCUAAAUGUCUUUCUGUGCAUAUUUUUUGUGUUA | |
| | | GGAAUCAAAAGUAUUUUAUAAAAGGAGAAAGAACAGCCU | |
| | | CAUUUUAGAUGUAGUCCUGUUGGAUUUUUUAUGCCUCCU | |
| | | CAGUAACCAGAAAUGUUUUAAAAAACUAAGUGUUUAGGA | |
| | | UUUCAAGACAACAUUAUACAUGGCUCUGAAAUAUCUGAC | |
| | | ACAAUGUAAACAUUGCAGGCACCUGCAUUUUAUGUUUUU | |
| | | UUUUUCAACAAAUGUGACUAAUUUGAAACUUUUAUGAAC | |
| | | UUCUGAGCUGUCCCCUUGCAAUUCAACCGCAGUUUGAAU | |
| | | UAAUCAUAUCAAAUCAGUUUUAAUUUUUUAAAUUGUACU | |
| | | UCAGAGUCUAUAUUUCAAGGGCACAUUUUCUCACUACUA | |
| | | UUUUAAUACAUUAAAGGACUAAAUAAUCUUUCAGAGAUG | |
| | | CUGGAAACAAAUCAUUUGCUUUAUAUGUUUCAUUAGAAU | |
| | | ACCAAUGAAACAUACAACUUGAAAAUUAGUAAUAGUAUU | |
| | | UUUGAAGAUCCCAUUUCUAAUUGGAGAUCUCUUUAAUUU | |
| | | CGAUCAACUUAUAAUGUGUAGUACUAUAUUAAGUGCACU | |
| | | UGAGUGGAAUUCAACAUUUGACUAAUAAAAUGAGUUCAU | |
| | | CAUGUUGGCAAGUGAUGUGGCAAUUAUCUCUGGUGACAA | |
| | | AAGAGUAAAAUCAAAUAUUUCUGCCUGUUACAAAUAUCA | |
| | | AGGAAGACCUGCUACUAUGAAAUAGAUGACAUUAAUCUG | |
| | | UCUUCACUGUUUAUAAUACGGAUGGAUUUUUUUUCAAAU | |
| | | CAGUGUGUGUUUUGAGGUCUUAUGUAAUUGAUGACAUUU | |
| | | GAGAGAAAUGGUGGCUUUUUUUAGCUACCUCUUUGUUCA | |
| | | UUUAAGCACCAGUAAAGAUCAUGUCUUUUUAUAGAAGUG | |
| | | UAGAUUUUCUUUGUGACUUUGCUAUCGUGCCUAAAGCUC | |
| | | UAAAUAUAGGUGAAUGUGUGAUGAAUACUCAGAUUAUUU | |
| | | GUCUCUCUAUAUAAUUAGUUUGGUACUAAGUUUCUCAAA | |
| | | AAAUUAUUAACACAUGAAAGACAAUCUCUAAACCAGAAA | |
| | | AAGAAGUAGUACAAAUUUUGUUACUGUAAUGCUCGCGUU | |
| | | UAGUGAGUUUAAAACACACAGUAUCUUUUGGUUUUAUAA | |
| | | UCAGUUUCUAUUUUGCUGUGCCUGAGAUUAAGAUCUGUG | |
| | | UAUGUGUGUGUGUGUGUGUGUGCGUUUGUGUGUUAAAGC | |
| | | AGAAAAGACUUUUUUAAAAGUUUUAAGUGAUAAAUGCAA | |
| | | UUUGUUAAUUGAUCUUAGAUCACUAGUAAACUCAGGGCU | |
| | | GAAUUAUACCAUGUAUAUUCUAUUAGAAGAAAGUAAACA | |
| | | CCAUCUUUAUUCCUGCCCUUUUUCUUCUCUCAAAGUAGU | |
| | | UGUAGUUAUAUCUAGAAAGAAGCAAAUUUGAUUUCUUGA | |
| | | AAAGGUAGUUCCUGCACUCAGUUUAAACUAAAAAUAAUC | |
| | | AUACUUGGAUUUUAUUUAUUUUUGUCAUAGUAAAAAUUU | |
| | | UAAUUUAUAUAUAUUUUUAUUUAGUAUUAUCUUAUUCUU | |
| | | UGCUAUUUGCCAAUCCUUUGUCAUCAAUUGUGUUAAAUG | |
| | | AAUUGAAAAUUCAUGCCCUGUUCAUUUUAUUUUACUUUA | |
| | | UUGGUUAGGAUAUUUAAAGGAUUUUUGUAUAUAUAAUUU | |
| | | CUUAAAUUAAUAUUCCAAAAGGUUAGUGGACUUAGAUUA | |
| | | UAAAUUAUGGCAAAAAUCUAAAAACAACAAAAAUGAUUU | |
| | | UUAUACAUUCUAUUUCAUUAUUCCUCUUUUUCCAAUAAG | |
| | | UCAUACAAUUGGUAGAUAUGACUUAUUUUAUUUUUGUAU | |
| | | UAUUCACUAUAUCUUUAUGAUAUUUAAGUAUAAAUAAUU | |
| | | AAAAAAAUUUAUUGUACCUUAUAGUCUGUCACCAAAAAA | |
| | | AAAAAAUUAUCUGUAGGUAGUGAAAUGCUAAUGUUGAUU | |
| | | UGUCUUUAAGGGCUUGUUAACUAUCCUUUAUUUUCUCAU | |
| | | UUGUCUUAAAUUAGGAGUUUGUGUUUAAAUUACUCAUCU | |
| | | AAGCAAAAAAUGUAUAUAAAUCCCAUUACUGGGUAUAUA | |
| | | CCCAAAGGAUUAUAAAUCAUGCUGCUAUAAAGACACAUG | |
| | | CACACGUAUGUUUAUUGCAGCACUAUUCACAAUAGCAAA | |
| | | GACUUGGAACCAACCCAAAUGUCCAUCAAUGAUAGACUU | |
| | | GAUUAAGAAAAUGUGCACAUAUACACCAUGGAAUACUAU | |
| | | GCAGCCAUAAAAAAGGAUGAGUUCAUGUCCUUUGUAGGG | |
| | | ACAUGGAUAAAGCUGGAAACCAUCAUUCUGAGCAAACUA | |
| | | UUGCAAGGACAGAAAACCAAACACUGCAUGUUCUCACUC | |
| | | AUAGGUGGGAAUUGAACAAUGAGAACACUUGGACACAAG | |
| | | GUGGGGAACACCACACACCAGGGCCUGUCAUGGGGUGGG | |
| | | GGGAGUGGGGAGGGAUAGCAUUAGGAGAUAUACCUAAUG | |
| | | UAAAUGAUGAGUUAAUGGGUGCAGCACACCAACAUGGCA | |
| | | CAUGUAUACAUAUGUAGCAAACCUGCACGUUGUGCACAU | |
| | | GUACCCUAGAACUUAAAGUAUAAUUAAAAAAAAAAAGAA | |
| | | AACAGAAGCUAUUUAUAAAGAAGUUAUUUGCUGAAAUAA | |
| | | AUGUGAUCUUUCCCAUUAAAAAAAUAAAGAAAUUUUGGG | |
| | | GUAAAAAAACACAAUAUAUUGUAUUCUUGAAAAAUUCUA | |
| | | AGAGAGUGGAUGUGAAGUGUUCUCACCACAAAAGUGAUA | |
| | | ACUAAUUGAGGUAAUGCACAUAUUAAUUAGAAAGAUUUU | |
| | | GUCAUUCCACAAUGUAUAUAUACUUAAAAAUAUGUUAUA | |
| | | CACAAUAAAUACAUACAUUAAAAAAUAAGUAAAUGUA | |
| 3UTR-012 | Col6a1; | CCCACCCUGCACGCCGGCACCAAACCCUGUCCUCCCACC | SEQ ID |
| | collagen, | CCUCCCCACUCAUCACUAAACAGAGUAAAAUGUGAUGCG | NO: 226 |
| | type VI, | AAUUUUCCCGACCAACCUGAUUCGCUAGAUUUUUUUUAA | |
| | alpha 1 | GGAAAAGCUUGGAAAGCCAGGACACAACGCUGCUGCCUG | |
| | | CUUUGUGCAGGGUCCUCCGGGGCUCAGCCCUGAGUUGGC | |
| | | AUCACCUGCGCAGGGCCCUCUGGGGCUCAGCCCUGAGCU | |
| | | AGUGUCACCUGCACAGGGCCCUCUGAGGCUCAGCCCUGA | |
| | | GCUGGCGUCACCUGUGCAGGGCCCUCUGGGGCUCAGCCC | |
| | | UGAGCUGGCCUCACCUGGGUUCCCCACCCCGGGCUCUCC | |
| | | UGCCCUGCCCUCCUGCCCGCCCUCCCUCCUGCCUGCGCA | |
| | | GCUCCUUCCCUAGGCACCUCUGUGCUGCAUCCCACCAGC | |
| | | CUGAGCAAGACGCCCUCUCGGGGCCUGUGCCGCACUAGC | |
| | | CUCCCUCUCCUCUGUCCCCAUAGCUGGUUUUUCCCACCA | |
| | | AUCCUCACCUAACAGUUACUUUACAAUUAAACUCAAAGC | |
| | | AAGCUCUUCUCCUCAGCUUGGGGCAGCCAUUGGCCUCUG | |
| | | UCUCGUUUUGGGAAACCAAGGUCAGGAGGCCGUUGCAGA | |
| | | CAUAAAUCUCGGCGACUCGGCCCCGUCUCCUGAGGGUCC | |
| | | UGCUGGUGACCGGCCUGGACCUUGGCCCUACAGCCCUGG | |
| | | AGGCCGCUGCUGACCAGCACUGACCCCGACCUCAGAGAG | |
| | | UACUCGCAGGGGCGCUGGCUGCACUCAAGACCCUCGAGA | |
| | | UUAACGGUGCUAACCCCGUCUGCUCCUCCCUCCCGCAGA | |
| | | GACUGGGGCCUGGACUGGACAUGAGAGCCCCUUGGUGCC | |
| | | ACAGAGGGCUGUGUCUUACUAGAAACAACGCAAACCUCU | |
| | | CCUUCCUCAGAAUAGUGAUGUGUUCGACGUUUUAUCAAA | |
| | | GGCCCCCUUUCUAUGUUCAUGUUAGUUUUGCUCCUUCUG | |
| | | UGUUUUUUUCUGAACCAUAUCCAUGUUGCUGACUUUUCC | |
| | | AAAUAAAGGUUUUCACUCCUCUC | |
| 3UTR-013 | Calr; | AGAGGCCUGCCUCCAGGGCUGGACUGAGGCCUGAGCGCU | SEQ ID |
| | calreticulin | CCUGCCGCAGAGCUGGCCGCGCCAAAUAAUGUCUCUGUG | NO: 227 |
| | | AGACUCGAGAACUUUCAUUUUUUUCCAGGCUGGUUCGGA | |
| | | UUUGGGGUGGAUUUUGGUUUUGUUCCCCUCCUCCACUCU | |
| | | CCCCCACCCCCUCCCCGCCCUUUUUUUUUUUUUUUUUUA | |
| | | AACUGGUAUUUUAUCUUUGAUUCUCCUUCAGCCCUCACC | |
| | | CCUGGUUCUCAUCUUUCUUGAUCAACAUCUUUUCUUGCC | |
| | | UCUGUCCCCUUCUCUCAUCUCUUAGCUCCCCUCCAACCU | |
| | | GGGGGGCAGUGGUGUGGAGAAGCCACAGGCCUGAGAUUU | |
| | | CAUCUGCUCUCCUUCCUGGAGCCCAGAGGAGGGCAGCAG | |
| | | AAGGGGGUGGUGUCUCCAACCCCCCAGCACUGAGGAAGA | |
| | | ACGGGGCUCUUCUCAUUUCACCCCUCCCUUUCUCCCCUG | |
| | | CCCCCAGGACUGGGCCACUUCUGGGUGGGGCAGUGGGUC | |
| | | CCAGAUUGGCUCACACUGAGAAUGUAAGAACUACAAACA | |
| | | AAAUUUCUAUUAAAUUAAAUUUUGUGUCUCC | |
| 3UTR-014 | Col1a1; | CUCCCUCCAUCCCAACCUGGCUCCCUCCCACCCAACCAA | SEQ ID |
| | collagen, | CUUUCCCCCCAACCCGGAAACAGACAAGCAACCCAAACU | NO: 228 |
| | type I, | GAACCCCCUCAAAAGCCAAAAAAUGGGAGACAAUUUCAC | |
| | alpha 1 | AUGGACUUUGGAAAAUAUUUUUUUCCUUUGCAUUCAUCU | |
| | | CUCAAACUUAGUUUUUAUCUUUGACCAACCGAACAUGAC | |
| | | CAAAAACCAAAAGUGCAUUCAACCUUACCAAAAAAAAAA | |
| | | AAAAAAAAAGAAUAAAUAAAUAACUUUUUAAAAAAGGAA | |
| | | GCUUGGUCCACUUGCUUGAAGACCCAUGCGGGGGUAAGU | |
| | | CCCUUUCUGCCCGUUGGGCUUAUGAAACCCCAAUGCUGC | |
| | | CCUUUCUGCUCCUUUCUCCACACCCCCCUUGGGGCCUCC | |
| | | CCUCCACUCCUUCCCAAAUCUGUCUCCCCAGAAGACACA | |
| | | GGAAACAAUGUAUUGUCUGCCCAGCAAUCAAAGGCAAUG | |
| | | CUCAAACACCCAAGUGGCCCCCACCCUCAGCCCGCUCCU | |
| | | GCCCGCCCAGCACCCCCAGGCCCUGGGGGACCUGGGGUU | |
| | | CUCAGACUGCCAAAGAAGCCUUGCCAUCUGGCGCUCCCA | |
| | | UGGCUCUUGCAACAUCUCCCCUUCGUUUUUGAGGGGGUC | |
| | | AUGCCGGGGGAGCCACCAGCCCCUCACUGGGUUCGGAGG | |
| | | AGAGUCAGGAAGGGCCACGACAAAGCAGAAACAUCGGAU | |
| | | UUGGGGAACGCGUGUCAAUCCCUUGUGCCGCAGGGCUGG | |
| | | GCGGGAGAGACUGUUCUGUUCCUUGUGUAACUGUGUUGC | |
| | | UGAAAGACUACCUCGUUCUUGUCUUGAUGUGUCACCGGG | |
| | | GCAACUGCCUGGGGGCGGGGAUGGGGGCAGGGUGGAAGC | |
| | | GGCUCCCCAUUUUAUACCAAAGGUGCUACAUCUAUGUGA | |
| | | UGGGUGGGGUGGGGAGGGAAUCACUGGUGCUAUAGAAAU | |
| | | UGAGAUGCCCCCCCAGGCCAGCAAAUGUUCCUUUUUGUU | |
| | | CAAAGUCUAUUUUUAUUCCUUGAUAUUUUUCUUUUUUUU | |
| | | UUUUUUUUUUUGUGGAUGGGGACUUGUGAAUUUUUCUAA | |
| | | AGGUGCUAUUUAACAUGGGAGGAGAGCGUGUGCGGCUCC | |
| | | AGCCCAGCCCGCUGCUCACUUUCCACCCUCUCUCCACCU | |
| | | GCCUCUGGCUUCUCAGGCCUCUGCUCUCCGACCUCUCUC | |
| | | CUCUGAAACCCUCCUCCACAGCUGCAGCCCAUCCUCCCG | |
| | | GCUCCCUCCUAGUCUGUCCUGCGUCCUCUGUCCCCGGGU | |
| | | UUCAGAGACAACUUCCCAAAGCACAAAGCAGUUUUUCCC | |
| | | CCUAGGGGUGGGAGGAAGCAAAAGACUCUGUACCUAUUU | |
| | | UGUAUGUGUAUAAUAAUUUGAGAUGUUUUUAAUUAUUUU | |
| | | GAUUGCUGGAAUAAAGCAUGUGGAAAUGACCCAAACAUA | |
| | | AUCCGCAGUGGCCUCCUAAUUUCCUUCUUUGGAGUUGGG | |
| | | GGAGGGGUAGACAUGGGGAAGGGGCUUUGGGGUGAUGGG | |
| | | CUUGCCUUCCAUUCCUGCCCUUUCCCUCCCCACUAUUCU | |
| | | CUUCUAGAUCCCUCCAUAACCCCACUCCCCUUUCUCUCA | |
| | | CCCUUCUUAUACCGCAAACCUUUCUACUUCCUCUUUCAU | |
| | | UUUCUAUUCUUGCAAUUUCCUUGCACCUUUUCCAAAUCC | |
| | | UCUUCUCCCCUGCAAUACCAUACAGGCAAUCCACGUGCA | |
| | | CAACACACACACACACUCUUCACAUCUGGGGUUGUCCAA | |
| | | ACCUCAUACCCACUCCCCUUCAAGCCCAUCCACUCUCCA | |
| | | CCCCCUGGAUGCCCUGCACUUGGUGGCGGUGGGAUGCUC | |
| | | AUGGAUACUGGGAGGGUGAGGGGAGUGGAACCCGUGAGG | |
| | | AGGACCUGGGGGCCUCUCCUUGAACUGACAUGAAGGGUC | |
| | | AUCUGGCCUCUGCUCCCUUCUCACCCACGCUGACCUCCU | |
| | | GCCGAAGGAGCAACGCAACAGGAGAGGGGUCUGCUGAGC | |
| | | CUGGCGAGGGUCUGGGAGGGACCAGGAGGAAGGCGUGCU | |
| | | CCCUGCUCGCUGUCCUGGCCCUGGGGGAGUGAGGGAGAC | |
| | | AGACACCUGGGAGAGCUGUGGGGAAGGCACUCGCACCGU | |
| | | GCUCUUGGGAAGGAAGGAGACCUGGCCCUGCUCACCACG | |
| | | GACUGGGUGCCUCGACCUCCUGAAUCCCCAGAACACAAC | |
| | | CCCCCUGGGCUGGGGUGGUCUGGGGAACCAUCGUGCCCC | |
| | | CGCCUCCCGCCUACUCCUUUUUAAGCUU | |
| 3UTR-015 | Plod1; | UUGGCC2GGCCUGACCCUCUUGGACCUUUCUUCUUUGCC | SEQ ID |
| | procollagen- | GACAACCACUGCCCAGCAGCCUCUGGGACCUCGGGGUCC | NO: 229 |
| | lysine, 2- | CAGGGAACCCAGUCCAGCCUCCUGGCUGUUGACUUCCCA | |
| | oxoglutarate | UUGCUCUUGGAGCCACCAAUCAAAGAGAUUCAAAGAGAU | |
| | 5- | UCCUGCAGGCCAGAGGCGGAACACACCUUUAUGGCUGGG | |
| | dioxygenase | GCUCUCCGUGGUGUUCUGGACCCAGCCCCUGGAGACACC | |
| | 1 | AUUCACUUUUACUGCUUUGUAGUGACUCGUGCUCUCCAA | |
| | | CCUGUCUUCCUGAAAAACCAAGGCCCCCUUCCCCCACCU | |
| | | CUUCCAUGGGGUGAGACUUGAGCAGAACAGGGGCUUCCC | |
| | | CAAGUUGCCCAGAAAGACUGUCUGGGUGAGAAGCCAUGG | |
| | | CCAGAGCUUCUCCCAGGCACAGGUGUUGCACCAGGGACU | |
| | | UCUGCUUCAAGUUUUGGGGUAAAGACACCUGGAUCAGAC | |
| | | UCCAAGGGCUGCCCUGAGUCUGGGACUUCUGCCUCCAUG | |
| | | GCUGGUCAUGAGAGCAAACCGUAGUCCCCUGGAGACAGC | |
| | | GACUCCAGAGAACCUCUUGGGAGACAGAAGAGGCAUCUG | |
| | | UGCACAGCUCGAUCUUCUACUUGCCUGUGGGGAGGGGAG | |
| | | UGACAGGUCCACACACCACACUGGGUCACCCUGUCCUGG | |
| | | AUGCCUCUGAAGAGAGGGACAGACCGUCAGAAACUGGAG | |
| | | AGUUUCUAUUAAAGGUCAUUUAAACCA | |
| 3UTR-016 | Nucb 1; | UCCUCCGGGACCCCAGCCCUCAGGAUUCCUGAUGCUCCA | SEQ ID |
| | nucleo- | AGGCGACUGAUGGGCGCUGGAUGAAGUGGCACAGUCAGC | NO: 230 |
| | bindin 1 | UUCCCUGGGGGCUGGUGUCAUGUUGGGCUCCUGGGGCGG | |
| | | GGGCACGGCCUGGCAUUUCACGCAUUGCUGCCACCCCAG | |
| | | GUCCACCUGUCUCCACUUUCACAGCCUCCAAGUCUGUGG | |
| | | CUCUUCCCUUCUGUCCUCCGAGGGGCUUGCCUUCUCUCG | |
| | | UGUCCAGUGAGGUGCUCAGUGAUCGGCUUAACUUAGAGA | |
| | | AGCCCGCCCCCUCCCCUUCUCCGUCUGUCCCAAGAGGGU | |
| | | CUGCUCUGAGCCUGCGUUCCUAGGUGGCUCGGCCUCAGC | |
| | | UGCCUGGGUUGUGGCCGCCCUAGCAUCCUGUAUGCCCAC | |
| | | AGCUACUGGAAUCCCCGCUGCUGCUCCGGGCCAAGCUUC | |
| | | UGGUUGAUUAAUGAGGGCAUGGGGUGGUCCCUCAAGACC | |
| | | UUCCCCUACCUUUUGUGGAACCAGUGAUGCCUCAAAGAC | |
| | | AGUGUCCCCUCCACAGCUGGGUGCCAGGGGCAGGGGAUC | |
| | | CUCAGUAUAGCCGGUGAACCCUGAUACCAGGAGCCUGGG | |
| | | CCUCCCUGAACCCCUGGCUUCCAGCCAUCUCAUCGCCAG | |
| | | CCUCCUCCUGGACCUCUUGGCCCCCAGCCCCUUCCCCAC | |
| | | ACAGCCCCAGAAGGGUCCCAGAGCUGACCCCACUCCAGG | |
| | | ACCUAGGCCCAGCCCCUCAGCCUCAUCUGGAGCCCCUGA | |
| | | AGACCAGUCCCACCCACCUUUCUGGCCUCAUCUGACACU | |
| | | GCUCCGCAUCCUGCUGUGUGUCCUGUUCCAUGUUCCGGU | |
| | | UCCAUCCAAAUACACUUUCUGGAACAAA | |
| 3UTR-017 | α-globin | GCUGGAGCCUCGGUGGCCAUGCUUCUUGCCCCUUGGGCC | SEQ ID |
| | | UCCCCCCAGCCCCUCCUCCCCUUCCUGCACCCGUACCCC | NO: 231 |
| | | CGUGGUCUUUGAAUAAAGUCUGAGUGGGCGGC | |
| 3UTR-018 | Downstream | UAAUAGGCUGGAGCCUCGGUGGCCAUGCUUCUUGCCCCU | SEQ ID |
| | UTR | UGGGCCUCCCCCCAGCCCCUCCUCCCCUUCCUGCACCCG | NO: 232 |
| | | UACCCCCGUGGUCUUUGAAUAAAGUCUGAGUGGGCGGC | |

In certain embodiments, the 3′ UTR sequence useful for the invention comprises a nucledotide sequence at least about 60%, at least about 70%, at least about 80%, at least about 90%, at least about 95%, at least about 96%, at least about 97%, at least about 98%, at least about 99%, or about 100% identical to a sequence selected from the group consisting of SEQ ID NOS: 45-62 and any combination thereof. In some embodiments, a 3′UTR sequence useful for the invention comprises 3′ UTR-018 (SEQ ID NO: 62).

11. Regions Having a 5′ Cap

The polynucleotides of the present invention (e.g., one or more mRNAs encoding an antibody or an antigen binding portion thereof which specifically binds to CTLA-4) can further comprise a 5′ cap. The 5′ cap can bind the mRNA Cap Binding Protein (CBP), thereby increasing mRNA stability. The cap can further assist the removal of 5′ proximal introns removal during mRNA splicing.

In some embodiments, the polynucleotides of the present invention (e.g., one or more mRNAs encoding an antibody or an antigen binding portion thereof which specifically binds to CTLA-4) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

In certain embodiments, the 5′ cap comprises 2′-O-methylation of the ribose sugars of 5′-terminal and/or 5′-anteterminal nucleotides on the 2′-hydroxyl group of the sugar ring. In other embodiments, the cap for a polynucleotide of the present invention (e.g., an mRNA encoding an antibody or an antigen binding portion thereof which specifically binds to CTLA-4) includes cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs can be chemically (i.e. non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In some embodiments, the cap is a dinucleotide cap analog. As a non-limiting example, the dinucleotide cap analog can be modified at different phosphate positions with a boranophosphate group or a phophoroselenoate group such as the dinucleotide cap analogs described in U.S. Pat. No. 8,519,110.

In another embodiment, the cap is a cap analog which is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G cap analog and a N7-(4-chlorophenoxyethyl)-m^{3′-O}G(5′)ppp(5′)G cap analog. See, e.g., the various cap analogs and the methods of synthesizing cap analogs described in Kore et al. Bioorganic & Medicinal Chemistry 21:4570-4574 (2013). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences between a cap analog and the endogenous 5′-cap structure of a nucleic acid produced by the endogenous, cellular transcription machinery, can lead to reduced translational competency and reduced cellular stability.

Polynucleotides of the present disclosure (e.g., one or more mRNAs encoding an antibody or an antigen binding portion thereof which specifically binds to CTLA-4) can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)N1mpNp (cap 1), and 7mG(5′)-ppp(5′)N1mpN2mp (cap 2).

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

12. Poly-A Tails

In some embodiments, the polynucleotides of the present disclosure (e.g., one or more mRNAs encoding an antibody or an antigen binding portion thereof which specifically binds to CTLA-4) further comprise a poly A tail. In further embodiments, terminal groups on the poly-A tail can be incorporated for stabilization. In other embodiments, a poly-A tail comprises des-3′ hydroxyl tails. The useful poly-A tails can also include structural moieties or 2′-Omethyl modifications as taught by Junjie Li, et al. (Current Biology, Vol. 15, 1501-1507, Aug. 23, 2005).

In one embodiment, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention (e.g., one or more mRNAs encoding an antibody or an antigen binding portion thereof which specifically binds to CTLA-4) are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

13. Start Codon Region

In some embodiments, the polynucleotide of the present disclosure (e.g., an mRNA encoding an antibody or an antigen binding portion thereof which specifically binds to CTLA-4) further comprises regions that are analogous to or function like a start codon region.

In some embodiments, the translation of a polynucleotide initiates on a codon which is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11). As a non-limiting example, the translation of a polynucleotide begins on the alternative start codon ACG. As another non-limiting example, polynucleotide translation begins on the alternative start codon CTG or CUG. As yet another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11). Masking any of the nucleotides flanking a codon that initiates translation can be used to alter the position of translation initiation, translation efficiency, length and/or structure of a polynucleotide.

In some embodiments, a masking agent is used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11)).

In another embodiment, a masking agent is used to mask a start codon of a polynucleotide in order to increase the likelihood that translation will initiate on an alternative start codon. In some embodiments, a masking agent is used to mask a first start codon or alternative start codon in order to increase the chance that translation will initiate on a start codon or alternative start codon downstream to the masked start codon or alternative start codon.

In some embodiments, a start codon or alternative start codon is located within a perfect complement for a miR binding site. The perfect complement of a miR binding site can help control the translation, length and/or structure of the polynucleotide similar to a masking agent. As a non-limiting example, the start codon or alternative start codon is located in the middle of a perfect complement for a miR binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide is removed from the polynucleotide sequence in order to have the translation of the polynucleotide begin on a codon which is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

14. Stop Codon Region

In some embodiments, a polynucleotide of the present disclosure (e.g., an mRNA encoding an antibody or an antigen binding portion thereof which specifically binds to CTLA-4) can further comprise at least one stop codon or at least two stop codons before the 3′ untranslated region (UTR). The stop codon can be selected from UGA, UAA, and UAG. In some embodiments, the polynucleotides of the present invention include the stop codon UGA and one additional stop codon. In a further embodiment the addition stop codon can be UAA. In another embodiment, the polynucleotides of the present invention include three stop codons, four stop codons, or more.

15. Polynucleotide Comprising One or More mRNAs Encoding an Antibody or an Antigen Binding Portion Thereof Which Specifically Binds to CTLA-4

In certain embodiments, a polynucleotide of the present disclosure (e.g., one or more mRNAs encoding an antibody or an antigen binding portion thereof which specifically binds to CTLA-4) comprises

(i) a 5′ cap provided above;

(ii) a 5′ UTR, such as the sequences provided in TABLE 3 above;

(iii) an open reading frame encoding an antibody or an antigen binding portion thereof which specifically binds to CTLA-4, e.g., a polynucleotide sequence disclosed in TABLE 1 or a polynucleotide sequence encoding any of the protein sequences provided in TABLE 1 above or a combination thereof;

(iv) a stop codon,

(v) a 3′ UTR, such as the sequences provided in TABLE 4 above; and,

(vi) a poly-A tail provided above.

In some embodiments, a polynucleotide of the present disclosure (e.g., a polynucleotide comprising one or more mRNAs encoding an antibody or an antigen binding portion thereof which specifically binds to CTLA-4) encodes a polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96% , at least 97%, at least 98%, at least 99%, or 100% identical to any of the antibody heavy chains (HC) or light chains (LC) of TABLE 1, or to a subsequence thereof comprising, consisting, or consisting essentially of:
(i) one, two or three VH-CDRs;
(ii) one, two or three VL-CDRs;
(iii) a VH;
(iv) a VL;
(v) a HC;
(vi) a LC;
(vii) a fragment thereof; or,
(viii) a combination thereof.

### V. Methods of Making Polynucleotides

The present disclosure also provides methods for making the polynucleotides of the present invention (e.g., one or more mRNAs encoding an antibody or an antigen binding portion thereof which specifically binds to CTLA-4), or a complement thereof.

In some aspects, a polynucleotide (e.g., an mRNA) disclosed herein, and encoding a binding molecule (e.g., an antibody or an antigen-binding portion thereof) which specifically binds to CTLA-4, can be constructed using in vitro transcription.

In other aspects, a polynucleotide (e.g., an mRNA) disclosed herein, and encoding a binding molecule (e.g., an antibody or an antigen-binding portion thereof) which specifically binds to CTLA-4, can be constructed by chemical synthesis using an oligonucleotide synthesizer.

In other aspects, a polynucleotide (e.g., an mRNA) disclosed herein, and encoding a binding molecule (e.g., an antibody or an antigen-binding portion thereof) which specifically binds to CTLA-4, is made by using a host cell.

In certain aspects, a polynucleotide (e.g., an mRNA) disclosed herein, and encoding a binding molecule (e.g., an antibody or an antigen-binding portion thereof) which specifically binds to CTLA-4, is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding binding molecule (e.g., an antibody or an antigen binding portion thereof) which specifically binds to CTLA-4. The resultant mRNAs can then be examined for their ability to produce protein and/or produce a therapeutic outcome.

1. In Vitro Transcription/Enzymatic Synthesis

The polynucleotides of the present invention (e.g., one or more mRNAs encoding an antibody or an antigen binding portion thereof which specifically binds to CTLA-4) can be transcribed using an in vitro transcription (IVT) system. The system typically comprises a transcription buffer, nucleotide triphosphates (NTPs), an RNase inhibitor and a polymerase. The NTPs can be selected from, but are not limited to, those described herein including natural and unnatural (modified) NTPs. The polymerase can be selected from, but is not limited to, T7 RNA polymerase, T3 RNA polymerase and mutant polymerases such as, but not limited to, polymerases able to incorporate modified nucleic acids. See U.S. Publ. No. US20130259923.

The IVT system typically comprises a transcription buffer, nucleotide triphosphates (NTPs), an RNase inhibitor and a polymerase. The NTPs can be selected from, but are not limited to, those described herein including natural and unnatural (modified) NTPs. The polymerase can be selected from, but is not limited to, T7 RNA polymerase, T3 RNA polymerase and mutant polymerases such as, but not limited to, polymerases able to incorporate polynucleotides disclosed herein.

Any number of RNA polymerases or variants can be used in the synthesis of the polynucleotides of the present invention.

RNA polymerases can be modified by inserting or deleting amino acids of the RNA polymerase sequence. As a non-limiting example, the RNA polymerase is modified to exhibit an increased ability to incorporate a 2′-modified nucleotide triphosphate compared to an unmodified RNA polymerase (see International Publication WO2008078180 and U.S. Pat. No. 8,101,385).

Variants can be obtained by evolving an RNA polymerase, optimizing the RNA polymerase amino acid and/or nucleic acid sequence and/or by using other methods known in the art. As a non-limiting example, T7 RNA polymerase variants are evolved using the continuous directed evolution system set out by Esvelt et al. (Nature (2011) 472(7344):499-503) where clones of T7 RNA polymerase can encode at least one mutation such as, but not limited to, lysine at position 93 substituted for threonine (K93T), I4M, A7T, E63V, V64D, A65E, D66Y, T76N, C125R, S128R, A136T, N165S, G175R, H176L, Y178H, F182L, L196F, G198V, D208Y, E222K, S228A, Q239R, T243N, G259D, M267I, G280C, H300R, D351A, A354S, E356D, L360P, A383V, Y385C, D388Y, S397R, M401T, N410S, K450R, P451T, G452V, E484A, H523L, H524N, G542V, E565K, K577E, K577M, N601S, S684Y, L699I, K713E, N748D, Q754R, E775K, A827V, D851N or L864F. As another non-limiting example, T7 RNA polymerase variants can encode at least mutation as described in U.S. Pub. Nos. 20100120024 and 20070117112. Variants of RNA polymerase can also include, but are not limited to, substitutional variants, conservative amino acid substitution, insertional variants, deletional variants and/or covalent derivatives.

In one aspect, the polynucleotide can be designed to be recognized by the wild type or variant RNA polymerases. In doing so, the polynucleotide can be modified to contain sites or regions of sequence changes from the wild type or parent chimeric polynucleotide.

Polynucleotide or nucleic acid synthesis reactions can be carried out by enzymatic methods utilizing polymerases. Polymerases catalyze the creation of phosphodiester bonds between nucleotides in a polynucleotide or nucleic acid chain. Currently known DNA polymerases can be divided into different families based on amino acid sequence comparison and crystal structure analysis. DNA polymerase I (pol I) or A polymerase family, including the Klenow fragments of E. Coli, Bacillus DNA polymerase I, Thermus aquaticus (Taq) DNA polymerases, and the T7 RNA and DNA polymerases, is among the best studied of these families. Another large family is DNA polymerase α (pol α) or B polymerase family, including all eukaryotic replicating DNA polymerases and polymerases from phages T4 and RB69. Although they employ similar catalytic mechanism, these families of polymerases differ in substrate specificity, substrate analog-incorporating efficiency, degree and rate for primer extension, mode of DNA synthesis, exonuclease activity, and sensitivity against inhibitors.

DNA polymerases are also selected based on the optimum reaction conditions they require, such as reaction temperature, pH, and template and primer concentrations. Sometimes a combination of more than one DNA polymerases is employed to achieve the desired DNA fragment size and synthesis efficiency. For example, Cheng et al. increase pH, add glycerol and dimethyl sulfoxide, decrease denaturation times, increase extension times, and utilize a secondary thermostable DNA polymerase that possesses a 3′ to 5′ exonuclease activity to effectively amplify long targets from cloned inserts and human genomic DNA. (Cheng et al., PNAS, Vol. 91, 5695-5699 (1994)). RNA polymerases from bacteriophage T3, T7, and SP6 have been widely used to prepare RNAs for biochemical and biophysical studies. RNA polymerases, capping enzymes, and poly-A polymerases are disclosed in the co-pending International Publication No. WO2014028429.

In one aspect, the RNA polymerase which can be used in the synthesis of the polynucleotides described herein is a Syn5 RNA polymerase (see Zhu et at Nucleic Acids Research 2013). The Syn5 RNA polymerase was recently characterized from marine cyanophage Syn5 by Zhu et at where they also identified the promoter sequence (see Zhu et al. Nucleic Acids Research 2013). Zhu et al. found that Syn5 RNA polymerase catalyzed RNA synthesis over a wider range of temperatures and salinity as compared to T7 RNA polymerase. Additionally, the requirement for the initiating nucleotide at the promoter was found to be less stringent for Syn5 RNA polymerase as compared to the T7 RNA polymerase making Syn5 RNA polymerase promising for RNA synthesis.

In one aspect, a Syn5 RNA polymerase can be used in the synthesis of the polynucleotides described herein. As a non-limiting example, a Syn5 RNA polymerase can be used in the synthesis of the polynucleotide requiring a precise 3′-terminus.

In one aspect, a Syn5 promoter can be used in the synthesis of the polynucleotides. As a non-limiting example, the Syn5 promoter can be 5′-ATTGGGCACCCGTAAGGG-3′ (SEQ ID NO: 233) as described by Zhu et al. (Nucleic Acids Research 2013).

In one aspect, a Syn5 RNA polymerase can be used in the synthesis of polynucleotides comprising at least one chemical modification described herein and/or known in the art. (see e.g., the incorporation of pseudo-UTP and 5Me-CTP described in Zhu et al. Nucleic Acids Research 2013).

In one aspect, the polynucleotides described herein can be synthesized using a Syn5 RNA polymerase which has been purified using modified and improved purification procedure described by Zhu et al. (Nucleic Acids Research 2013).

Various tools in genetic engineering are based on the enzymatic amplification of a target gene which acts as a template. For the study of sequences of individual genes or specific regions of interest and other research needs, it is necessary to generate multiple copies of a target gene from a small sample of polynucleotides or nucleic acids. Such methods can be applied in the manufacture of the polynucleotides of the invention.

Polymerase chain reaction (PCR) has wide applications in rapid amplification of a target gene, as well as genome mapping and sequencing. The key components for synthesizing DNA comprise target DNA molecules as a template, primers complementary to the ends of target DNA strands, deoxynucleoside triphosphates (dNTPs) as building blocks, and a DNA polymerase. As PCR progresses through denaturation, annealing and extension steps, the newly produced DNA molecules can act as a template for the next circle of replication, achieving exponentially amplification of the target DNA. PCR requires a cycle of heating and cooling for denaturation and annealing. Variations of the basic PCR include asymmetric PCR [Innis et al., PNAS, vol. 85, 9436-9440 (1988)], inverse PCR [Ochman et al., Genetics, vol. 120(3), 621-623, (1988)], reverse transcription PCR (RT-PCR) (Freeman et al., BioTechniques, vol. 26(1), 112-22, 124-5 (1999)). In RT-PCR, a single stranded RNA is the desired target and is converted to a double stranded DNA first by reverse transcriptase.

A variety of isothermal in vitro nucleic acid amplification techniques have been developed as alternatives or complements of PCR. For example, strand displacement amplification (SDA) is based on the ability of a restriction enzyme to form a nick. (Walker et al., PNAS, vol. 89, 392-396 (1992), the contents of which are incorporated herein by reference in their entirety)). A restriction enzyme recognition sequence is inserted into an annealed primer sequence. Primers are extended by a DNA polymerase and dNTPs to form a duplex. Only one strand of the duplex is cleaved by the restriction enzyme. Each single strand chain is then available as a template for subsequent synthesis. SDA does not require the complicated temperature control cycle of PCR.

Nucleic acid sequence-based amplification (NASBA), also called transcription mediated amplification (TMA), is also an isothermal amplification method that utilizes a combination of DNA polymerase, reverse transcriptase, RNAse H, and T7 RNA polymerase. [Compton, Nature, vol. 350, 91-92 (1991)]. A target RNA is used as a template and a reverse transcriptase synthesizes its complementary DNA strand. RNAse H hydrolyzes the RNA template, making space for a DNA polymerase to synthesize a DNA strand complementary to the first DNA strand which is complementary to the RNA target, forming a DNA duplex. T7 RNA polymerase continuously generates complementary RNA strands of this DNA duplex. These RNA strands act as templates for new cycles of DNA synthesis, resulting in amplification of the target gene.

Rolling-circle amplification (RCA) amplifies a single stranded circular polynucleotide and involves numerous rounds of isothermal enzymatic synthesis where Φ29 DNA polymerase extends a primer by continuously progressing around the polynucleotide circle to replicate its sequence over and over again. Therefore, a linear copy of the circular template is achieved. A primer can then be annealed to this linear copy and its complementary chain can be synthesized. [See Lizardi et al., Nature Genetics, vol. 19, 225-232 (1998)]. A single stranded circular DNA can also serve as a template for RNA synthesis in the presence of an RNA polymerase. (Daubendiek et al., JACS, vol. 117, 7818-7819 (1995)). An inverse rapid amplification of cDNA ends (RACE) RCA is described by Polidoros et al. A messenger RNA (mRNA) is reverse transcribed into cDNA, followed by RNAse H treatment to separate the cDNA. The cDNA is then circularized by CircLigase into a circular DNA. The amplification of the resulting circular DNA is achieved with RCA. (Polidoros et al., BioTechniques, vol. 41, 35-42 (2006)).

Any of the foregoing methods can be utilized in the manufacture of one or more regions of the polynucleotides of the present invention.

Assembling polynucleotides or nucleic acids by a ligase is also widely used. DNA or RNA ligases promote intermolecular ligation of the 5′ and 3′ ends of polynucleotide chains through the formation of a phosphodiester bond. Ligase chain reaction (LCR) is a promising diagnosing technique based on the principle that two adjacent polynucleotide probes hybridize to one strand of a target gene and couple to each other by a ligase. If a target gene is not present, or if there is a mismatch at the target gene, such as a single-nucleotide polymorphism (SNP), the probes cannot ligase. (Wiedmann et al., PCR Methods and Application, vol.3 (4), s51-s64 (1994)). LCR can be combined with various amplification techniques to increase sensitivity of detection or to increase the amount of products if it is used in synthesizing polynucleotides and nucleic acids.

Several library preparation kits for nucleic acids are now commercially available. They include enzymes and buffers to convert a small amount of nucleic acid samples into an indexed library for downstream applications. For example, DNA fragments can be placed in a NEBNEXT® ULTRA™ DNA Library Prep Kit by NEWENGLAND BIOLABS® for end preparation, ligation, size selection, clean-up, PCR amplification and final clean-up.

Continued development is going on to improvement the amplification techniques. For example, U.S. Pat. No. 8,367,328 to Asada et al., teaches utilizing a reaction enhancer to increase the efficiency of DNA synthesis reactions by DNA polymerases. The reaction enhancer comprises an acidic substance or cationic complexes of an acidic substance. U.S. Pat. No. 7,384,739 to Kitabayashi et al., teaches a carboxylate ion-supplying substance that promotes enzymatic DNA synthesis, wherein the carboxylate ion-supplying substance is selected from oxalic acid, malonic acid, esters of oxalic acid, esters of malonic acid, salts of malonic acid, and esters of maleic acid. U.S. Pat. No. 7,378,262 to Sobek et al., discloses an enzyme composition to increase fidelity of DNA amplifications. The composition comprises one enzyme with 3′ exonuclease activity but no polymerase activity and another enzyme that is a polymerase. Both of the enzymes are thermostable and are reversibly modified to be inactive at lower temperatures.

U.S. Pat. No. 7,550,264 to Getts et al. teaches multiple round of synthesis of sense RNA molecules are performed by attaching oligodeoxynucleotides tails onto the 3′ end of cDNA molecules and initiating RNA transcription using RNA polymerase. US Pat. Publication No. 2013/0183718 to Rohayem teaches RNA synthesis by RNA-dependent RNA polymerases (RdRp) displaying an RNA polymerase activity on single-stranded DNA templates. Oligonucleotides with non-standard nucleotides can be synthesized with enzymatic polymerization by contacting a template comprising non-standard nucleotides with a mixture of nucleotides that are complementary to the nucleotides of the template as disclosed in U.S. Pat. No. 6,617,106 to Benner.

2. Chemical Synthesis

Standard methods can be applied to synthesize an isolated polynucleotide sequence (e.g., an mRNA) encoding an antibody or an antigen binding portion thereof which specifically binds to CTLA-4. For example, a single DNA or RNA oligomer (e.g., an mRNA) containing a codon-optimized nucleotide sequence coding for the particular isolated polypeptide can be synthesized. In other aspects, several small oligonucleotides coding for portions of the desired polypeptide can be synthesized and then ligated. In some aspects, the individual oligonucleotides typically contain 5′ or 3′ overhangs for complementary assembly.

A polynucleotide disclosed herein (e.g., an mRNA) can be chemically synthesized using chemical synthesis methods and potential nucleobase substitutions known in the art. See, for example, International Publication Nos. WO2014093924, WO2013052523; WO2013039857, WO2012135805, WO2013151671; U.S. Publ. No. US20130115272; or U.S. Pat. Nos. 8,999,380, 8,710,200.

3. Purification

Purification of the polynucleotides of the present disclosure (e.g., one or more mRNAs encoding an antibody or an antigen binding portion thereof which specifically binds to CTLA-4) can include, but is not limited to, polynucleotide clean-up, quality assurance and quality control. Clean-up can be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC).

The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide of the present disclosure (e.g., one or more mRNAs encoding an antibody or an antigen binding portion thereof which specifically binds to CTLA-4) removes impurities that can reduce or remove an unwanted immune response, e.g., reducing cytokine activity.

In some embodiments, the polynucleotide of the present disclosure (e.g., one or more mRNAs encoding an antibody or an antigen binding portion thereof which specifically binds to CTLA-4) is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)).

In some embodiments, a polynucleotide of the present disclosure (e.g., one or more mRNAs encoding an antibody or an antigen binding portion thereof which specifically binds to CTLA-4) purified using column chromatography (e.g., strong anion exchange HPLC, weak anion exchange HPLC, reverse phase HPLC (RP-HPLC), hydrophobic interaction HPLC (HIC-HPLC), or (LCMS)) presents increased expression of the encoded antibody or an antigen binding portion thereof which specifically binds to CTLA-4 compared to the expression level of the same polynucleotide of the present disclosure purified by a different purification method.

In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide encodes a binding molecule or a fragment thereof (e.g., an antibody or an antigen binding portion thereof) which specifically binds to CTLA-4.

In some embodiments, the purified polynucleotide encodes a murine binding molecule or a fragment thereof (e.g., an antibody or an antigen binding portion thereof) which specifically binds to CTLA-4.

In some embodiments, the purified polynucleotide encodes a human binding molecule or a fragment thereof (e.g., an antibody or an antigen binding portion thereof) which specifically binds to CTLA-4.

In some embodiments, the purified polynucleotide encodes an antibody or an antigen binding portion thereof which specifically binds to CTLA-4 comprising a polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96% , at least 97%, at least 98%, at least 99%, or 100% identical to any of the antibody heavy chains (HC) or light chains (LC) of TABLE 1, or to a subsequence thereof comprising, consisting, or consisting essentially of:
(i) one, two or three VH-CDRs;
(ii) one, two or three VL-CDRs;
(iii) a VH;
(iv) a VL;
(v) a HC;
(vi) a LC;
(vii) a fragment thereof; or,
(viii) a combination thereof.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC.

In another embodiment, the polynucleotides can be sequenced by methods including, but not limited to reverse-transcriptase-PCR.

VI. mRNA Modifications

As used herein in a polynucleotide of the present disclosure (e.g., one or more mRNAs encoding an antibody or an antigen binding portion thereof which specifically binds to CTLA-4), the terms “chemical modification” or, as appropriate, “chemically modified” refer to modification with respect to adenosine (A), guanosine (G), uridine (U), thymidine (T) or cytidine (C) ribo- or deoxyribnucleosides in one or more of their position, pattern, percent or population. Generally, herein, these terms are not intended to refer to the ribonucleotide modifications in naturally occurring 5′-terminal mRNA cap moieties.

In a polypeptide, the term “modification” refers to a modification as compared to the canonical set of 20 amino acids.

The modifications can be various distinct modifications. In some embodiments, the regions can contain one, two, or more (optionally different) nucleoside or nucleotide (nucleobase) modifications. In some embodiments, a modified polynucleotide, introduced to a cell can exhibit reduced degradation in the cell, as compared to an unmodified polynucleotide. In other embodiments, the modification is in the nucleobase and/or the sugar structure. In yet other embodiments, the modification is in the backbone structure.

1. Chemical Modifications

Some embodiments of the present disclosure provide one or more mRNAs encoding an antibody or an antigen binding portion thereof which specifically binds to CTLA-4), wherein the one or more mRNAs include at least one chemical modification.

In some embodiments, the chemical modification is selected from pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine, and 2′-O-methyl uridine.

A “nucleoside” as used herein refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” as used herein refers to a nucleoside, including a phosphate group. Modified nucleotides can be synthesized by any useful method, such as, for example, chemically, enzymatically, or recombinantly, to include one or more modified or non-natural nucleosides. Polynucleotides can comprise a region or regions of linked nucleosides. Such regions can have variable backbone linkages. The linkages can be standard phosphdioester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s.

Modifications of the polynucleotide of the present disclosure (e.g., one or more mRNAs encoding an antibody or an antigen binding portion thereof which specifically binds to CTLA-4) include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methyl cytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methyl pseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine,), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouri dine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine;1-(aza)-2-(thio)-3-(aza)-phenoxazin-1-yl; 1,3-(diaza)-2-(oxo)-phenthiazin-1-yl;1,3-(diaza)-2-(oxo)-phenoxazin-1-yl; 1,3,5-(triaza)-2,6-(dioxa)-naphthalene; 2 (amino)purine;2,4,5-(trimethyl)phenyl; 2′ methyl, 2′amino, 2′azido, 2′fluro-cytidine;2′ methyl, 2′amino, 2′azido, 2′fluro-adenine;2′methyl, 2′amino, 2′azido, 2′fluro-uridine;2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazin-1-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; O6-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, the polynucleotide of the present disclosure (e.g., one or more mRNAs encoding an antibody or an antigen binding portion thereof which specifically binds to CTLA-4) includes a combination of at least two (e.g., 2, 3, 4 or more) of the aforementioned modified nucleobases.

In some embodiments, modified nucleobases in the polynucleotide of the present disclosure (e.g., one or more mRNAs encoding an antibody or an antigen binding portion thereof which specifically binds to CTLA-4) are selected from the group consisting of pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine and 2′-O-methyl uridine. In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, modified nucleobases in the polynucleotide of the present disclosure (e.g., one or more mRNAs encoding an antibody or an antigen binding portion thereof which specifically binds to CTLA-4) are selected from the group consisting of 1-methyl-pseudouridine (m1ψ), 5-methoxy-uridine (mo5U), 5-methyl-cytidine (m5C), pseudouridine (ψ), α-thio-guanosine and α-thio-adenosine. In some embodiments, the polynucleotide includes a combination of at least two (e.g., 2, 3, 4 or more) of the aforementioned modified nucleobases.

In some embodiments, the polynucleotide of the present disclosure (e.g., one or more mRNAs encoding an antibody or an antigen binding portion thereof which specifically binds to CTLA-4) comprises pseudouridine (ψ) and 5-methyl-cytidine (m5C). In some embodiments, the polynucleotide of the present disclosure (e.g., one or more mRNAs encoding an antibody or an antigen binding portion thereof which specifically binds to CTLA-4) comprises 1-methyl-pseudouridine (m1ψ).

In some embodiments, the polynucleotide of the present disclosure (e.g., one or more mRNAs encoding an antibody or an antigen binding portion thereof which specifically binds to CTLA-4) comprises 1-methyl-pseudouridine (m1ψ) and 5-methyl-cytidine (m5C). In some embodiments, the polynucleotide of the present disclosure (e.g., one or more mRNAs encoding an antibody or an antigen binding portion thereof which specifically binds to CTLA-4) comprises 2-thiouridine (s2U).

In some embodiments, the polynucleotide of the present disclosure (e.g., one or more mRNAs encoding an antibody or an antigen binding portion thereof which specifically binds to CTLA-4) comprises 2-thiouridine and 5-methyl-cytidine (m5C). In some embodiments, the polynucleotide of the present disclosure (e.g., one or more mRNAs encoding an antibody or an antigen binding portion thereof which specifically binds to CTLA-4) comprises methoxy-uridine (mo5U).

In some embodiments, the polynucleotide of the present disclosure (e.g., one or more mRNAs encoding an antibody or an antigen binding portion thereof which specifically binds to CTLA-4) comprises 5-methoxy-uridine (mo5U) and 5-methyl-cytidine (m5C). In some embodiments, the polynucleotide of the present disclosure (e.g., one or more mRNAs encoding an antibody or an antigen binding portion thereof which specifically binds to CTLA-4) comprises 2′-O-methyl uridine.

In some embodiments, the polynucleotide of the present disclosure (e.g., one or more mRNAs encoding an antibody or an antigen binding portion thereof which specifically binds to CTLA-4) comprises 2′-O-methyl uridine and 5-methyl-cytidine (m5C). In some embodiments, the polynucleotide of the present disclosure (e.g., one or more mRNAs encoding an antibody or an antigen binding portion thereof which specifically binds to CTLA-4) comprises N6-methyl-adenosine (m6A). In some embodiments, the polynucleotide of the present disclosure (e.g., one or more mRNAs encoding an antibody or an antigen binding portion thereof which specifically binds to CTLA-4) comprises N6-methyl-adenosine (m6A) and 5-methyl-cytidine (m5C).

In some embodiments, the polynucleotides of the present disclosure (e.g., one or more mRNAs encoding an antibody or an antigen binding portion thereof which specifically binds to CTLA-4) are uniformly modified (e.g., fully modified, modified throughout the entire sequence) for a particular modification. For example, a polynucleotide of the present disclosure can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above.

In some embodiments, the modified nucleobase is a modified cytosine. Examples of nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine.

In some embodiments, a modified nucleobase is a modified uridine. Example nucleobases and nucleosides having a modified uridine include 5-cyano uridine or 4′-thio uridine.

In some embodiments, a modified nucleobase is a modified adenine. Example nucleobases and nucleosides having a modified adenine include 7-deaza-adenine, 1-methyl-adenosine (m1A), 2-methyl-adenine (m2A), N6-methyl-adenosine (m6A), and 2,6-Diaminopurine.

In some embodiments, a modified nucleobase is a modified guanine. Example nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, or 7-methyl-8-oxo-guanosine.

Other modifications which can be useful in the polynucleotides of the present disclosure (e.g., one or more mRNAs encoding an antibody or an antigen binding portion thereof which specifically binds to CTLA-4) are listed in TABLE 5.

TABLE 5
Additional Modification types

| Name | Type |
|---|---|
| 2,6-(diamino)purine | Other |
| 1-(aza)-2-(thio)-3-(aza)-phenoxazin-1-yl | Other |
| 1,3-(diaza)-2-(oxo)-phenthiazin-l-yl | Other |
| 1,3-(diaza)-2-(oxo)-phenoxazin-1-yl | Other |
| 1,3,5-(triaza)-2,6-(dioxa)-naphthalene | Other |
| 2 (amino)purine | Other |
| 2,4,5-(trimethyl)phenyl | Other |
| 2′ methyl, 2′amino, 2′azido, 2′fluro-cytidine | Other |
| 2′ methyl, 2′amino, 2′azido, 2′fluro-adenine | Other |
| 2′methyl, 2′amino, 2′azido, 2′fluro-uridine | Other |
| 2′-amino-2′-deoxyribose | Other |
| 2-amino-6-Chloro-purine | Other |
| 2-aza-inosinyl | Other |
| 2′-azido-2′-deoxyribose | Other |
| 2′fluoro-2′-deoxyribose | Other |
| 2′-fluoro-modified bases | Other |
| 2′-O-methyl-ribose | Other |
| 2-oxo-7-aminopyridopyrimidin-3-yl | Other |
| 2-oxo-pyridopyrimidine-3-yl | Other |
| 2-pyridinone | Other |
| 3 nitropyrrole | Other |
| 3-(methyl)-7-(propynyl)isocarbostyrilyl | Other |
| 3-(methyl)isocarbostyrilyl | Other |
| 4-(fluoro)-6-(methyl)benzimidazole | Other |
| 4-(methyl)benzimidazole | Other |
| 4-(methyl)indolyl | Other |
| 4,6-(dimethyl)indolyl | Other |
| 5 nitroindole | Other |
| 5 substituted pyrimidines | Other |
| 5-(methyl)isocarbostyrilyl | Other |
| 5-nitroindole | Other |
| 6-(aza)pyrimidine | Other |
| 6-(azo)thymine | Other |
| 6-(methyl)-7-(aza)indolyl | Other |
| 6-chloro-purine | Other |
| 6-phenyl-pyrrolo-pyrimidin-2-on-3-yl | Other |
| 7-(aminoalkylhydroxy)-1-(aza)-2-(thio)-3-(aza)-phenthiazin-l-yl | Other |
| 7-(aminoalkylhydroxy)-1-(aza)-2-(thio)-3-(aza)-phenoxazin-1-yl | Other |
| 7-(aminoalkylhydroxy)-1,3-(diaza)-2-(oxo)-phenoxazin-1-yl | Other |
| 7-(aminoalkylhydroxy)-l,3-(diaza)-2-(oxo)-phenthiazin-l-yl | Other |
| 7-(aminoalkylhydroxy)-l,3-(diaza)-2-(oxo)-phenoxazin-l-yl | Other |
| 7-(aza)indolyl | Other |
| 7-(guanidiniumalkylhydroxy)-1-(aza)-2-(thio)-3-(aza)-phenoxazinl-yl | Other |
| 7-(guanidiniumalkylhydroxy)-1-(aza)-2-(thio)-3-(aza)-phenthiazin-l-yl | Other |
| 7-(guanidiniumalkylhydroxy)-1-(aza)-2-(thio)-3-(aza)-phenoxazin-1-yl | Other |
| 7-(guanidiniumalkylhydroxy)-1,3-(diaza)-2-(oxo)-phenoxazin-1-yl | Other |
| 7-(guanidiniumalkyl-hydroxy)-l,3-(diaza)-2-(oxo)-phenthiazin-l-yl | Other |
| 7-(guanidiniumalkylhydroxy)-l,3-(diaza)-2-(oxo)-phenoxazin-l-yl | Other |
| 7-(propynyl)isocarbostyrilyl | Other |
| 7-(propynyl)isocarbostyrilyl, propynyl-7-(aza)indolyl | Other |
| 7-deaza-inosinyl | Other |
| 7-substituted 1-(aza)-2-(thio)-3-(aza)-phenoxazin-1-yl | Other |
| 7-substituted 1,3-(diaza)-2-(oxo)-phenoxazin-1-yl | Other |
| 9-(methyl)-imidizopyridinyl | Other |
| Aminoindolyl | Other |
| Anthracenyl | Other |
| bis-ortho-(aminoalkylhydroxy)-6-phenyl-pyrrolo-pyrimidin-2-on-3-yl | Other |
| bis-ortho-substituted-6-phenyl-pyrrolo-pyrimidin-2-on-3-yl | Other |
| Difluorotolyl | Other |
| Hypoxanthine | Other |
| Imidizopyridinyl | Other |
| Inosinyl | Other |
| Isocarbostyrilyl | Other |
| Isoguanisine | Other |
| N2-substituted purines | Other |
| N6-methyl-2-amino-purine | Other |
| N6-substituted purines | Other |
| N-alkylated derivative | Other |
| Napthalenyl | Other |
| Nitrobenzimidazolyl | Other |
| Nitroimidazolyl | Other |
| Nitroindazolyl | Other |
| Nitropyrazolyl | Other |
| Nubularine | Other |
| O6-substituted purines | Other |
| O-alkylated derivative | Other |
| ortho-(aminoalkylhydroxy)-6-phenyl-pyrrolo-pyrimidin-2-on-3-yl | Other |
| ortho-substituted-6-phenyl-pyrrolo-pyrimidin-2-on-3-yl | Other |
| Oxoformycin TP | Other |
| para-(aminoalkylhydroxy)-6-phenyl-pyrrolo-pyrimidin-2-on-3-yl | Other |
| para-substituted-6-phenyl-pyrrolo-pyrimidin-2-on-3-yl | Other |
| Pentacenyl | Other |
| Phenanthracenyl | Other |
| Phenyl | Other |
| propynyl-7-(aza)indolyl | Other |
| Pyrenyl | Other |
| pyridopyrimidin-3-yl | Other |
| pyridopyrimidin-3-yl, 2-oxo-7-amino-pyridopyrimidin-3-yl | Other |
| pyrrolo-pyrimidin-2-on-3-yl | Other |
| Pyrrolopyrimidinyl | Other |
| Pyrrolopyrizinyl | Other |
| Stilbenzyl | Other |
| substituted 1,2,4-triazoles | Other |
| Tetracenyl | Other |
| Tubercidine | Other |
| Xanthine | Other |
| Xanthosine-5′-TP | Other |
| 2-thio-zebularine | Other |
| 5-aza-2-thio-zebularine | Other |
| 7-deaza-2-amino-purine | Other |
| pyridin-4-one ribonucleoside | Other |
| 2-Amino-riboside-TP | Other |
| Formycin A TP | Other |
| Formycin B TP | Other |
| Pyrrolosine TP | Other |
| 2′-OH-ara-adenosine TP | Other |
| 2′-OH-ara-cytidine TP | Other |
| 2′-OH-ara-uridine TP | Other |
| 2′-OH-ara-guanosine TP | Other |
| 5-(2-carbomethoxyvinyl)uridine TP | Other |
| N6-(19-Amino-pentaoxanonadecyl)adenosine TP | Other |

The polynucleotides of the present disclosure (e.g., one or more mRNAs encoding an antibody or an antigen binding portion thereof which specifically binds to CTLA-4) can include any useful linker between the nucleosides. Such linkers, including backbone modifications are given in TABLE 6.

TABLE 6
Linker modifications

| Name | TYPE |
|---|---|
| 3′-alkylene phosphonates | Linker |
| 3′-amino phosphoramidate | Linker |
| alkene containing backbones | Linker |
| Aminoalkylphosphoramidates | Linker |
| Aminoalkylphosphotriesters | Linker |
| Boranophosphates | Linker |
| —CH2-0-N(CH3)—CH2— | Linker |
| —CH2—N(CH3)—N(CH3)—CH2— | Linker |
| —CH2—NH—CH2— | Linker |
| chiral phosphonates | Linker |
| chiral phosphorothioates | Linker |
| formacetyl and thioformacetyl backbones | Linker |
| methylene (methylimino) | Linker |
| methylene formacetyl and thioformacetyl backbones | Linker |
| methyleneimino and methylenehydrazino backbones | Linker |
| morpholino linkages | Linker |
| —N(CH3)—CH2—CH2— | Linker |
| oligonucleosides with heteroatom internucleoside linkage | Linker |
| Phosphinates | Linker |
| phosphoramidates | Linker |
| Phosphorodithioates | Linker |
| phosphorothioate internucleoside linkages | Linker |
| Phosphorothioates | Linker |
| Phosphotriesters | Linker |
| PNA | Linker |
| siloxane backbones | Linker |
| sulfamate backbones | Linker |
| sulfide sulfoxide and sulfone backbones | Linker |
| sulfonate and sulfonamide backbones | Linker |
| Thionoalkylphosphonates | Linker |
| Thionoalkylphosphotriesters | Linker |
| Thionophosphoramidates | Linker |

The polynucleotides of the present disclosure (e.g., one or more mRNAs encoding an antibody or an antigen binding portion thereof which specifically binds to CTLA-4) can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase can be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention can be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs), hexitol nucleic acids (HNAs), or hybrids thereof. Additional modifications are described herein. Modified nucleic acids and their synthesis are disclosed in co-pending International Patent Publication No. WO2013052523.

Any of the regions of a polynucleotide of the present disclosure (e.g., one or more mRNAs encoding an antibody or an antigen binding portion thereof which specifically binds to CTLA-4) can be chemically modified as taught herein or as taught in International Application Publication Number WO2013/052523.

In some embodiments, a modified polynucleotide, e.g., mRNA comprising at least one modification described herein, of the invention encodes a binding molecule or antigen-binding portion thereof which specifically binds to CTLA-4 (e.g., an antibody or an antigen binding portion thereof). In some embodiments, a modified polynucleotide, e.g., mRNA comprising at least one modification described herein, of the invention encodes a binding molecule or antigen-binding portion thereof which specifically binds to human CTLA-4 (e.g., an antibody or an antigen binding portion thereof).

In some embodiments, the modified polynucleotide, e.g., mRNA comprising at least one modification described herein, of the invention encodes a binding molecule or antigen-binding portion thereof which specifically binds to CTLA-4 (e.g., an antibody or an antigen binding portion thereof) comprising a polynucleotide sequence at least 60%, at least 70%, at least 80%, at least 85%, at least 90%, at least 95%, at least 96%, at least 97%, at least 98%, at least 99%, or 100% identical to any of the antibody heavy chain (HC) or light chain (LC) of TABLE 1, or to a subsequence thereof which encodes:
(i) one, two or three VH-CDRs;
(ii) one, two or three VL-CDR.s;
(iii) a VH;
(iv) a VL;
(v) a HC;
(vi) a LC;
(vii) a fragment thereof; or,
(viii) a combination thereof.

In some embodiments, the modified polynucleotide, e.g., mRNA comprising at least one modification described herein, encodes at least one binding molecule or antigen-binding portion thereof (e.g., an antibody or an antigen binding portion thereof) which specifically binds to CTLA-4.

In some embodiments, the modified polynucleotide of the invention, e.g., one or more mRNAs encoding an antibody or an antigen binding portion thereof which specifically binds to CTLA-4 comprising at least one modification described herein, is selected from the nucleic acid sequences listed in TABLE 1 and nucleic acid sequences encoding the protein sequences listed in TABLE 1.

The polynucleotide of the present invention (e.g., one or more mRNAs encoding an antibody or an antigen binding portion thereof which specifically binds to CTLA-4) can also include building blocks, e.g., modified ribonucleosides, and modified ribonucleotides, of polynucleotide molecules. For example, these building blocks can be useful for preparing the polynucleotides of the invention. Such building blocks are taught in International Patent Publication No. WO2013052523 and International Application Publication No. WO 2014/093924 A1.

2. Modifications on the Sugar

The modified nucleosides and nucleotides (e.g., building block molecules), which can be incorporated into a polynucleotide of the present invention (e.g., one or more mRNAs encoding an antibody or an antigen binding portion thereof which specifically binds to CTLA-4), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclic); and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are taught international Patent Publication No. WO2013052523 and International Patent Application No. PCT/US2013/75177.

3. Combinations of Modified Sugars, Nucleobases, and Internucleoside Linkages

The polynucleotides of the present invention (e.g., one or more mRNAs encoding an antibody or an antigen binding portion thereof which specifically binds to CTLA-4) can include a combination of modifications to the sugar, the nucleobase, and/or the internucleoside linkage. These combinations can include any one or more modifications described herein.

Examples of modified nucleotides and modified nucleotide combinations are provided below in TABLE 7. These combinations of modified nucleotides can be used to form the polynucleotides of the invention. Unless otherwise noted, the modified nucleotides can be completely substituted for the natural nucleotides of the polynucleotides of the invention (e.g., one or more mRNAs encoding an antibody or an antigen binding portion thereof which specifically binds to CTLA-4). As a non-limiting example, the natural nucleotide uridine can be substituted with a modified nucleoside described herein. In another non-limiting example, the natural nucleotide uridine can be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9%) with at least one of the modified nucleoside disclosed herein. Any combination of base/sugar or linker can be incorporated into the polynucleotides of the invention (e.g., one or more mRNAs encoding an antibody or an antigen binding portion thereof which specifically binds to CTLA-4) and such modifications are taught in International Patent Publication Nos. WO 2013/052523 and WO 2014/093924 A1.

TABLE 7
Combinations

| MODIFIED | MODIFIED NUCLEOTIDE |
|---|---|
| NUCLEOTIDE | COMBINATION |
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are |
| | N1-methyl-pseudouridine and about 50% of |
| | uridines are pseudouridine |
| | pseudoisocytidine/about 25% of uridines are |
| | N1-methyl-pseudouridine and about 25% of |
| | uridines are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines |
| | are 2-thio-uridine |
| | about 50% of uridines are 5-methyl-cytidine/ |
| | about 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/ |
| | about 50% of uridines are 2-thio-uridine |

Additional examples of modified nucleotides and modified nucleotide combinations are provided below in TABLE 8.

TABLE 8
Additional combinations

| URACIL | CYTOSINE | ADENINE | GUANINE |
|---|---|---|---|
| 5-methoxy-UTP | CTP | ATP | GTP |
| 5-Methoxy-UTP | N4Ac-CTP | ATP | GTP |
| 5-Methoxy-UTP | 5-Methyl-CTP | ATP | GTP |
| 5-Methoxy-UTP | 5-Trifluoromethyl-CTP | ATP | GTP |
| 5-Methoxy-UTP | 5-Hydroxymethyl-CTP | ATP | GTP |
| 5-Methoxy-UTP | 5-Bromo-CTP | ATP | GTP |
| 5-Methoxy-UTP | N4Ac-CTP | ATP | GTP |
| 5-Methoxy-UTP | CTP | ATP | GTP |
| 5-Methoxy-UTP | 5-Methyl-CTP | ATP | GTP |
| 5-Methoxy-UTP | 5-Trifluoromethyl-CTP | ATP | GTP |
| 5-Methoxy-UTP | 5-Hydroxymethyl-CTP | ATP | GTP |
| 5-Methoxy-UTP | 5-Bromo-CTP | ATP | GTP |
| 5-Methoxy-UTP | N4—Ac-CTP | ATP | GTP |
| 5-Methoxy-UTP | 5-Iodo-CTP | ATP | GTP |
| 5-Methoxy-UTP | 5-Bromo-CTP | ATP | GTP |
| 5-Methoxy-UTP | CTP | ATP | GTP |
| 5-Methoxy-UTP | 5-Methyl-CTP | ATP | GTP |
| 75% 5-Methoxy-UTP + 25% | 5-Methyl-CTP | ATP | GTP |
| UTP | | | |
| 50% 5-Methoxy-UTP + 50% | 5-Methyl-CTP | ATP | GTP |
| UTP | | | |
| 25% 5-Methoxy-UTP + 75% | 5-Methyl-CTP | ATP | GTP |
| UTP | | | |
| 5-Methoxy-UTP | 75% 5-Methyl-CTP + 25% CTP | ATP | GTP |
| 5-Methoxy-UTP | 50% 5-Methyl-CTP + 50% CTP | ATP | GTP |
| 5-Methoxy-UTP | 25% 5-Methyl-CTP + 75% CTP | ATP | GTP |
| 75% 5-Methoxy-UTP + 25% | 75% 5-Methyl-CTP + 25% CTP | ATP | GTP |
| UTP | | | |
| 75% 5-Methoxy-UTP + 25% | 50% 5-Methyl-CTP + 50% CTP | ATP | GTP |
| UTP | | | |
| 75% 5-Methoxy-UTP + 25% | 25% 5-Methyl-CTP + 75% CTP | ATP | GTP |
| UTP | | | |
| 50% 5-Methoxy-UTP + 50% | 75% 5-Methyl-CTP + 25% CTP | ATP | GTP |
| UTP | | | |
| 50% 5-Methoxy-UTP + 50% | 50% 5-Methyl-CTP + 50% CTP | ATP | GTP |
| UTP | | | |
| 50% 5-Methoxy-UTP + 50% | 25% 5-Methyl-CTP + 75% CTP | ATP | GTP |
| UTP | | | |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | CTP | ATP | GTP |
| UTP | | | |
| 50% 5-Methoxy-UTP + 50% | CTP | ATP | GTP |
| UTP | | | |
| 25% 5-Methoxy-UTP + 75% | CTP | ATP | GTP |
| UTP | | | |
| 5-Methoxy-UTP | CTP | ATP | GTP |
| 5-Methoxy-UTP | CTP | ATP | GTP |
| 5-Methoxy-UTP | CTP | ATP | GTP |
| 5-Methoxy-UTP | 5-Methyl-CTP | ATP | GTP |
| 5-Methoxy-UTP | 5-Methyl-CTP | ATP | GTP |
| 5-Methoxy-UTP | 5-Methyl-CTP | ATP | GTP |
| 5-Methoxy-UTP | CTP | Alpha-thio- | GTP |
| | | ATP | |
| 5-Methoxy-UTP | 5-Methyl-CTP | Alpha-thio- | GTP |
| | | ATP | |
| 5-Methoxy-UTP | CTP | ATP | Alpha- |
| | | | thio-GTP |
| 5-Methoxy-UTP | 5-Methyl-CTP | ATP | Alpha- |
| | | | thio-GTP |
| 5-Methoxy-UTP | CTP | N6-Me- | GTP |
| | | ATP | |
| 5-Methoxy-UTP | 5-Methyl-CTP | N6-Me- | GTP |
| | | ATP | |
| 5-Methoxy-UTP | CTP | ATP | GTP |
| 5-Methoxy-UTP | 5-Methyl-CTP | ATP | GTP |
| 75% 5-Methoxy-UTP + 25% | 5-Methyl-CTP | ATP | GTP |
| UTP | | | |
| 50% 5-Methoxy-UTP + 50% | 5-Methyl-CTP | ATP | GTP |
| UTP | | | |
| 25% 5-Methoxy-UTP + 75% | 5-Methyl-CTP | ATP | GTP |
| UTP | | | |
| 5-Methoxy-UTP | 75% 5-Methyl-CTP + 25% CTP | ATP | GTP |
| 5-Methoxy-UTP | 50% 5-Methyl-CTP + 50% CTP | ATP | GTP |
| 5-Methoxy-UTP | 25% 5-Methyl-CTP + 75% CTP | ATP | GTP |
| 75% 5-Methoxy-UTP + 25% | 75% 5-Methyl-CTP + 25% CTP | ATP | GTP |
| UTP | | | |
| 75% 5-Methoxy-UTP + 25% | 50% 5-Methyl-CTP + 50% CTP | ATP | GTP |
| UTP | | | |
| 75% 5-Methoxy-UTP + 25% | 25% 5-Methyl-CTP + 75% CTP | ATP | GTP |
| UTP | | | |
| 50% 5-Methoxy-UTP + 50% | 75% 5-Methyl-CTP + 25% CTP | ATP | GTP |
| UTP | | | |
| 50% 5-Methoxy-UTP + 50% | 50% 5-Methyl-CTP + 50% CTP | ATP | GTP |
| UTP | | | |
| 50% 5-Methoxy-UTP + 50% | 25% 5-Methyl-CTP + 75% CTP | ATP | GTP |
| UTP | | | |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | CTP | ATP | GTP |
| UTP | | | |
| 50% 5-Methoxy-UTP + 50% | CTP | ATP | GTP |
| UTP | | | |
| 25% 5-Methoxy-UTP + 75% | CTP | ATP | GTP |
| UTP | | | |
| 5-Methoxy-UTP | 5-Ethyl-CTP | ATP | GTP |
| 5-Methoxy-UTP | 5-Methoxy-CTP | ATP | GTP |
| 5-Methoxy-UTP | 5-Ethynyl-CTP | ATP | GTP |
| 5-Methoxy-UTP | CTP | ATP | GTP |
| 5-Methoxy-UTP | 5-Methyl-CTP | ATP | GTP |
| 5-Methoxy-UTP | CTP | ATP | GTP |
| 5-Methoxy-UTP | 5-Methyl-CTP | ATP | GTP |
| 75% 5-Methoxy-UTP + 25% 1- | 5-Methyl-CTP | ATP | GTP |
| Methyl-pseudo-UTP | | | |
| 50% 5-Methoxy-UTP + 50% 1- | 5-Methyl-CTP | ATP | GTP |
| Methyl-pseudo-UTP | | | |
| 25% 5-Methoxy-UTP + 75% 1- | 5-Methyl-CTP | ATP | GTP |
| Methyl-pseudo-UTP | | | |
| 5-Methoxy-UTP | 75% 5-Methyl-CTP + 25% CTP | ATP | GTP |
| 5-Methoxy-UTP | 50% 5-Methyl-CTP + 50% CTP | ATP | GTP |
| 5-Methoxy-UTP | 25% 5-Methyl-CTP + 75% CTP | ATP | GTP |
| 75% 5-Methoxy-UTP + 25% 1- | 75% 5-Methyl-CTP + 25% CTP | ATP | GTP |
| Methyl-pseudo-UTP | | | |
| 75% 5-Methoxy-UTP + 25% 1- | 50% 5-Methyl-CTP + 50% CTP | ATP | GTP |
| Methyl-pseudo-UTP | | | |
| 75% 5-Methoxy-UTP + 25% 1- | 25% 5-Methyl-CTP + 75% CTP | ATP | GTP |
| Methyl-pseudo-UTP | | | |
| 50% 5-Methoxy-UTP + 50% 1- | 75% 5-Methyl-CTP + 25% CTP | ATP | GTP |
| Methyl-pseudo-UTP | | | |
| 50% 5-Methoxy-UTP + 50% 1- | 50% 5-Methyl-CTP + 50% CTP | ATP | GTP |
| Methyl-pseudo-UTP | | | |
| 50% 5-Methoxy-UTP + 50% 1- | 25% 5-Methyl-CTP + 75% CTP | ATP | GTP |
| Methyl-pseudo-UTP | | | |
| 25% 5-Methoxy-UTP + 75% 1- | 75% 5-Methyl-CTP + 25% CTP | ATP | GTP |
| Methyl-pseudo-UTP | | | |
| 25% 5-Methoxy-UTP + 75% 1- | 50% 5-Methyl-CTP + 50% CTP | ATP | GTP |
| Methyl-pseudo-UTP | | | |
| 25% 5-Methoxy-UTP + 75% 1- | 25% 5-Methyl-CTP + 75% CTP | ATP | GTP |
| Methyl-pseudo-UTP | | | |
| 75% 5-Methoxy-UTP + 25% 1- | CTP | ATP | GTP |
| Methyl-pseudo-UTP | | | |
| 50% 5-Methoxy-UTP + 50% 1- | CTP | ATP | GTP |
| Methyl-pseudo-UTP | | | |
| 25% 5-Methoxy-UTP + 75% 1- | CTP | ATP | GTP |
| Methyl-pseudo-UTP | | | |
| 5-methoxy-UTP (In House) | CTP | ATP | GTP |
| 5-methoxy-UTP (Hongene) | CTP | ATP | GTP |
| 5-methoxy-UTP (Hongene) | 5-Methyl-CTP | ATP | GTP |
| 5-Methoxy-UTP | CTP | ATP | GTP |
| 5-Methoxy-UTP | 5-Methyl-CTP | ATP | GTP |
| 75% 5-Methoxy-UTP + 25% | 5-Methyl-CTP | ATP | GTP |
| UTP | | | |
| 50% 5-Methoxy-UTP + 50% | 5-Methyl-CTP | ATP | GTP |
| UTP | | | |
| 25% 5-Methoxy-UTP + 75% | 5-Methyl-CTP | ATP | GTP |
| UTP | | | |
| 5-Methoxy-UTP | 75% 5-Methyl-CTP + 25% CTP | ATP | GTP |
| 5-Methoxy-UTP | 50% 5-Methyl-CTP + 50% CTP | ATP | GTP |
| 5-Methoxy-UTP | 25% 5-Methyl-CTP + 75% CTP | ATP | GTP |
| 75% 5-Methoxy-UTP + 25% | 75% 5-Methyl-CTP + 25% CTP | ATP | GTP |
| UTP | | | |
| 75% 5-Methoxy-UTP + 25% | 50% 5-Methyl-CTP + 50% CTP | ATP | GTP |
| UTP | | | |
| 75% 5-Methoxy-UTP + 25% | 25% 5-Methyl-CTP + 75% CTP | ATP | GTP |
| UTP | | | |
| 50% 5-Methoxy-UTP + 50% | 75% 5-Methyl-CTP + 25% CTP | ATP | GTP |
| UTP | | | |
| 50% 5-Methoxy-UTP + 50% | 50% 5-Methyl-CTP + 50% CTP | ATP | GTP |
| UTP | | | |
| 50% 5-Methoxy-UTP + 50% | 25% 5-Methyl-CTP + 75% CTP | ATP | GTP |
| UTP | | | |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | CTP | ATP | GTP |
| UTP | | | |
| 50% 5-Methoxy-UTP + 50% | CTP | ATP | GTP |
| UTP | | | |
| 25% 5-Methoxy-UTP + 75% | CTP | ATP | GTP |
| UTP | | | |
| 5-Methoxy-UTP | CTP | ATP | GTP |
| 5-Methoxy-UTP | 5-Methyl-CTP | ATP | GTP |
| 75% 5-Methoxy-UTP + 25% | 5-Methyl-CTP | ATP | GTP |
| UTP | | | |
| 50% 5-Methoxy-UTP + 50% | 5-Methyl-CTP | ATP | GTP |
| UTP | | | |
| 25% 5-Methoxy-UTP + 75% | 5-Methyl-CTP | ATP | GTP |
| UTP | | | |
| 5-Methoxy-UTP | 75% 5-Methyl-CTP + 25% CTP | ATP | GTP |
| 5-Methoxy-UTP | 50% 5-Methyl-CTP + 50% CTP | ATP | GTP |
| 5-Methoxy-UTP | 25% 5-Methyl-CTP + 75% CTP | ATP | GTP |
| 75% 5-Methoxy-UTP + 25% | 75% 5-Methyl-CTP + 25% CTP | ATP | GTP |
| UTP | | | |
| 75% 5-Methoxy-UTP + 25% | 50% 5-Methyl-CTP + 50% CTP | ATP | GTP |
| UTP | | | |
| 75% 5-Methoxy-UTP + 25% | 25% 5-Methyl-CTP + 75% CTP | ATP | GTP |
| UTP | | | |
| 50% 5-Methoxy-UTP + 50% | 75% 5-Methyl-CTP + 25% CTP | ATP | GTP |
| UTP | | | |
| 50% 5-Methoxy-UTP + 50% | 50% 5-Methyl-CTP + 50% CTP | ATP | GTP |
| UTP | | | |
| 50% 5-Methoxy-UTP + 50% | 25% 5-Methyl-CTP + 75% CTP | ATP | GTP |
| UTP | | | |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | CTP | ATP | GTP |
| UTP | | | |
| 50% 5-Methoxy-UTP + 50% | CTP | ATP | GTP |
| UTP | | | |
| 25% 5-Methoxy-UTP + 75% | CTP | ATP | GTP |
| UTP | | | |
| 5-Methoxy-UTP | CTP | ATP | GTP |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | 75% 5-Methyl-CTP + 25% CTP | ATP | GTP |
| UTP | | | |
| 5-Methoxy-UTP | CTP | ATP | GTP |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | 75% 5-Methyl-CTP + 25% CTP | ATP | GTP |
| UTP | | | |
| 5-Methoxy-UTP | CTP | ATP | GTP |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | 75% 5-Methyl-CTP + 25% CTP | ATP | GTP |
| UTP | | | |
| 5-Methoxy-UTP | CTP | ATP | GTP |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | 75% 5-Methyl-CTP + 25% CTP | ATP | GTP |
| UTP | | | |
| 5-Methoxy-UTP | CTP | ATP | GTP |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | 75% 5-Methyl-CTP + 25% CTP | ATP | GTP |
| UTP | | | |
| 5-Methoxy-UTP | CTP | ATP | GTP |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | 75% 5-Methyl-CTP + 25% CTP | ATP | GTP |
| UTP | | | |
| 5-Methoxy-UTP | CTP | ATP | GTP |
| 25% 5-Methoxy-UTP + 75% | 75% 5-Methyl-CTP + 25% CTP | ATP | GTP |
| UTP | | | |
| 25% 5-Methoxy-UTP + 75% | 50% 5-Methyl-CTP + 50% CTP | ATP | GTP |
| UTP | | | |
| 25% 5-Methoxy-UTP + 75% | 25% 5-Methyl-CTP + 75% CTP | ATP | GTP |
| UTP | | | |
| 75% 5-Methoxy-UTP + 25% | 75% 5-Methyl-CTP + 25% CTP | ATP | GTP |
| UTP | | | |
| 5-Methoxy-UTP | 5-Fluoro-CTP | ATP | GTP |
| 5-Methoxy-UTP | 5-Phenyl-CTP | ATP | GTP |
| 5-Methoxy-UTP | N4-Bz-CTP | ATP | GTP |
| 5-Methoxy-UTP | CTP | N6- | GTP |
| | | Isopentenyl- | |
| | | ATP | |
| 5-Methoxy-UTP | N4—Ac-CTP | ATP | GTP |
| 25% 5-Methoxy-UTP + 75% | 25% N4—Ac-CTP + 75% CTP | ATP | GTP |
| UTP | | | |
| 25% 5-Methoxy-UTP + 75% | 75% N4—Ac-CTP + 25% CTP | ATP | GTP |
| UTP | | | |
| 75% 5-Methoxy-UTP + 25% | 25% N4—Ac-CTP + 75% CTP | ATP | GTP |
| UTP | | | |
| 75% 5-Methoxy-UTP + 25% | 75% N4—Ac-CTP + 25% CTP | ATP | GTP |
| UTP | | | |
| 5-Methoxy-UTP | 5-Hydroxymethyl-CTP | ATP | GTP |
| 25% 5-Methoxy-UTP + 75% | 25% 5-Hydroxymethyl-CTP + | ATP | GTP |
| UTP | 75% CTP | | |
| 25% 5-Methoxy-UTP + 75% | 75% 5-Hydroxymethyl-CTP + | ATP | GTP |
| UTP | 25% CTP | | |
| 75% 5-Methoxy-UTP + 25% | 25% 5-Hydroxymethyl-CTP + | ATP | GTP |
| UTP | 75% CTP | | |
| 75% 5-Methoxy-UTP + 25% | 75% 5-Hydroxymethyl-CTP + | ATP | GTP |
| UTP | 25% CTP | | |
| 5-Methoxy-UTP | N4-Methyl CTP | ATP | GTP |
| 25% 5-Methoxy-UTP + 75% | 25% N4-Methyl CTP + 75% | ATP | GTP |
| UTP | CTP | | |
| 25% 5-Methoxy-UTP + 75% | 75% N4-Methyl CTP + 25% | ATP | GTP |
| UTP | CTP | | |
| 75% 5-Methoxy-UTP + 25% | 25% N4-Methyl CTP + 75% | ATP | GTP |
| UTP | CTP | | |
| 75% 5-Methoxy-UTP + 25% | 75% N4-Methyl CTP + 25% | ATP | GTP |
| UTP | CTP | | |
| 5-Methoxy-UTP | 5-Trifluoromethyl-CTP | ATP | GTP |
| 25% 5-Methoxy-UTP + 75% | 25% 5-Trifluoromethyl-CTP + | ATP | GTP |
| UTP | 75% CTP | | |
| 25% 5-Methoxy-UTP + 75% | 75% 5-Trifluoromethyl-CTP + | ATP | GTP |
| UTP | 25% CTP | | |
| 75% 5-Methoxy-UTP + 25% | 25% 5-Trifluoromethyl-CTP + | ATP | GTP |
| UTP | 75% CTP | | |
| 75% 5-Methoxy-UTP + 25% | 75% 5-Trifluoromethyl-CTP + | ATP | GTP |
| UTP | 25% CTP | | |
| 5-Methoxy-UTP | 5-Bromo-CTP | ATP | GTP |
| 25% 5-Methoxy-UTP + 75% | 25% 5-Bromo-CTP + 75% CTP | ATP | GTP |
| UTP | | | |
| 25% 5-Methoxy-UTP + 75% | 75% 5-Bromo-CTP + 25% CTP | ATP | GTP |
| UTP | | | |
| 75% 5-Methoxy-UTP + 25% | 25% 5-Bromo-CTP + 75% CTP | ATP | GTP |
| UTP | | | |
| 75% 5-Methoxy-UTP + 25% | 75% 5-Bromo-CTP + 25% CTP | ATP | GTP |
| UTP | | | |
| 5-Methoxy-UTP | 5-Iodo-CTP | ATP | GTP |
| 25% 5-Methoxy-UTP + 75% | 25% 5-Iodo-CTP + 75% CTP | ATP | GTP |
| UTP | | | |
| 25% 5-Methoxy-UTP + 75% | 75% 5-Iodo-CTP + 25% CTP | ATP | GTP |
| UTP | | | |
| 75% 5-Methoxy-UTP + 25% | 25% 5-Iodo-CTP + 75% CTP | ATP | GTP |
| UTP | | | |
| 75% 5-Methoxy-UTP + 25% | 75% 5-Iodo-CTP + 25% CTP | ATP | GTP |
| UTP | | | |
| 5-Methoxy-UTP | 5-Ethyl-CTP | ATP | GTP |
| 25% 5-Methoxy-UTP + 75% | 25% 5-Ethyl-CTP + 75% CTP | ATP | GTP |
| UTP | | | |
| 25% 5-Methoxy-UTP + 75% | 75% 5-Ethyl-CTP + 25% CTP | ATP | GTP |
| UTP | | | |
| 75% 5-Methoxy-UTP + 25% | 25% 5-Ethyl-CTP + 75% CTP | ATP | GTP |
| UTP | | | |
| 75% 5-Methoxy-UTP + 25% | 75% 5-Ethyl-CTP + 25% CTP | ATP | GTP |
| UTP | | | |
| 5-Methoxy-UTP | 5-Methoxy-CTP | ATP | GTP |
| 25% 5-Methoxy-UTP + 75% | 25% 5-Methoxy-CTP + 75% | ATP | GTP |
| UTP | CTP | | |
| 25% 5-Methoxy-UTP + 75% | 75% 5-Methoxy-CTP + 25% | ATP | GTP |
| UTP | CTP | | |
| 75% 5-Methoxy-UTP + 25% | 25% 5-Methoxy-CTP + 75% | ATP | GTP |
| UTP | CTP | | |
| 75% 5-Methoxy-UTP + 25% | 75% 5-Methoxy-CTP + 25% | ATP | GTP |
| UTP | CTP | | |
| 5-Methoxy-UTP | 5-Ethynyl-CTP | ATP | GTP |
| 25% 5-Methoxy-UTP + 75% | 25% 5-Ethynyl-CTP + 75% | ATP | GTP |
| UTP | CTP | | |
| 25% 5-Methoxy-UTP + 75% | 75% 5-Ethynyl-CTP + 25% | ATP | GTP |
| UTP | CTP | | |
| 75% 5-Methoxy-UTP + 25% | 25% 5-Ethynyl-CTP + 75% | ATP | GTP |
| UTP | CTP | | |
| 75% 5-Methoxy-UTP + 25% | 75% 5-Ethynyl-CTP + 25% | ATP | GTP |
| UTP | CTP | | |
| 5-Methoxy-UTP | 5-Pseudo-iso-CTP | ATP | GTP |
| 25% 5-Methoxy-UTP + 75% | 25% 5-Pseudo-iso-CTP + 75% | ATP | GTP |
| UTP | CTP | | |
| 25% 5-Methoxy-UTP + 75% | 75% 5-Pseudo-iso-CTP + 25% | ATP | GTP |
| UTP | CTP | | |
| 75% 5-Methoxy-UTP + 25% | 25% 5-Pseudo-iso-CTP + 75% | ATP | GTP |
| UTP | CTP | | |
| 75% 5-Methoxy-UTP + 25% | 75% 5-Pseudo-iso-CTP + 25% | ATP | GTP |
| UTP | CTP | | |
| 5-Methoxy-UTP | 5-Formyl-CTP | ATP | GTP |
| 25% 5-Methoxy-UTP + 75% | 25% 5-Formyl-CTP + 75% CTP | ATP | GTP |
| UTP | | | |
| 25% 5-Methoxy-UTP + 75% | 75% 5-Formyl-CTP + 25% CTP | ATP | GTP |
| UTP | | | |
| 75% 5-Methoxy-UTP + 25% | 25% 5-Formyl-CTP + 75% CTP | ATP | GTP |
| UTP | | | |
| 75% 5-Methoxy-UTP + 25% | 75% 5-Formyl-CTP + 25% CTP | ATP | GTP |
| UTP | | | |
| 5-Methoxy-UTP | 5-Aminoallyl-CTP | ATP | GTP |
| 25% 5-Methoxy-UTP + 75% | 25% 5-Aminoallyl-CTP + 75% | ATP | GTP |
| UTP | CTP | | |
| 25% 5-Methoxy-UTP + 75% | 75% 5-Aminoallyl-CTP + 25% | ATP | GTP |
| UTP | CTP | | |
| 75% 5-Methoxy-UTP + 25% | 25% 5-Aminoallyl-CTP + 75% | ATP | GTP |
| UTP | CTP | | |
| 75% 5-Methoxy-UTP + 25% | 75% 5-Aminoallyl-CTP + 25% | ATP | GTP |
| UTP | CTP | | |

### VII. Pharmaceutical Compositions

1. Formulation, Administration, Delivery and Dosing

The present invention provides use of a pharmaceutical composition comprising a polynucleotide disclosed herein (e.g., one or more mRNAs encoding an antibody or an antigen binding portion thereof which specifically binds to CTLA-4) to reduce the size of a tumor or inhibiting the growth of a tumor in a subject in need thereof. The present invention further provides use of a pharmaceutical composition comprising a one or more mRNAs encoding an antibody or an antigen binding portion thereof which specifically binds to CTLA-4 to (i) release inhibitory controls on T cell activation and proliferation, thereby inducing antitumor immunity; (ii) decrease tumor growth; (iii) achieve tumor inhibition (e.g., complete tumor inhibition), (iv) increase survival rates, or (v) any combination thereof.

In some embodiments of the invention, the polynucleotides disclosed herein (e.g., reducing the size of a tumor or inhibiting the growth of a tumor in a subject in need thereof) are formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present invention can be sterile and/or pyrogen-free. General considerations in the formulation and/or manufacture of pharmaceutical agents can be found, for example, in Remington: The Science and Practice of Pharmacy 21^{st} ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotides of the present disclosure (e.g., one or more mRNAs encoding an antibody or an antigen binding portion thereof which specifically binds to CTLA-4) are formulated for subcutaneous, intravenous, intraperitoneal, intratumroal, intramuscular, intra-articular, intra-synovial, intrasternal, intrathecal, intrahepatic, intralesional, intracranial, intraventricular, oral, inhalation spray, topical, rectal, nasal, buccal, vaginal, intratumoral, or implanted reservoir intramuscular, subcutaneous, intratumoral, or intradermal delivery.

In other embodiments, the polynucleotides disclosed herein (e.g., one or more mRNAs encoding an antibody or an antigen binding portion thereof which specifically binds to CTLA-4) are formulated for intratumoral, intraperitoneal, or intravenous delivery. In a particular embodiment, the polynucleotides of the present disclosure (e.g., one or more mRNAs encoding an antibody or an antigen binding portion thereof which specifically binds to CTLA-4) are formulated for intratumoral delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition can comprise between 0.1% and 100%, e.g., between 0.5 and 50%, between 1-30%, between 5-80%, at least 80% (w/w) active ingredient.

2. Formulations

The polynucleotides disclosed herein (e.g., one or more mRNAs encoding an antibody or an antigen binding portion thereof which specifically binds to CTLA-4) can be formulated using one or more excipients to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo.

In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the invention can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present invention can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide (e.g., one mRNA encoding an antibody or an antigen binding portion thereof which specifically binds to CTLA-4). As a non-limiting example, the formulations contain 1, 2, 3, 4 or 5 polynucleotides (e.g., more than one mRNAs encoding an antibody or an antigen binding portion thereof which specifically binds to CTLA-4).

In some embodiments, the polynucleotides of the invention (e.g., one or more mRNAs encoding an antibody or an antigen binding portion thereof which specifically binds to CTLA-4) are formulated for intratumoral delivery in a tumor of a patient in need thereof.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the invention.

In some embodiments, the polynucleotides is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core.

3. Lipidoids

The polynucleotides disclosed herein (e.g., one or more mRNAs encoding an antibody or an antigen binding portion thereof which specifically binds to CTLA-4) can be formulated with lipidoids. The synthesis of lipidoids has been extensively described and formulations containing these compounds are particularly suited for delivery of polynucleotides (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001).

While these lipidoids have been used to effectively deliver double stranded small interfering RNA molecules in rodents and non-human primates (see Akinc et al., Nat Biotechnol. 2008 26:561-569; Frank-Kamenetsky et al., Proc Natl Acad Sci USA. 2008 105:11915-11920; Akinc et al., Mol Ther. 2009 17:872-879; Love et al., Proc Natl Acad Sci USA. 2010 107:1864-1869; Leuschner et al., Nat Biotechnol. 2011 29:1005-1010), the present disclosure describes their formulation and use in delivering polynucleotides.

Complexes, micelles, liposomes or particles can be prepared containing these lipidoids and therefore, can result in an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intraperitoneal, intratumoral, intramuscular, or subcutaneous routes.

In vivo delivery of nucleic acids can be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009 17:872-879). As an example, small changes in the anchor chain length of poly(ethylene glycol) (PEG) lipids can result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010)), C12-200 (including derivatives and variants), and MDI, can be tested for in vivo activity.

The lipidoid referred to herein as “98N12-5” is disclosed by Akinc et al., Mol Ther. 2009 17:872-879.

The lipidoid referred to herein as “C12-200” is disclosed by Love et al., Proc Natl Acad Sci USA. 2010 107:1864-1869 and Liu and Huang, Molecular Therapy. 2010 669-670. The lipidoid formulations can include particles comprising either 3 or 4 or more components in addition to polynucleotides.

Lipidoids and polynucleotide formulations comprising lipidoids are described in International Patent Application No. PCT/US2014/097077.

4. Liposomes, Lipoplexes, and Lipid Nanoparticles

The polynucleotides of the invention (e.g., one or more mRNAs encoding an antibody or an antigen binding portion thereof which specifically binds to CTLA-4) can be formulated using one or more liposomes, lipoplexes, or lipid nanoparticles. In one embodiment, pharmaceutical compositions of one or more mRNAs encoding an antibody or an antigen binding portion thereof which specifically binds to CTLA-4 include liposomes. Liposomes are artificially-prepared vesicles which can primarily be composed of a lipid bilayer and can be used as a delivery vehicle for the administration of pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations.

The formation of liposomes can depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles are prepared by the methods, apparatus and devices described in U.S. Patent Publication Nos. US20130177638, US20130177637, US20130177636, US20130177635, US20130177634, US20130177633, US20130183375, US20130183373 and US20130183372.

In one embodiment, pharmaceutical compositions described herein include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (as described in US20100324120) and liposomes which can deliver small molecule drugs such as, but not limited to, DOXIL® from Janssen Biotech, Inc. (Horsham, Pa.).

In one embodiment, pharmaceutical compositions described herein can include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 1 19:661-673; deFougerolles Hum Gene Ther. 2008 19:125-132; U.S. Patent Publication No US20130122104).

The original manufacture method by Wheeler et al was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In other embodiments, formulations comprise a percentage of cholesterol selected from the group consisting of 28.5%, 31.5%, 33.5%, 36.5%, 37.0%, 38.5%, 39.0% and 43.5%. In some embodiments, formulations comprise from about 5.0% to about 10.0% DSPC and/or from about 7.0% to about 15.0% DSPC.

In one embodiment, pharmaceutical compositions include liposomes which are formed to deliver one or more mRNAs encoding an antibody or an antigen binding portion thereof which specifically binds to CTLA-4. The polynucleotides can be encapsulated by the liposome and/or it can be contained in an aqueous core which can then be encapsulated by the liposome (see International Pub. Nos. WO2012031046, WO2012031043, WO2012030901 and. WO2012006378 and US Patent Publication No. US20130189351, US20130195969 and US20130202684).

In another embodiment, liposomes are formulated for targeted delivery. As a non-limiting example, the liposome is formulated for targeted delivery to the liver. The liposome used for targeted delivery can include, but is not limited to, the liposomes described in and methods of making liposomes described in US Patent Publication No. US20130195967.

In another embodiment, the one or more mRNAs encoding an antibody or an antigen binding portion thereof which specifically binds to CTLA-4 are formulated in a cationic oil-in-water emulsion where the emulsion particle comprises an oil core and a cationic lipid which can interact with the polynucleotide anchoring the molecule to the emulsion particle (see International Pub. No. WO2012006380).

In one embodiment, the one or more mRNAs encoding an antibody or an antigen binding portion thereof which specifically binds to CTLA-4 are formulated in a water-in-oil emulsion comprising a continuous hydrophobic phase in which the hydrophilic phase is dispersed. As a non-limiting example, the emulsion can be made by the methods described in International Publication No. WO201087791.

In another embodiment, the lipid formulation includes at least cationic lipid, a lipid which can enhance transfection and a least one lipid which contains a hydrophilic head group linked to a lipid moiety (International Pub. No. WO2011076807 and U.S. Pub. No. 20110200582). In another embodiment, the one or more mRNAs encoding an antibody or an antigen binding portion thereof which specifically binds to CTLA-4 are formulated in a lipid vesicle which can have crosslinks between functionalized lipid bilayers (see U.S. Pub. No. 20120177724).

In one embodiment, the one or more mRNAs encoding an antibody or an antigen binding portion thereof which specifically binds to CTLA-4 are formulated in a liposome as described in International Patent Publication No. WO2013086526. The one or more mRNAs encoding an antibody or an antigen binding portion thereof which specifically binds to CTLA-4 can be encapsulated in a liposome using reverse pH gradients and/or optimized internal buffer compositions as described in International Patent Publication No. WO2013086526.

In one embodiment, the pharmaceutical compositions comprising one or more mRNAs encoding an antibody or an antigen binding portion thereof which specifically binds to CTLA-4 are formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713)) and hyaluronan-coated liposomes (Quiet Therapeutics, Israel).

In one embodiment, the cationic lipid is a low molecular weight cationic lipid such as those described in US Patent Application No. 20130090372. In another embodiment, the one or more mRNAs encoding an antibody or an antigen binding portion thereof which specifically binds to CTLA-4 are formulated in a lipid vesicle which can have crosslinks between functionalized lipid bilayers.

In other embodiments, the one or more mRNAs encoding an antibody or an antigen binding portion thereof which specifically binds to CTLA-4 disclosed herein are formulated in a liposome comprising a cationic lipid. The liposome can have a molar ratio of nitrogen atoms in the cationic lipid to the phophates in the polynucleotide (N:P ratio) of between 1:1 and 20:1 as described in international Publication No. WO2013006825. In another embodiment, the liposome can have a N:P ratio of greater than 20:1 or less than 1:1.

In one embodiment, the one or more mRNAs encoding an antibody or an antigen binding portion thereof which specifically binds to CTLA-4 disclosed herein are formulated in a lipid-polycation complex. The formation of the lipid-polycation complex can be accomplished by methods known in the art and/or as described in U.S. Pub. No. 20120178702. As a non-limiting example, the polycation includes a cationic peptide or a polypeptide such as, but not limited to, polylysine, polyornithine and/or polyarginine and the cationic peptides described in International Pub. No. WO2012013326 or US Patent Pub. No. US20130142818. In another embodiment, the polynucleotides are formulated in a lipid-polycation complex which can further include a non-cationic lipid such as, but not limited to, cholesterol or dioleoyl phosphatidylethanolamine (DOPE).

In one embodiment, the one or more mRNAs encoding an antibody or an antigen binding portion thereof which specifically binds to CTLA-4 disclosed herein are formulated in an aminoalcohol lipidoid. Aminoalcohol lipidoids which can be used in the present invention can be prepared by the methods described in U.S. Pat. No. 8,450,298.

The liposome formulation can be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha. et al. Mol Ther. 2011 19:2186-2200). In some embodiments, liposome formulations comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes is from about 5:1 to about 20:1, from about 10:1 to about 25:1, from about 15:1 to about 30:1 and/or at least 30:1.

In some embodiments, the ratio of PEG in the lipid nanoparticle (LNP) formulations is increased or decreased and/or the carbon chain length of the PEG lipid is modified from C14 to C18 to alter the pharmacokinetics and/or biodistribution of the LNP formulations. As a non-limiting example, LNP formulations contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In another embodiment the PEG-c-DOMG can be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid can be selected from any lipid known in the art such as, but not limited to, DLin-MC3-DMA, DLin-DMA, C12-200 and DLin-KC2-DMA.

In one embodiment, the one or more mRNAs encoding an antibody or an antigen binding portion thereof which specifically binds to CTLA-4 are formulated in a lipid nanoparticle such as those described in International Publication No. WO2012170930.

In another embodiment, the formulation comprising the polynucleotide is a nanoparticle which can comprise at least one lipid. The lipid can be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid is a cationic lipid such as, but not limited to, DLin-DMA, DLin-D-DMA, DLin-MC3-DMA, DLin-KC2-DMA, DODMA and amino alcohol lipids. The amino alcohol cationic lipid can be the lipids described in and/or made by the methods described in US Patent Publication No. US20130150625. As a non-limiting example, the cationic lipid can be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

In some embodiments, the polynucleotide of the invention is formulated in a lipid nanoparticle, wherein the polynucleotide comprises an mRNA encoding a polypeptide comprising any one of the amino acid sequences set forth in TABLE1. In some embodiments, the polynucleotide of the invention is formulated in a lipid nanoparticle, wherein the polynucleotide encodes any one of the amino acid sequences set forth in TABLE1.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In one embodiment, the lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In one embodiment, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In one embodiment, the formulation includes from about 0.5% to about 15% on a molar basis of the neutral lipid e.g., from about 3 to about 12%, from about 5 to about 10% or about 15%, about 10%, or about 7.5% on a molar basis. Exemplary neutral lipids include, but are not limited to, DSPC, POPC, DPPC, DOPE and SM. In one embodiment, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In one embodiment, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis.

In one embodiment, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Exemplary PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005).

In one embodiment, the foiinulations of the inventions include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In one embodiment, the formulations of the inventions include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the formulations of the inventions include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the formulations of the inventions include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the formulations of the inventions include about 50% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the formulations of the inventions include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC 3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the formulations of the inventions include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the formulations of the inventions include about 57.2% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In one embodiment, the formulations of the inventions include about 57.5% of a cationic lipid selected from the PEG lipid is PEG-cDNA (PEG-cDMA is further discussed in Reyes et al. (J. Controlled Release, 107, 276-287 (2005)), about 7.5% of the neutral lipid, about 31.5% of the sterol, and about 3.5% of the PEG or PEG-modified lipid on a molar basis.

In some embodiments, lipid nanoparticle formulation consists essentially of a lipid mixture in molar ratios of about 20-70% cationic lipid: 5-45% neutral lipid: 20-55% cholesterol: 0.5-15% PEG-modified lipid; e.g., in a molar ratio of about 20-60% cationic lipid: 5-25% neutral lipid: 25-55% cholesterol: 0.5-15% PEG-modified lipid.

In particular embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-CDMA).

Exemplary lipid nanoparticle compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578.

In one embodiment, the lipid nanoparticle formulations described herein comprise a cationic lipid, a PEG lipid and a structural lipid and optionally comprise a non-cationic lipid. As a non-limiting example, the lipid nanoparticle comprises about 40-60% of cationic lipid, about 5-15% of a non-cationic lipid, about 1-2% of a PEG lipid and about 30-50% of a structural lipid. As another non-limiting example, the lipid nanoparticle comprises about 50% cationic lipid, about 10% non-cationic lipid, about 1.5% PEG lipid and about 38.5% structural lipid. As yet another non-limiting example, the lipid nanoparticle comprises about 55% cationic lipid, about 10% non-cationic lipid, about 2.5% PEG lipid and about 32.5% structural lipid. In one embodiment, the cationic lipid is any cationic lipid described herein such as, but not limited to, DLin-KC2-DMA, DLin-MC3-DMA and L319.

In one embodiment, the lipid nanoparticle formulations described herein is 4 component lipid nanoparticles. The lipid nanoparticle can comprise a cationic lipid, a non-cationic lipid, a PEG lipid and a structural lipid. As a non-limiting example, the lipid nanoparticle can comprise about 40-60% of cationic lipid, about 5-15% of a non-cationic lipid, about 1-2% of a PEG lipid and about 30-50% of a structural lipid. As another non-limiting example, the lipid nanoparticle can comprise about 50% cationic lipid, about 10% non-cationic lipid, about 1.5% PEG lipid and about 38.5% structural lipid. As yet another non-limiting example, the lipid nanoparticle can comprise about 55% cationic lipid, about 10% non-cationic lipid, about 2.5% PEG lipid and about 32.5% structural lipid. In one embodiment, the cationic lipid can be any cationic lipid described herein such as, but not limited to, DLin-KC2-DMA, DLin-MC3-DMA and L319.

In one embodiment, the lipid nanoparticle formulations described herein comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

In one embodiment, the cationic lipid is selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and US20130225836. In another embodiment, the cationic lipid can be selected from, but not limited to, formula A described in International Publication Nos. WO2012040184, WO2011153120, WO2011149733, WO2011090965, WO2011043913, WO2011022460, WO2012061259, WO2012054365, WO2012044638 and WO2013116126 or US Patent Publication No. US20130178541 and US20130225836.

In yet another embodiment, the cationic lipid can be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115, formula I of US Patent Publication No US20130123338. As a non-limiting example, the cationic lipid can be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl] pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In one embodiment, the lipid is a cleavable lipid such as those described in International Publication No. WO2012170889. In another embodiment, the lipid is a cationic lipid such as, but not limited to, Formula (I) of U.S. Patent Application No. US20130064894.

In one embodiment, the cationic lipid is synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724, WO201021865, WO2013086373 and WO2013086354.

In another embodiment, the cationic lipid is a trialkyl cationic lipid. Non-limiting examples of trialkyl cationic lipids and methods of making and using the trialkyl cationic lipids are described in International Patent Publication No. WO2013126803.

In one embodiment, the LNP formulations of the polynucleotides contain PEG-c-DOMG at 3% lipid molar ratio. In another embodiment, the LNP formulations of the polynucleotides contains PEG-c-DOMG at 1.5% lipid molar ratio.

In one embodiment, the pharmaceutical compositions of the one or more mRNAs encoding an antibody or an antigen binding portion thereof which specifically binds to CTLA-4 include at least one of the PEGylated lipids described in International Publication No. WO2012099755.

In one embodiment, the LNP formulation contains PEG-DMG 2000 (1,2-dimyristoyl-sn-glycero-3-phophoethanolamine-N-[methoxy(polyethylene glycol)-2000). In one embodiment, the LNP formulation can contain PEG-DMG 2000, a cationic lipid known in the art and at least one other component. In another embodiment, the LNP formulation contains PEG-DMG 2000, a cationic lipid known in the art, DSPC and cholesterol. As a non-limiting example, the LNP formulation contains PEG-DMG 2000, DLin-DMA, DSPC and cholesterol. As another non-limiting example the LNP formulation contains PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294).

In one embodiment, the LNP formulation is formulated by the methods described in International Publication Nos. WO2011127255 or WO2008103276. As a non-limiting example, the one or more mRNAs encoding an antibody or an antigen binding portion thereof which specifically binds to CTLA-4 described herein are encapsulated in LNP formulations as described in WO2011127255 and/or WO2008103276.

In one embodiment, the one or more mRNAs encoding an antibody or an antigen binding portion thereof which specifically binds to CTLA-4 described herein are formulated in a nanoparticle to be delivered by a parenteral route as described in U.S. Pub. No. US20120207845.

In one embodiment, the one or more mRNAs encoding an antibody or an antigen binding portion thereof which specifically binds to CTLA-4 are formulated in a lipid nanoparticle made by the methods described in US Patent Publication No US20130156845 or International Publication No WO2013093648 or WO2012024526.

The lipid nanoparticles described herein can be made in a sterile environment by the system and/or methods described in US Patent Publication No. US20130164400.

In one embodiment, the LNP formulation is formulated in a nanoparticle such as a nucleic acid-lipid particle described in U.S. Pat. No. 8,492,359. As a non-limiting example, the lipid particle comprises one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle. The nucleic acid in the nanoparticle can be the polynucleotides described herein and/or are known in the art.

In one embodiment, the LNP formulation is formulated by the methods described in International Publication Nos. WO2011127255 or WO2008103276. As a non-limiting example, modified mRNA described herein is encapsulated in LNP formulations as described in WO2011127255 and/or WO2008103276.

In one embodiment, LNP formulations described herein comprise a polycationic composition. As a non-limiting example, the polycationic composition is selected from formula 1-60 of US Patent Publication No. US20050222064. In another embodiment, the LNP formulations comprising a polycationic composition are used for the delivery of the modified mRNA described herein in vivo and/or in vitro.

In one embodiment, the LNP formulations described herein additionally comprise a permeability enhancer molecule. Non-limiting permeability enhancer molecules are described in US Patent Publication No. US20050222064.

In one embodiment, the polynucleotide pharmaceutical compositions are formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713)) and hyaluronan-coated liposomes (Quiet Therapeutics, Israel).

In one embodiment, the one or more mRNAs encoding an antibody or an antigen binding portion thereof which specifically binds to CTLA-4 are formulated in a lyophilized gel-phase liposomal composition as described in US Publication No. US2012060293.

The nanoparticle formulations can comprise a phosphate conjugate. The phosphate conjugate can increase in vivo circulation times and/or increase the targeted delivery of the nanoparticle. Phosphate conjugates for use with the present invention can be made by the methods described in International Application No. WO2013033438 or US Patent Publication No. US20130196948. As a non-limiting example, the phosphate conjugates can include a compound of any one of the formulas described in international Application No. WO2013033438.

The nanoparticle formulation can comprise a polymer conjugate. The polymer conjugate can be a water soluble conjugate. The polymer conjugate can have a structure as described in U.S. Patent Application No. 20130059360.

In one aspect, polymer conjugates with one or more mRNAs encoding an antibody or an antigen binding portion thereof which specifically binds to CTLA-4 can be made using the methods and/or segmented polymeric reagents described in U.S. Patent Application No. 20130072709. In another aspect, the polymer conjugate can have pendant side groups comprising ring moieties such as, but not limited to, the polymer conjugates described in US Patent Publication No. US20130196948.

The nanoparticle formulations can comprise a conjugate to enhance the delivery of nanoparticles of the present invention in a subject. Further, the conjugate can inhibit phagocytic clearance of the nanoparticles in a subject. In one aspect, the conjugate is a “self” peptide designed from the human membrane protein CD47 (e.g., the “self” particles described by Rodriguez et al (Science 2013 339, 971-975)). As shown by Rodriguez et al. the self peptides delayed macrophage-mediated clearance of nanoparticles which enhanced delivery of the nanoparticles. In another aspect, the conjugate is the membrane protein CD47 (e.g., see Rodriguez et al. Science 2013 339, 971-975). Rodriguez et al. showed that, similarly to “self” peptides, CD47 can increase the circulating particle ratio in a subject as compared to scrambled peptides and PEG coated nanoparticles.

In one embodiment, the one or more mRNAs encoding an antibody or an antigen binding portion thereof which specifically binds to CTLA-4 of the present invention are formulated in nanoparticles which comprise a conjugate to enhance the delivery of the nanoparticles of the present invention in a subject. The conjugate can be the CD47 membrane or the conjugate can be derived from the CD47 membrane protein, such as the “self” peptide described previously. In another aspect the nanoparticle can comprise PEG and a conjugate of CD47 or a derivative thereof. In yet another aspect, the nanoparticle comprises both the “self” peptide described above and the membrane protein CD47.

In another aspect, a “self” peptide and/or CD47 protein is conjugated to a virus-like particle or pseudovirion, as described herein for delivery of the polynucleotides of the present invention.

In another embodiment, pharmaceutical compositions comprise one or more mRNAs encoding an antibody or an antigen binding portion thereof which specifically binds to CTLA-4 disclosed herein and a conjugate which can have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in US Patent Publication No. US20130184443.

The nanoparticle formulations can be a carbohydrate nanoparticle comprising a carbohydrate carrier and a polynucleotide. As a non-limiting example, the carbohydrate carrier includes, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121).

Nanoparticle formulations of the present invention can be coated with a surfactant or polymer in order to improve the delivery of the particle. In one embodiment, the nanoparticle is coated with a hydrophilic coating such as, but not limited to, PEG coatings and/or coatings that have a neutral surface charge. The hydrophilic coatings can help to deliver nanoparticles with larger payloads such as, but not limited to, polynucleotides within the central nervous system. As a non-limiting example nanoparticles comprising a hydrophilic coating and methods of making such nanoparticles are described in US Patent Publication No. US20130183244.

In one embodiment, the lipid nanoparticles of the present invention are hydrophilic polymer particles. Non-limiting examples of hydrophilic polymer particles and methods of making hydrophilic polymer particles are described in US Patent Publication No. US20130210991.

In another embodiment, the lipid nanoparticles of the present invention are hydrophobic polymer particles. Lipid nanoparticle formulations can be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and can be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it can be terminally located at the terminal end of the lipid chain. The internal ester linkage can replace any carbon in the lipid chain.

In one embodiment, the internal ester linkage is located on either side of the saturated carbon.

In one embodiment, an immune response is elicited by delivering a lipid nanoparticle which can include a nanospecies, a polymer and an immunogen. (U.S. Publication No. 20120189700 and International Publication No. WO2012099805). The polymer can encapsulate the nanospecies or partially encapsulate the nanospecies. The immunogen can be a recombinant protein, a modified RNA and/or an polynucleotide encoding an antibody or antigen binding portion thereof which specifically binds to CTLA-4 described herein.

Lipid nanoparticles can be engineered to alter the surface properties of particles the lipid nanoparticles can penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles can be removed from the mucosal tissue within seconds or within a few hours.

Large polymeric nanoparticles (200 nm -500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171). The transport of nanoparticles can be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier can be made as described in U.S. Pat. No. 8,241,670 or International Patent Publication No. WO2013110028.

The lipid nanoparticle engineered to penetrate mucus can comprise a polymeric material (i.e. a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material can be biodegradable and/or biocompatible. Non-limiting examples of biocompatible polymers are described in International Patent Publication No. WO2013116804. The polymeric material can additionally be irradiated. As a non-limiting example, the polymeric material can be gamma irradiated (See e.g., International App. No. WO201282165).

Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone.

The lipid nanoparticle can be coated or associated with a co-polymer such as, but not limited to, a block co-polymer (such as a branched polyether-polyamide block copolymer described in International Publication No. WO2013012476), and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665).

The co-polymer can be a polymer that is generally regarded as safe (GRAS) and the formation of the lipid nanoparticle can be in such a way that no new chemical entities are created. For example, the lipid nanoparticle can comprise poloxamers coating PLGA nanoparticles without forming new chemical entities which are still able to rapidly penetrate human mucus (Yang et al. Angew. Chem. Int. Ed. 2011 50:2597-2600). A non-limiting scalable method to produce nanoparticles which can penetrate human mucus is described by Xu et al. (See e.g., J Control Release 2013, 170(2):279-86).

The vitamin of the polymer-vitamin conjugate can be vitamin E. The vitamin portion of the conjugate can be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus can include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent can be embedded or enmeshed in the particle's surface or disposed (e.g., by coating, adsorption, covalent linkage, or other process) on the surface of the lipid nanoparticle (see, e.g., US Publication 20100215580 and US Publication 20080166414 and US20130164343).

In one embodiment, the mucus penetrating lipid nanoparticles comprises at least one polynucleotide described herein (e.g., one or more mRNAs encoding an antibody or an antigen binding portion thereof which specifically binds to CTLA-4). The polynucleotide can be encapsulated in the lipid nanoparticle and/or disposed on the surface of the particle. The polynucleotide can be covalently coupled to the lipid nanoparticle. Formulations of mucus penetrating lipid nanoparticles can comprise a plurality of nanoparticles. Further, the formulations can contain particles which can interact with the mucus and alter the structural and/or adhesive properties of the surrounding mucus to decrease mucoadhesion which can increase the delivery of the mucus penetrating lipid nanoparticles to the mucosal tissue.

In another embodiment, the mucus penetrating lipid nanoparticles are a hypotonic formulation comprising a mucosal penetration enhancing coating. The formulation can be hypotonice for the epithelium to which it is being delivered. Non-limiting examples of hypotonic formulations can be found in International Patent Publication No. WO2013110028.

In one embodiment, in order to enhance the delivery through the mucosal barrier the formulation comprises or is a hypotonic solution. Hypotonic solutions were found to increase the rate at which mucoinert particles such as, but not limited to, mucus-penetrating particles, were able to reach the vaginal epithelial surface (See e.g., Ensign et al. Biomaterials 2013 34(28):6922-9).

In one embodiment, the one or more mRNAs encoding an antibody or an antigen binding portion thereof which specifically binds to CTLA-4 are formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci U S A. 2007 6;104:4095-4100; deFougerolles Hum Gene Ther. 2008 19:125-132).

In one embodiment such formulations are also constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133).

One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci U S A. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133).

In one embodiment, the one or more mRNAs encoding an antibody or an antigen binding portion thereof which specifically binds to CTLA-4 of the invention are formulated as a solid lipid nanoparticle. A solid lipid nanoparticle (SLN) can be spherical with an average diameter between 10 to 1000 nm. SLN possess a solid lipid core matrix that can solubilize lipophilic molecules and can be stabilized with surfactants and/or emulsifiers. In a further embodiment, the lipid nanoparticle can be a self-assembly lipid-polymer nanoparticle (see Zhang et al., ACS Nano, 2008, 2 (8), pp 1696-1702). As a non-limiting example, the SLN can be the SLN described in International Patent Publication No. WO2013105101. As another non-limiting example, the SLN can be made by the methods or processes described in International Patent Publication No. WO2013105101.

Liposomes, lipoplexes, or lipid nanoparticles can be used to improve the efficacy of the one or more mRNAs encoding an antibody or an antigen binding portion thereof which specifically binds to CTLA-4 as these formulations can be able to increase cell transfection by the polynucleotides; and/or increase the translation of encoded antibody or an antigen binding portion thereof which specifically binds to CTLA-4. One such example involves the use of lipid encapsulation to enable the effective systemic delivery of polyplex plasmid DNA (Heyes et al., Mol Ther. 2007 15:713-720). The liposomes, lipoplexes, or lipid nanoparticles can also be used to increase the stability of the polynucleotide.

In one embodiment, the one or more mRNAs encoding an antibody or an antigen binding portion thereof which specifically binds to CTLA-4 of the present invention are formulated for controlled release and/or targeted delivery. As used herein, “controlled release” refers to a pharmaceutical composition or compound release profile that conforms to a particular pattern of release to effect a therapeutic outcome. In one embodiment, the polynucleotides are encapsulated into a delivery agent described herein and/or known in the art for controlled release and/or targeted delivery. As used herein, the term “encapsulate” means to enclose, surround or encase.

As it relates to the formulation of the compounds of the invention, encapsulation can be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent. Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In one embodiment, the controlled release formulation includes, but is not limited to, tri-block co-polymers. As a non-limiting example, the formulation includes two different types of tri-block co-polymers (International Pub. No. WO2012131104 and WO2012131106).

In another embodiment, the one or more mRNAs encoding an antibody or an antigen binding portion thereof which specifically binds to CTLA-4 are encapsulated into a lipid nanoparticle or a rapidly eliminated lipid nanoparticle and the lipid nanoparticles or a rapidly eliminated lipid nanoparticle can then be encapsulated into a polymer, hydrogel and/or surgical sealant described herein and/or known in the art. As a non-limiting example, the polymer, hydrogel or surgical sealant is PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, or COSEAL® (Baxter International, Inc Deerfield, Ill.).

In another embodiment, the lipid nanoparticle is encapsulated into any polymer known in the art which can form a gel when injected into a subject. As another non-limiting example, the lipid nanoparticle is encapsulated into a polymer matrix which can be biodegradable.

In one embodiment, the formulation for controlled release and/or targeted delivery comprising one or more mRNAs encoding an antibody or an antigen binding portion thereof which specifically binds to CTLA-4 also includes at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the polynucleotide controlled release and/or targeted delivery formulation comprises at least one degradable polyester which can contain polycationic side chains. Degradeable polyesters include, but are not limited to, poly(serine ester), poly(L-lactide-co-L-lysine), polly(4-hydroxy-L-proline ester), and combinations thereof. In another embodiment, the degradable polyesters can include a PEG conjugation to form a PEGylated polymer.

In one embodiment, the polynucleotide controlled release and/or targeted delivery formulation comprising at least one polynucleotide comprises at least one PEG and/or PEG related polymer derivatives as described in U.S. Pat. No. 8,404,222.

In another embodiment, the polynucleotide controlled release delivery formulation comprising at least one polynucleotide is the controlled release polymer system described in US20130130348.

In one embodiment, the one or more mRNAs encoding an antibody or an antigen binding portion thereof which specifically binds to CTLA-4 of the present invention is encapsulated in a therapeutic nanoparticle. Therapeutic nanoparticles can be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211. In another embodiment, therapeutic polymer nanoparticles can be identified by the methods described in US Pub No. US20120140790.

In one embodiment, the one or more mRNAs encoding an antibody or an antigen binding portion thereof which specifically binds to CTLA-4 are formulated for sustained release. As used herein, “sustained release” refers to a pharmaceutical composition or compound that conforms to a release rate over a specific period of time. The period of time can include, but is not limited to, hours, days, weeks, months and years.

As a non-limiting example, the sustained release nanoparticle comprises a polymer and a therapeutic agent such as, but not limited to, the one or more mRNAs encoding an antibody or an antigen binding portion thereof which specifically binds to CTLA-4 of the present invention (see International Pub No. 2010075072 and US Pub No. US20100216804, US20110217377 and US20120201859). In another non-limiting example, the sustained release formulation comprises agents which permit persistent bioavailability such as, but not limited to, crystals, macromolecular gels and/or particulate suspensions (see US Patent Publication No US20130150295).

In one embodiment, the one or more mRNAs encoding an antibody or an antigen binding portion thereof which specifically binds to CTLA-4 can be formulated to be target specific. As a non-limiting example, the therapeutic nanoparticles include a corticosteroid (see International Pub. No. WO2011084518). As a non-limiting example, the therapeutic nanoparticles are formulated in nanoparticles described in international Pub No. WO2008121949, WO2010005726, WO2010005725, WO2011084521 and US Pub No. US20100069426, US20120004293 and US20100104655.

In one embodiment, the nanoparticles of the present invention comprise a polymeric matrix. As a non-limiting example, the nanoparticle comprises two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer includes PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In yet another embodiment, the diblock copolymer is a high-X diblock copolymer such as those described in International Patent Publication No. WO2013120052.

As a non-limiting example the therapeutic nanoparticle comprises a PLGA-PEG block copolymer (see US Pub. No. US20120004293 and U.S. Pat. No. 8,236,330). In another non-limiting example, the therapeutic nanoparticle is a stealth nanoparticle comprising a diblock copolymer of PEG and PLA or PEG and PLGA (see U.S. Pat. No 8,246,968 and International Publication No. WO2012166923). In yet another non-limiting example, the therapeutic nanoparticle is a stealth nanoparticle or a target-specific stealth nanoparticle as described in US Patent Publication No. US20130172406.

In one embodiment, the therapeutic nanoparticle comprises a multiblock copolymer (See e.g., U.S. Pat. Nos. 8,263,665 and 8,287,910 and US Patent Pub. No. US20130195987).

In yet another non-limiting example, the lipid nanoparticle comprises the block copolymer PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253). The polynucleotides comprising an mRNA encoding an antibody or antigen binding portion thereof which specifically binds to CTLA-4 can be formulated in lipid nanoparticles comprising the PEG-PLGA-PEG block copolymer.

In one embodiment, the therapeutic nanoparticle comprises a multiblock copolymer (See e.g., U.S. Pat. Nos. 8,263,665 and 8,287,910 and US Patent Pub. No. US20130195987).

In one embodiment, the block copolymers described herein are included in a polyion complex comprising a non-polymeric micelle and the block copolymer. (See e.g., U.S. Pub. No. 20120076836).

In one embodiment, the therapeutic nanoparticle comprises at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticle comprises at least one poly(vinyl ester) polymer. The poly(vinyl ester) polymer can be a copolymer such as a random copolymer. As a non-limiting example, the random copolymer has a structure such as those described in International Application No. WO2013032829 or US Patent Publication No US20130121954. In one aspect, the poly(vinyl ester) polymers can be conjugated to the polynucleotides described herein.

In one embodiment, the therapeutic nanoparticle comprises at least one diblock copolymer. The diblock copolymer can be, but it not limited to, a poly(lactic) acid-poly(ethylene)glycol copolymer (see e.g., International Patent Publication No. WO2013044219 and U.S. Patent Application Publ. No. US20150017245). As a non-limiting example, the therapeutic nanoparticle are used to treat cancer (see international publication No. WO2013044219).

In one embodiment, the therapeutic nanoparticles comprise at least one cationic polymer described herein and/or known in the art.

In one embodiment, the therapeutic nanoparticles comprise at least one amine-containing polymer such as, but not limited to polylysine, polyethylene imine, poly(amidoamine) dendrimers, poly(beta-amino esters) (See e.g., U.S. Pat. No. 8,287,849) and combinations thereof.

In another embodiment, the nanoparticles described herein comprise an amine cationic lipid such as those described in International Patent Application No. WO2013059496 and U.S. Patent Application Publ. No. US20140371293. In one aspect the cationic lipids have an amino-amine or an amino-amide moiety.

In one embodiment, the therapeutic nanoparticles comprise at least one degradable polyester which can contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters can include a PEG conjugation to form a PEGylated polymer.

In another embodiment, the therapeutic nanoparticle include a conjugation of at least one targeting ligand. The targeting ligand can be any ligand known in the art such as, but not limited to, a monoclonal antibody. (Kirpotin et al, Cancer Res. 2006 66:6732-6740).

In one embodiment, the therapeutic nanoparticle is formulated in an aqueous solution which can be used to target cancer (see International Publ. No. WO2011084513 and U.S. Patent Application Publ. No. US20110294717).

In one embodiment, the one or more mRNAs encoding an antibody or an antigen binding portion thereof which specifically binds to CTLA-4 is formulated using the methods described in U.S. Pat. No. 8,404,799.

In one embodiment, the one or more mRNAs encoding an antibody or an antigen binding portion thereof which specifically binds to CTLA-4 is encapsulated in, linked to and/or associated with synthetic nanocarriers. Synthetic nanocarriers include, but are not limited to, those described in International Publ. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and U.S. Patent Application Publ. Nos. US20110262491, US20100104645, US20100087337 and US20120244222. The synthetic nanocarriers can be formulated using methods known in the art and/or described herein. As a non-limiting example, the synthetic nanocarriers can be formulated by the methods described in International Publ. Nos. WO2010005740, WO2010030763 and WO201213501and U.S. Patent Application Publ. Nos. US20110262491, US20100104645, US20100087337 and US2012024422. In another embodiment, the synthetic nanocarrier formulations can be lyophilized by methods described in International Publ. No. WO2011072218 and U.S. Pat. No. 8,211,473. In yet another embodiment, formulations of the present invention, including, but not limited to, synthetic nanocarriers, can be lyophilized or reconstituted by the methods described in U.S. Patent Application Publication No. US20130230568.

In one embodiment, the synthetic nanocarriers contain reactive groups to release the polynucleotides described herein (see International Publ. No. WO20120952552 and U.S. Patent Application Publ. No. US20120171229).

In one embodiment, the synthetic nanocarriers contain an immunostimulatory agent to enhance the immune response from delivery of the synthetic nanocarrier. As a non-limiting example, the synthetic nanocarrier can comprise a Th1 immunostimulatory agent which can enhance a Th1-based response of the immune system (see International Pub No. WO2010123569 and U.S. Patent Application Publ. No. US20110223201).

In one embodiment, the synthetic nanocarriers are formulated for targeted release. In one embodiment, the synthetic nanocarrier is formulated to release the polynucleotides at a specified pH and/or after a desired time interval. As a non-limiting example, the synthetic nanoparticle are formulated to release the polynucleotides after 24 hours and/or at a pH of 4.5 (see International Publ. Nos. WO2010138193 and WO2010138194 and U.S. Patent Application Publ. Nos. US20110020388 and US20110027217).

In one embodiment, the synthetic nanocarriers are formulated for controlled and/or sustained release of the polynucleotides described herein. As a non-limiting example, the synthetic nanocarriers for sustained release are formulated by methods known in the art, described herein and/or as described in International Publ. No. WO2010138192 and U.S. Patent Application Publ. No. US20100303850.

In one embodiment, the one or more mRNAs encoding an antibody or an antigen binding portion thereof which specifically binds to CTLA-4 are formulated for controlled and/or sustained release wherein the formulation comprises at least one polymer that is a crystalline side chain (CYSC) polymer. CYSC polymers are described in U.S. Pat. No. 8,399,007.

In one embodiment, the one or more mRNAs encoding an antibody or an antigen binding portion thereof which specifically binds to CTLA-4 are encapsulated in, linked to and/or associated with zwitterionic lipids. Non-limiting examples of zwitterionic lipids and methods of using zwitterionic lipids are described in U.S. Patent Application Publication No. US20130216607. In one aspect, the zwitterionic lipids can be used in the liposomes and lipid nanoparticles described herein.

In one embodiment, the one or more mRNAs encoding an antibody or an antigen binding portion thereof which specifically binds to CTLA-4 are formulated in colloid nanocarriers as described in U.S. Patent Application Publication No. US20130197100.

In one embodiment, the nanoparticle is optimized for oral administration. The nanoparticle can comprise at least one cationic biopolymer such as, but not limited to, chitosan or a derivative thereof. As a non-limiting example, the nanoparticle can be formulated by the methods described in U.S. Patent Appl. Pub. No. US20120282343.

In some embodiments, LNPs comprise the lipid. KL52 (an amino-lipid disclosed in U.S. Application Publication No. US20120295832). Activity and/or safety (as measured by examining one or more of ALT/AST, white blood cell count and cytokine induction) of LNP administration can be improved by incorporation of such lipids. LNPs comprising KL52 can be administered intravenously and/or in one or more doses. In some embodiments, administration of LNPs comprising KL52 results in equal or improved. mRNA and/or protein expression as compared to LNPs comprising MC3.

In some embodiments, the one or more mRNAs encoding an antibody or an antigen binding portion thereof which specifically binds to CTLA-4 are delivered using smaller LNPs. Such particles can comprise a diameter from below 0.1 um up to 100 nm such as, but not limited to, less than 0.1 um, less than 1.0 um, less than 5 um, less than 10 um, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

In another embodiment, the one or more mRNAs encoding an antibody or an antigen binding portion thereof which specifically binds to CTLA-4 are delivered using smaller LNPs which can comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 um to about 30 nm, from about 5 nm to about 40 nm, from about 5 nm to about 50 nm, from about 5 nm to about 60 nm, from about 5 nm to about 70 nm, from about 5 nm to about 80 nm, from about 5 um to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 um, from about 60 to about 70 nm, from about 20 to about 80 nm, from about 30 to about 80 nm, from about 40 to about 80 nm, from about 50 to about 80 nm, from about 60 to about 80 nm, from about 20 to about 90 um, from about 30 to about 90 nm, from about 40 to about 90 nm, from about 50 to about 90 nm, from about 60 to about 90 nm and/or from about 70 to about 90 nm.

In some embodiments, such LNPs are synthesized using methods comprising microfluidic mixers. Exemplary microfluidic mixers can include, but are not limited to a slit interdigitial micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1:e37; Chen, D. et al., Rapid discovery of potent siRNA-containing lipid nanoparticles enabled by controlled microfluidic formulation. J Am Chem Soc. 2012. 134(16):6948-51). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Patent Application Publication Nos. US20040262223 and US20120276209.

In one embodiment, the one or more mRNAs encoding an antibody or an antigen binding portion thereof which specifically binds to CTLA-4 of the present invention is formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany).

In one embodiment, the one or more mRNAs encoding an antibody or an antigen binding portion thereof which specifically binds to CTLA-4 of the present invention are formulated in lipid nanoparticles created using microfluidic technology (see Whitesides, George M. The Origins and the Future of Microfluidics. Nature, 2006 442: 368-373; and Abraham et al. Chaotic Mixer for Microchannels. Science, 2002 295: 647-651). As a non-limiting example, controlled microfluidic formulation includes a passive method for mixing streams of steady pressure-driven flows in micro channels at a low Reynolds number (See e.g., Abraham et al. Chaotic Mixer for Microchannels. Science, 2002 295: 647-651).

In one embodiment, the one or more mRNAs encoding an antibody or an antigen binding portion thereof which specifically binds to CTLA-4 can be formulated in lipid nanoparticles created using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In one embodiment, the one or more mRNAs encoding an antibody or an antigen binding portion thereof which specifically binds to CTLA-4 are formulated for delivery using the drug encapsulating microspheres described in International Patent Publication No. WO2013063468 or U.S. Pat. No. 8,440,614. The microspheres can comprise a compound of the formula (I), (II), (III), (IV), (V) or (VI) as described in International Patent Publication No. WO2013063468. In another aspect, the amino acid, peptide, polypeptide, lipids (APPL) are useful in delivering the polynucleotides of the invention to cells (see International Patent Publication No. WO2013063468 and U.S. Patent Appl. Publ. No. US20130158021).

In one embodiment, the one or more mRNAs encoding an antibody or an antigen binding portion thereof which specifically binds to CTLA-4 are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the lipid nanoparticles have a diameter from about 10 to 500 nm. In one embodiment, the lipid nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In one aspect, the lipid nanoparticle is a limit size lipid nanoparticle described in International Patent Publication No. WO2013059922 and U.S. Patent Appl. Publ. No US20140328759. The limit size lipid nanoparticle can comprise a lipid bilayer surrounding an aqueous core or a hydrophobic core; where the lipid bilayer can comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In another aspect the limit size lipid nanoparticle can comprise a polyethylene glycol-lipid such as, but not limited to, DLPE-PEG, DMPE-PEG, DPPC-PEG and DSPE-PEG.

In one embodiment, the one or more mRNAs encoding an antibody or an antigen binding portion thereof which specifically binds to CTLA-4 are delivered, localized and/or concentrated in a specific location using the delivery methods described in International Patent Publication No. WO2013063530 and U.S. Patent Appl Publ. No. US20140323907. As a non-limiting example, a subject can be administered an empty polymeric particle prior to, simultaneously with or after delivering the polynucleotides to the subject. The empty polymeric particle undergoes a change in volume once in contact with the subject and becomes lodged, embedded, immobilized or entrapped at a specific location in the subject.

In one embodiment, the one or more mRNAs encoding an antibody or an antigen binding portion thereof which specifically binds to CTLA-4 are formulated in an active substance release system (See e.g., U.S. Patent Application Publication No. US20130102545). The active substance release system can comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In one embodiment, the polynucleotides comprising an mRNA encoding an antibody or antigen binding portion thereof which specifically binds to CTLA-4 are formulated in a nanoparticle comprising an inner core comprising a non-cellular material and an outer surface comprising a cellular membrane. The cellular membrane can be derived from a cell or a membrane derived from a virus. As a non-limiting example, the nanoparticle is made by the methods described in international Patent Publication No. WO2013052167 and U.S. Patent Appl. Publ. No. US20130337066. As another non-limiting example, the nanoparticle described in international Patent Publication No. WO2013052167, is used to deliver the polynucleotides described herein.

In one embodiment, the one or more mRNAs encoding an antibody or an antigen binding portion thereof which specifically binds to CTLA-4 are formulated in porous nanoparticle-supported lipid bilayers (protocells). Protocells are described in International Patent Publication No. WO2013056132 and U.S. Patent Appl. Publ. No. US20150272885.

In one embodiment, the one or more mRNAs encoding an antibody or an antigen binding portion thereof which specifically binds to CTLA-4 described herein are formulated in polymeric nanoparticles as described in or made by the methods described in U.S. Pat. Nos. 8,420,123 and 8,518,963 and European Patent No. EP2073848B1. As a non-limiting example, the polymeric nanoparticle has a high glass transition temperature such as the nanoparticles described in or nanoparticles made by the methods described in U.S. Pat. No. 8,518,963. As another non-limiting example, the polymer nanoparticle for oral and parenteral formulations is made by the methods described in European Patent No. EP2073848B1 and U.S. Pat. No. 8,715,741.

In another embodiment, the one or more mRNAs encoding an antibody or an antigen binding portion thereof which specifically binds to CTLA-4 described herein are formulated in nanoparticles used in imaging. The nanoparticles can be liposome nanoparticles such as those described in U.S. Patent Publication No US20130129636. As a non-limiting example, the liposome can comprise gadolinium(III)2-{4,7-bis-carboxymethyl-10-[(N,N-distearylamidomethyl-N′-amidomethyl]-1,4,7,10-tetra-azacyclododec-1-yl}-acetic acid and a neutral, fully saturated phospholipid component (see e.g., US Patent Publication No US20130129636).

In one embodiment, the nanoparticles which can be used in the present invention are formed by the methods described in U.S. Patent Application Publ. No. US20130130348.

The nanoparticles of the present invention can further include nutrients such as, but not limited to, those which deficiencies can lead to health hazards from anemia to neural tube defects (see e.g., the nanoparticles described in International Patent Publication No WO2013072929 and U.S. Patent Appl. Publ. No. US20150224035). As a non-limiting example, the nutrient is iron in the form of ferrous, ferric salts or elemental iron, iodine, folic acid, vitamins or micronutrients.

In one embodiment, the one or more mRNAs encoding an antibody or an antigen binding portion thereof which specifically binds to CTLA-4 are formulated in a swellable nanoparticle. The swellable nanoparticle can be, but is not limited to, those described in U.S. Pat. No. 8,440,231. As a non-limiting embodiment, the swellable nanoparticle is used for delivery of the polynucleotides of the present invention to the pulmonary system (see e.g., U.S. Pat. No. 8,440,231).

The one or more mRNAs encoding an antibody or an antigen binding portion thereof which specifically binds to CTLA-4 are formulated in polyanhydride nanoparticles such as, but not limited to, those described in U.S. Pat. No. 8,449,916.

The nanoparticles and microparticles of the present invention can be geometrically engineered to modulate macrophage and/or the immune response. In one aspect, the geometrically engineered particles can have varied shapes, sizes and/or surface charges in order to incorporated the polynucleotides of the present invention for targeted delivery such as, but not limited to, pulmonary delivery (see e.g., International Publication No WO2013082111 and U.S. Patent Appl. Publ. No. US20150037428). Other physical features the geometrically engineering particles can have include, but are not limited to, fenestrations, angled arms, asymmetry and surface roughness, charge which can alter the interactions with cells and tissues. As a non-limiting example, nanoparticles of the present invention are made by the methods described in International Publication No WO2013082111.

In one embodiment, the nanoparticles of the present invention are water soluble nanoparticles such as, but not limited to, those described in International Publication No. WO2013090601 and U.S. Pat. No. 9,078,920. The nanoparticles can be inorganic nanoparticles which have a compact and zwitterionic ligand in order to exhibit good water solubility. The nanoparticles can also have small hydrodynamic diameters (HD), stability with respect to time, pH, and salinity and a low level of non-specific protein binding.

In one embodiment the nanoparticles of the present invention are developed by the methods described in US Patent Publication No. US20130172406.

In one embodiment, the nanoparticles of the present invention are stealth nanoparticles or target-specific stealth nanoparticles such as, but not limited to, those described in US Patent Publication No. US20130172406. The nanoparticles of the present invention can be made by the methods described in US Patent Publication No. US20130172406.

In another embodiment, the stealth or target-specific stealth nanoparticles comprise a polymeric matrix. The polymeric matrix can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In one embodiment, the nanoparticle is a nanoparticle-nucleic acid hybrid structure having a high density nucleic acid layer. As a non-limiting example, the nanoparticle-nucleic acid hybrid structure is made by the methods described in US Patent Publication No. US20130171646. The nanoparticle can comprise a nucleic acid such as, but not limited to, polynucleotides described herein and/or known in the art.

At least one of the nanoparticles of the present invention can be embedded in in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Patent Publication No. WO2013123523 and U.S. Patent Application Publ. No. US20150037249.

5. Hyaluronidase

The injection, e.g., intramuscular, intratumoral, or subcutaneous localized injection, of one or more mRNAs encoding an antibody or an antigen binding portion thereof which specifically binds to CTLA-4 can include hyaluronidase, which catalyzes the hydrolysis of hyaluronan. By catalyzing the hydrolysis of hyaluronan, a constituent of the interstitial barrier, hyaluronidase lowers the viscosity of hyaluronan, thereby increasing tissue permeability (Frost, Expert Opin. Drug Deliv. (2007) 4:427-440). It is useful to speed their dispersion and systemic distribution of encoded proteins produced by transfected cells. Alternatively, the hyaluronidase can be used to increase the number of cells exposed to a polynucleotide of the invention administered intramuscularly, intratumorally, or subcutaneously.

6. Nanoparticle Mimics

The one or more mRNAs encoding an antibody or an antigen binding portion thereof which specifically binds to CTLA-4 can be encapsulated within and/or absorbed to a nanoparticle mimic. A nanoparticle mimic can mimic the delivery function organisms or particles such as, but not limited to, pathogens, viruses, bacteria, fungus, parasites, prions and cells. As a non-limiting example the polynucleotides of the invention can be encapsulated in a non-virion particle which can mimic the delivery function of a virus (see International Pub. No. WO2012006376 and U.S. Patent Application Publication Nos. US20130171241 and US20130195968).

7. Nanotubes

The one or more mRNAs encoding an antibody or an antigen binding portion thereof which specifically binds to CTLA-4 can be attached or otherwise bound to at least one nanotube such as, but not limited to, rosette nanotubes, rosette nanotubes having twin bases with a linker, carbon nanotubes and/or single-walled carbon nanotubes. The polynucleotides can be bound to the nanotubes through forces such as, but not limited to, steric, ionic, covalent and/or other forces. Nanotubes and nanotube formulations comprising polynucleotides are described in International Patent Application Publ. No. WO2014152211.

8. Self-Assembled Nanoparticles

The one or more mRNAs encoding an antibody or an antigen binding portion thereof which specifically binds to CTLA-4 can be formulated in self-assembled nanoparticles. Nucleic acid self-assembled nanoparticles are described in International Patent Application Publ. No. WO2014152211, such as in paragraphs [000740]-[000743]. Polymer-based self-assembled nanoparticles are described in International Patent Application Publ. No. WO2014152211.

9. Self-Assembled Macromolecules

The one or more mRNAs encoding an antibody or an antigen binding portion thereof which specifically binds to CTLA-4 can be formulated in amphiphilic macromolecules (AMs) for delivery. AMs comprise biocompatible amphiphilic polymers which have an alkylated sugar backbone covalently linked to poly(ethylene glycol). In aqueous solution, the AMs self-assemble to form micelles. Non-limiting examples of methods of forming AMs and AMs are described in US Patent Publication No. US20130217753.

10. Inorganic Nanoparticles

The one or more mRNAs encoding an antibody or an antigen binding portion thereof which specifically binds to CTLA-4 can be formulated in inorganic nanoparticles (U.S. Pat. No. 8,257,745). The inorganic nanoparticles can include, but are not limited to, clay substances that are water swellable. As a non-limiting example, the inorganic nanoparticle includes synthetic smectite clays which are made from simple silicates (See e.g., U.S. Pat. No. 5,585,108 and 8,257,745).

In some embodiments, the inorganic nanoparticle comprises a core of the polynucleotides disclosed herein and a polymer shell. The polymer shell can be any of the polymers described herein and are known in the art. In an additional embodiment, the polymer shell can be used to protect the polynucleotides in the core.

11. Semi-Conductive and Metallic Nanoparticles

The one or more mRNAs encoding an antibody or an antigen binding portion thereof which specifically binds to CTLA-4 can be formulated in water-dispersible nanoparticle comprising a semiconductive or metallic material (U.S. Patent Application Publ. No. US20120228565) or formed in a magnetic nanoparticle (U.S. Patent Application Publ. Nos. US20120265001 and US20120283503). The water-dispersible nanoparticles can be hydrophobic nanoparticles or hydrophilic nanoparticles.

In some embodiments, the semi-conductive and/or metallic nanoparticles can comprise a core of the polynucleotides disclosed herein and a polymer shell. The polymer shell can be any of the polymers described herein and are known in the art. In an additional embodiment, the polymer shell can be used to protect the polynucleotides in the core.

12. Surgical Sealants: Gels and Hydrogels

In some embodiments, the one or more mRNAs encoding an antibody or an antigen binding portion thereof which specifically binds to CTLA-4 are encapsulated into any hydrogel known in the art which forms a gel when injected into a subject. Surgical sealants such as gels and hydrogels are described in International Patent Application Publication No. WO2014152211.

13. Suspension Formulations

In some embodiments, suspension formulations are provided comprising polynucleotides, water immiscible oil depots, surfactants and/or co-surfactants and/or co-solvents. Combinations of oils and surfactants can enable suspension formulation with polynucleotides. Delivery of polynucleotides in a water immiscible depot can be used to improve bioavailability through sustained release of mRNA from the depot to the surrounding physiologic environment and prevent polynucleotides degradation by nucleases.

In some embodiments, suspension formulations of mRNA are prepared using combinations of polynucleotides, oil-based solutions and surfactants. Such formulations can be prepared as a two-part system comprising an aqueous phase comprising polynucleotides and an oil-based phase comprising oil and surfactants. Exemplary oils for suspension formulations can include, but are not limited to sesame oil and Miglyol (comprising esters of saturated coconut and palmkernel oil-derived caprylic and capric fatty acids and glycerin or propylene glycol), corn oil, soybean oil, peanut oil, beeswax and/or palm seed oil. Exemplary surfactants can include, but are not limited to Cremophor, polysorbate 20, polysorbate 80, polyethylene glycol, transcutol, CAPMUL®, labrasol, isopropyl myristate, and/or Span 80. In some embodiments, suspensions can comprise co-solvents including, but not limited to ethanol, glycerol and/or propylene glycol.

Suspensions can be formed by first preparing polynucleotides formulation comprising an aqueous solution of polynucleotide and an oil-based phase comprising one or more surfactants. Suspension formation occurs as a result of mixing the two phases (aqueous and oil-based). In some embodiments, such a suspension can be delivered to an aqueous phase to form an oil-in-water emulsion. In some embodiments, delivery of a suspension to an aqueous phase results in the formation of an oil-in-water emulsion in which the oil-based phase comprising polynucleotides forms droplets that can range in size from nanometer-sized droplets to micrometer-sized droplets. In some embodiments, specific combinations of oils, surfactants, cosurfactants and/or co-solvents can be utilized to suspend polynucleotides in the oil phase and/or to form oil-in-water emulsions upon delivery into an aqueous environment.

In some embodiments, suspensions provide modulation of the release of polynucleotides into the surrounding environment. In such embodiments, polynucleotides release can be modulated by diffusion from a water immiscible depot followed by resolubilization into a surrounding environment (e.g. an aqueous environment).

In some embodiments, polynucleotides within a water immiscible depot (e.g. suspended within an oil phase) result in altered polynucleotides stability (e.g. altered degradation by nucleases).

In some embodiments, the one or more mRNAs encoding an antibody or an antigen binding portion thereof which specifically binds to CTLA-4 are formulated such that upon injection, an emulsion forms spontaneously (e.g. when delivered to an aqueous phase). Such particle formation can provide a high surface area to volume ratio for release of polynucleotides from an oil phase to an aqueous phase.

In some embodiments, the one or more mRNAs encoding an antibody or an antigen binding portion thereof which specifically binds to CTLA-4 are formulated in a nanoemulsion such as, but not limited to, the nanoemulsions described in U.S. Pat. No. 8,496,945. The nanoemulsions can comprise nanoparticles described herein. As a non-limiting example, the nanoparticles can comprise a liquid hydrophobic core which can be surrounded or coated with a lipid or surfactant layer. The lipid or surfactant layer can comprise at least one membrane-integrating peptide and can also comprise a targeting ligand (see e.g., U.S. Pat. No. 8,496,945).

14. Cations and Anions

Formulations of one or more mRNAs encoding an antibody or an antigen binding portion thereof which specifically binds to CTLA-4 can include cations or anions. In some embodiments, the formulations include metal cations such as, but not limited to, Zn²⁺, Ca²⁺, Cu²⁺, Mg²⁺ and combinations thereof. As a non-limiting example, formulations include polymers and a polynucleotides complexed with a metal cation (See e.g., U.S. Pat. Nos. 6,265,389 and 6,555,525).

In some embodiments, cationic nanoparticles comprising combinations of divalent and monovalent cations are formulated with polynucleotides. Such nanoparticles can form spontaneously in solution over a given period (e.g. hours, days, etc). Such nanoparticles do not form in the presence of divalent cations alone or in the presence of monovalent cations alone. The delivery of polynucleotides in cationic nanoparticles or in one or more depot comprising cationic nanoparticles can improve polynucleotide bioavailability by acting as a long-acting depot and/or reducing the rate of degradation by nucleases.

15. Molded Nanoparticles and Microparticles

The one or more mRNAs encoding an antibody or an antigen binding portion thereof which specifically binds to CTLA-4 can be formulated in nanoparticles and/or microparticles. As an example, the nanoparticles and/or microparticles can be made using the PRINT® technology by LIQUIDA TECHNOLOGIES® (Morrisville, N.C.) (See, e.g., International Pub. No. WO2007024323 and U.S. Patent Publication No. US20110182805).

In some embodiments, the nanoparticles comprise a core of the polynucleotides disclosed herein and a polymer shell. The polymer shell can be any of the polymers described herein and are known in the art. In an additional embodiment, the polymer shell can be used to protect the polynucleotides in the core.

In some embodiments, the one or more mRNAs encoding an antibody or an antigen binding portion thereof which specifically binds to CTLA-4 are formulated in microparticles. The microparticles can contain a core of the polynucleotides and a cortex of a biocompatible and/or biodegradable polymer. As a non-limiting example, the microparticles which can be used with the present invention can be those described in U.S. Pat. No. 8,460,709, U.S. Patent Publication No. US20130129830 and International Patent Publication No WO2013075068. As another non-limiting example, the microparticles can be designed to extend the release of the polynucleotides of the present invention over a desired period of time (see e.g., extended release of a therapeutic protein in U.S. Patent Publication No. US20130129830).

16. NanoJackets and NanoLiposomes

The one or more mRNAs encoding an antibody or an antigen binding portion thereof which specifically binds to CTLA-4 can be formulated in NanoJackets and NanoLiposomes by Keystone Nano (State College, Pa.). NanoJackets are made of compounds that are naturally found in the body including calcium, phosphate and can also include a small amount of silicates. Nanojackets can range in size from 5 to 50 nm and can be used to deliver hydrophilic and hydrophobic compounds such as, but not limited to, polynucleotides.

NanoLiposomes are made of lipids such as, but not limited to, lipids which naturally occur in the body. NanoLiposomes can range in size from 60-80 nm and can be used to deliver hydrophilic and hydrophobic compounds such as, but not limited to, polynucleotides.

In one aspect, the one or more mRNAs encoding an antibody or an antigen binding portion thereof which specifically binds to CTLA-4 are formulated in a NanoLiposome such as, but not limited to, Ceramide NanoLiposomes.

17. Minicells

In one aspect, the one or more mRNAs encoding an antibody or an antigen binding portion thereof which specifically binds to CTLA-4 can be formulated in bacterial minicells. As a non-limiting example, bacterial minicells are those described in International Publication No. WO2013088250 or U.S. Patent Publication No. US20130177499. The bacterial minicells comprising therapeutic agents such as polynucleotides described herein can be used to deliver the therapeutic agents to brain tumors.

18. Semi-Solid Compositions

In some embodiments, the one or more mRNAs encoding an antibody or an antigen binding portion thereof which specifically binds to CTLA-4 are formulated with a hydrophobic matrix to form a semi-solid composition. As a non-limiting example, the semi-solid composition or paste-like composition is made by the methods described in International Patent Publication No. WO2013079604. The semi-solid composition can be a sustained release formulation as described in International Patent Publication No WO2013079604.

In another embodiment, the semi-solid composition further has a micro-porous membrane or a biodegradable polymer formed around the composition (see e.g., International Patent Publication No. WO2013079604).

The semi-solid composition using the one or more mRNAs encoding an antibody or an antigen binding portion thereof which specifically binds to CTLA-4 can have the characteristics of the semi-solid mixture as described in International Patent Publication No WO2013079604 (e.g., a modulus of elasticity of at least 10⁻⁴ N·mm⁻², and/or a viscosity of at least 100 mPa·s).

19. Exosomes

In some embodiments, the one or more mRNAs encoding an antibody or an antigen binding portion thereof which specifically binds to CTLA-4 are formulated in exosomes. The exosomes can be loaded with at least one polynucleotide and delivered to cells, tissues and/or organisms. As a non-limiting example, the one or more mRNAs encoding an antibody or an antigen binding portion thereof which specifically binds to CTLA-4 can be loaded in the exosomes described in International Publication No. WO2013084000 and U.S. Patent Publication No. US20140356382.

20. Silk-Based Delivery

In some embodiments, the one or more mRNAs encoding an antibody or an antigen binding portion thereof which specifically binds to CTLA-4 are formulated in a sustained release silk-based delivery system. The silk-based delivery system can be formed by contacting a silk fibroin solution with a therapeutic agent such as, but not limited to, the one or more mRNAs encoding an antibody or an antigen binding portion thereof which specifically binds to CTLA-4. As a non-limiting example, the sustained release silk-based delivery system which can be used in the present invention and methods of making such system are described in U.S. Patent Publication No. US20130177611.

21. Microparticles

In some embodiments, formulations comprising one or more mRNAs encoding an antibody or an antigen binding portion thereof which specifically binds to CTLA-4 comprise microparticles. The microparticles can comprise a polymer described herein and/or known in the art such as, but not limited to, poly(a-hydroxy acid), a polyhydroxy butyric acid, a polycaprolactone, a polyorthoester and a polyanhydride. The microparticle can have adsorbent surfaces to adsorb biologically active molecules such as polynucleotides. As a non-limiting example microparticles for use with the present invention and methods of making microparticles are described in U.S. Patent Publication No. US2013195923 and US20130195898 and U.S. Pat. Nos. 8,309,139 and 8,206,749.

In another embodiment, the formulation is a microemulsion comprising microparticles and polynucleotides. As a non-limiting example, microemulsions comprising microparticles are described in U.S. Patent Publication No. US2013195923 and US20130195898 and U.S. Pat. Nos. 8,309,139 and 8,206,749.

22. Amino Acid Lipids

In some embodiments, the one or more mRNAs encoding an antibody or an antigen binding portion thereof which specifically binds to CTLA-4 are formulated in amino acid lipids. Amino acid lipids are lipophilic compounds comprising an amino acid residue and one or more lipophilic tails. Non-limiting examples of amino acid lipids and methods of making amino acid lipids are described in U.S. Pat. No. 8,501,824.

In some embodiments, the amino acid lipids have a hydrophilic portion and a lipophilic portion. The hydrophilic portion can be an amino acid residue and a lipophilic portion can comprise at least one lipophilic tail.

In some embodiments, the amino acid lipid formulations are used to deliver the polynucleotides to a subject.

In another embodiment, the amino acid lipid formulations deliver a polynucleotide in releasable form which comprises an amino acid lipid that binds and releases the polynucleotides. As a non-limiting example, the release of the polynucleotides can be provided by an acid-labile linker such as, but not limited to, those described in U.S. Pat. Nos. 7,098,032, 6,897,196, 6,426,086, 7,138,382, 5,563,250, and 5,505,931, which are herein incorporated by reference in their entireties.

23. Microvesicles

In some embodiments, the one or more mRNAs encoding an antibody or an antigen binding portion thereof which specifically binds to CTLA-4 are formulated in microvesicles. Non-limiting examples of microvesicles include those described in US Patent Publication No. US20130209544, which is herein incorporated by reference in its entirety.

In some embodiments, the microvesicle is an ARRDC1-mediated microvesicles (ARMMs). Non-limiting examples of ARMMs and methods of making ARMMs are described in International Patent Publication No. WO2013119602 and US Patent Publication No. US20150037421.

24. Interpolyelectrolyte Complexes

In some embodiments, the one or more mRNAs encoding an antibody or an antigen binding portion thereof which specifically binds to CTLA-4 are formulated in an interpolyelectrolyte complex. Interpolyelectrolyte complexes are formed when charge-dynamic polymers are complexed with one or more anionic molecules. Non-limiting examples of charge-dynamic polymers and interpolyelectrolyte complexes and methods of making interpolyelectrolyte complexes are described in U.S. Pat. No. 8,524,368.

25. Crystalline Polymeric Systems

In some embodiments, the one or more mRNAs encoding an antibody or an antigen binding portion thereof which specifically binds to CTLA-4 are formulated in crystalline polymeric systems. Crystalline polymeric systems are polymers with crystalline moieties and/or terminal units comprising crystalline moieties. Non-limiting examples of polymers with crystalline moieties and/or terminal units comprising crystalline moieties termed “CYC polymers,” crystalline polymer systems and methods of making such polymers and systems are described in U.S. Pat. No. 8,524,259.

26. Excipients

Pharmaceutical formulations can additionally comprise a pharmaceutically acceptable excipient, which, as used herein, includes, but are not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, antioxidants, osmolality adjusting agents, pH adjusting agents and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro (Lippincott, Williams & Wilkins, Baltimore, Md., 2006). The use of a conventional excipient medium can be contemplated within the scope of the present disclosure, except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use for humans and for veterinary use. In some embodiments, an excipient can be approved by United States Food and Drug Administration. In some embodiments, an excipient can be of pharmaceutical grade. In some embodiments, an excipient can meet the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients can optionally be included in pharmaceutical compositions. The composition can also include excipients such as cocoa butter and suppository waxes, coloring agents, coating agents, sweetening, flavoring, and/or perfuming agents.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); amino acids (e.g., glycine); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives can include, but are not limited to, antioxidants, chelating agents, antimicrobial preservatives, antifungal preservatives, alcohol preservatives, acidic preservatives, and/or other preservatives. Oxidation is a potential degradation pathway for mRNA, especially for liquid mRNA formulations. In order to prevent oxidation, antioxidants can be added to the formulation. Exemplary antioxidants include, but are not limited to, alpha tocopherol, ascorbic acid, acorbyl palmitate, benzyl alcohol, butylated hydroxyanisole, EDTA, m-cresol, methionine, butylated hydroxytoluene, monothioglycerol, potassium metabisulfite, propionic acid, propyl gallate, sodium ascorbate, sodium bisulfite, sodium metabisulfite, thioglycerol and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

In some embodiments, the pH of polynucleotide solutions is maintained between pH 5 and pH 8 to improve stability. Exemplary buffers to control pH can include, but are not limited to sodium phosphate, sodium citrate, sodium succinate, histidine (or histidine-HCl), sodium carbonate, and/or sodium malate. In another embodiment, the exemplary buffers listed above can be used with additional monovalent counterions (including, but not limited to potassium). Divalent cations can also be used as buffer counterions; however, these are not preferred due to complex formation and/or mRNA degradation.

Exemplary buffering agents can also include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

Exemplary additives include physiologically biocompatible buffers (e.g., trimethylamine hydrochloride), addition of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). In addition, antioxidants and suspending agents can be used.

27. Cryoprotectants for mRNA

In some embodiments, the polynucleotide formulations comprise cyroprotectants. As used herein, the term “cryoprotectant” refers to one or more agent that when combined with a given substance, helps to reduce or eliminate damage to that substance that occurs upon freezing. In some embodiments, cryoprotectants are combined with polynucleotides in order to stabilize them during freezing. Frozen storage of mRNA between −20° C. and −80° C. can be advantageous for long term (e.g. 36 months) stability of polynucleotide. In some embodiments, cryoprotectants are included in polynucleotide formulations to stabilize polynucleotide through freeze/thaw cycles and under frozen storage conditions. Cryoprotectants of the present invention can include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

28. Bulking Agents

In some embodiments, the polynucleotide formulations comprise bulking agents. As used herein, the term “bulking agent” refers to one or more agents included in formulations to impart a desired consistency to the formulation and/or stabilization of formulation components. In some embodiments, bulking agents are included in lyophilized polynucleotide formulations to yield a “pharmaceutically elegant” cake, stabilizing the lyophilized polynucleotides during long term (e.g. 36 month) storage. Bulking agents of the present invention can include, but are not limited to sucrose, trehalose, mannitol, glycine, lactose and/or raffinose. In some embodiments, combinations of cryoprotectants and bulking agents (for example, sucrose/glycine or trehalose/mannitol) can be included to both stabilize polynucleotides during freezing and provide a bulking agent for lyophilization.

Non-limiting examples of formulations and methods for formulating the polynucleotides of the present invention are also provided in International Publication No WO2013090648 and U.S. Pat. No. 8,664,194.

29. Naked Delivery

The one or more mRNAs encoding an antibody or an antigen binding portion thereof which specifically binds to CTLA-4 can be delivered to a cell (e.g., to a tumor cell) naked. As used herein in, “naked” refers to delivering polynucleotides free from agents which promote transfection. For example, the polynucleotides delivered to the cell, e.g., tumor cell, can contain no modifications. The naked one or more mRNAs encoding an antibody or an antigen binding portion thereof which specifically binds to CTLA-4 can be delivered to the tumor cell using routes of administration known in the art, e.g., intratumoral administration, and described herein.

30. Parenteral and Injectable Administration

Liquid dosage forms for parenteral administration, e.g. intratumoral, include, but are not limited to, pharmaceutically acceptable emulsions, microemulsions, solutions, suspensions, syrups, and/or elixirs. In addition to active ingredients, liquid dosage forms can comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

A pharmaceutical composition for parenteral administration, e.g., intratumoral administration, can comprise at least one inactive ingredient. Any or none of the inactive ingredients used can have been approved by the US Food and Drug Administration (FDA). A non-exhaustive list of inactive ingredients for use in pharmaceutical compositions for parenteral administration includes hydrochloric acid, mannitol, nitrogen, sodium acetate, sodium chloride and sodium hydroxide.

Injectable preparations, for example, sterile injectable aqueous or oleaginous suspensions can be formulated according to the known art using suitable dispersing agents, wetting agents, and/or suspending agents. Sterile injectable preparations can be sterile injectable solutions, suspensions, and/or emulsions in nontoxic parenterally acceptable diluents and/or solvents, for example, as a solution in 1,3-butanediol. Among the acceptable vehicles and solvents that can be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. The sterile formulation can also comprise adjuvants such as local anesthetics, preservatives and buffering agents.

Injectable formulations, e.g., intratumoral, can be sterilized, for example, by filtration through a bacterial-retaining filter, and/or by incorporating sterilizing agents in the form of sterile solid compositions which can be dissolved or dispersed in sterile water or other sterile injectable medium prior to use.

Injectable formulations, e.g., intratumoral, can be for direct injection into a region of a tissue, organ and/or subject, e.g., tumor.

In order to prolong the effect of an active ingredient, it is often desirable to slow the absorption of the active ingredient from intratumoral injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

31. Dosage Forms

A pharmaceutical composition described herein can be formulated into a dosage form described herein, such as a topical, intranasal, intratracheal, or injectable (e.g., intravenous, intratumoral, intraocular, intravitreal, intramuscular, intracardiac, intraperitoneal, subcutaneous).

32. Liquid Dosage Forms

Liquid dosage forms for parenteral administration (e.g., intratumoral) include, but are not limited to, pharmaceutically acceptable emulsions, microemulsions, solutions, suspensions, syrups, and/or elixirs. In addition to active ingredients, liquid dosage forms can comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions can be mixed with solubilizing agents such as CREMOPHOR®, alcohols, oils, modified oils, glycols, polysorbates, cyclodextrins, polymers, and/or combinations thereof.

33. Injectable

Injectable preparations (e.g., intratumoral), for example, sterile injectable aqueous or oleaginous suspensions can be formulated according to the known art and can include suitable dispersing agents, wetting agents, and/or suspending agents. Sterile injectable preparations can be sterile injectable solutions, suspensions, and/or emulsions in nontoxic parenterally acceptable diluents and/or solvents, for example, a solution in 1,3-butanediol. Among the acceptable vehicles and solvents that can be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it can be desirable to slow the absorption of the active ingredient from subcutaneous or intramuscular injection. This can be accomplished by the use of a liquid suspension of crystalline or amorphous material with poor water solubility. The rate of absorption of the polynucleotides then depends upon its rate of dissolution which, in turn, can depend upon crystal size and crystalline form. Alternatively, delayed absorption of parenterally administered polynucleotides can be accomplished by dissolving or suspending the polynucleotides in an oil vehicle. Injectable depot forms are made by forming microencapsule matrices of the polynucleotides in biodegradable polymers such as polylactide-polyglycolide. Depending upon the ratio of polynucleotides to polymer and the nature of the particular polymer employed, the rate of polynucleotides release can be controlled. Examples of other biodegradable polymers include, but are not limited to, poly(orthoesters) and poly(anhydrides). Depot injectable formulations can be prepared by entrapping the polynucleotides in liposomes or microemulsions which are compatible with body tissues.

### VIII. Kits and Devices

1. Kits

The invention provides a variety of kits for conveniently and/or effectively carrying out methods of the present invention. Typically kits will comprise sufficient amounts and/or numbers of components to allow a user to perform multiple treatments of a subjects) and/or to perform multiple experiments.

In one aspect, the present invention provides kits comprising the polynucleotides of the invention. In some embodiments, the kit comprises one or more polynucleotides.

The kits can be for protein production, comprising a one or more mRNAs encoding an antibody or an antigen binding portion thereof which specifically binds to CTLA-4. The kit can further comprise packaging and instructions and/or a delivery agent to form a formulation composition. The delivery agent can comprise a saline, a buffered solution, a lipidoid or any delivery agent disclosed herein.

In some embodiments, the buffer solution includes sodium chloride, calcium chloride, phosphate and/or EDTA. In another embodiment, the buffer solution includes, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See e.g., U.S. Pub. No. 20120258046). In a further embodiment, the buffer solutions is precipitated or it is lyophilized. The amount of each component can be varied to enable consistent, reproducible higher concentration saline or simple buffer formulations. The components can also be varied in order to increase the stability of modified RNA in the buffer solution over a period of time and/or under a variety of conditions. In one aspect, the present invention provides kits for protein production, comprising: a polynucleotide comprising a translatable region, provided in an amount effective to produce a desired amount of a protein encoded by the translatable region when introduced into a target cell; a second polynucleotide comprising an inhibitory nucleic acid, provided in an amount effective to substantially inhibit the innate immune response of the cell; and packaging and instructions.

In one aspect, the present invention provides kits for protein production, comprising a one or more mRNAs encoding an antibody or an antigen binding portion thereof which specifically binds to CTLA-4, wherein the polynucleotide exhibits reduced degradation by a cellular nuclease, and packaging and instructions.

2. Devices

The present invention provides for devices which can incorporate one or more mRNAs encoding an antibody or an antigen binding portion thereof which specifically binds to CTLA-4. These devices contain in a stable formulation the reagents to synthesize a polynucleotide in a formulation available to be immediately delivered to a subject in need thereof, such as a human patient.

Devices for administration can be employed to deliver one or more mRNAs encoding an antibody or an antigen binding portion thereof which specifically binds to CTLA-4 according to single, multi- or split-dosing regimens taught herein. Such devices are taught in, for example, International Publication No. WO2013151666.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

According to the present invention, these multi-administration devices can be utilized to deliver the single, multi- or split doses contemplated herein. Such devices are taught for example in, International Publication No. WO 2013151666.

### IX. Codon Optimized Antibodies

The present disclosure also provides codon-optimized mRNA sequence encoding anti-CTLA-4 antibodies. Compositions comprising these codon optimized mRNAs can be administered to a subject in need thereof to facilitate in vivo expression and assembly of a therapeutic antibody. Each of the codon-optimized nucleotide sequences disclosed herein is not a wild type nucleotide sequence encoding a therapeutic antibody known in the art.

The present disclosure provides polynucleotides comprising codon-optimized mRNA sequences encoding anti-CTLA-4 antibodies which can be used to express the antibodies, for example, in vivo in a host organism (e.g., in a particular tissue or cell). The codon-optimized mRNA sequences presented in the instant disclosure can present improved properties related to expression efficacy after administration in vivo to a subject in need thereof. Such properties include, but are not limited to, improving nucleic acid stability (e.g., mRNA stability), increasing translation efficacy in the target tissue, reducing the number of truncated proteins expressed, improving the folding or prevent misfolding of the expressed proteins, reducing toxicity of the expressed products, reducing cell death caused by the expressed products, increasing or decreasing protein aggregation, etc.

The codon-optimized nucleotide sequences disclosed herein have been optimized for expression in human subjects, having structural and/or chemical features that avoid one or more of the problems in the art, for example, features which are useful for optimizing formulation and delivery of nucleic acid-based therapeutics while retaining structural and functional integrity, overcoming the threshold of expression, improving expression rates, half-life and/or protein concentrations, optimizing protein localization, and avoiding deleterious bio-responses such as the immune response and/or degradation pathways.

Codon optimized sequences are shown in TABLE 9. These codon optimized sequences can be used to practice the methods disclosed elsewhere in the present application, for example, as alternatives or complementing the sequences disclosed in TABLE 1.

TABLE 9
Codon optimized Sequences

| SEQ ID | | |
|---|---|---|
| NO | Construct Name | Description |
| 251 | aCTLA4_Ab1_HC_IgG1_001 | Sequence, NT (5′ UTR, ORF, 3′ UTR) |
| 252 | aCTLA4_Ab1_HC_IgG1_001 | ORF Sequence, AA |
| 253 | aCTLA4_Ab1_HC_IgG1_001 | ORF Sequence, NT |
| 254 | aCTLA4_Ab1_HC_IgG1_001 | mRNA Sequence (assumes T100 tail) |
| 255 | aCTLA4_Ab1_HC_IgG1_001 | ORF Sequence, AA (without leader sequence) |
| 256 | aCTLA4_Ab1_HC_IgG1_002 | Sequence, NT (5′ UTR, ORF, 3′ UTR) |
| 257 | aCTLA4_Ab1_HC_IgG1_002 | ORF Sequence, AA |
| 258 | aCTLA4_Ab1_HC_IgG1_002 | ORF Sequence, NT |
| 259 | aCTLA4_Ab1_HC_IgG1_002 | mRNA Sequence (assumes T100 tail) |
| 260 | aCTLA4_Ab1_HC_IgG1_002 | ORF Sequence, AA (without leader sequence) |
| 261 | aCTLA4_Ab1_HC_IgG1_003 | Sequence, NT (5′ UTR, ORF, 3′ UTR) |
| 262 | aCTLA4_Ab1_HC_IgG1_003 | ORF Sequence, AA |
| 263 | aCTLA4_Ab1_HC_IgG1_003 | ORF Sequence, NT |
| 264 | aCTLA4_Ab1_HC_IgG1_003 | mRNA Sequence (assumes T100 tail) |
| 265 | aCTLA4_Ab1_HC_IgG1_003 | ORF Sequence, AA (without leader sequence) |
| 266 | aCTLA4_Ab1_LC_K_001 | Sequence, NT (5′ UTR, ORF, 3′ UTR) |
| 267 | aCTLA4_Ab1_LC_K_001 | ORF Sequence, AA |
| 268 | aCTLA4_Ab1_LC_K_001 | ORF Sequence, NT |
| 269 | aCTLA4_Ab1_LC_K_001 | mRNA Sequence (assumes T100 tail) |
| 270 | aCTLA4_Ab1_LC_K_001 | ORF Sequence, AA (without leader sequence) |
| 271 | aCTLA4_Ab1_LC_K_002 | Sequence, NT (5′ UTR, ORF, 3′ UTR) |
| 272 | aCTLA4_Ab1_LC_K_002 | ORF Sequence, AA |
| 273 | aCTLA4_Ab1_LC_K_002 | ORF Sequence, NT |
| 274 | aCTLA4_Ab1_LC_K_002 | mRNA Sequence (assumes T100 tail) |
| 275 | aCTLA4_Ab1_LC_K_002 | ORF Sequence, AA (without leader sequence) |
| 276 | aCTLA4_Ab1_LC_K_003 | Sequence, NT (5′ UTR, ORF, 3′ UTR) |
| 277 | aCTLA4_Ab1_LC_K_003 | ORF Sequence, AA |
| 278 | aCTLA4_Ab1_LC_K_003 | ORF Sequence, NT |
| 279 | aCTLA4_Ab1_LC_K_003 | mRNA Sequence (assumes T100 tail) |
| 280 | aCTLA4_Ab1_LC_K_003 | ORF Sequence, AA (without leader sequence) |
| 281 | aCTLA4_Ab2_HC_IgG1_001 | Sequence, NT (5′ UTR, ORF, 3′ UTR) |
| 282 | aCTLA4_Ab2_HC_IgG1_001 | ORF Sequence, AA |
| 283 | aCTLA4_Ab2_HC_IgG1_001 | ORF Sequence, NT |
| 284 | aCTLA4_Ab2_HC_IgG1_001 | mRNA Sequence (assumes T100 tail) |
| 285 | aCTLA4_Ab2_HC_IgG1_001 | ORF Sequence, AA (without leader sequence) |
| 286 | aCTLA4_Ab2_HC_IgG1_002 | Sequence, NT (5′ UTR, ORF, 3′ UTR) |
| 287 | aCTLA4_Ab2_HC_IgG1_002 | ORF Sequence, AA |
| 288 | aCTLA4_Ab2_HC_IgG1_002 | ORF Sequence, NT |
| 289 | aCTLA4_Ab2_HC_IgG1_002 | mRNA Sequence (assumes T100 tail) |
| 290 | aCTLA4_Ab2_HC_IgG1_002 | ORF Sequence, AA (without leader sequence) |
| 291 | aCTLA4_Ab2_HC_IgG1_003 | Sequence, NT (5′ UTR, ORF, 3′ UTR) |
| 292 | aCTLA4_Ab2_HC_IgG1_003 | ORF Sequence, AA |
| 293 | aCTLA4_Ab2_HC_IgG1_003 | ORF Sequence, NT |
| 294 | aCTLA4_Ab2_HC_IgG1_003 | mRNA Sequence (assumes T100 tail) |
| 295 | aCTLA4_Ab2_HC_IgG1_003 | ORF Sequence, AA (without leader sequence) |
| 296 | aCTLA4_Ab2_HC_IgG2_001 | Sequence, NT (5′ UTR, ORF, 3′ UTR) |
| 297 | aCTLA4_Ab2_HC_IgG2_001 | ORF Sequence, AA |
| 298 | aCTLA4_Ab2_HC_IgG2_001 | ORF Sequence, NT |
| 299 | aCTLA4_Ab2_HC_IgG2_001 | mRNA Sequence (assumes T100 tail) |
| 300 | aCTLA4_Ab2_HC_IgG2_001 | ORF Sequence, AA (without leader sequence) |
| 301 | aCTLA4_Ab2_HC_IgG2_002 | Sequence, NT (5′ UTR, ORF, 3′ UTR) |
| 302 | aCTLA4_Ab2_HC_IgG2_002 | ORF Sequence, AA |
| 303 | aCTLA4_Ab2_HC_IgG2_002 | ORF Sequence, NT |
| 304 | aCTLA4_Ab2_HC_IgG2_002 | mRNA Sequence (assumes T100 tail) |
| 305 | aCTLA4_Ab2_HC_IgG2_002 | ORF Sequence, AA (without leader sequence) |
| 306 | aCTLA4_Ab2_HC_IgG2_003 | Sequence, NT (5′ UTR, ORF, 3′ UTR) |
| 307 | aCTLA4_Ab2_HC_IgG2_003 | ORF Sequence, AA |
| 308 | aCTLA4_Ab2_HC_IgG2_003 | ORF Sequence, NT |
| 309 | aCTLA4_Ab2_HC_IgG2_003 | mRNA Sequence (assumes T100 tail) |
| 310 | aCTLA4_Ab2_HC_IgG2_003 | ORF Sequence, AA (without leader sequence) |
| 311 | aCTLA4_Ab2_LC_K_001 | Sequence, NT (5′ UTR, ORF, 3′ UTR) |
| 312 | aCTLA4_Ab2_LC_K_001 | ORF Sequence, AA |
| 313 | aCTLA4_Ab2_LC_K_001 | ORF Sequence, NT |
| 314 | aCTLA4_Ab2_LC_K_001 | mRNA Sequence (assumes T100 tail) |
| 315 | aCTLA4_Ab2_LC_K_001 | ORF Sequence, AA (without leader sequence) |
| 316 | aCTLA4_Ab2_LC_K_002 | Sequence, NT (5′ UTR, ORF, 3′ UTR) |
| 317 | aCTLA4_Ab2_LC_K_002 | ORF Sequence, AA |
| 318 | aCTLA4_Ab2_LC_K_002 | ORF Sequence, NT |
| 319 | aCTLA4_Ab2_LC_K_002 | mRNA Sequence (assumes T100 tail) |
| 320 | aCTLA4_Ab2_LC_K_002 | ORF Sequence, AA (without leader sequence) |
| 321 | aCTLA4_Ab2_LC_K_003 | Sequence, NT (5′ UTR, ORF, 3′ UTR) |
| 322 | aCTLA4_Ab2_LC_K_003 | ORF Sequence, AA |
| 323 | aCTLA4_Ab2_LC_K_003 | ORF Sequence, NT |
| 324 | aCTLA4_Ab2_LC_K_003 | mRNA Sequence (assumes T100 tail) |
| 325 | aCTLA4_Ab2_LC_K_003 | ORF Sequence, AA (without leader sequence) |

### X. Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In addition, it is to be understood that any particular embodiment of the present invention that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, patents, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1

### Characterization of mRNA-encoded Therapeutic Antibodies

1.1 Design of IgG mRNA Molecules

Checkpoint inhibitor antibodies have been the focus on much recent attention in the scientific and medical communities given their widely-reported efficacy in cancer immunotherapy. The anti-CTLA-4 antibodies, ipilimumab and tremelimumab, have achieved considerable success in the clinic. In 2011, ipilimumab was approved for the treatment of late-stage melanoma that cannot be removed by surgery. In April 2015, tremelimumab was granted Orphan Drug Designation by US FDA for treatment of malignant mesothelioma. Despite its potential for fatal immune-mediated adverse reactions and unusual severe side effects, ipilimumab has more recently (October 2015) been approved for use as adjuvant therapy in stage III melanoma patients who are at high risk of melanoma recurrence following surgical intervention.

CTLA-4 is a negative regulatory surface molecule on T cells that competitively inhibits the CD28 co-stimulatory pathway by binding to the costimulatory molecules B7-1 and B7-2. Anti-CTLA-4 antibodies block this inhibitory signaling mechanism and allow T lymphocytes to destroy cancer cells.

The concept of using anti-CTLA-4 antibodies to treat cancer was first developed by James Allison and colleagues at the University of California, Berkeley. Much of this seminal work featured the CTLA-4 antagonist antibody 9D9 which was demonstrated to induce rejection of several tumor types in different mouse strains. The effectiveness of this CTLA-4 blockade was shown to correlate with the inherent immunogenicity of the tumor. These data led investigators to hypothesize that removing the “brakes” on a T cell response via CTLA-4 blockade would effectively allow the immune system to eliminate cancer cells and induce long-lasting anti-tumor immunity.

The efficacy of mRNA-produced 9D9 antibodies was tested in a widely employed in vivo model of cancer, the syngeneic colon cancer CT26 model. We designed mRNAs encoding both the 9D9 (IgG2b isotype) antibody and an IgG2a variant of 9D9 (termed “9D92a” or “9D92a^{a}”). mRNA sequences were generated encoding the full IgG antibody molecules, i.e., mRNAs encoding both the full heavy chain (HC) and full light chain (LC) of these antibodies.

The mRNA for these sequences was designed by adding proprietary 5′ and 3′ Untranslated Regions (UTRs) to the Open Reading Frames (ORF), which encoded the amino acid sequence of either the HC or the LC of the antibodies. The resulting HC and LC sequences were synthesized by In Vitro Transcription (IVT), using proprietary chemically modified nucleotides and formulated in Lipofectamine 2000 or Lipid Nanoparticles (LNP) using DLin-MC3-DMA (MC3) LNPs (Jayaraman et al., 2012) for in vitro and in vivo studies, respectively.

### 1.2 Confirmation of Anti-Tumor Efficacy of 9D9 Antibodies in the Mouse CT26 Cancer Model

The efficacy of systemically administered recombinant 9D9 antibodies was tested in the mouse CT26 tumor model. Murine CT26 cells, developed in 1975 by exposing BAT B/c mice to N-nitroso-N-methylurethane (NMU), result in a rapid-growing carcinoma that is easily implanted and readily metastasizes (Griswold and Corbett, Cancer 36:2441-2444 (1975)).

CT26 is one of the most extensively studied syngeneic mouse tumor models, used in the screening for and evaluation of small molecule cytotoxic agents and biological response modifiers for use in cancer immunotherapy. Unlike conventional xenograft models, which lack relevance due to the animals' immunocompromised status, this syngeneic mouse model provides an effective approach for studying how cancer therapies perform in the presence of a functional immune system.

For in vivo treatment studies, BALB/c mice were injected in the flank with 10⁵ tumor cells subcutaneously (SC) to establish SC tumors. Mice (n=14) were administered 5 mg/kg anti-CTLA-4 9D9 antibody at day 3, 6 and 9 following induction of tumors. Negative control mice (n=14) received no treatment. Mice were followed for several weeks and tumor burden and long-term survival were monitored. Mice were euthanized when tumors reached 2000 mm³.

Clear activity, i.e., 90% survival, was seen in mice treated with 5 mg/kg dose of 9D9 (IgG2b) antibody as compared to 0% survival in untreated, control mice confirming the efficacy of these anti-CTLA-4 antibodies when systemically administered in the CT26 mouse cancer model.

1.3 In Vitro Characterization of mRNA-Encoded Anti-CTLA-4 Antibodies

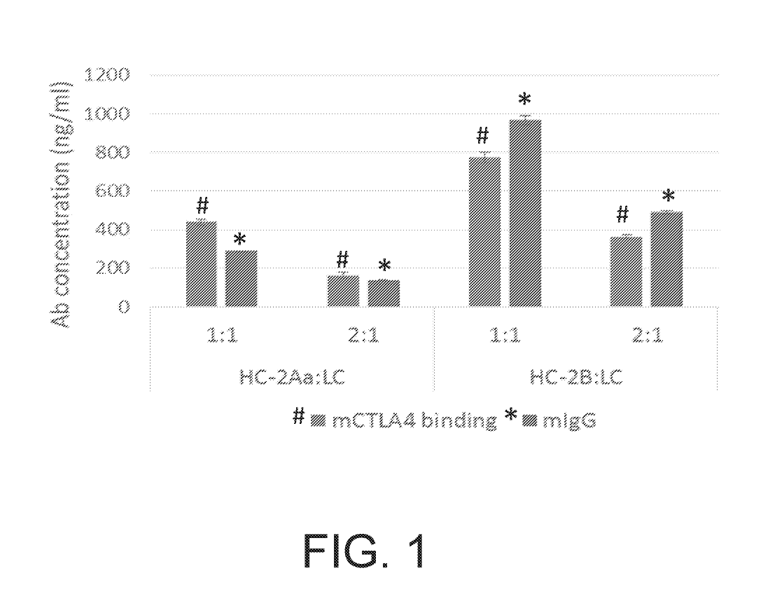

The following mRNAs encoding anti-CTLA-4 antibody heavy and light chains (HC and LC mRNAs) were designed and assayed for expression by measuring both CTLA-4-bound antibody (FIG. 1, bars marked with #) and total IgG produced (FIG. 1, bars marked with *): (1) 9D9 VH:mIgG2a (IgG2a^{a} allele) plus 9D9 VL:mIgGk; and (2) 9D9 VH:mIgG2b plus 9D9 VL:mIgGk. HeLa cells were transfected in 6-well plates with mRNA formulated in Lipofectamine 2000. Cell supernatants were harvested at 18 hours post transfection. Supernatants were assayed for binding to CTLA-4-coated plates (mCTLA-4-His (Life Technologies)) and detection was with HRP-Goat anti-mouse IgG Fc, 1:10000 (JIR). Purified 9D9 antibody (BioXcell) was used for the standard curve in the CTLA-4-binding ELISA. The Mouse IgG total ELISA Ready-SET-Go® assay (eBioscience) was used for quantitating total mouse IgG, with a mouse IgG internal standard.

The data show significant expression and antigen-binding of both IgG2a and IgG2b forms of 9D9 antibody in HeLa cells transfected with IVT mRNAs encoding full LC and HC. Moreover, the data demonstrated higher 9D9 expression/activity when mRNAs are delivered at a molar ratio of 1:1 (HC:LC).

Experimental data also showed that full sequence antibodies expressed better than scFv's. Anti CTLA-4 antibodies 9D9 IgG2a (HC and LC mRNAs at a molar ration of 1:1) and IgG2b (HC and LC mRNAs at a molar ratio of 1:1) were transfected into cells in 6-well plates using 3 mg mRNA and 4.5 mg L2K per well. Supernatants were harvested after 24 h. Active antibody levels were 657 ng/well for 9D9 IgG2a and 1161 ng/well for 9D9 IgG2b. In contrast, expression of active antibodies after transfection with 3 different scFv constructs under the same experimental condition resulted in expression levels of 6.2 ng/well, 0 ng/well, and 94 ng/well, respectively.

### Example 2

### Therapeutic Efficacy of mRNA-Encoded CTLA-4 Antibodies

2.1 Therapeutic Efficacy of mRNA-Encoded 9D9 Antibodies in a CT26 Mouse Colon Carcinoma Model

To evidence the therapeutic efficacy of the mRNA-encoded anti-CTLA-4 antibodies described supra, we studied these molecules in BALB/c mice challenged with subcutaneous (SC) tumors. The mRNAs were formulated in MC3 LNPs by mixing mRNAs encoding the HC and the LC at a molar ratio of 1:1, prior to encapsulation. LNPs routinely had a mean particle diameter of ˜65-85 nM, a polydispersity index (PDI) of ˜0.02-0.2 and an encapsulation efficiency (EE) of >95%.

### 2.1.1 Study Design

Animals were distributed into treatment groups according to body weight such that the mean body weight in each group was within 10% of the overall mean. Tumor cells were implanted subcutaneously in the low flank (between the right hip and sacral region) at Day 0. Mice were dosed individually by body weight on the day of treatment as described above. Intravenous (IV) dosing (0.5 mg mRNA per kg) was ˜1.75 mass excess of HC mRNA compared to LC mRNA (i.e., 1:1 molar ratio) co-formulated in MC3 lipid nanoparticles (LNPs) (DLin-MC3-DMA:Cholesterol:DSPC:PEG-DMG—50:38.5:10:1.5 molar ratios).

Animals were evaluated for volume, body weight and general health assessments. The defined end point to sacrifice animals due to excessive tumor burden was a tumor volume ≥2,000 mm³. The monitoring of tumor growth delay/inhibition and survival were incorporated into the study design.

Recombinant 9D9 antibody was included as an internal control. Whole blood was isolated and serum samples were retained for further analysis. An overview of the study design is set forth in TABLE 10.

TABLE 10
Overview of the Therapeutic anti-CTLA-4 Study Design

| | # | | | | Dose |
|---|---|---|---|---|---|
| Group | Animals | Compound | Route | Schedule | (mg/kg/inj) |
| 1 | 14 | Untreated Control | NA | NA | NA |
| 2 | 14 | anti-CTLA-4 protein | IP | D 3, 6, 9 | 5 |
| | | (BioXcell; IgG2b) | | | |
| 3 | 14 | 9D9 IgG2b | IV | D 3 | 0.5 |
| 4 | 14 | 9D9 IgG2b | IV | D 3, 9 | 0.5 |
| 5 | 14 | 9D9 IgG2b | IV | D 3, 6, 9 | 0.5 |
| 6 | 14 | 9D9 IgG2a^{a} | IV | D 3 | 0.5 |
| 7 | 14 | 9D9 IgG2a^{a} | IV | D 3, 9 | 0.5 |
| 8 | 14 | 9D9 IgG2a^{a} | IV | D 3, 6, 9 | 0.5 |
| 9 | 14 | NST (non-start) FIX | IV | D 3 | 0.5 |
| 10 | 14 | NST (non-start) FIX | IV | D 3, 9 | 0.5 |
| 11 | 14 | NST (non-start) FIX | IV | D 3, 6, 9 | 0.5 |
| 12 | 14 | PBS | IV | D 3 | 5 ml/kg |
| 13 | 14 | PBS | IV | D 3, 9 | 5 ml/kg |
| 14 | 14 | PBS | IV | D 3, 6, 9 | 5 ml/kg |

### 2.1.2 Results

### 2.1.2.1 Efficacy of Anti-CTLA-4 Protein Control Treatment

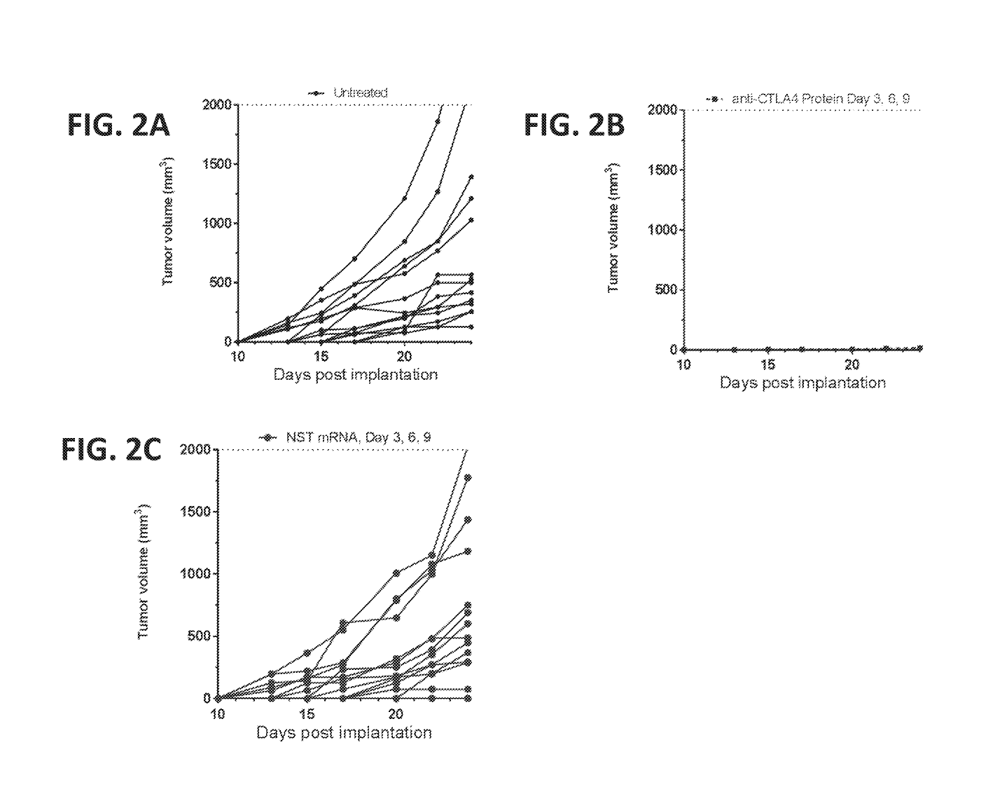

Mice treated with three doses of recombinant anti-CTLA-4 antibody (5 mg/kg at 3, 6 and 9 days post implantation) were assayed for tumor growth as described supra. Data are depicted for 24 days post-implantation (FIGS. 2A-2C). Control animals receiving negative control mRNA (i.e., mRNA encoding non-translated (“non-start”) Factor IX (“NST FIX”) developed tumors at a similar rate as compared to untreated controls, as expected.

As previously demonstrated, tumor growth is delayed in animals treated with therapeutic dosing of anti-CTLA-4 antibodies as compared to untreated animals or animals treated with negative-control mRNA.

2.1.2.2. Efficacy of Treatment with mRNAs Designed to Encode Anti-CTLA Antibodies

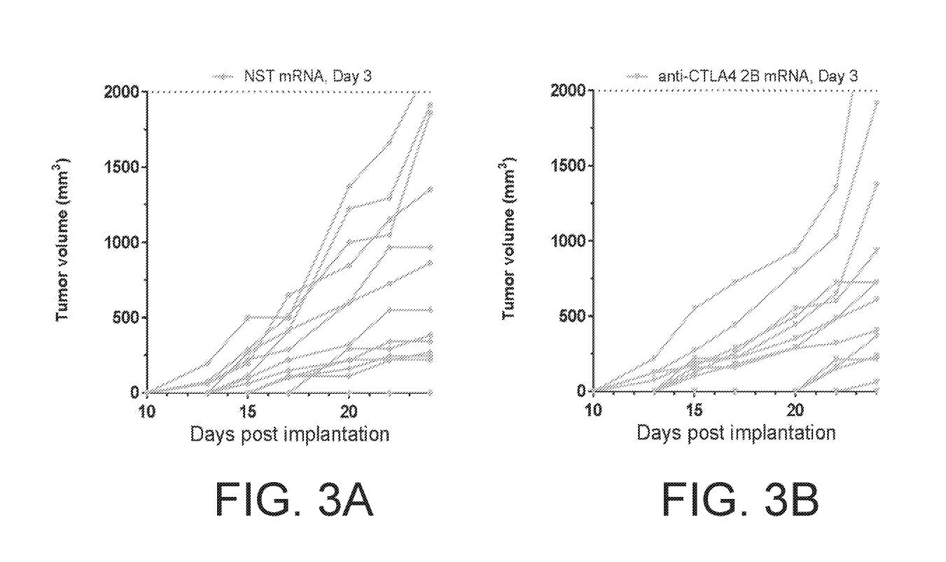

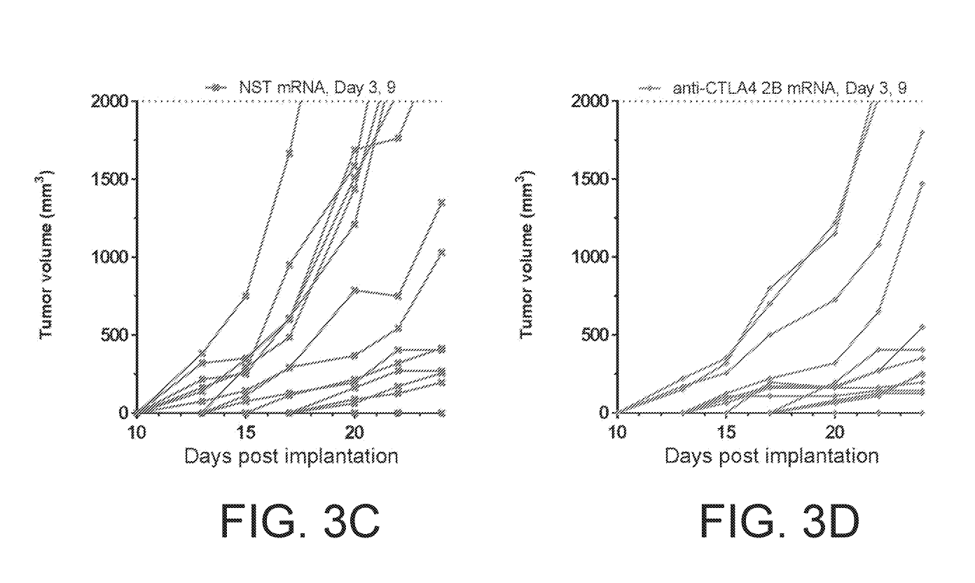

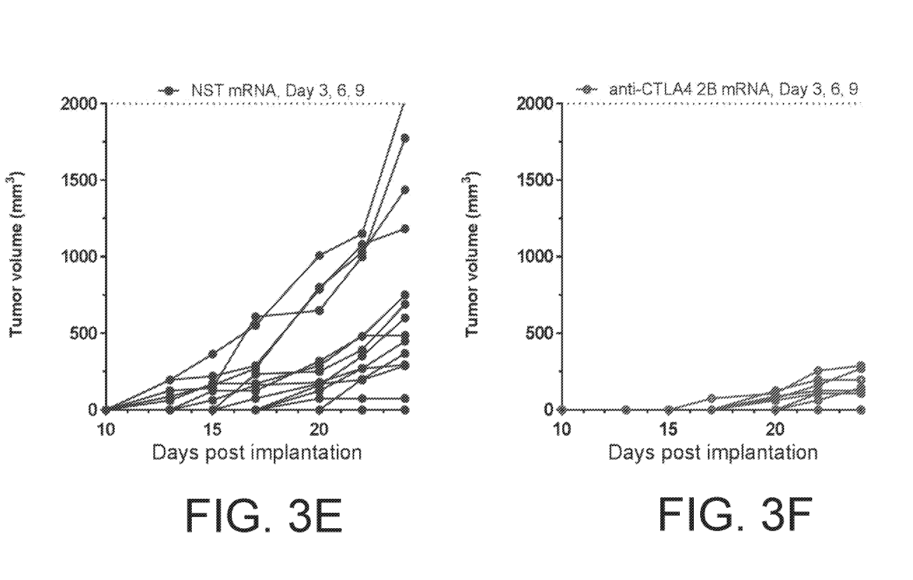

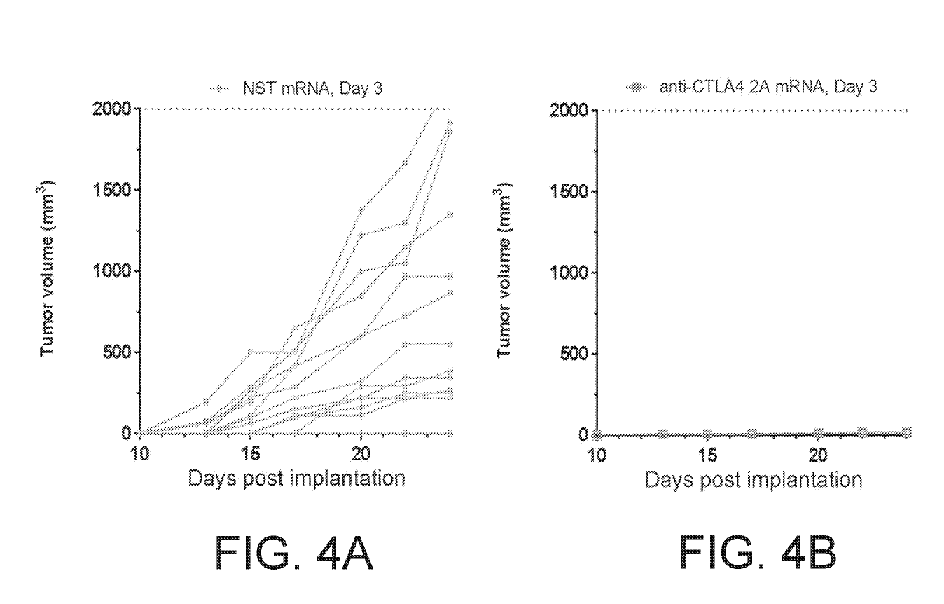

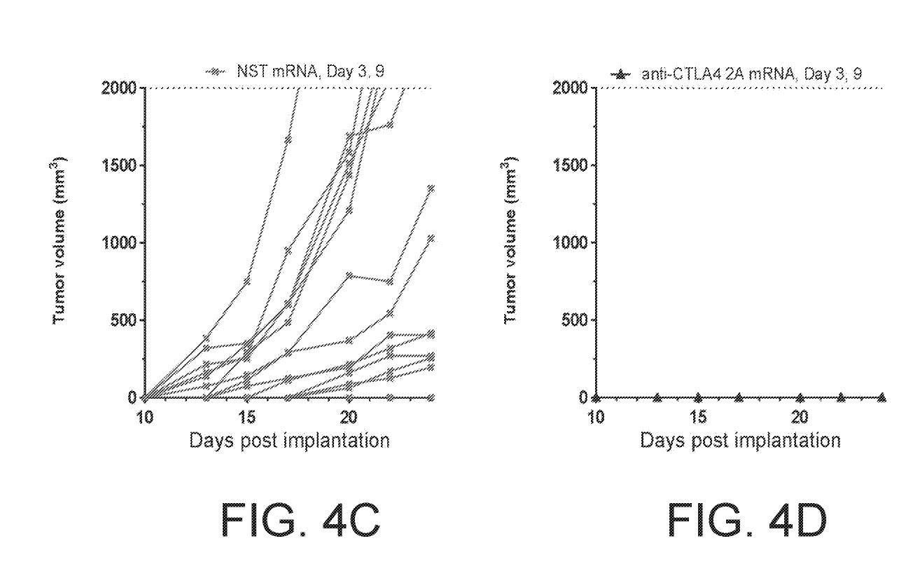

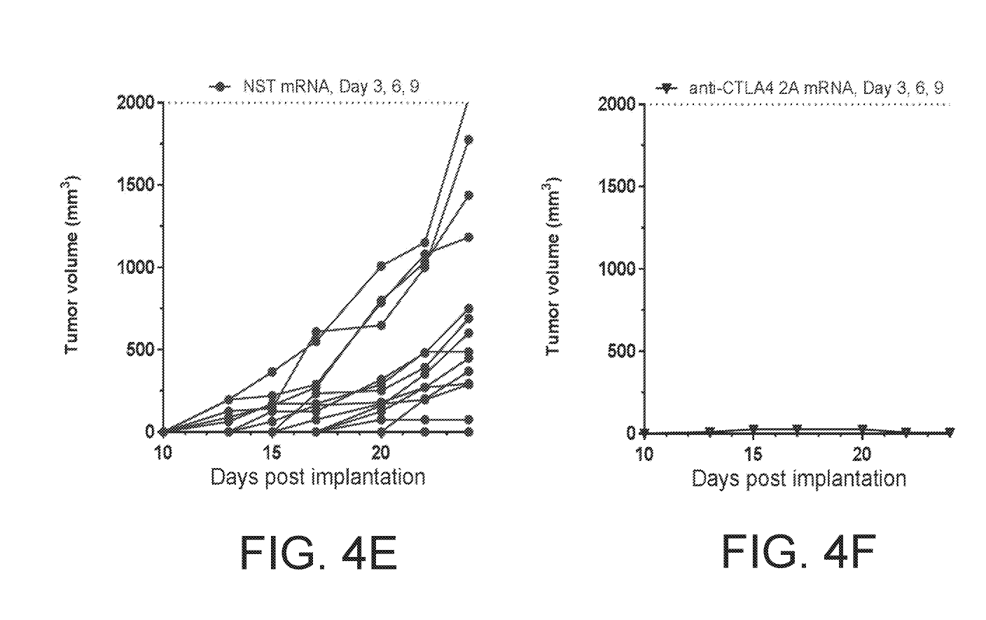

To demonstrate the therapeutic efficacy of mRNAs encoding 9D9 anti-CTLA-4 antibodies, mRNAs encoding the IgG2b and IgG2a^{a} variant 9D9 antibodies were administered following implantation and tumor growth was evaluated as compared to animals receiving negative control (NST-FIX) mRNA treatment described supra. Animals received a single dose or multiple doses of mRNA encoding 9D9 IgG2b or 9D9 IgG2a^{a} (FIGS. 3A-3F and FIGS. 4A-4F, respectively) (single dose=dosing at day 3; two doses=dosing at day 3 and day 9; three doses=dosing at days 3, 6 and 9). Negative control data for animals receiving 3 doses of negative control mRNA are as depicted supra and are reproduced for comparison purposes.

These data demonstrate a reduction in tumor burden in mice treated with mRNA encoding 9D9 (IgG2b) with mice receiving two systemic doses of MC3-LNP-formulated mRNA (0.5 mg/kg per dose) showing detectable reduction in tumor growth, and mice receiving three doses of mRNA exhibiting significant reduction in tumor growth, at 24 days post tumor induction.

Mice treated with only a single systemic (IV) dose of MC3-LNP-formulated mRNA encoding 9D9 (IgG2a) showed almost complete inhibition of tumor growth at 24 days post induction and mice receiving two or three doses of this mRNA exhibited complete inhibition of tumor growth.

All treatments (as well as each treatment regimen) were well-tolerated resulting in no overall morbidity or mortality. Each treatment was associated with an overall mean body weight gain.

### 2.1.3 Conclusions

This study was conducted to assess the activity of mRNAs encoding anti-CTLA-4 antibodies when administered systemically in a CT26 mouse carcinoma model. The studies presented herein demonstrate the therapeutic efficacy of mRNAs encoding two variants of the anti-CTLA-4 antibody 9D9 against lethal challenge with CT26 tumors in mice. Tumor burden, survival rates, changes in body weight and clinically relevant disease signs were investigated.

Mice treated with mRNA encoding 9D9 antibodies exhibited significant inhibition of tumor growth, with near complete inhibition of tumor growth observed at 24 days post tumor induction in mice receiving just a single dose (0.5 mg/kg of mRNA formulated in MC3) of mRNA encoding 9D9 (IgG2a) as well as in mice receiving multiple doses of mRNA encoding 9D9 (IgG2b) as compared to untreated mice and to mice receiving placebo (PBS) or non-translated mRNA treatment.

These data demonstrate a significant therapeutic effect for mRNA-encoded anti-CTLA-4 antibodies when mRNA is systemically administered at doses as low as 0.5 mg/kg in an in vivo CT26 mouse carcinoma model.

The studies presented herein utilize MC3-based LNPs. These LNPs have significant pre-clinical and clinical documentation and have been found to be acceptable for use in clinical trials by Alnylam for their ALN-TTR02 program (Coelho et al., 2013). LNPs in general have been shown to be very useful for mRNA delivery across several parenteral administration routes including intravenous (IV), intramuscular (IM), and subcutaneous (SC).

MC3-based LNPs have documented toxicity profiles across various species and may require pre-dosing and/or co-administration with anti-inflammatory (anti-histamines, acetaminophen, and dexamethasone) treatment. The majority of research with LNP formulated drugs has utilized IV administration although some SC and intradermal (ID) work has been reported. LNP-encapsulated mRNA-encoded antagonistic antibodies (administered systemically (IV)) have shown sufficient expression of two active IgG antibodies (9D9 IgG2b and 9D9 IgG2a), a therapeutic response as demonstrated by a reduction in growth of tumor tissue, and in addition survival against lethal induction of tumors with carcinoma cells (in this case, highly tumorigenic CT26 colon carcinoma cells).

### Example 2

### Extended Therapeutic Efficacy of mRNA-Encoded CTLA-4 Antibodies

The study described in TABLE 10 was followed out for more than 60 days.

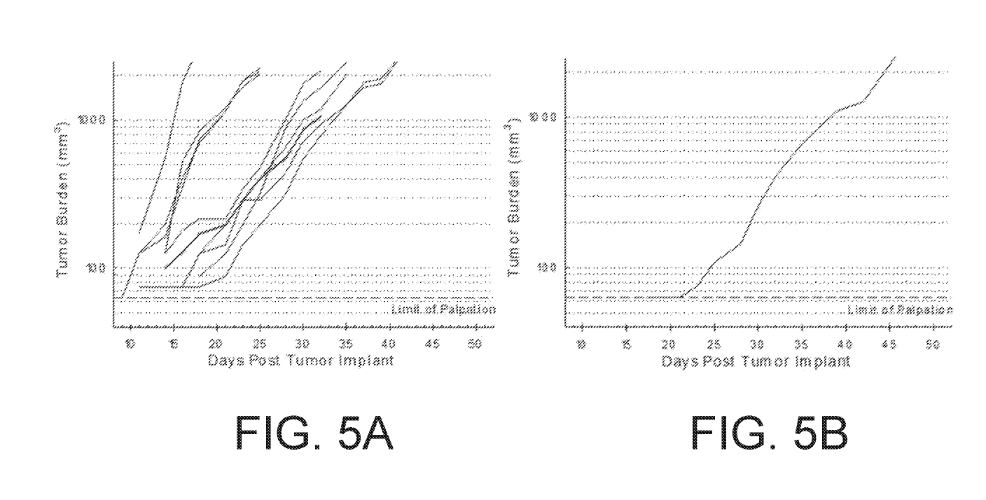

Mice treated with three doses of recombinant anti-CTLA-4 antibody (5 mg/kg at 3, 6 and 9 days post implantation) were assayed for tumor growth as described supra. The data shown in FIG. 5A shows tumor growth in control animals, in which survival was 0%, compared to the tumor growth in animals treated with 3 doses (administered at days 3, 6 and 9) of 9D9 IgG2b antibody protein, which had a survival rate of 90%. Thus, as previously demonstrated, tumor growth was delayed in animals treated with therapeutic dosing of anti-CTLA-4 antibodies as compared to untreated animals.

When plasma levels of anti-CTLA-4 antibody were measured after administration of either anti-CTLA-4 antibody (protein) or mRNA encoding an anti-CTLA-4 antibody, it was noticeable that the serum levels of anti-CTLA-4 antibody were much higher after the administration of the anti-CTLA4 antibody (protein). The administration of 5 mg/kg of anti-CTLA-4 protein resulted in serum levels of approximately 50 μg/mL. Serum protein levels of injected antibody protein remained more or less constant at the 24 hours, 48 hours, and 72 hours timepoints after administration.

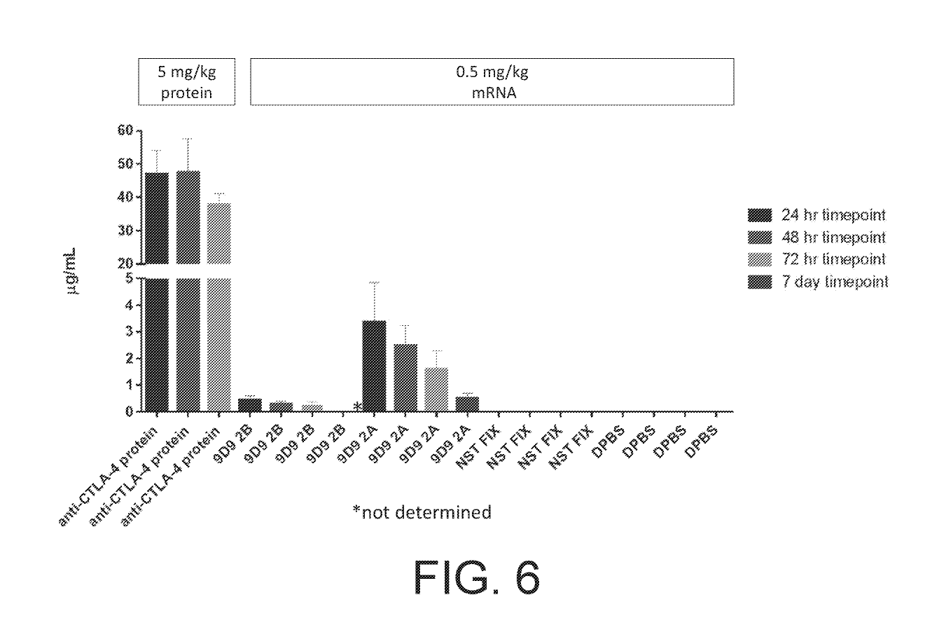

In contrast, the administration of 0.5 mg/kg mRNA encoding anti-CTLA-4 antibodies resulted in serum levels as low as 0.5 μg/mL (see FIG. 6). Serum levels of 9D9 2a antibody expressed after mRNA injection decreased from approximately 3.3 μg/mL at the 24 hours timepoint to approximately 2.8 μg/mL at the 48 hours timepoint. At the 72 hours timepoint the serum concentration had further decreased to approximately 1.7 μg/mL, and at the 7 day timepoint the serum concentration was approximately 0.5 μg/mL. Expression levels for 9D9 2b were considerably lower than the expression levels observed for the 9D9 2a antibody, e.g., at the 24 hours timepoint the total expression level of 9D9 IgG2b was approximately 0.5 μg/mL. See FIG. 6.

When measurements as described above in Example 2 were taken through Day 69 post cancer cell implantation/disease induction, there were only 2 animals remaining (“on study”) that appeared to have actively growing tumors. The mRNA encoding the 9D9 2a variant appeared to have yielded a 93-100% apparent complete response (CR) rate (depending on dose regimen). This is compared to a 57% apparent complete response rate after 3 doses of the 9D9 2b antibody protein, and a 21% complete response rate after 3 doses of the 9D9 2b mRNA.

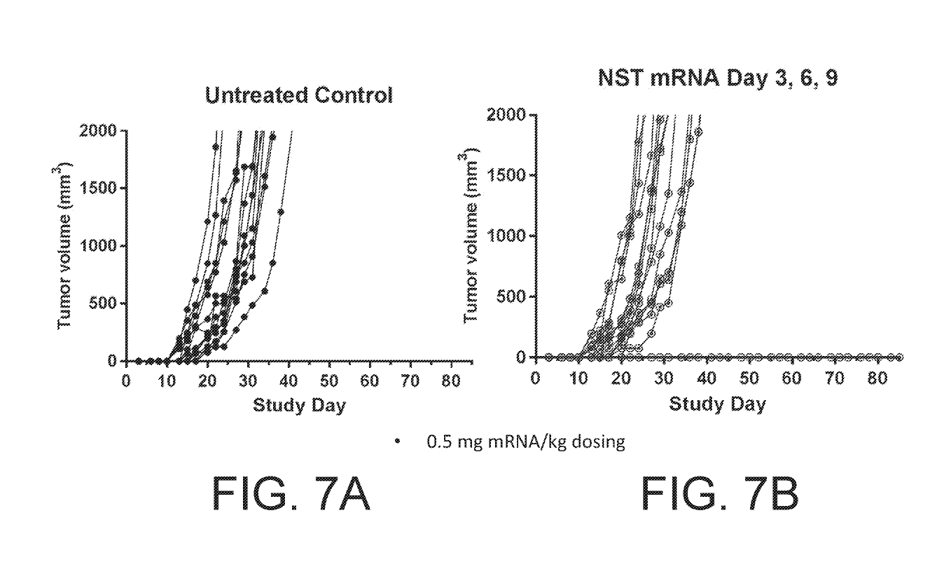

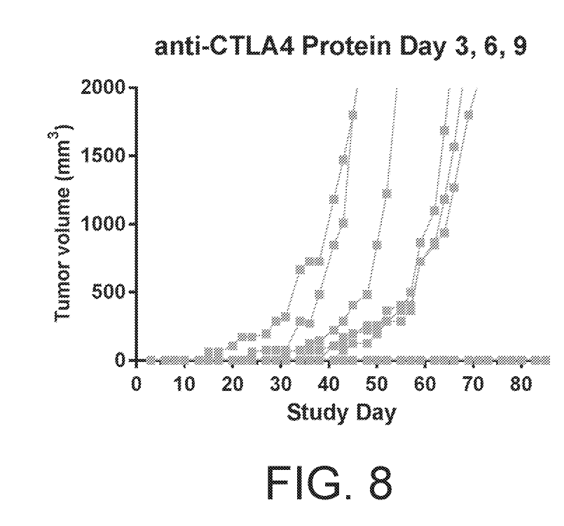

The measurements as described above in Example 2 were also taken through Day 90 (post cancer cell implantation). FIG. 7A and 7B show individual growth curves of untreated (FIG. 7A) and negative control NST mRNA/LNP (FIG. 7B) arms of the study. None of the untreated controls (0/14) survived to day 90. Only one of the NSP mRNA/LNP controls (1/14, i.e., 7% rate) survived to Day 90. When anti-CTLA-4 9D9 native antibody was administered in protein form in three 5 mg/kg doses at post-implatation days 3, 6 and 9, eight out of 14 animals survived to Day 90, i.e., the survival rate was 57%. See FIG. 8.

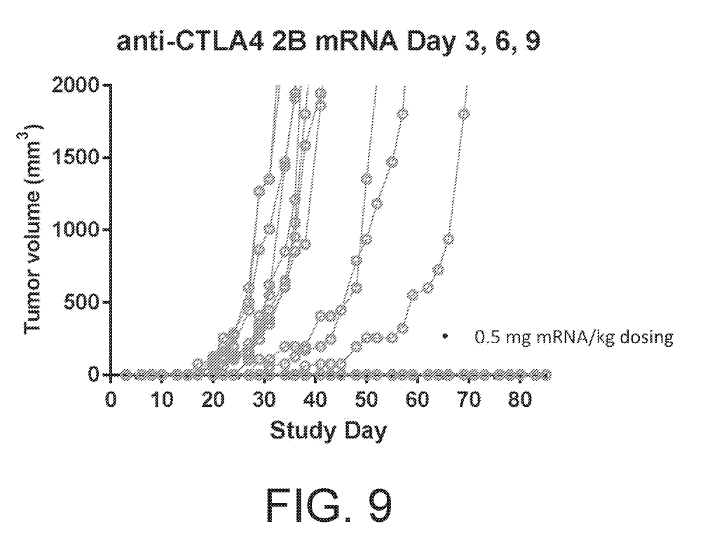

When mRNA encoding the anti-CTLA-4 9D9 2b antibody was administered in three 0.5 mg mRNA/kg doses at post-implatation days 3, 6, and 9, three out of 14 animals survived to Day 90, i.e., their survival rate was 21%. Note, however, that whereas none of the control animals survived past Day 40, three of the mRNA-treated animals survived past Day 40. See FIG. 9.

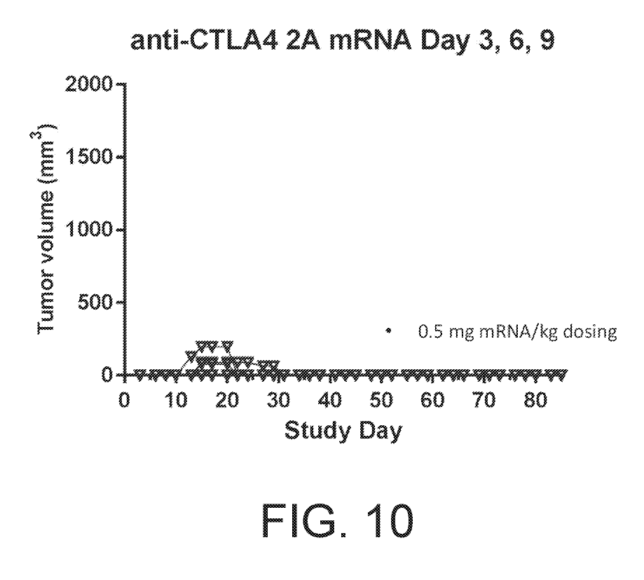

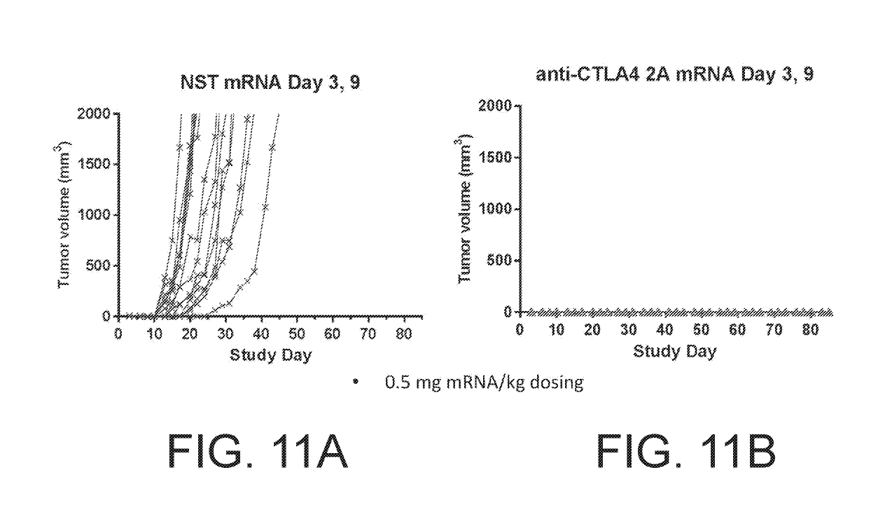

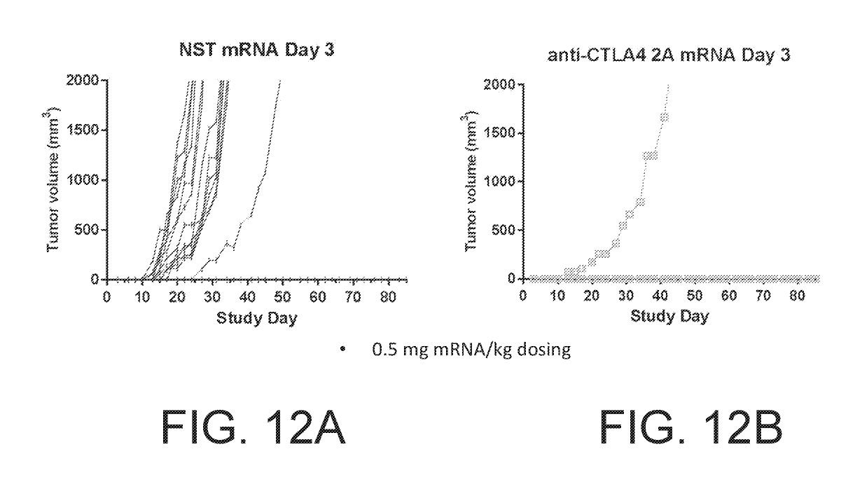

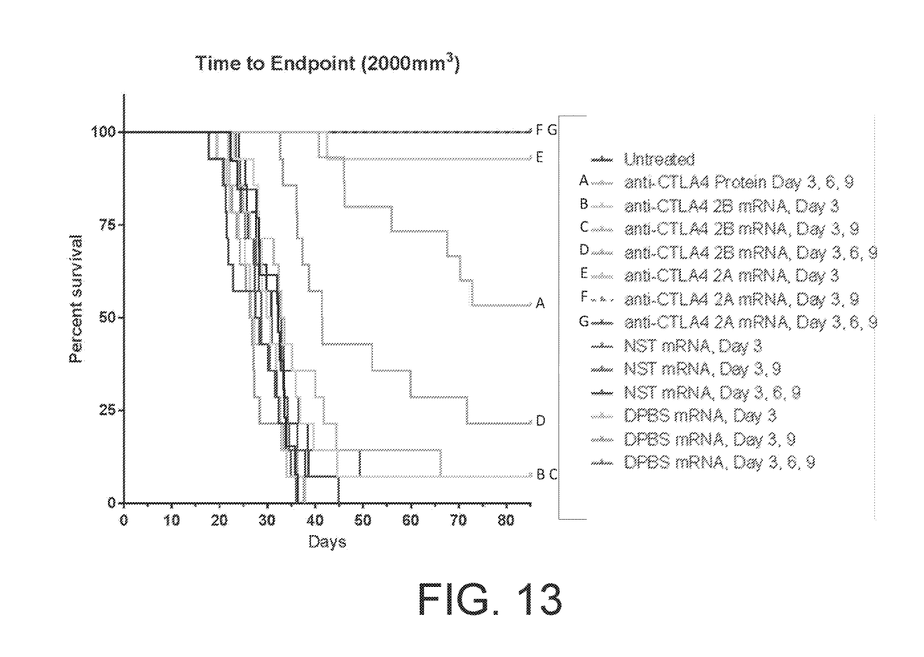

When mRNA encoding the anti-CTLA-4 9D9 2b was administered under the same experimental conditions, 100% of the animals survived to Day 90. See FIG. 10. The same survival rate was observed when only two doses of mRNA were administered (FIGS. 11A-11B). Even when the mRNA administration was reduced to a single dose, the survival rate was still 93% (13 out of 14).

When comparing response rates after administration of anti-CTLA-4 9D9 protein or mRNA form, it is important to note that the serum antibody concentration following protein administration was approximately 50 μg/ml, whereas the antibody concentration following mRNA administration was ˜0.5-1.0 μg/mL (9B9 2a). Thus, even at serum antibody concentrations 50-100-fold lower, the efficacy for the mRNA-encoded antibody was far superior.

Again, these data demonstrate that a significant therapeutic effect for mRNA-encoded anti-CTLA-4 antibodies is achieved when mRNA is systemically administered at doses as low as 0.5 mg/kg in an in vivo CT26 mouse carcinoma model, and the such therapeutic effect when serum concentrations as low as 0.5 μg/mL.

These results indicate that targeting of CTLA-4 using mRNA-encoded antagonistic antibodies provides for effective immune responses against tumor cells and provides significant therapeutic benefit in combatting tumor growth.

### Other Embodiments

It is to be understood that the words which have been used are words of description rather than limitation, and that changes can be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of reducing the size of a tumor or inhibiting the growth of a tumor in a subject in need thereof comprising administering to said subject one or more mRNAs encoding an antibody or an antigen binding portion thereof which specifically binds to CTLA-4 (cytotoxic T-lymphocyte-associated protein 4).

2-3. (canceled)

4. The method of claim 1, wherein the antibody or antigen binding portion thereof which specifically binds to CTLA-4 comprises:
(i) a VH of SEQ ID NO: 9, 28, or 39 and a VL of SEQ ID NO: 11, 29 or 41, or
(ii) a VH of SEQ ID NO: 183 or 240 and a VL of SEQ ID NO: 185 or 238.

5-7. (canceled)

8. The method of claim 1, wherein the antibody or antigen binding portion thereof which specifically binds to CTLA 4 comprises a VL and a VH, wherein the VL comprises an amino acid sequence having at least 80%, at least 85%, at least 90%, at least 95%, at least 96%, at least 97%, at least 98%, or at least 99% sequence identity to SEQ ID NO: 11, SEQ ID NO: 29, SEQ ID NO:41, SEQ ID NO: 185, or SEQ ID NO: 238.

9. The method of claim 1, wherein the antibody or antigen binding portion thereof which specifically binds to CTLA 4 comprises a VL and a VH, wherein the VH comprises an amino acid sequence having at least 80%, at least 85%, at least 90%, at least 95%, at least 96%, at least 97%, at least 98%, or at least 99% sequence identity to SEQ ID NO: 14, SEQ ID NO: 30, SEQ ID NO:44, SEQ ID NO: 183, or SEQ ID NO: 240.

10-17. (canceled)

18. The method of claim 1, wherein the antibody or antigen binding portion thereof which specifically binds to CTLA 4 comprises a heavy chain (HC) a light chain (LC), wherein the HC comprises, consists, or consists essentially of the amino acid sequence of SEQ ID NO: 20 or SEQ ID NO:24, and the LC comprises, consists, or consists essentially of the amino acid sequence of SEQ ID NO: 22 or SEQ ID NO: 26.

19-22. (canceled)

23. The method according to claim 1, wherein binding of the antibody or antigen binding portion thereof to CTLA-4:
(i) reduces the size of the tumor;
(ii) inhibits the growth of the tumor;
(iii) reduces tumor cell proliferation in the subject;
(iv) increases survival rate; or,
(v) a combination thereof.

24. The method according to claim 1, wherein the tumor is selected from the group consisting of melanoma tumor, lung cancer tumor, bladder cancer tumor, colon cancer tumor, and prostate cancer tumor.

25. The method according to claim 24, wherein the tumor is a melanoma tumor and wherein the melanoma tumor is a metastatic melanoma tumor.

26. The method according to claim 24, wherein the tumor is lung cancer tumor and wherein the lung cancer tumor is a non-small cell lung carcinoma (NSCLC) tumor or a small cell lung cancer (SCLC) tumor.

27. The method according to claim 24, wherein the tumor is a prostate cancer tumor and wherein the prostate cancer tumor is a metastatic hormone-refractory prostate cancer tumor.

28. (canceled)

29. The method according to claim 1, wherein the antibody or antigen binding portion thereof which specifically binds to CTLA-4 is encoded by a polynucleotide sequence at least 60%, at least 70%, at least 80%, at least 85%, at least 90%, at least 95%, at least 96%, at least 97%, at least 98%, at least 99%, or 100% identical to SEQ ID NO: 20, SEQ ID NO:24, SEQ ID NO:22, or SEQ ID NO:26, a subsequence thereof, or a combination thereof.

30. (canceled)

31. The method according to claim 1, wherein:
(i) at least one of the one or more mRNAs encoding an antibody or an antigen binding portion thereof which specifically binds to CTLA-4 comprises at least one chemically modified nucleoside; or,
(ii) each one of the one or more mRNAs encoding an antibody or an antigen binding portion thereof which specifically binds to CTLA-4 comprises at least one chemically modified nucleoside.

32. The method according to claim 31, wherein the at least one chemically modified nucleoside is selected from the group consisting of pseudouridine (ψ), N1-methylpseudouridine (m1ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof.

33. The method according to claim 31, wherein the at least one chemically modified nucleoside is selected from the group consisting of pseudouridine (ψ), N1-methylpseudouridine (m1ψ), 5-methylcytosine, 5-methoxyuridine, and a combination thereof.

34. The method according to claim 31, wherein the one or more mRNAs encoding an antibody or an antigen binding portion thereof which specifically binds to CTLA-4 comprise at least 10%, at least 20%, at least 30%, at least 40%, at least 50%, at least 60%, at least 70%, at least 80%, at least 90%, at least 95%, at least 99%, or 100% chemically modified nucleosides.

35-39. (canceled)

40. The method according to claim 1, wherein one or more mRNAs encoding an antibody or an antigen binding portion thereof which specifically binds to CTLA-4 further comprise a 5′ UTR.

41. The method according to claim 40, wherein the 5′ UTR comprises a nucleic acid sequence at least 90%, at least 95%, at least 96%, at least 97%, at least 98%, at least 99%, or 100% identical to a sequence selected from SEQ ID NOS: 197-214.

42. (canceled)

43. The method according to claim 1, wherein one or more mRNAs encoding an antibody or an antigen binding portion thereof which specifically binds to CTLA-4 further comprise a 3′ UTR.

44. The method according to claim 43, wherein the 3′ UTR comprises a nucleic acid sequence at least 90%, 95%, 96%, 97%, 98%, 99%, or 100% identical to a sequence selected from SEQ ID NO: 215-232.

45-52. (canceled)

53. The method according to claim 1, wherein the one or more mRNAs encoding an antibody or an antigen binding portion thereof which specifically binds to CTLA-4 are formulated with a delivery agent.

54. The method according to claim 53, wherein the delivery agent comprises a lipidoid, a liposome, a lipoplex, a lipid nanoparticle, a polymeric compound, a peptide, a protein, a cell, a nanoparticle mimic, a nanotube, or a conjugate.

55. The method according to claim 54, wherein the delivery agent is a lipid nanoparticle.

56. The method according to claim 55, wherein the lipid nanoparticle comprises the lipid selected from the group consisting of DLin-DMA, DLin-K-DMA, 98N12-5, C12-200, DLin-MC3-DMA, DLin-KC2-DMA, DODMA, PLGA, PEG, PEG-DMG, PEGylated lipids, amino alcohol lipids, KL22, and combinations thereof.

57-59. (canceled)

60. A polynucleotide comprising one or more mRNAs which encode an antibody or an antigen binding portion thereof which specifically binds to CTLA-4 and which specifically binds to the same CTLA-4 epitope as:
(i) an antibody or antigen-binding portion thereof comprising a heavy chain variable region (VH) of SEQ ID NO: 9, 28, or 39, and a light chain variable region (VL) of SEQ ID NO: 11, 29 or 41, or
(ii) an antibody or antigen-binding portion comprising a VH of SEQ ID NO: 183 or SEQ ID NO: 240 and a VL of SEQ ID NO: 185 or SEQ ID NO: 238.

61-67. (canceled)

68. A polynucleotide comprising one or more mRNAs which encode an antibody or an antigen binding portion thereof which specifically binds to CTLA-4, and which comprises a VL and a VH, wherein
(i) the VL comprises an amino acid sequence having at least 80%, at least 85%, at least 90%, at least 95%, at least 96%, at least 97%, at least 98%, at least 99% sequence identity to SEQ ID NO: 11, SEQ ID NO: 29, or SEQ ID NO:41, and the VH comprises an amino acid sequence having at least 80%, at least 85%, at least 90%, at least 95%, at least 96%, at least 97%, at least 98%, at least 99% sequence identity to SEQ ID NO: 9, SEQ ID NO: 28, or SEQ ID NO:39; or,
(ii) the VL comprises an amino acid sequence having at least 80%, at least 85%, at least 90%, at least 95%, at least 96%, at least 97%, at least 98%, at least 99% sequence identity to SEQ ID NO: 185 or SEQ ID NO: 238, and the VH comprises an amino acid sequence having at least 80%, at least 85%, at least 90%, at least 95%, at least 96%, at least 97%, at least 98%, at least 99% sequence identity to SEQ ID NO: 183 or SEQ ID NO: 240.

69-111. (canceled)

112. A composition comprising the polynucleotide according to claim 60 and a delivery agent.

113. The composition according to claim 112, wherein the delivery agent comprises a lipidoid, a liposome, a lipoplex, a lipid nanoparticle, a polymeric compound, a peptide, a protein, a cell, a nanoparticle mimic, a nanotube, or a conjugate.

114. The composition according to claim 113, wherein the delivery agent is a lipid nanoparticle.

115. The composition according to claim 114, wherein the lipid nanoparticle comprises the lipid selected from the group consisting of DLin-DMA, DLin-K-DMA, 98N12-5, C12-200, DLin-MC3-DMA, DLin-KC2-DMA, DODMA, PLGA, PEG, PEG-DMG, PEGylated lipids, amino alcohol lipids, KL22, and combinations thereof.

116-117. (canceled)

118. A host cell comprising a polynucleotide according to claim 60.

119. A method of making a polynucleotide comprising enzymatically or chemically synthesizing the polynucleotide according to claim 60.

120-121. (canceled)

122. A method of treating cancer in a subject in a subject in need thereof, the method comprising administering to the subject an effective amount of the polynucleotide according to claim 60.

123-125. (canceled)

126. A method of reducing the size of a tumor or inhibiting the growth of a tumor in a subject in need thereof comprising administering to said subject at least one effective dose of the polynucleotide according to claim 60, wherein the at least one effective dose is such that
(i) a plasma level of the antibody or an antigen binding portion thereof which specifically binds to CTLA-4 of at least 3 μg/mL is reached within 24 hours after administration of the dose;
(ii) a plasma level of the antibody or an antigen binding portion thereof which specifically binds to CTLA-4 of at least 2 μg/mL is reached within 48 hours after administration of the dose;
(iii) a plasma level of the antibody or an antigen binding portion thereof which specifically binds to CTLA-4 of at least 1 μg/mL is reached within 72 hours after administration of the dose; or,
(iv) a plasma level of the antibody or an antigen binding portion thereof which specifically binds to CTLA-4 of at least 0.5 μg/mL is reached within 7 days after administration of the dose.

127. (canceled)

128. The method according to claim 126, wherein the at least one effective dose comprises one, two, or three doses.

129. The method according to claim 128, wherein the doses are administered within at least 3 day intervals.

130. The method according to claim 126, wherein the polynucleotide is administered at 0.5 mg/kg/dose.

131. The method according to claim 126, where the administration of the at least one effective dose results in a remission rate of at least 60%.

132. The method according to claim 126, where the administration of the at least one effective dose results in (i) reduction of the size of a tumor by at least 50%, (ii) inhibition of the growth of the tumor by at least 50%, or (iii) a combination thereof.
